(19) United States
(12) Patent Application Publication
(10) Pub. No.: US 2026/0027339 A1
(43) Pub. Date: Jan. 29, 2026

(54) FLEXIBLE IMPLANTABLE DEPOTS FOR CONTROLLED DRUG RELEASE

(71) Applicant: Foundry Therapeutics, Inc., Menlo Park, CA (US)

(72) Inventors: Jackie Joe Hancock, Albany, CA (US); Esi B. Ghartey-Tagoe, San Jose, CA (US); David J. Hewitt, Menlo Park, CA (US); Cynthia R. Lee, Belmont, CA (US); Wei Li Lee, Singapore (SG); Ming Siew Lim, Singapore (SG); Ee Theng Ng, Singapore (SG); Patrick H. Ruane, El Dorado Hills, CA (US); Nurfatin Samsudin, Singapore (SG); Daniel Boon Lim Seet, Singapore (SG); Claire Aileen Stewart, Ottawa (CA); James Su, Newark, CA (US); Koon Kiat Teu, Singapore (SG)

(21) Appl. No.: 19/341,797

(22) Filed: Sep. 26, 2025

Related U.S. Application Data
(63) Continuation of application No. PCT/US2024/022082, filed on Mar. 28, 2024.
(60) Provisional application No. 63/492,980, filed on Mar. 29, 2023.

(51) Int. Cl. A61M 37/00; A61K 31/445; A61K 47/34; A61P 25/04

(52) U.S. Cl. CPC A61M 37/00; A61K 31/445; A61K 47/34; A61P 25/04; A61M 2205/04

(57) ABSTRACT

Implantable depots for delivering therapeutic agents and associated systems and methods are provided. In some embodiments, an implantable depot for treating pain includes a therapeutic region including a polymer, an analgesic agent, and a plasticizer. When implanted in vivo, the therapeutic region can be configured to release the analgesic agent for a treatment period of at least 3 days. The implantable depot can have a flexural modulus within a range from 1 MPa to 400 MPa.

### CROSS-REFERENCE TO RELATED APPLICATIONS

This application is a continuation of International Patent Application No. PCT/US24/22082, filed Mar. 28, 2024, which claims the benefit of priority to U.S. Provisional Application No. 63/492,980, filed Mar. 29, 2023, each of which is incorporated by reference herein in its entirety.

### TECHNICAL FIELD

The present technology relates generally to implantable depots, and in particular, to implantable depots for delivering therapeutic agents and associated systems and methods.

### BACKGROUND

Implantable systems for the controlled release of therapeutic agents offer advantages over other drug delivery methods, such as oral or parenteral methods. Devices made of biocompatible and/or biodegradable polymers and therapeutic agents can be implanted in clinically desirable anatomic locations, thereby providing localized delivery of select agents. This localized delivery enables a substantial proportion of the agent to reach the intended target and undesirable systemic side effects can be avoided. However, these systems often suffer from a lack of a true controlled release mechanism in that they typically provide a burst release of therapeutic agent upon contact with surrounding physiologic fluids, followed by a residual release of agent.

A controlled, sustained release of a therapeutic agent can be of clinical benefit in certain circumstances. In particular, it may be desirable to implant a biodegradable carrier holding a large dose of a therapeutic agent for controlled, sustained release over time. This may have particular value when the carrier loaded with therapeutic agent is implanted in conjunction with an interventional or surgical procedure and, optionally, alongside or as part of an implantable medical device. Thus, a need exists for biocompatible implantable systems capable of providing a highly controlled release of a therapeutic agent.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a partially schematic view of an implantable depot configured in accordance with embodiments of the present technology.

FIG. 1B is a partially schematic view of another implantable depot configured in accordance with embodiments of the present technology.

FIG. 1C is a partially schematic view of yet another implantable depot configured in accordance with embodiments of the present technology.

FIG. 2A is a top view of a rectangular depot configured in accordance with embodiments of the present technology.

FIG. 2B is a side view of the rectangular depot of FIG. 2A.

FIG. 3A is a top view of a triangular depot with a hole configured in accordance with embodiments of the present technology.

FIG. 3B is a side view of the triangular depot of FIG. 3A.

FIG. 4A is a top view of a triangular depot with holes configured in accordance with embodiments of the present technology.

FIG. 4B is a side view of the triangular depot of FIG. 4A.

FIG. 4C is a top view of another triangular depot with holes configured in accordance with embodiments of the present technology.

FIG. 4D is a top view of yet another triangular depot with holes configured in accordance with embodiments of the present technology.

FIG. 4E is a top view of a triangular depot with holes configured in accordance with embodiments of the present technology.

FIG. 4F is a top view of another triangular depot with holes configured in accordance with embodiments of the present technology.

FIG. 4G is a top view of a triangular depot with holes configured in accordance with embodiments of the present technology.

FIG. 4H is a top view of yet another triangular depot with holes configured in accordance with embodiments of the present technology.

FIG. 5A is a top view of an arrowhead-shaped depot configured in accordance with embodiments of the present technology.

FIG. 5B is a top view of a diamond-shaped depot configured in accordance with embodiments of the present technology.

FIG. 5C is a top view of a rectangular depot configured in accordance with embodiments of the present technology.

FIG. 5D is a top view of a cross-shaped depot configured in accordance with embodiments of the present technology.

FIG. 5E is a top view of an L-shaped depot configured in accordance with embodiments of the present technology.

FIG. 5F is a top view of a circular depot configured in accordance with embodiments of the present technology.

FIG. 5G is a perspective view of a spherical depot configured in accordance with embodiments of the present technology.

FIG. 5H is a top view of a diamond-shaped depot configured in accordance with embodiments of the present technology.

FIG. 6 is a scanning electron microscope image of a portion of an implantable depot.

FIG. 7A is a graph showing in vitro elution data for an implantable depot.

FIG. 7B is another graph showing in vitro elution data for an implantable depot.

FIG. 8A is a scanning electron microscope image of an implantable depot that is approximately 25% eluted.

FIG. 8B is a scanning electron microscope image of an implantable depot that is approximately 75% eluted.

FIG. 9A is a graph showing mean bupivacaine plasma concentrations in subjects treated with implantable depots versus other formulations, after total knee arthroplasty.

FIG. 9B is a graph showing the mean bupivacaine plasma concentration in subjects treated with implantable depots overlaid with postoperative pain data, after total knee arthroplasty.

FIG. 9C is a graph showing the area under the curve (AUC) of bupivacaine plasma concentration in subjects treated with implantable depots versus other formulations, after total knee arthroplasty.

FIG. 9D is a graph showing the mean bupivacaine plasma concentrations in subjects receiving varying doses of bupivacaine from implantable depots.

FIG. 9E is a graph showing the relationship between Cₘₐₓ and bupivacaine dose for implantable depots.

FIG. 9F is a graph showing the relationship between AUC_{0-14d} and bupivacaine dose for implantable depots.

FIG. 9G is a graph showing the in vivo bupivacaine release profile in subjects receiving implantable depots.

FIG. 10 is a graph showing mean pain intensity scores for subjects treated with implantable depots (not adjusted for opioid consumption).

FIG. 11A is a graph showing simulated bupivacaine plasma concentrations for subjects treated with implantable depots versus actual bupivacaine plasma concentrations in subjects treated with other bupivacaine formulations, following shoulder surgery.

FIG. 11B is a graph showing simulated bupivacaine plasma concentrations for subjects treated with an implantable depot versus actual bupivacaine plasma concentrations in subjects treated with another bupivacaine formulation, following bunionectomy.

FIG. 11C is a graph showing simulated bupivacaine plasma concentrations for subjects treated with implantable depots versus actual bupivacaine plasma concentrations in subjects treated with other bupivacaine formulations, following open inguinal hernia repair.

FIG. 12 is a graph illustrating cumulative in vitro release of bupivacaine from an implantable depot formulated with bupivacaine free base and without control regions.

FIG. 13 is a graph illustrating cumulative in vitro release of bupivacaine from implantable depots with varying control regions.

FIG. 14A is a graph illustrating cumulative in vitro release of bupivacaine from implantable depots formulated with bupivacaine in free base and salt forms.

FIG. 14B is a graph illustrating cumulative in vitro release of bupivacaine from implantable depots with bupivacaine in free base and salt forms.

FIG. 15 is a semilog graph illustrating in vivo release of bupivacaine from implantable depots formulated with bupivacaine in free base and salt forms in a rabbit subcutaneous model.

FIG. 16 is a graph illustrating cumulative in vitro release of bupivacaine from implantable depots with varying therapeutic loading.

FIG. 17 is a graph illustrating cumulative in vitro release of bupivacaine from implantable depots formulated with varying free base:salt ratios.

FIGS. 18A and 18B illustrate a Monte Carlo approach for modeling travel distances of different depot geometries.

FIGS. 18C and 18D illustrate a geometrical/calculus approach for modeling travel distances of different depot geometries.

FIG. 19 is a graph illustrating cumulative in vitro release of bupivacaine from implantable depots having different geometries at pH 5.8.

FIG. 20 is a graph illustrating cumulative in vitro release of bupivacaine from implantable depots formulated with different bupivacaine particle sizes at pH 5.8.

FIG. 21A is a graph illustrating cumulative in vitro release of bupivacaine from implantable depots with control regions versus depots without control regions at pH 5.8.

FIG. 21B is a graph illustrating cumulative in vitro release of bupivacaine from implantable depots without control regions and having different geometries and thicknesses at pH 5.8.

FIG. 21C is a graph illustrating cumulative in vitro release of bupivacaine from implantable depots without control regions and having different thicknesses at pH 7.4.

FIG. 21D is a graph illustrating cumulative in vitro release from implantable depots without control regions that are formulated with different bupivacaine particle sizes at pH 5.8.

FIG. 22A is a graph illustrating in vivo release of bupivacaine from different combinations of configurations of implantable depots in a rabbit subcutaneous model.

FIG. 22B is a graph illustrating in vivo release of bupivacaine from different configurations of implantable depots in a minipig abdominal hernia repair model.

FIG. 22C is a graph illustrating in vivo release of bupivacaine from different configurations of implantable depots in a dog subcutaneous model.

FIG. 22D is a graph illustrating cumulative AUCₗₐₛₜ profiles for different configurations of implantable depots in various animal models.

FIG. 23 is a graph illustrating in vivo release of bupivacaine from different configurations of implantable depots in a minipig abdominal hernia repair model.

FIG. 24 is a graph illustrating in vivo release of bupivacaine from implantable depots with varying dosages in a rat subcutaneous model.

FIG. 25 illustrates chemical structures of plasticizers.

FIGS. 26A-26C are graphs illustrating in vitro release at pH 7.4 for depots formulated with triacetin (FIG. 26A), diethyl phthalate (FIG. 26B), and benzyl benzoate (FIG. 26C) at 14 wt % loading.

FIGS. 27A-27C are graphs illustrating in vitro release at pH 7.4 for depots formulated with triacetin (FIG. 27A), diethyl phthalate (FIG. 27B), and benzyl benzoate (FIG. 27C) at 3.2 wt % loading.

FIG. 28A-28C are graphs illustrating in vitro release at pH 5.8 for depots with various plasticizer loadings and varying thicknesses.

FIG. 29 is a graph illustrating in vitro release at pH 5.8 for depots with no plasticizer, a single plasticizer, or dual plasticizers.

FIG. 30 is a graph illustrating in vitro release at pH 5.8 for depots with no plasticizer, a single plasticizer, dual plasticizers, or triple plasticizers.

FIG. 31 is a graph illustrating in vitro release at pH 5.8 for depots with no plasticizer or dual plasticizers.

FIG. 32A illustrates a setup for mechanical testing of depots.

FIG. 32B is an image of a plasticizer-loaded depot during mechanical testing.

FIGS. 33A-33H are graphs illustrating force-displacement curves for depots formulated with benzyl benzoate (FIG. 33A), diethyl phthalate (FIG. 33B), tributyl O-acetyl citrate (FIG. 33C), isopropyl myristate (FIG. 33D), PEG400 (FIG. 33E), triacetin (FIG. 33F), benzyl alcohol (FIG. 33G), and propylene glycol (FIG. 33H) at 14 wt % loading.

FIG. 34 is a graph illustrating force-displacement curves for depots formulated with 3.2 wt % loading of various plasticizers.

FIG. 35 is a graph illustrating force-displacement curves for depots with single or dual plasticizers at various time points post-manufacturing.

FIG. 36 is a graph illustrating force-displacement curves for depots with triple plasticizers at various time points post-manufacturing.

### DETAILED DESCRIPTION

The present technology relates to implantable depots for the sustained, controlled release of therapeutic agents, and associated systems and methods. For example, in some embodiments, an implantable depot for treating a subject includes a therapeutic region including a polymer (e.g., poly(lactide-co-glycolide)), an analgesic agent (e.g., bupivacaine), and at least one plasticizer (e.g., triacetin, diethyl phthalate, benzyl benzoate, and/or glycerol). The presence of the plasticizer can enhance the flexibility of the depot. For instance, the depot can have a flexural modulus within a range from 1 MPa to 400 MPa. When implanted in vivo, the therapeutic region can release the analgesic agent for a desired treatment period, such as a treatment period of at least 3 days. The depot may be configured to provide short-term release of the analgesic agent (e.g., the treatment period is no more than 7 days) or long-term release of the analgesic agent (e.g., the treatment period is at least 14 days), as desired.

As another example, in some embodiments, an implantable depot for treating a subject includes a therapeutic region having a first surface, a second surface opposite the first surface, and a lateral surface between the first and second surfaces. The therapeutic region can include a polymer and a therapeutic agent (e.g., bupivacaine). When implanted in the subject, the depot can release the therapeutic agent from the first surface, second surface, and lateral surface of the therapeutic region. The release profile of the therapeutic agent can be tuned by altering various parameters of the depot, such as the composition (e.g., amounts and/or types of therapeutic agent, polymer, and/or other components such as releasing agents) and/or geometry (e.g., thickness of the therapeutic region). Accordingly, the depots described herein can be adapted to provide sustained, controlled release of the therapeutic agent suitable for many different types of applications, such as treating postoperative pain following a surgical procedure.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Implantable Depots for Delivering Therapeutic Agents

### A. Overview

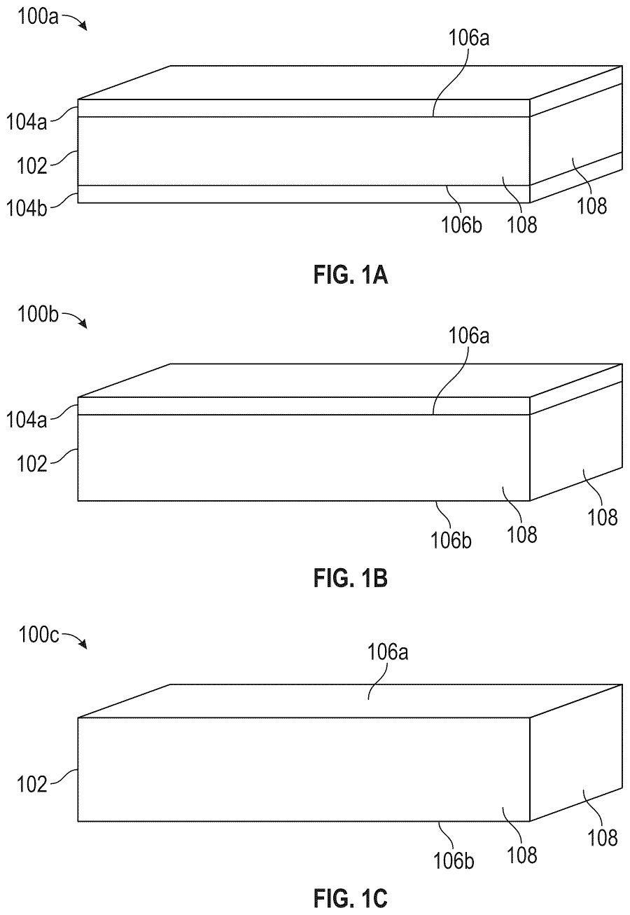

FIG. 1A is a partially schematic view of an implantable depot 100a configured in accordance with embodiments of the present technology. The depot 100a is configured to be implanted at a treatment site in a patient's body and, once implanted, release at least one therapeutic agent at the treatment site in a controlled manner, e.g., according to a desired release profile. The therapeutic agent can be any substance suitable for treating a patient's disease or condition. For example, the therapeutic agent can be or include an analgesic (e.g., bupivacaine) for addressing postoperative pain or other types of pain (e.g., chronic pain). Additional examples and features of therapeutic agents that can be included in the depot 100a are provided in Section I.C.1. below.

The depot 100a can be any suitable structure or device suitable for carrying and controllably releasing the therapeutic agent, such as a film, sheet, strip, ribbon, capsule, coating, matrix, wafer, pill, pellet, bead, scaffold, or a combination thereof. In the illustrated embodiment, the depot 100a is a multilayered, monolithic structure including a therapeutic region 102 disposed between a first control region 104a and a second control region 104b. The therapeutic region 102 (also known as a “core region,” “drug core,” or “drug layer”) includes the therapeutic agent, while the control regions 104a, 104b (also known as the “control layers”) can modulate the release of the therapeutic agent from the therapeutic region 102. As discussed in detail below, the geometry and composition of the therapeutic region 102 and the control regions 104a, 104b can be configured to produce a desired release profile of the therapeutic agent.

In some embodiments, the therapeutic region 102 includes the therapeutic agent and at least one polymer (e.g., poly(lactide-co-glycolide) (PLGA)). The polymer can be combined with the therapeutic agent to form a solid central core of the depot 100a. In some embodiments, the therapeutic agent and the polymer are discrete phases within the therapeutic region 102, with the polymer acting as a “glue” to hold the therapeutic agent together. In such embodiments, the therapeutic agent can form discrete crystals, particles, etc., that are adhered to each other by the polymer to form a monolithic material. In other embodiments, however, the therapeutic agent can instead be dissolved in the polymer to form a single-phase drug-polymer matrix. In some embodiments, the polymer is a bioresorbable polymer that is configured to degrade when exposed to physiologic fluids. The degradation characteristics of the bioresorbable polymer can be selected to modulate the release rate of the therapeutic agent from the therapeutic region 102. Additional examples and features of polymers that can be included in the therapeutic region 102 are provided in Section I.C.2 below.

Optionally, the therapeutic region 102 can include additional components, such as a releasing agent (e.g., polysorbate). The releasing agent can also affect the release rate of the therapeutic agent. In some embodiments, when exposed to a fluid (e.g., physiologic fluid), the releasing agent can have a dissolution rate that is faster than the degradation rate of the polymer in the therapeutic region 102. Accordingly, when a fluid contacts the therapeutic region 102 (e.g., after implantation of the depot 100a in a treatment site), the releasing agent dissolves within the surrounding polymer of the therapeutic region 102, thus forming openings (e.g., channels, voids, pores, etc.) in the surrounding polymer region that promote infiltration of fluid into the therapeutic region 102 and/or diffusion of the therapeutic agent out of the therapeutic region 102. Thus, increasing the amount of releasing agent in the therapeutic region 102 can increase the release rate of the therapeutic agent after implantation, as discussed in further detail elsewhere herein. Alternatively or in combination, the releasing agent can function as a surfactant to increase water uptake into the depot 100a, or otherwise alter the interfaces between the therapeutic agent, the polymer, and water to enhance release rate. Optionally, the releasing agent can modulate the mechanical properties of the therapeutic region 102 (e.g., increasing flexibility and/or reducing brittleness), which can facilitate manufacturing, storage, and/or handling of the depot 100a. Additional examples and features of releasing agents that can be included in the therapeutic region 102 are provided in Section I.C.3 below. In other embodiments, however, the therapeutic region 102 can be provided without any releasing agent.

As shown in FIG. 1A, the therapeutic region 102 is disposed between the control regions 104a, 104b. The first control region 104a can partially or fully cover a first surface 106a (e.g., an upper surface) of the therapeutic region 102. The second control region 104b can partially or fully cover a second surface 106b (e.g., lower surface) of the therapeutic region 102 opposite the first surface 106a. The therapeutic region 102 can include one or more lateral surfaces 108 that are not covered by the control regions 104a, 104b. In the illustrated embodiment, for example, all four lateral surfaces 108 of the therapeutic region 102 are exposed. In other embodiments, the therapeutic region 102 can instead include three, two, or a single exposed lateral surface 108. Alternatively, the therapeutic region 102 can be entirely encapsulated by one or more control regions, such that there are no exposed lateral surfaces 108.

The control regions 104a, 104b can each include at least one polymer (e.g., PLGA). The first control region 104a can be made of the same polymer as the second control region 104b, or can be made of a different polymer. Additionally, the polymers used in the first and/or second control regions 104a, 104b can be the same as the polymer used in the therapeutic region 102, or can be different polymers. In some embodiments, the polymers of the first and/or second control regions 104a, 104b are bioresorbable polymers. Additional examples and features of polymers that can be included in the first and second control regions 104a, 104b are provided in Section I.C.2 below.

Optionally, the control regions 104a, 104b can include additional components, such as a releasing agent (e.g., polysorbate). The first control region 104a can include the same releasing agent as the second control region 104b, or can include a different releasing agent. Additionally, the releasing agent used in the first and/or second control regions 104a, 104b can be the same as the releasing agent used in the therapeutic region 102, or can be different releasing agents. Additional examples and features of releasing agents that can be included in the first and second control regions 104a, 104b are provided in Section I.C.3 below. In other embodiments, however, the first and/or second control regions 104a, 104b can be provided without any releasing agent.

The configuration (e.g., position and/or geometry) and composition of the control regions 104a, 104b can modulate the release profile of the therapeutic agent from the therapeutic region 102. For example, when the depot 100a is implanted at a treatment site, the control regions 104a, 104b can be positioned between the first and second surfaces 106a, 106b of the therapeutic region 102 and physiologic fluids at the treatment site. Accordingly, the control regions 104a, 104b can reduce or prevent diffusion of fluids toward the first and second surfaces 106a, 106b. In some embodiments, the polymer within the control regions 104a and 104b creates a barrier that is partially or completely impenetrable to fluid infiltration, such that any additional components within the control regions 104a, 104b (e.g., releasing agent) are sequestered within the polymer and not exposed to fluids.

The control regions 104a, 104b can reduce or prevent diffusion of the therapeutic agent from the first and second surfaces 106a, 106b. In some embodiments, the therapeutic agent is released from a surface of the therapeutic region 102 only if that surface is exposed to fluid, thus providing a route for the therapeutic agent to diffuse out of the therapeutic region 102 and into the surrounding environment. The control regions 104a, 104b can be configured to block all or substantially all release of the therapeutic agent from the first and second surfaces 106a, 106b, such that all or substantially all of the therapeutic agent delivered from the depot 100a is released through the exposed lateral surfaces 108 of the therapeutic region 102. For example, at least 80%, 85%, 90%, 95%, 99%, or 100% of the therapeutic agent delivered from the depot 100a can be released through the lateral surfaces 108, while less than 20%, 15%, 10%, 5%, or 1% of the therapeutic agent delivered from the depot 100a can be released through the first and second surfaces 106a, 106b. In some embodiments, the overall release rate of the therapeutic agent depends at least in part on the distance (e.g., maximum, minimum, and/or average distance) between individual molecules of the therapeutic agent and the nearest exposed surface of the therapeutic region 102, also referred to herein as the “travel distance” of the therapeutic agent. For example, therapeutic agent located at the periphery of the depot 100a near the lateral surfaces 108 may release more quickly than therapeutic agent located within the interior of the depot 100a away from the lateral surfaces 108, thus creating a sustained release profile, as described in greater detail below.

In some embodiments, the control regions 104a, 104b also serve other functions, such as increasing the mechanical integrity of the depot 100a. For example, the control regions 104a, 104b can have a higher tensile strength and/or fracture resistance than the therapeutic region 102. Accordingly, the presence of the control regions 104a, 104b can improve the handling and storage characteristics of the depot 100a.

The depot 100a is configured to release a therapeutic agent in a highly controlled, predetermined manner that is specifically tailored to the medical condition being treated and/or the therapeutic agent used. As described in greater detail below, the release kinetics of the depot 100 can be customized for a particular application by varying one or more aspects of the depot's composition and/or structure, such as any of the following: the geometry (e.g., size and/or shape) of the depot 100a, therapeutic region 102, and/or control regions 104a, 104b; the types of therapeutic agent, polymer, and/or releasing agent used; and the amounts of therapeutic agent, polymer, and/or releasing agent included in the depot 100a (e.g., in the therapeutic region 102 and/or the control regions 104a, 104b).

FIG. 1B is a partially schematic view of another implantable depot 100b configured in accordance with embodiments of the present technology. The depot 100b is generally similar to the depot 100a of FIG. 1A, except that the depot 100b includes a single control region rather than two control regions. In the illustrated embodiment, the depot 100b includes the first control region 104a covering the first surface 106a of the depot 100b, such that the second surface 106b and lateral surfaces 108 are exposed. Accordingly, the control region 104a can block all or substantially all release of the therapeutic agent from the first surface 106a, such that all or substantially all of the therapeutic agent delivered from the depot 100b is released from the exposed second surface 106b and lateral surfaces 108 of the therapeutic region 102. Alternatively, the depot 100b can instead include the second control region 104b covering the second surface 106b of the depot 100b, such that the first surface 106a and lateral surfaces 108 are exposed. The depot 100b can be used in embodiments where a faster release rate is desired (relative to the release rate of the depot 100a), and/or where the therapeutic agent is relatively hydrophobic, as described further below.

FIG. 1C is a partially schematic view of yet another implantable depot 100c configured in accordance with embodiments of the present technology. The depot 100c is generally similar to the depot 100a of FIG. 1A, except that the depot 100c does not include any control regions, such that the first surface 106a, second surface 106b, and lateral surfaces 108 are exposed. Accordingly, the therapeutic agent can be released from the first surface 106a, second surface 106b, and lateral surfaces 108 of the depot 100c. The depot 100c can be used in embodiments where a faster release rate is desired (relative to the release rate of the depot 100a or the depot 100b), and/or where the therapeutic agent is relatively hydrophobic, as described further below.

### B. Geometry

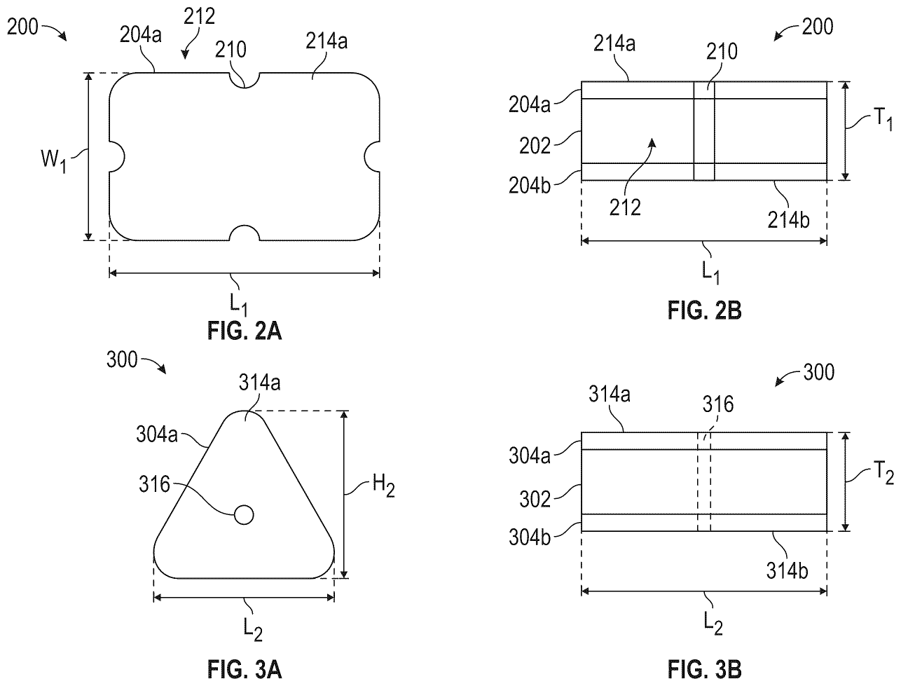

FIGS. 2A-5H illustrate representative examples of depots 200-570 with various geometries configured in accordance with embodiments of the present technology. The features of the depots 200-570 can be generally similar to the features of the depots 100a-100c of FIGS. 1A-1C. Accordingly, like numbers (e.g., therapeutic region 102 versus therapeutic region 202) are used to identify similar or identical components in FIGS. 1A-5H, and the discussion of the depots 200-570 of FIGS. 2A-5H will be limited to those features that differ from the depots 100a-100c of FIGS. 1A-1C. Additionally, any of the features of the depots 200-570 of FIGS. 2A-5H can be combined with each other and/or with the features of the depots 100a-100c of FIGS. 1A-1C. Although some embodiments of the depots 200-570 of FIGS. 2A-5H are depicted as having two control regions (similar to the depot 100a of FIG. 1A), in other embodiments, any of the depots 200-570 can have a single control region (similar to the depot 100b of FIG. 1B) or no control regions (similar to the depot 100c of FIG. 1C).

FIG. 2A is a top view of a rectangular depot 200 and FIG. 2B is a side view of the rectangular depot 200. As best seen in FIG. 2A, the depot 200 has a generally rectangular shape with rounded corners. The depot 200 can have a length L₁ within a range from 10 mm to 50 mm, 15 mm to 45 mm, 20 mm to 30 mm, or 25 mm to 35 mm. In some embodiments, the length L₁ is at least 10 mm, 12.5 mm, 15 mm, 17.5 mm, 20 mm, 22.5 mm, 25 mm, 27.5 mm, 30 mm, 32.5 mm, 35 mm, 37.5 mm, 40 mm, 42.5 mm, 45 mm, 47.5 mm, or 50 mm. The depot 200 can have a width W₁ within a range from 5 mm to 30 mm, 10 mm to 25 mm, 10 mm to 20 mm, or 15 mm to 25 mm. In some embodiments, the width W₁ is greater than or equal to 5 mm, 7.5 mm, 10 mm, 11 mm, 12 mm, 12.5 mm, 13 mm, 14 mm, 15 mm, 16 mm, 17 mm, 17.5 mm, 18 mm, 19 mm, 20 mm, 22.5 mm, 25 mm, 27.5 mm, or 30 mm.

Referring next to FIG. 2B, the depot 200 can have a total thickness T₁ within a range from 100 μm to 5 mm, 500 μm to 2.5 mm, 1 mm to 2 mm, 750 μm to 1.25 mm, 1 mm to 1.5 mm, 1.25 mm to 1.75 mm, 1.75 mm to 2.25 mm, or 2 mm to 2.5 mm. For example, the total thickness T₁ can be greater than or equal to 100 μm, 200 μm, 300 μm, 400 μm, 500 μm, 600 μm, 700 μm, 800 μm, 900 μm, 1 mm, 1.1 mm, 1.2 mm, 1.25 mm, 1.3 mm, 1.4 mm, 1.5 mm, 1.6 mm, 1.7 mm, 1.75 mm, 1.8 mm, 1.9 mm, 2 mm, 2.1 mm, 2.2 mm, 2.25 mm, 2.3 mm, 2.4 mm, 2.5 mm, 2.6 mm, 2.7 mm, 2.75 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, or 5 mm.

The therapeutic region 202 can have a thickness that is greater than or equal to 50%, 60%, 70%, 75%, 80%, 85%, 90%, 91%, 92%, 93%, 94%, 95%, 96%, 97%, 97.5%, 98%, 98.5%, 98.8%, 99%, or 99.5% of the total thickness T₁ of the depot 200. In some embodiments, the thickness of the therapeutic region 202 is within a range from 100 μm to 5 mm, 500 μm to 2.5 mm, 1 mm to 2 mm, 750 μm to 1.25 mm, 1 mm to 1.5 mm, 1.25 mm to 1.75 mm, 1.75 mm to 2.25 mm, 1.8 mm to 2.2 mm, 1.9 mm to 2.1 mm, 1.5 mm to 2.5 mm, or 2 mm to 2.5 mm. For example, the thickness of the therapeutic region 202 can be greater than or equal to 100 μm, 200 μm, 300 μm, 400 μm, 500 μm, 600 μm, 700 μm, 800 μm, 900 μm, 910 μm, 920 μm, 930 μm, 940 μm, 950 μm, 960 μm, 970 μm, 980 μm, 990 μm, 1 mm, 1.1 mm, 1.2 mm, 1.25 mm, 1.3 mm, 1.4 mm, 1.5 mm, 1.6 mm, 1.61 mm, 1.62 mm, 1.63 mm, 1.64 mm, 1.65 mm, 1.66 mm, 1.67 mm, 1.68 mm, 1.69 mm, 1.7 mm, 1.75 mm, 1.8 mm, 1.9 mm, 1.91 mm, 1.92 mm, 1.93 mm, 1.94 mm, 1.95 mm, 1.96 mm, 1.97 mm, 1.98 mm, 1.99 mm, 2 mm, 2.1 mm, 2.2 mm, 2.25 mm, 2.3 mm, 2.4 mm, 2.5 mm, 2.6 mm, 2.7 mm, 2.75 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, or 5 mm.

In the illustrated embodiment, the control regions 204a, 204b have the same thickness. In other embodiments, however, the control regions 204a, 204b can have different thicknesses (e.g., the first control region 204a can have a greater thickness than the second control region 204b, or vice-versa). The control regions 204a, 204b can each have a respective thickness that is less than or equal to 50%, 40%, 30%, 25%, 20%, 15%, 10%, 9%, 8%, 7%, 6%, 5%, 4%, 3%, 2.5%, 2%, 1.5%, 1.2%, 1%, or 0.5% of the total thickness T₁ of the depot 200. In some embodiments, each control region 204a, 204b has a thickness within a range from 1 μm to 100 μm, m to 50 μm, 10 μm to 20 μm, 5 μm to 15 μm, or 15 μm to 25 μm. For example, each control region 204a, 204b can have a thickness less than or equal to 100 μm, 95 μm, 90 μm, 85 μm, 80 am, 75 μm, 70 μm, 65 μm, 60 μm, 55 μm, 50 μm, 40 μm, 35 μm, 30 μm, 29 μm, 28 μm, 27 μm, 26 μm, 25 μm, 24 μm, 23 μm, 22 μm, 21 μm, 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm. In some embodiments, thicker control regions are more resistant to fracture, cracking, or other damage during manufacturing, handling, and/or storage, and thus may produce a more consistent release profile of the therapeutic agent across different lots or batches. However, the control regions 204a, 204b can still be sufficiently thin so that the depot 200 still has a compact size suitable for placement in a treatment site.

The combined thickness of the control regions 204a, 204b can be less than or equal to 50%, 40%, 30%, 25%, 20%, 15%, 10%, 9%, 8%, 7%, 6%, 5%, 4%, 3%, 2.5%, 2%, 1.5%, 1.2%, 1%, or 0.5% of the total thickness T₁ of the depot 200 and/or the thickness of the therapeutic region 202. In some embodiments, the ratio of the combined thickness of the control regions 204a, 204b to the thickness of the therapeutic region 202 is no more than 1/10, 1/20, 1/25, 1/30, 1/35, 1/39, 1/40, 1/45, 1/49, 1/50, 1/55, 1/60, 1/65, 1/70, 1/75, 1/80, 1/84, 1/85, 1/90, 1/95, or 1/100. The combined thickness of the control regions 204a, 204b can be within a range from 1 μm to 100 μm, 5 μm to 50 μm, 10 μm to 20 μm, 5 μm to 15 μm, 15 μm to 25 μm, 40 μm to 60 μm, or 45 μm to 55 μm. For example, the combined thickness of the control regions 204a, 204b can be less than or equal to 100 μm, 95 μm, 90 μm, 85 μm, 80 μm, 75 μm, 70 μm, 65 μm, 60 μm, 55 μm, 50 μm, 40 μm, 35 μm, 30 μm, 29 μm, 28 μm, 27 μm, 26 μm, 25 μm, 24 μm, 23 μm, 22 μm, 21 μm, 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm.

In some embodiments, the volume of the therapeutic region 202 is greater than or equal to 50%, 60%, 70%, 75%, 80%, 85%, 90%, 91%, 92%, 93%, 94%, 95%, 96%, 97%, 97.5%, 98%, 98.5%, 98.8%, 99%, or 99.5% of the total volume of the depot 200. The combined volume of the control regions 204a, 204b can be less than or equal to 50%, 40%, 30%, 25%, 20%, 15%, 10%, 9%, 8%, 7%, 6%, 5%, 4%, 3%, 2.5%, 2%, 1.5%, 1.2%, 1%, or 0.5% of the total volume of the depot 200. In some embodiments, the depot 200 has a volume of at least 100 mm³, 150 mm³, 200 mm³, 250 mm³, 300 mm³, 350 mm³, 400 mm³, 450 mm³, or 500 mm³. The therapeutic region 202 can have a volume of at least 100 mm³, 150 mm³, 200 mm³, 250 mm³, 300 mm³, 350 mm³, 400 mm³, 450 mm³, or 500 mm³. The control regions 204a, 204b can collectively have a volume of no more than 100 mm³, 75 mm³, 50 mm³, 25 mm³, 10 mm³, 9 mm³, 8 mm³, 7 mm³, 6 mm³, 5 mm³, 4 mm³, 3 mm³, 2 mm³, or 1 mm³.

Referring to FIGS. 2A and 2B together, in some embodiments, the depot 200 includes one or more notches 210 (e.g., cutouts, indentations, recesses, etc.) formed in one or more lateral surfaces 212 of the depot 200. The notches 210 can be configured to modulate the release characteristics of the depot 200 by altering the amount of surface area exposed to fluids. Alternatively or in combination, the notches 210 can be configured to accommodate sutures or other fasteners for securing the depot 200 in place at a treatment site. In other embodiments, however, the depot 200 can be provided without any notches 210.

In the illustrated embodiment, the depot 200 includes four notches 210, one at each of the four lateral surfaces 212 of the depot 200. Alternatively, the depot 200 can include a different number of notches 210 (e.g., one, two, three, five, or more notches 210). Some lateral surfaces 212 of the depot 200 can include more than one notch 210 (e.g., two, three, four, or more notches 210) and/or some lateral surfaces 212 may not include any notches 210 (e.g., the notches 210 can be located at three, two, or a single lateral surface 212 of the depot 200). Additionally, although FIGS. 2A and 2B illustrate each notch 210 as being located at or near the center of the corresponding lateral surface 212, in other embodiments, some or all of the notches 210 can be at different locations (e.g., at or near the corners of the depot 200).

In the illustrated embodiment, each notch 210 extends along the entire thickness T₁ of the depot 200, e.g., from a first surface 214a (e.g., uppermost surface) of the depot 200, through the first control region 204a, therapeutic region 202, and second control region 204b, to a second surface 214b (e.g., lowermost surface) of the depot 200. Alternatively, some or all of the notches 210 can extend only partially along the thickness T₁ of the depot 200 (e.g., the notch 210 can be localized to the first control region 204a, the second control region 204b, the therapeutic region 202, the first control region 204a and the therapeutic region 202, the therapeutic region 202 and the second control region 204b, etc.).

The geometry (e.g., size, shape) of the notches 210 can be varied as desired. For example, in the embodiment of FIG. 2A, each of the notches 210 has a semi-circular shape. In other embodiments, some or all of the notches 210 can have a different shape, such as triangular, square, rectangular, semi-oval, or combinations thereof. All or a portion of some or all of the notches 210 can form a curved portion of the corresponding lateral surface, and/or all or a portion of some or all of the notches 210 can form a linear portion of the corresponding lateral surface. Each notch 210 can have a diameter or width (e.g., maximum width) within a range from 0.5 mm to 10 mm, 1 mm to 5 mm, or 2.5 mm to 3.5 mm. For example, each notch 210 can have a diameter or width less than or equal to 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, or 0.5 mm. In some embodiments, all of the notches 210 have the same size and/or shape. In other embodiments, some or all of the notches 210 can have different sizes and/or shapes.

The depot 200 can be manufactured in many different ways. In some embodiments, for example, the therapeutic region 202 is formed first using a heat compression process. The heat compression process can be performed at a temperature above room temperature (e.g., at least 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., or 120° C.) and a pressure within a range from about 0.01 MPa to about 1 MPa, or about 0.1 MPa to about 0.8 MPa, or about 0.3 MPa to about 0.6 MPa. Subsequently, the control regions 204a, 204b can be applied to the therapeutic region 202 using spray coating, dip coating, solvent casting, laser melting, or other suitable processes known to those of skill in the art. The notches 210 can then be cut into the depot 200 using a blade, laser cutting, ultrasonic cutting, air knife, or other suitable techniques. In some embodiments, each depot 200 is formed individually. In other embodiments, the therapeutic region 202 and control regions 204a, 204b can be formed as a larger material sheet, which can then be cut into individual depots 200.

FIG. 3A is a top view of a triangular depot 300 and FIG. 3B is a side view of the triangular depot 300. As best seen in FIG. 3A, the depot 300 has a generally triangular shape with rounded corners. The triangular shape may be advantageous for conforming to the shape of certain surgical sites, such as the femoral gutters and/or suprapatellar pouch of the knee. In the illustrated embodiment, the depot 300 is shaped as an equilateral triangle, such that all three sides of the depot 300 have the same length L₂. The length L₂ can be within a range from 10 mm to 50 mm, 15 mm to 45 mm, 20 mm to 30 mm, or 25 mm to 35 mm. In some embodiments, the length L₂ is at least 10 mm, 12.5 mm, 15 mm, 17.5 mm, 20 mm, 22.5 mm, 25 mm, 27.5 mm, 30 mm, 30.5 mm, 32.5 mm, 35 mm, 37.5 mm, 40 mm, 42.5 mm, 45 mm, 47.5 mm, or 50 mm. In other embodiments, however, some or all of the sides of the depot 300 can have different respective lengths. The depot 300 can have a height H₂ within a range from 10 mm to 40 mm, 15 mm to 35 mm, 20 mm to 30 mm, or 25 mm to 35 mm. In some embodiments, the height H₂ is greater than or equal to 10 mm, 11 mm, 12 mm, 13 mm, 14 mm, 15 mm, 16 mm, 17 mm, 18 mm, 19 mm, 20 mm, 25 mm, 25.5 mm, 26 mm, 2.65 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, or 35 mm.

Referring next to FIG. 3B, the depot 300 can have a total thickness T₂. The values and ranges for the thickness T₂ of the depot 300 and for the thicknesses of the therapeutic region 302 and control regions 304a, 304b (and the ratios between the various thicknesses) can be the same or similar to the corresponding values and ranges for the depot 200 of FIGS. 2A and 2B.

In some embodiments, the volume of the therapeutic region 302 of the depot 300 is greater than or equal to 50%, 60%, 70%, 75%, 80%, 85%, 90%, 91%, 92%, 93%, 94%, 95%, 96%, 97%, 97.5%, 98%, 98.5%, 98.8%, 99%, or 99.5% of the total volume of the depot 300. The combined volume of the control regions 304a, 304b can be less than or equal to 50%, 40%, 30%, 25%, 20%, 15%, 10%, 9%, 8%, 7%, 6%, 5%, 4%, 3%, 2.5%, 2%, 1.5%, 1.2%, 1%, or 0.5% of the total volume of the depot 300. In some embodiments, the depot 300 has a volume of at least 100 mm³, 200 mm³, 300 mm³, 400 mm³, 500 mm³, 550 mm³, 600 mm³, 650 mm³, 700 mm³, 750 mm³, 800 mm³, 850 mm³, 900 mm³, 950 mm³, or 1000 mm³. The therapeutic region 302 can have a volume of at least 100 mm³, 200 mm³, 300 mm³, 400 mm³, 500 mm³, 550 mm³, 600 mm³, 650 mm³, 700 mm³, 750 mm³, 800 mm³, 850 mm³, 900 mm³, 950 mm³, or 1000 mm³. The control regions 304a, 304b can collectively have a volume of no more than 100 mm³, 75 mm³, 50 mm³, 45 mm³, 40 mm³, 35 mm³, 30 mm³, 25 mm³, 20 mm³, 25 mm³, 15 mm³, 10 mm³, 9 mm³, 8 mm³, 7 mm³, 6 mm³, 5 mm³, 4 mm³, 3 mm³, 2 mm³, or 1 mm³.

As best seen in FIG. 3A, the depot 300 can include a hole 316 (e.g., aperture, opening, channel) formed therein. The hole 316 can be configured to modulate the release characteristics of the depot 300, such as by altering the amount of surface area of the therapeutic region 302 that is exposed to fluids. For example, the hole 316 can expose portions of the therapeutic region 302 located away from the periphery of the depot 300, thus promoting fluid infiltration into and/or therapeutic agent release from the surfaces of the therapeutic region 302 exposed at the sidewalls of the hole 316. When the depot 300 is implanted, the hole 316 can also facilitate diffusion of the therapeutic agent to target tissues located directly above and/or below the depot 300. In other embodiments, however, the depot 300 can be provided without any holes 316.

In the illustrated embodiment, the depot 300 includes a single hole 316 at or near the center (e.g., centroid) of the depot 300. In other embodiments, the hole 316 can be at a different location in the depot 300. The location of the hole 316 can be selected to reduce (e.g., minimize) the average and/or maximum travel distance between individual molecules of the therapeutic agent and the nearest exposed surface of the therapeutic region 302. For example, the average and/or maximum travel distance of the therapeutic agent in the depot 300 can be less than or equal to 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, 0.5 mm, 0.25 mm, or 0.1 mm.

The hole 316 can extend through the entire thickness T₂ of the depot 300, e.g., from a first surface 314a (e.g., uppermost surface) of the depot 300, through the first control region 304a, therapeutic region 302, and second control region 304b, to a second surface 314b (e.g., lowermost surface) of the depot 300. Alternatively, the hole 316 can extend only partially through the thickness T₂ of the depot 300 (e.g., the hole 316 can extend through the first control region 304a only, the second control region 304b only, the first control region 304a and the therapeutic region 302 only, the therapeutic region 302 and the second control region 304b only, etc.).

The geometry (e.g., size, shape) of the hole 316 can be varied as desired. For example, as shown in FIG. 3A, the hole 316 can have a circular shape. In other embodiments, the hole 316 can have a different shape, such as an oval, triangular, square, or rectangular shape, or combinations thereof. The hole 316 can have a diameter or width (e.g., maximum width) within a range from 0.5 mm to 10 mm, 1 mm to 5 mm, or 2.5 mm to 3.5 mm. For example, the hole 316 can have a diameter or width less than or equal to 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, or 0.5 mm.

Alternatively or in combination, the hole 316 can serve other functions, such as accommodating fasteners (e.g., sutures) for securing the depot 300 in place at a treatment site. Optionally, the presence of the hole 316 can increase the overall flexibility of the depot 300, which can reduce the likelihood of inadvertent breakage during manufacturing, storage, and/or handling of the depot 300.

The depot 300 can be manufactured using techniques similar to those described above with respect to the depot 200 of FIGS. 2A and 2B. For example, the therapeutic region 302 can be formed using a heat compression process, and the control regions 304a, 304b can be applied to the therapeutic region 302 using spray coating, dip coating, solvent casting, laser melting, etc. The hole 316 can then be cut into the depot 300 using a blade, laser cutting, ultrasonic cutting, air knife, or suitable techniques known to those of skill in the art.

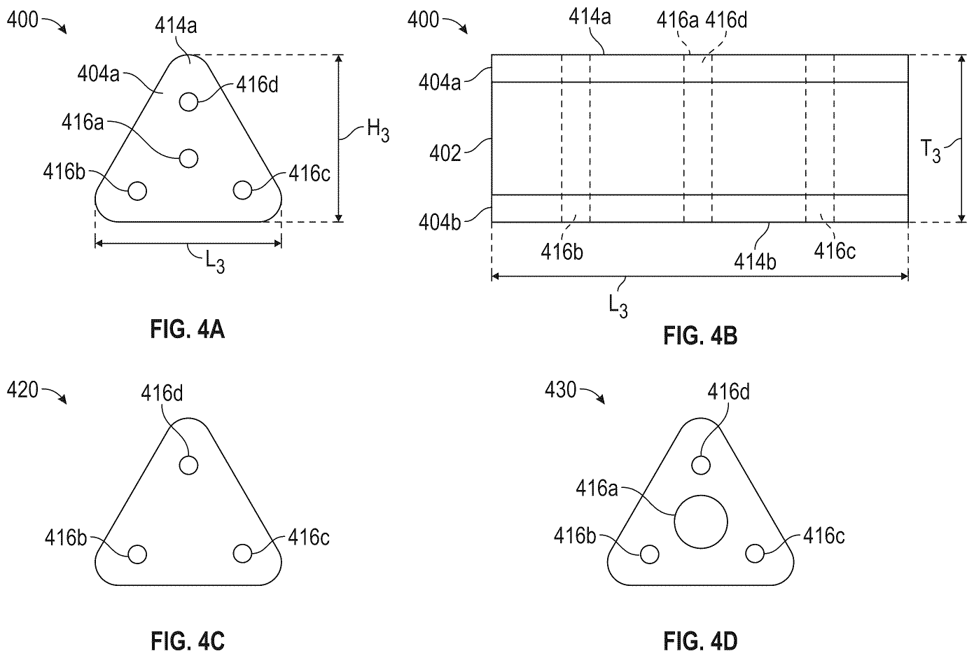

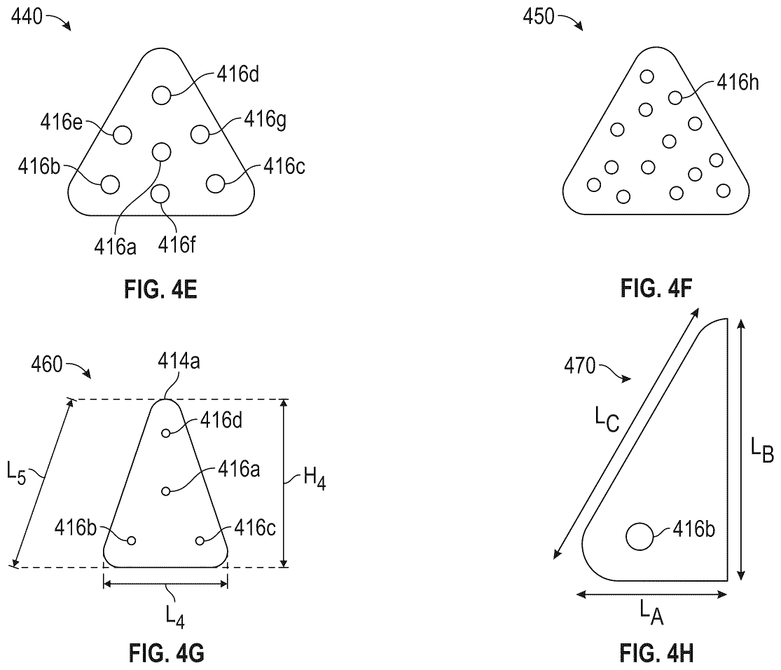

FIG. 4A is a top view of another triangular depot 400 and FIG. 4B is a side view of the triangular depot 400. The depot 400 can be generally similar to the depot 300 of FIGS. 3A and 3B. For example, the values and ranges for the dimensions of the depot 400 (e.g., length L₃, height H₃, and thickness T₃) can be the same or similar as the corresponding values and ranges described above in connection with the depot 300. Accordingly, the discussion of the depot 400 will be limited to those features that differ from the depot 300.

As best seen in FIG. 4A, the depot 400 includes a plurality of holes 416a-416d (e.g., apertures, openings, channels) formed therein. The holes 416a-416d can serve the same or a similar function as the hole 316 of the depot 300 of FIGS. 3A and 3B (e.g., modulating the release characteristics of the depot 400, accommodating fasteners for securing the depot 400, and/or increasing flexibility of the depot 400). In the illustrated embodiment, the depot 400 includes four holes 416a-416d: one hole 416a at the center or centroid of the depot 400, and three holes 416b-416d spaced apart from the central hole 416a and located near the three corners of the depot 400. Alternatively, the depot 400 can include a different number of holes (e.g., two, three, five, or more holes). For example, any of the holes 416a-416d can be omitted, e.g., the depot 400 only includes the central hole 416a, only includes the peripheral holes 416b-416d, only includes the central hole 416a and one peripheral hole (e.g., the top hole 416d), includes one or more holes in addition or alternatively to the holes 416a-416d, etc. Moreover, any of the holes 416a-416d can be located at different portions of the depot 400, such as at or near an edge of the depot 400, randomly distributed across the depot 400, etc.

The use of multiple holes 416a-416d can reduce the average and/or maximum travel distance for the therapeutic agent, e.g., compared to depots having fewer or no holes (e.g., the depot 300 of FIGS. 3A and 3B). In some embodiments, the average and/or maximum travel distance for the therapeutic agent in the depot 400 is less than or equal to 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2 mm, 1.5 mm, 1 mm, or 0.5 mm.

Each of the holes 416a-416d can extend through the entire thickness T₃ of the depot 400, e.g., from a first surface 414a (e.g., uppermost surface) of the depot 400, through the first control region 404a, therapeutic region 402, and second control region 404b, to a second surface 414b (e.g., lowermost surface) of the depot 400. Alternatively, some or all of the holes 416a-416d can extend only partially through the thickness T₃ of the depot 400 (e.g., through the first control region 404a only, the second control region 404b only, the first control region 404a and the therapeutic region 402 only, the therapeutic region 402 and the second control region 404b only, etc.).

In the illustrated embodiment, each of the holes 416a-416d extends through the therapeutic region 402 and exposes a surface thereof, such that the therapeutic agent can elute out of the depot via the holes 416a-416d. In other embodiments, a barrier material can be positioned over the surfaces of the therapeutic region 402 at some or all of the holes 416a-416d to reduce or prevent release of the therapeutic agent from a certain hole or holes. The barrier material can be or include any material that inhibits diffusion of therapeutic agent, such as a polymer layer or coating. For example, the barrier material can be made of a material identical or similar to the material of the control regions 404a, 404b. The barrier material can be located at the central hole 416a only, the peripheral holes 416b-416d only, or any other selected subset of the holes 416a-416d. For example, the barrier material can be located at holes that are intended to be used for suturing, while holes intended to modulate the release profile of the therapeutic agent may not include any barrier agent.

The geometry (e.g., size, shape) of the holes 416a-416d can be varied as desired. For example, as shown in FIG. 4A, the holes 416a-416d can each have a circular shape. In other embodiments, some or all of the holes 416a-416d can have a different shape, such as an oval, triangular, square, or rectangular shape, or combinations thereof. The holes 416a-416d can each have a diameter or width (e.g., maximum width) within a range from 0.5 mm to 10 mm, 1 mm to 5 mm, or 2.5 mm to 3.5 mm. For example, the holes 416a-416d can each have a diameter or width less than or equal to 10 mm, 9.5 mm, 9 mm, 8.5 mm, 8 mm, 7.5 mm, 7 mm, 6.5 mm, 6 mm, 5.5 mm, 5 mm, 4.5 mm, 4 mm, 3.5 mm, 3 mm, 2.5 mm, 2.25 mm, 2 mm, 1.75 mm, 1.5 mm, 1.25 mm, 1 mm, 0.75 mm, 0.5 mm, or 0.25 mm. In some embodiments, all of the holes 416a-416d have the same size and/or shape. In other embodiments, some or all of the holes 416a-416d can have different sizes and/or shapes. For example, the central hole 416a can have a different size and/or shape than the peripheral holes 416b-416d.

The depot 400 can be manufactured using techniques similar to those described above with respect to the depot 300 of FIGS. 3A and 3B. For example, the therapeutic region 402 can be formed using a heat compression process, and the control regions 404a, 404b can be applied to the therapeutic region 402 using spray coating, dip coating, solvent casting, laser melting, etc. The holes 416a-416d can then be cut into the depot 400 using a blade, laser cutting, ultrasonic cutting, air knife, or suitable techniques known to those of skill in the art. In embodiments where some or all of the holes 416a-416d include a barrier material, those holes can be formed in the therapeutic region 402 before the control regions 404a, 404b are applied, such that the material of the control regions 404a, 404b serves as the barrier material. Alternatively, the holes can be formed after the control regions 404a, 404b are applied, with the barrier material being applied to the holes in a subsequent processing step.

As another example, the depot 400 can be manufactured by first forming a large sheet or film of therapeutic region material. The sheet can then be coated (e.g., spray coated or dip coated) with control region material. After the coating process, the upper, lower, and lateral surfaces of the sheet can all be covered with the control region material. The sheet can then be cut into individual depots. In the resulting depot 400, the therapeutic region 402 can be exposed at the lateral surfaces of the depot 400 where the cuts were made, and can remain covered with the control region material at all other lateral surfaces. Accordingly, depending on the locations of the cuts, each depot 400 can include one, two, or three lateral surfaces where the therapeutic region 402 is exposed. For example, a depot 400 produced by cutting a square sheet in half along the diagonal can have one lateral surface where the therapeutic region 402 is exposed, and two lateral surfaces where the therapeutic region 402 is covered.

FIGS. 4C-4H illustrate additional examples of triangular depots 420-470 configured in accordance with embodiments of the present technology. The features of the depots 420-470 of FIGS. 4C-4H can be generally similar to the corresponding features of the depot 400 of FIGS. 4A and 4B. Accordingly, like numbers are used to identify similar or identical components in FIGS. 4A-4H, and the discussion of the depots 420-470 will be limited to those features that differ from the depot 400 of FIGS. 4A and 4B. Additionally, any of the features of the depots 420-470 can be combined with each other and/or with the features of the depot 400 of FIGS. 4A and 4B.

FIG. 4C is a top view of another triangular depot 420 configured in accordance with embodiments of the present technology. The depot 420 only includes the peripheral holes 416b-416d and does not include a central hole. In other embodiments, the depot 420 can include only a subset of the holes 416b-416d, such as the hole 416b only, the holes 416b and 416c only, etc.

FIG. 4D is a top view of yet another triangular depot 430 configured in accordance with embodiments of the present technology. In the illustrated embodiment, the central hole 416a of the depot 430 has a different geometry than the peripheral holes 416b-416d. For example, as shown in FIG. 4D, the central hole 416a can be larger than the peripheral holes 416b-416d. In other embodiments, the central hole 416a can instead be smaller than some or all of the peripheral holes 416b-416d. Additionally, although the central hole 416a is illustrated as having the same shape as the peripheral holes 416b-416d, the central hole 416a can alternatively have a different shape than some or all of the peripheral holes 416b-416d.

FIG. 4E is a top view of a triangular depot 440 configured in accordance with embodiments of the present technology. In the illustrated embodiment, the depot 440 includes additional holes 416e-416g. For example, as shown in FIG. 4E, the depot 440 includes three additional holes 416e-416g, each located near a respective side of the depot 440 (e.g., near a midpoint of the respective side). In other embodiments, the depot 440 can include a different number of additional holes, e.g., some of the holes 416e-416g can be omitted and/or the depot 440 can include additional holes at other locations. Alternatively or in combination, some or all of the holes 416a-416d can be omitted.

FIG. 4F is a top view of another triangular depot 450 configured in accordance with embodiments of the present technology. In the illustrated embodiment, the depot 450 includes a plurality of randomly distributed holes 416h. The holes 416h can each have the same geometry (e.g., size and/or shape), or some or all of the holes 416h can have different geometries. In some embodiments, the holes 416h can be localized to specific portions of the depot 450, such as near the corners only, near the center only, near the sides only, or any other suitable configuration.

Optionally, the control regions 404a, 404b of the depot 450 can extend over the lateral surfaces of the depot 450 (not visible in FIG. 4F), such that the therapeutic region 402 of the depot 450 is entirely enclosed by the control regions 404a, 404b, and is only exposed through the holes 416h. In such embodiments, the depot 450 can include a relatively large number of holes 416h (e.g., tens, hundreds, or thousands of holes 416h) to allow for release of the therapeutic agent.

FIG. 4G is a top view of a triangular depot 460 configured in accordance with embodiments of the present technology. The depot 460 is shaped as an isosceles triangle, such that one side of the depot 460 (e.g., the base) has a first length L₄, and the other two sides each have a second length L₅. In the illustrated embodiment, the second length L₅ is greater than the first length L₄, e.g., at least 1.1 times, 1.2 times, 1.3 times, 1.4 times, 1.5 times, 1.6 times, 1.7 times, 1.8 times, 1.9 times, 2 times, 2.5 times, 3 times, 4 times, or 5 times greater. In other embodiments, however, the first length L₄ can be greater than the second length L₅. The first length L₄ can be within a range from 10 mm to 40 mm, 15 mm to 35 mm, 20 mm to 30 mm, or 25 mm to 35 mm. In some embodiments, the first length L₁ is at least 10 mm, 12.5 mm, 15 mm, 17.5 mm, 20 mm, 22.5 mm, 25 mm, 27.5 mm, 30 mm, 32.5 mm, 35 mm, 37.5 mm, or 40 mm. The second length L₅ can be within a range from 20 mm to 60 mm, 30 mm to 50 mm, 35 mm to 45 mm, or 40 mm to 50 mm. In some embodiments, the second length L₅ is at least 20 mm, 25 mm, 30 mm, 32.5 mm, 35 mm, 37.5 mm, 40 mm, 42.5 mm, 45 mm, 47.5 mm, 50 mm, 55 mm, or 60 mm. The depot 460 can have a height H₄ within a range from 20 mm to 60 mm, 30 mm to 50 mm, or 35 mm to 45 mm. In some embodiments, the height H₄ is greater than or equal to 20 mm, 25 mm, 30 mm, 32.5 mm, 35 mm, 37.5 mm, 40 mm, 41 mm, 42 mm, 43 mm, 44 mm, 45 mm, 47.5 m, 50 mm, 55 mm, or 60 mm.

FIG. 4H is a top view of a triangular depot 470 configured in accordance with embodiments of the present technology. The depot 470 is shaped as a right triangle, in which a first side of the depot 470 has a first length L_{A}, a second side of the depot 470 has a second length L_{B}, and a third side of the depot 470 has a third length Lc. For example, the first length L_{A} can be The first length L_{A} can be within a range from 5 mm to 25 mm, 7.5 mm to 22.5 mm, 10 mm to 15 mm, or 12.5 mm to 17.5 mm. The second length L_{B} can be within a range from 8.5 mm to 35 mm, 13 mm to 30 mm, 17.5 mm to 26 mm, or 21.5 mm to 30 mm. The third length Lc can be within a range from 10 mm to 50 mm, 15 mm to 45 mm, 20 mm to 30 mm, or 25 mm to 35 mm.

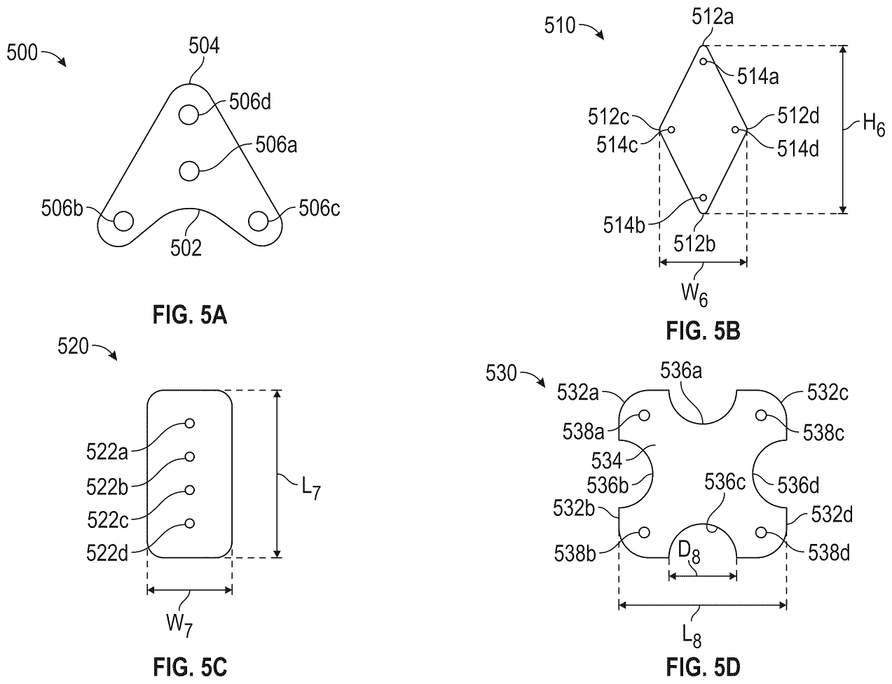

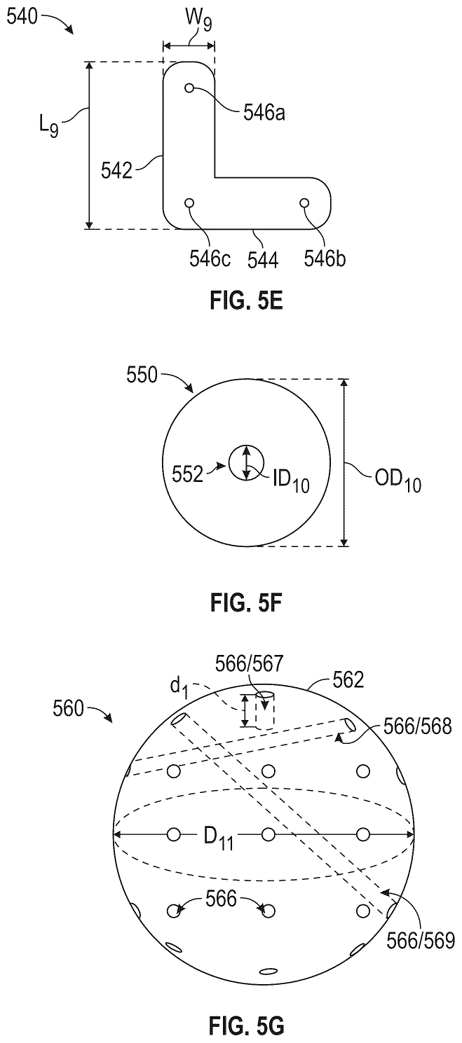

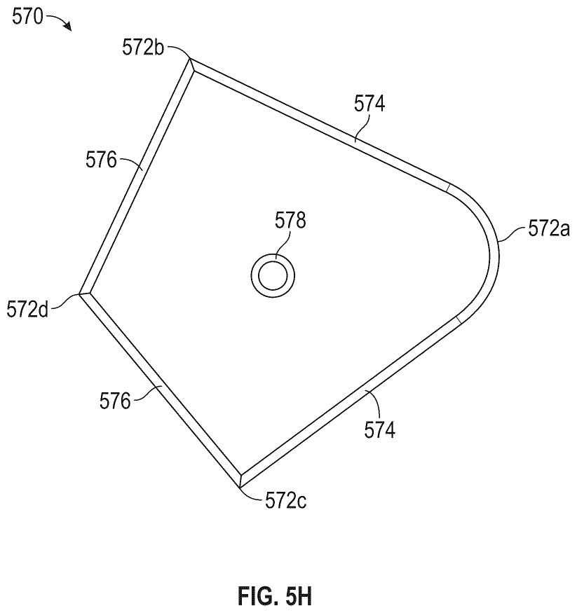

FIGS. 5A-5H illustrate additional examples of depots 500-570 with various geometries. The features of the depots 500-570 of FIGS. 5A-5H can be generally similar to the other depots described herein (e.g., the depot 400 of FIGS. 4A and 4B). Accordingly, the discussion of the depots 500-570 will be limited to those features that differ from the other embodiments of depots described herein. Additionally, any of the features of the depots 500-570 can be combined with each other and/or with the features of the other embodiments described herein.

FIG. 5A is a top view of an arrowhead-shaped depot 500 configured in accordance with embodiments of the present technology. As shown in FIG. 5A, the depot 500 is generally triangular, except that one edge 502 of the depot 500 is curved toward the center of the depot 500 to form an arrowhead or chevron shape. This geometry can facilitate insertion of the depot 500 into a treatment site. For example, the surgeon can orient the apex 504 of the depot 500 toward the treatment site, then apply force to the edge 502 of the depot 500 to push the depot 500 into the site. Although the depot 500 is depicted as including four holes 506a-506d configured similarly to the holes 416a-416d of FIGS. 4A and 4B (e.g., including a central hole 506a and three peripheral holes 506b-506d near the corners of the depot 500), the holes of the depot 500 can instead be configured according to any of the other embodiments described herein, or can be omitted altogether.

FIG. 5B is a top view of a diamond-shaped depot 510 configured in accordance with embodiments of the present technology. As shown in FIG. 5B, the depot 510 includes two corners 512a, 512b having a smaller angle (e.g., an angle less than or equal to 90°, 80°, 70°, 60°, 50°, 45°, 40°, 35°, 30°, 25°, 20°, 15°, or 10°) and two corners 512c, 512d having a larger angle (e.g., an angle greater than or equal to 90°, 100°, 110°, 120°, 125°, 130°, 135°, 140°, 145°, 150°, 155°, 160°, 165°, 170°, or 175°). Although the depot 510 is illustrated as having four sides of equal length, in other embodiments some of the sides of the depot 510 can have different lengths (e.g., the two upper sides can be longer or shorter than the two lower sides). The depot 510 can have a height H₆ within a range from 10 mm to 70 mm, 20 mm to 60 mm, 30 mm to 50 mm, 35 mm to 45 mm, or 40 mm to 45 mm. The depot 510 can have a width W₆ within a range from 5 mm to 50 mm, 10 mm to 30 mm, 15 mm to 25 mm, or 20 mm to 25 mm.

In the illustrated embodiment, the depot 510 includes four holes 514a-514d near the four corners 512a-512d. In other embodiments, the number and locations of the holes 514a-514d can be varied, e.g., the depot 510 can include fewer or more holes 514a-514d, the holes 514a-514d can be at different locations (e.g., at or near the center of the depot 510), etc. The geometry (e.g., shape, size) and function of the holes 514a-514d can be identical or similar to the holes 416a-416d of FIGS. 4A and 4B. Optionally, some or all of the holes 514a-514d can be omitted altogether.

FIG. 5C is a top view of a rectangular depot 520 configured in accordance with embodiments of the present technology. The depot 520 can have a length L₇ within a range from 10 mm to 50 mm, 20 mm to 40 mm, 25 mm to 35 mm, or 30 mm to 35 mm. The depot 520 can have a width W₇ within a range from 5 mm to 25 mm, 10 mm to 20 mm, 10 mm to 15 mm, or 15 mm to 20 mm. In the illustrated embodiment, the depot 520 includes four holes 522a-522d spaced evenly along the central vertical axis of the depot 520. In other embodiments, however, the depot 520 can include fewer or more holes 522a-522d. Additionally, the holes 522a-522d can be arranged differently, e.g., the spacing between the holes 522a-522d can be varied, the holes 522a-522d can be spaced along the central horizontal axis of the depot 520, the holes 522a-522d can be located near the four corners of the depot 520, etc. The geometry (e.g., shape, size) and function of the holes 522a-522d can be identical or similar to the holes 416a-416d of FIGS. 4A and 4B. Optionally, some or all of the holes 522a-522d can be omitted altogether.

FIG. 5D is a top view of a cross-shaped depot 530 configured in accordance with embodiments of the present technology. As shown in FIG. 5D, the depot 530 includes four arms 532a-532d extending from a central body 534. The depot 530 can be considered equivalent to a square with four cutouts 536a-536d in the four sides of the square. In the illustrated embodiment, all four sides of the depot 530 have the same length L₈, e.g., within a range from 10 mm to 40 mm, 15 mm to 35 mm, 20 mm to 30 mm, 20 mm to 25 mm, or 25 mm to 30 mm. In other embodiment, some of the sides of the depot 530 can have different lengths, e.g., the horizontal sides can have a greater or smaller length than the vertical sides.

The geometry of the cutouts 536a-536d can be varied as desired. In the illustrated embodiment, for example, the cutouts 536a-536d each have a semi-circular shape. In other embodiments, however, some or all of the cutouts 536a-536d can have a different shape, such as a square, rectangular, triangular, semi-oval, or other shape. The cutouts 536a-536d can each independently have any suitable size, such as a diameter D₈ or width within a range from 1 mm to 20 mm, 5 mm to 15 mm, or 8 mm to 12 mm.

In the illustrated embodiment, the depot 530 includes four holes 538a-538d located near the ends of the four arms 532a-532d. In other embodiments, the number and locations of the holes 538a-538d can be varied, e.g., the depot 530 can include fewer or more holes 538a-538d, the holes 538a-538d can be at different locations (e.g., at or near the center of the depot 530), etc. The geometry (e.g., shape, size) and function of the holes 538a-538d can be identical or similar to the holes 416a-416d of FIGS. 4A and 4B. Optionally, some or all of the holes 538a-538d can be omitted altogether.

FIG. 5E is a top view of an L-shaped depot 540. The depot 540 includes a first elongate arm 542 connected to a second elongate arm 544. The angle between the first and second elongate arms 542 can be greater than or equal to 10°, 15°, 20°, 30°, 40°, 45°, 50°, 60°, 70°, 80°, 90°, 110°, 120°, 130°, 140°, or 150°. In the illustrated embodiment, the first and second elongate arms 542 are generally rectangular structures and have the same length L₉ and width W₉. The length L₉ can be within a range from 10 mm to 50 mm, 20 mm to 40 mm, 25 mm to 35 mm, 25 mm to 30 mm, or 30 mm to 35 mm. The width W₉ can be within a range from 1 mm to 20 mm, 5 mm to 15 mm, 5 mm to 10 mm, or 10 mm to 15 mm. In other embodiments, the first elongate arm 542 can have a different (e.g., longer or shorter) length and/or width than the second elongate arm 544.

In the illustrated embodiment, the depot 540 includes three holes 546a-546c: one hole 546a near the end of the first elongate arm 542, one hole 546b located near the end of second elongate arm 544, and one hole 546c located near the connection between the first and second elongate arms 542, 544. In other embodiments, the number and locations of the holes 546a-546c can be varied, e.g., the depot 540 can include fewer or more holes 546a-546c, the holes 546a-546c can be at different locations (e.g., spaced along the length of the first elongate arm 542 and/or second elongate arm 544, etc. The geometry (e.g., shape, size) and function of the holes 546a-546c can be identical or similar to the holes 416a-416d of FIGS. 4A and 4B. Optionally, some or all of the holes 546a-546c can be omitted altogether.

FIG. 5F is a top view of a circular depot 550. The depot 550 can have a diameter OD₁₀ within a range from 1 mm to 100 mm, 5 mm to 50 mm, 10 mm to 30 mm, or 10 mm to 15 mm. In some embodiments, the diameter OD₁₀ is at least 5 mm, 10 mm, 15 mm, 20 mm, 25 mm, 30 mm, 35 mm, 40 mm, 45 mm, or 50 mm. Optionally, the depot 550 can include a central hole 552. The hole 552 can increase the release rate of the therapeutic agent by increasing the surface area of the depot 550 that is exposed to physiologic fluids and/or reducing the distance that the therapeutic agent travels to reach an exposed surface. In some embodiments, the hole 552 has a diameter ID₁₀ within a range from 1 mm to 20 mm, 2 mm to 15 mm, 5 mm to 10 mm, or 1 mm to 5 mm. For example, the diameter ID₁₀ can be less than or equal to 20 mm, 15 mm, 10 mm, 5 mm, 2 mm, or 1 mm. In some embodiments, the depot 550 has a thickness that is within a range from 100 μm to 5 mm, 500 μm to 2.5 mm, 1 mm to 2 mm, 750 μm to 1.25 mm, 1 mm to 1.5 mm, 1.25 mm to 1.75 mm, 1.75 mm to 2.25 mm, 1.8 mm to 2.2 mm, 1.9 mm to 2.1 mm, 1.5 mm to 2.5 mm, or 2 mm to 2.5 mm. For example, the thickness of the depot 550 can be greater than or equal to 100 μm, 200 μm, 300 μm, 400 μm, 500 μm, 600 μm, 700 μm, 800 μm, 900 μm, 910 μm, 920 μm, 930 μm, 940 μm, 950 μm, 960 μm, 970 μm, 980 μm, 990 μm, 1 mm, 1.1 mm, 1.2 mm, 1.25 mm, 1.3 mm, 1.4 mm, 1.5 mm, 1.6 mm, 1.61 mm, 1.62 mm, 1.63 mm, 1.64 mm, 1.65 mm, 1.66 mm, 1.67 mm, 1.68 mm, 1.69 mm, 1.7 mm, 1.75 mm, 1.8 mm, 1.9 mm, 1.91 mm, 1.92 mm, 1.93 mm, 1.94 mm, 1.95 mm, 1.96 mm, 1.97 mm, 1.98 mm, 1.99 mm, 2 mm, 2.1 mm, 2.2 mm, 2.25 mm, 2.3 mm, 2.4 mm, 2.5 mm, 2.6 mm, 2.7 mm, 2.75 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, or 5 mm.

FIG. 5G shows a depot 560 configured in accordance with embodiments of the present technology. The depot 560 has a body 562 having a spherical shape. The spherical shape may be advantageous for increasing the mechanical strength of the depot 560, as well as for packing irregularly-shaped spaces within a patient's body. The diameter D₁₁ of the body 560 can be within a range from 1 mm to 100 mm, 5 mm to 75 mm, 10 mm to 50 mm, 15 mm to 45 mm, 20 mm to 30 mm, 25 mm to 35 mm, 1 mm to 10 mm, or 1 mm to 5 mm. In some embodiments, the diameter D₁₁ is at least 1 mm, 2 mm, 5 mm, 10 mm, 15 mm, 20 mm, 25 mm, 30 mm, 35 mm, 40 mm, 45 mm, or 50 mm. In the illustrated embodiment, the depot 520 includes a plurality of holes 566 (only two labeled). The holes 566 can be spaced evenly along about the outer surface of the depot 560. In some embodiments, the holes 566 are only disposed at certain portions of the body 562, such as only within one hemisphere, only at a certain quadrant, etc. Optionally, the holes 566 can be randomly distributed over the outer surface of the body 562. In these and other embodiments, the holes 566 may not be evenly spaced. The geometry (e.g., shape, size) and function of the holes 566 can be identical or similar to the holes 416a-416d of FIGS. 4A and 4B. Optionally, some or all of the holes 566 can be omitted altogether. One, some, or all of the holes 566 can have a depth di that is less than or equal to the diameter D₁₁ of the body 562 (e.g., hole 567 and hole 568). In such embodiments, the hole can have an opening at the surface of the body 562, and can terminate at another opening at the surface of the body 562 (e.g., hole 568) or can terminate within the body 562 (e.g., hole 567). Additionally or alternatively, one, some, or all of the holes 566 can have a depth equal to the diameter D₁₁ of the body 562 (e.g., hole 569) such that the hole extends between two openings at the surface of the body 562 that are diametrically opposed. In some embodiments, at least one hole has a depth that is less than the diameter D₁₁ of the body 562, and at least another hole has a depth equivalent to the diameter D ii of the body 562.

FIG. 5H is a top view of a diamond-shaped depot 570 configured in accordance with embodiments of the present technology. The depot 570 includes one corner 572a having a having a smaller angle (e.g., an angle less than or equal to 90°, 80°, 70°, 60°, 50°, 45°, 40°, 35°, 30°, 25°, 20°, 15°, or 10°), two corners 572b, 572c having an intermediate angle (e.g., an angle less than or equal to 120°, 110°, 100°, 90°; and/or greater than 60°, 70°, 80°, 90°, 100°, 110°), and one corner 572d having a larger angle (e.g., an angle greater than or equal to 90°, 100°, 110°, 120°, 125°, 130°, 135°, 140°, 145°, 150°, 155°, 160°, 165°, 170°, or 175°). The depot 570 can have a pair of first sides 574 having the same or similar length L₁₂, and a pair of second sides 576 having the same or similar length L₁₃. For example, the length L₁₂ can be within a range from 5 mm to 30 mm, 10 mm to 20 mm, 10 mm to 15 mm, 15 mm to 20 mm, or 12 mm to 15 mm. The length L₁₃ can be within a range from 1 mm to 20 mm, 5 mm to 15 mm, or 5 mm to 10 mm. Although the length L₁₃ is depicted as being shorter than the length L₁₂ in FIG. 5H, in other embodiments, the length L₁₃ can be the same as or greater than the length L₁₂. Additionally, although the depot 570 is illustrated as having a single hole 578, in other embodiments, the depot 570 can have a greater number of holes 578, or the hole 578 can be omitted.

In some embodiments, the depots of the present technology are configured to be cut, fractured, or otherwise divided into smaller pieces before use. For example, a depot shaped as an equilateral triangle (e.g., the depot 300 of FIG. 3A or the depot 400 of FIG. 4A) can be designed to be broken into two smaller right triangles (e.g., similar to the depot 470 of FIG. 4H) or into three smaller diamonds (e.g., similar to the depot 570 of FIG. 5H). In such embodiments, the depot can include perforations, grooves, thinned portions, etc., defining the separation locations to facilitate controlled breakage of the depot. This approach can make it easier to fracture the depot while avoiding undesirable cracking in the control regions. Alternatively or in combination, a template can be provided for use as a guide while cutting the depot into smaller pieces (e.g., with a blade).

### C. Composition

The depots of the present technology (e.g., the depots 100a-570 of FIGS. 1A-5H) can have a composition configured to provide a desired release profile of a therapeutic agent. As discussed above, the depots described herein can include a therapeutic agent and one or more additional components such as polymers and/or releasing agents. Each of these components is described in greater detail below.

### 1. Therapeutic Agents

The therapeutic agent carried by the depots of the present technology (e.g., the depots 100a-570 of FIGS. 1A-5H) can be any biologically active substance (or combination of substances) that provides a therapeutic effect in a patient in need thereof. As used herein, “therapeutic agent” or “drug” may refer to a single therapeutic agent, or may refer to a combination of therapeutic agents. In some embodiments, the therapeutic agent includes only a single therapeutic agent. In other embodiments, the therapeutic agent can include two or more therapeutic agents for simultaneous or sequential release.

In some embodiments, the therapeutic agent is or includes an analgesic agent. The term “analgesic agent” or “analgesic” includes one or more local or systemic agents that are administered to reduce, prevent, alleviate, or remove pain entirely. The analgesic agent may comprise a systemic and/or local anesthetic, narcotics, and/or anti-inflammatory agents. The analgesic agent can include the pharmacologically active drug or a pharmaceutically acceptable salt thereof. Suitable analgesic agents include, but are not limited to, bupivacaine (e.g., bupivacaine hydrochloride monohydrate, bupivacaine hydrochloride, bupivacaine free base), ropivacaine, mepivacaine, etidocaine, levobupivacaine, trimecaine, carticaine, articaine, lidocaine, prilocaine, benzocaine, procaine, tetracaine, chloroprocaine, dexamethasone, tetrodotoxin, saxitoxin, neosaxitoxin, capsaicin, and combinations thereof.

In some embodiments, the therapeutic agent includes narcotics, for example, cocaine or anti-inflammatory agents. Examples of appropriate anti-inflammatory agents include steroids, such as prednisone, betamethasone, cortisone, dexamethasone, hydrocortisone, and methylprednisolone. Other appropriate anti-inflammatory agents include non-steroidal anti-inflammatory drugs (NSAIDs), such as aspirin, ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, and other COX-2 inhibitors, and combinations thereof.

In some embodiments, the therapeutic agent is or includes an antibiotic, an antimicrobial or antifungal agent, or combinations thereof. For example, suitable antibiotics and antimicrobials include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline, minocycline, tigecycline, doxycycline, rifampin, triclosan, chlorhexidine, penicillin, aminoglycides, quinolones, fluoroquinolones, vancomycin, gentamycin, cephalosporin, carbapenem, imipenem, ertapenem, antimicrobial peptides, cecropin-mellitin, magainin, dermaseptin, cathelicidin, α-defensins, and α-protegrins. Antifungal agents include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griseofulvin, haloprogin, butenafine, tolnaftate, nystatin, cyclohexamide, ciclopirox, flucytosine, terbinafine, and amphotericin B.

In some embodiments, the therapeutic agent is or includes an adrenocorticostatic, a β-adrenolytic, an androgen or antiandrogen, an antianemic, an antiparasitic, an anabolic, an anesthetic or analgesic, an analeptic, an antiallergic, an antiarrhythmic, an anti-arteriosclerotic, an antibiotic, an antidiabetic, an antifibrinolytic, an anticonvulsive, an angiogenesis inhibitor, an anticholinergic, an enzyme, a coenzyme or a corresponding inhibitor, an antihistaminic, an antihypertensive, an antihypotensive, an anticoagulant, an antimycotic, an antiseptic, an anti-infective, an antihemorrhagic, a β-receptor antagonist, a calcium channel antagonist, an antimyasthenic, an antiphlogistic, an antipyretic, an antirheumatic, a cardiotonic, a chemotherapeutic, a coronary dilator, a cytostatic, a glucocorticoid, a hemostatic, an immunoglobulin or its fragment, a chemokine, a cytokine, a mitogen, a cell differentiation factor, a cytotoxic agent, a hormone, an immunosuppressant, an immunostimulant, a morphine antagonist, an muscle relaxant, a narcotic, a vector, a peptide, a (para)sympathicomimetic, a (para)sympatholytic, a protein, a cell, a selective estrogen receptor modulator (SERM), a sedating agent, an antispasmodic, a substance that inhibits the resorption of bone, a vasoconstrictor or vasodilator, a virustatic, or a wound-healing agent. In some embodiments, the therapeutic agent can include a hemostatic agent (e.g., aluminum sulfate, fibrin, micronized gelfoam, etc.), which can be especially beneficial when implanting the depot in areas with high vascular flow and potentially above-average post-operative bleeding (e.g., thoracic, abdominal, anorectal, head and neck, etc.).

In some embodiments, the therapeutic agent is or includes a drug used in the treatment of cancer or a pharmaceutically acceptable salt thereof. Such chemotherapeutic agents include antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, vindesine, and tamoxifen.

In some embodiments, the therapeutic agent is or includes a botulinum toxin or other neurotoxin used in the treatment of various neuromuscular and/or neuroglandular disorders and neuropathies associated with pain. The botulinum toxin or other neurotoxin can include the pharmacologically active drug or a pharmaceutically acceptable salt thereof. The botulinum toxin can be selected from a variety of strains of Clostridium botulinum and may comprise the pharmacologically active drug or a pharmaceutically acceptable salt thereof. In some embodiment, the botulinum toxin is selected from the group consisting of botulinum toxin types A, B, C, D, E, F, and G.

A pharmaceutically acceptable salt refers to those salts that retain the biological effectiveness and properties of neutral therapeutic agents and that are not otherwise unacceptable for pharmaceutical use. Pharmaceutically acceptable salts include salts of acidic or basic groups, which groups may be present in the therapeutic agents. The therapeutic agents used in the present technology that are basic in nature are capable of forming a wide variety of salts with various inorganic and organic acids. Pharmaceutically acceptable acid addition salts of basic therapeutic agents used in the present technology can include those that form non-toxic acid addition salts, i.e., salts comprising pharmacologically acceptable anions, such as hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (1,1′-methylene-bis-(2-hydroxy-3-naphthoate)) salts. The therapeutic agents of the present technology that include an amino moiety can form pharmaceutically acceptable salts with various amino acids, in addition to the acids mentioned above. Suitable base salts can be formed from bases which form non-toxic salts, and can include aluminum, calcium, lithium, magnesium, potassium, sodium, zinc, or diethanolamine salts.

A pharmaceutically acceptable salt can include another molecule, such as water or another biologically compatible solvent (a solvate), an acetate ion, a succinate ion, or other counterion. The counterion can be any organic or inorganic moiety that stabilizes the charge on the parent compound. Optionally, a pharmaceutically acceptable salt can include multiple counterions.

The therapeutic agent or pharmaceutically acceptable salt thereof can be an essentially pure compound, or can be formulated with a pharmaceutically acceptable carrier such as diluents, adjuvants, excipients, or vehicles known to one skilled in the art. The carrier(s) can be “acceptable” in the sense of being compatible with the other ingredients of the formulations and not deleterious to the recipient thereof. For example, diluents can include lactose, dextrose, sucrose, mannitol, sorbitol, cellulose, glycine, and the like.

The therapeutic agent or pharmaceutically acceptable salt form can be micronized, jet milled, or passed through a sieve to form consistent particle sizes, which can further facilitate the controlled release of the therapeutic agent. This process can be helpful for highly insoluble therapeutic agents, for example. In some embodiments, the particle size of the therapeutic agent (e.g., the D50 value) is less than or equal to 500 μm, 450 μm, 400 μm, 350 μm, 300 μm, 250 μm, 200 μm, 150 μm, 100 μm, 90 μm, 80 μm, 70 μm, 60 μm, 50 μm, 40 μm, 30 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 km.

Suitable dosage ranges utilizing the depot of the present technology are dependent on the potency of the particular therapeutic agent, but can be within a range from about 0.001 mg to about 500 mg of drug per kilogram body weight, for example, within a range from about 0.1 mg to about 200 mg of drug per kilogram body weight, or within a range from about 1 to about 100 mg per kg body weight. Dosage ranges may be readily determined by methods known to one skilled in the art. Dosage unit forms can contain between about 1 mg to about 500 mg of active ingredient.

In some embodiments, the therapeutic agent constitutes at least 10%, 15%, 20%, 25%, 30%, 35%, 40%, 45%, 50%, 55%, 60%, 65%, 70%, 75%, 80%, 85%, 90%, 95%, or 100% of the total mass of the depot (also referred to herein as the “mass percent” or “weight percent” of the therapeutic agent in the depot). The mass percent of the therapeutic agent in the depot can be within a range from 25% to 75%, 40% to 80%, 50% to 65%, or 60% to 65%. In some embodiments, the therapeutic agent constitutes at least 10%, 15%, 20%, 25%, 30%, 35%, 40%, 45%, 50%, 55%, 60%, 65%, 70%, 75%, 80%, 85%, 90%, 95%, or 100% of the total mass of the therapeutic region. The mass percent of the therapeutic agent in the therapeutic region can be within a range from 25% to 75%, 40% to 80%, 50% to 65%, or 60% to 65% of the total mass of the therapeutic region.

In some embodiments, the depots described herein have a total mass (e.g., total dry mass) within a range from 100 mg to 1500 mg, 100 mg to 1000 mg, 100 mg to 500 mg, 300 mg to 500 mg, 500 mg to 1000 mg, or 800 mg to 1000 mg. For example, the total mass can be greater than or equal to 100 mg, 150 mg, 200 mg, 250 mg, 300 mg, 350 mg, 400 mg, 425 mg, 450 mg, 475 mg, 500 mg, 525 mg, 550 mg, 575 mg, 600 mg, 650 mg, 700 mg, 750 mg, 800 mg, 825 mg, 850 mg, 875 mg, 900 mg, 925 mg, 950 mg, 975 mg, or 1000 mg.

The total mass of the therapeutic agent within the depot can be within a range from 100 mg to 1800 mg, 100 mg to 1500 mg, 100 mg to 1000 mg, 200 mg to 800 mg, 300 mg to 600 mg, 500 mg to 700 mg, 540 mg to 660 mg, or 570 mg to 630 mg. In some embodiments, the total mass of the therapeutic agent within an individual depot is greater than or equal to 25 mg, 50 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, or 1800 mg.

The properties of the therapeutic agent can be selected to provide a desired release profile in vivo. For example, the therapeutic agent can be sufficiently hydrophobic to elute from the depot in a controlled, sustained manner when exposed to physiologic fluids at a treatment site in vivo, even when the depot includes fewer control regions (e.g., the depot 100b of FIG. 1B) or no control regions (e.g., the depot 100c of FIG. 1C). In some embodiments, the therapeutic agent has multiple forms with varying degrees of hydrophobicity, such as at least one hydrophobic form and at least one hydrophilic form. For example, the therapeutic agent can be or include an amine compound having a hydrophobic free base form and a hydrophilic salt form. The amine compound can be an amine-containing analgesic, such as an amino amide local anesthetic (e.g., bupivacaine, ropivacaine, lidocaine, mepivacaine, prilocaine, etidocaine, levobupivacaine, trimecaine, articaine) or an amino ester local anesthetic (e.g., benzocaine, procaine, tetracaine, chloroprocaine). The amine-containing analgesic can have a free base form (e.g., bupivacaine free base) in which the amine group is deprotonated, and a salt form (e.g., bupivacaine hydrochloride, bupivacaine hydrochloride monohydrate) in which the amine is protonated and associated with a counterion (e.g., chloride, bromide, sulfate, phosphate, nitrate, acetate, oxalate, citrate, tartrate). As another example, the hydrophobic form can be a salt form of the therapeutic agent that uses a relatively hydrophobic salt (e.g., a palmitate salt rather than a chloride salt). The amine-containing analgesic can contain salt forms of varying counterion combinations that alter the hydrophobicity and dissolution rate of the amine-containing analgesic.

The therapeutic agent in the implantable depot can be provided partially or entirely in the hydrophobic (e.g., free base) form. For example, at least 10%, 20%, 30%, 40%, 50%, 55%, 60%, 65%, 70%, 75%, 80%, 85%, 90%, 95%, 99%, 99.5%, or 100% of the therapeutic agent by mass can be in the hydrophobic form. Alternatively or in combination, no more than 95%, 90%, 85%, 80%, 75%, 70%, 65%, 60%, 55%, 50%, 40%, 30%, or 20% of the therapeutic agent by mass can be in the hydrophobic form. Optionally, the implantable depot can include a combination of a hydrophobic form and a hydrophilic form of the therapeutic agent. The relative amounts of the hydrophobic form and the hydrophilic form can be selected to produce a desired release profile, e.g., increasing the relative amount of the hydrophobic form can produce a slower release rate, while increasing the relative amount of the hydrophilic form can produce a faster release rate. In some embodiments, the ratio of the total mass of the hydrophobic form to the total mass of the hydrophilic form is greater than or equal to 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or 20:1.

The use of the hydrophobic form of the therapeutic agent can provide various benefits. For example, when the hydrophobic form is used, the depot can be fabricated with fewer control regions or even no control regions, thus simplifying the manufacturing process and/or reducing the likelihood of inadvertent uncontrolled release due to manufacturing defects in the control regions. This approach can also increase the amount of therapeutic agent that can be loaded into a single depot and/or decrease the depot size. Additionally, when the hydrophobic form of the therapeutic agent is used in combination with control regions, the release duration of the therapeutic agent can be extended even further, thus allowing for controlled release over extended time periods that would otherwise be difficult or impossible with conventional systems. Moreover, the hydrophobic form may exhibit a different melting point (e.g., a lower melting point) and/or different solubility (e.g., improved solubility in organic solvents) than the hydrophilic form. This may make manufacturing of the depot easier and/or allow for additional manufacturing options, compared to depots formulated primarily or entirely out of the hydrophilic form.

In some embodiments, the implantable depot is configured to release multiple therapeutic agents in a simultaneous or sequential manner, e.g., to provided added clinical benefits. For example, in the context of pain management, the depot can release a first analgesic having a faster onset (e.g., lidocaine) and a second analgesic having a slower onset (e.g., bupivacaine). As another example, the depot can release a first therapeutic agent having a first type of therapeutic effect (e.g., an analgesic effect), and a second therapeutic agent having a second type of therapeutic effect (e.g., increasing or decreasing blood flow, reducing inflammation, altering water uptake, affecting pH within the depot and/or in the surrounding environment). The second therapeutic agent can enhance the efficacy of the first therapeutic agent or can independently provide a therapeutic benefit for the patient. The implantable depots described herein can include any suitable number of therapeutic agents, such as one, two, three, four, five, or more different therapeutic agents.

### 2. Polymers

The depots of the present technology (e.g., the depots 100a-570 of FIGS. 1A-5H) can be made of one or more polymers. In some embodiments, the therapeutic region and the control regions of a depot each include a polymer (or combination of polymers), which can be the same or different polymer (or combination of polymers) in the same or different amount, concentration, and/or mass percentage. In some embodiments, the control regions include a polymer and therapeutic region does not include a polymer. In some embodiments, the therapeutic region includes a polymer and the control regions do not include a polymer.

In some embodiments, the polymer(s) used in the depots of the present technology are bioresorbable polymers. The bioresorbable polymers used in the present technology can have a predetermined degradation rate. The terms “bioresorbable” or “bioabsorbable” can mean that a polymer will be absorbed within the patient's body, for example, by a cell or tissue. These polymers can be “biodegradable” in that all or parts of the polymer will degrade over time by the action of enzymes, by hydrolytic action, and/or by other similar mechanisms in the patient's body. In some embodiments, the bioresorbable polymer breaks down or degrades within the body to non-toxic components while a therapeutic agent is being released. Bioresorbable polymers used as base components of the depots of the present technology may break down or degrade after the therapeutic agent is fully released. The bioresorbable polymers can also be “bioerodible,” in that they will erode or degrade over time due, at least in part, to contact with substances found in the surrounding tissue, fluids or by cellular action.

Suitable polymers for use in the depots of the present technology include, but are not limited to: polyglycolide (PGA), polylactide (PLA) (e.g., poly(L-lactic acid) (PLLA), poly(D-lactic acid) (PDLA), meso-poly(lactic acid), poly(D,L-lactic acid) (PDLLA), poly(L-lactide-co-D,L-lactide) (PLDLLA)), poly(lactide-co-glycolide) (PLGA) (e.g., poly(L-lactide-co-glycolide), poly(D,L-lactide-co-glycolide)), PLA-PLGA, polycaprolactone (PCL), poly(glycolide-co-caprolactone) (PGCL), poly(lactide-co-caprolactone) (PLCL), poly(DL-lactide-co-caprolactone) (DL-PLCL), poly(α-hydroxy acid) (PAHA), poly(trimethylene carbonate) (PTMC), polydioxanone (PDO), polyhydroxyalkanoate (PHA), polyhydroxybutyrate (PHB) (e.g., poly(4-hydroxy butyrate)), poly(phosphazene) (e.g., ethyl glycinate poly(phosphazene)), poly(phosphate ester), poly(amino acid), poly(depsipeptide), poly(butylene succinate) (PBS), poly(ethylene oxide) (PEO), poly(propylene oxide) (PPO), poly(ethylene glycol) (PEG), a poloxamer (e.g., PEO-PPO-PEO), a PEO-PPO-poly(acrylic acid) copolymer (PEO-PPO-PAA), PLGA-PEO-PLGA, PEG-PLG, PEG-PLGA-PEG, poly(vinylpyrrolidone) (PVP), polyvinyl alcohol (PVA), PVA-grafted PLGA (PVA-g-PLGA), poly(N-isopropylacrylamide), poly(methacrylate), poly(hydroxyethylmethacrylate), poly(methoxyethylmethacrylate), poly(methoxyethoxy-ethylmethacrylate), polymethylmethacrylate (PMMA), poly(propylene fumarate), poly(iminocarbonate), poly(glycolide-co-trimethylene carbonate), poly(ethyl glutamate-co-glutamic acid), poly(tert-butyloxy-carbonylmethyl glutamate), poly(glycerol sebacate), tyrosine-derived polycarbonate, poly(1,3-bis-(p-carboxyphenoxy) hexane-co-sebacic acid), poly(caprolactone co-butylacrylate), a copolymer of maleic anhydride, cellulose or a cellulose derivative (e.g., hydroxypropyl cellulose, hydroxypropyl methylcellulose, hydroxyethyl methylcellulose, carboxymethylcellulose or a salt thereof), a copolymer of poly(ethylene glycol terephthalate) and poly(butylene terephthalate) (PEGT-PBT) (e.g., PolyActive), a polysaccharide (e.g., hyaluronic acid, chitosan, starch, pre-gelatinized starch, alginate, dextran), sucrose acetate isobutyrate (SAIB), poly(aspirin), a polymer incorporating vitamin E or a vitamin E analog (e.g., alpha tocopheryl acetate, D-alpha tocopheryl succinate), Carbopol®, or a protein (e.g., gelatin, collagen, albumin), or a copolymer derivative, or combination thereof.

Optionally, the polymers described herein can be modified to include functional side groups or chains. For example, the polymer can be grafted with, crosslinked to, or otherwise covalently bonded to a hydrophilic side chain, such as PEG. This approach can be advantageous for ensuring consistent, controlled release of the therapeutic agent. In some situations, when the therapeutic agent elutes from the therapeutic region, the voids or spaces in the polymer that were previously occupied by the therapeutic agent may collapse to form partially or completely impermeable polymer regions. If the collapse occurs near the portions of the therapeutic region that are in contact with physiologic fluid, this can create a barrier that partially or completely inhibits further elution of therapeutic agent from those locations. However, polymers including hydrophilic side chains can swell when exposed to fluid, thus reducing the likelihood of collapse and allowing continued release of the therapeutic agent.

In some embodiments, the properties of the polymer are selected to modulate the release profile of the therapeutic agent from the depot. For example, the hydrophobicity or hydrophilicity of the polymer may impact water uptake into the depot, which in turn can alter the release rate of the therapeutic agent. More hydrophilic polymers (e.g., PLGA with a higher glycolic acid content, polymers incorporating PEG covalently into the polymer backbone) may produce higher release rates than more hydrophobic polymers. In some embodiments, the different end groups of the polymer can be selected to affect the hydrophilicity of the polymer. For example, polymers having an acid terminal group can be more hydrophilic than polymers having an ester terminal group.

In some embodiments, the mass percent of the polymer in the depot is no more than 10%, 15%, 20%, 25%, 30%, 35%, 40%, 45%, 50%, 55%, 60%, 65%, 70%, 75%, 80%, 85%, 90%, or 95%. The mass percent of the polymer in the depot can be within a range from 10% to 60%, 20% to 50%, 25% to 40%, or 30% to 35%. In some embodiments, the mass percent of the polymer in the therapeutic region is no more than 10%, 15%, 20%, 25%, 30%, 35%, 40%, 45%, 50%, 55%, 60%, 65%, 70%, 75%, 80%, 85%, 90%, or 95%. The mass percent of the polymer in the therapeutic region can be within a range from 10% to 60%, 20% to 50%, 25% to 40%, or 30% to 35%. In some embodiments, the mass percent of the polymer in an individual control region is at least 10%, 15%, 20%, 25%, 30%, 35%, 40%, 45%, 50%, 55%, 60%, 65%, 70%, 75%, 80%, 85%, 90%, 95%, or 100%. The mass percent of the polymer in the control region can be within a range from 25% to 75%, 40% to 80%, 50% to 65%, 60% to 65%, 50% to 75%, or 75% to 100%.

The total mass of the polymer within the depot can be within a range from 100 mg to 1000 mg, 100 mg to 500 mg, 150 mg to 350 mg, 250 mg to 350 mg, or 300 mg to 350 mg. In some embodiments, the total mass of the polymer is less than or equal to 1000 mg, 900 mg, 800 mg, 700 mg, 600 mg, 500 mg, 475 mg, 450 mg, 425 mg, 400 mg, 375 mg, 350 mg, 325 mg, 300 mg, 275 mg, 250 mg, 225 mg, 200 mg, 175 mg, 150 mg, 125 mg, or 100 mg.

In some embodiments, the ratio of the mass of the therapeutic agent in the depot to the mass of the polymer in the depot is at least 3:1, 3.5:1, 4:1, 4.5:1, 5:1, 5.5:1, 6:1, 6.5:1, 7:1, 8:1, 9:1, 10:1, 11:1, 12:1, 13:1, 14:1, 15:1, or 16:1. In some embodiments, the ratio of the mass of the polymer in the therapeutic region to the mass of the therapeutic agent in the therapeutic region is no more than 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10.

In some embodiments, the polymers disclosed herein are configured to degrade at a sufficiently slow rate so that the depot maintains sufficient flexural strength and/or mechanical integrity in vivo for at least a predetermined period of time or until a predetermined proportion of therapeutic agent has been released from the depot. The depot can be considered to maintain its structural integrity if the depot remains largely intact with only partial or gradual reduction due to elution of therapeutic agent or dissolution of the control regions or releasing agent. The depot can be considered to lose its structural integrity if it separates (e.g., fractures) into multiple component pieces, for example, with two or more of the resulting pieces being at least 5% of the previous size of the depot. Alternatively, or additionally, the depot can be considered to lose its structural integrity if the release rate of the therapeutic agent increases by more than a factor of three as compared to the release rate of therapeutic agent in a control depot submerged in a buffered solution. In some embodiments, the molecular weight of the polymer can be selected to account for a loss in molecular weight that occurs during the manufacturing process such that the post-manufacturing molecular weight remains above a minimum weight required to achieve a desired sustained release profile.

In some embodiments, the depot is configured to maintain its structural integrity in vivo for at least a predetermined length of time. For example, the depot can be configured to maintain its structural integrity in vivo for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days. In some embodiments, the depot is configured to maintain its structural integrity in vivo until at least a predetermined proportion of therapeutic agent payload has been released from the depot. For example, the depot can be configured to maintain its structural integrity in vivo until at least 5%, 10%, 15%, 20%, 25%, 30%, 35%, 40%, 45%, 50%, 55%, 60%, 65%, 70%, 75%, 80%, 85%, 90%, 95%, or 100% of the original mass of the therapeutic agent in the depot has been released.

### 3. Releasing Agents

The depots of the present technology (e.g., the depots 100a-570 of FIGS. 1A-5H) can optionally include one or more releasing agents. In some embodiments, the therapeutic region and the control regions each include a releasing agent (or combination of releasing agents), which can be the same or different releasing agent (or combination of releasing agents) in the same or different amount, concentration, and/or mass percentage. In some embodiments, the control regions include a releasing agent and the therapeutic region does not include a releasing agent. In some embodiments, the therapeutic region includes a releasing agent and the control regions do not include a releasing agent.

In some embodiments, the releasing agent is a polysorbate, such as Polysorbate 80, Polysorbate 60, Polysorbate 40, or Polysorbate 20 (Tween 20™). Other releasing agents suitable for use in the present technology include polyethylene glycol (e.g., PEG 3000, PEG 6000, PEG 10,000, etc.), polyvinyl alcohols, sorbitan fatty acid esters (e.g., sorbitan monostearate (Span 60), sorbitan tristearate (Span 65), sorbitane trioleate (Span 85), sorbitan monooleate (Span 80), sorbitan monopalmitate, sorbitan monostearate, sorbitan monolaurate, sorbitan monopalmitate, sorbitan trioleate, sorbitan tribehenate), sucrose esters (e.g., sucrose monodecanoate, sucrose monolaurate, sucrose distearate, sucrose stearate), castor oils (e.g., polyethoxylated castor oil, polyoxyl hydrogenated castor oil, Polyoxyl 35 castor oil, Polyoxyl 40 Hydrogenated castor oil, Polyoxyl 40 castor oil, Cremophor® RH60, Cremophor® RH40), polyethylene glycol ester glycerides (e.g., Labrasol®, Labrifil® 1944), poloxamers, polyoxyethylene polyoxypropylene 1800, polyoxyethylene fatty acid esters (e.g., Polyoxyl 20 Stearyl Ether, diethylene glycol octadecyl ether, glyceryl monostearate, triglycerol monostearate, Polyoxyl 20 stearate, Polyoxyl 40 stearate, polyoxyethylene sorbitan monoisostearate, polyethylene glycol 40 sorbitan diisostearate), oleic acid, sodium desoxycholate, sodium lauryl sulfate, myristic acid, stearic acid, vitamin E D-alpha-tocopherol polyethylene glycol succinate (vitamin E-TPGS), saturated polyglycolized glycerides (e.g., Gelucire® 44/14, Gelucire® 50/13), polypropoxylated stearyl alcohols (e.g., Acconon® MC-8, Acconon® CC-6), or derivatives or combinations thereof.

In some embodiments, the mass percent of the releasing agent in the depot is no more than 50%, 20%, 15%, 10%, 9%, 8%, 7%, 6%, 5%, 4.5%, 3.5%, 3%, 2.5%, 2%, 1.5%, 1%, 0.75%, 0.5%, 0.25%, or 0.1%. The mass percent of the releasing agent in the depot can be within a range from 0.1% to 20%, 0.5% to 10%, or 1% to 5%. In some embodiments, the mass percent of the releasing agent in the therapeutic region is no more than 50%, 20%, 15%, 10%, 9%, 8%, 7%, 6%, 5%, 4.5%, 3.5%, 3%, 2.5%, 2%, 1.5%, 1%, 0.75%, 0.5%, 0.25%, or 0.1%. The mass percent of the releasing agent in the therapeutic region can be within a range from 0.1% to 20%, 0.5% to 10%, or 1% to 5%. In some embodiments, the mass percent of the releasing agent in an individual control region is no more than 50%, 45%, 40%, 35%, 30%, 25%, 20%, 15%, 10%, 9%, 8%, 7%, 6%, 5%, 4.5%, 3.5%, 3%, 2.5%, 2%, 1.5%, 1%, 0.75%, 0.5%, 0.25%, or 0.1%. The mass percent of the releasing agent in the control region can be within a range from 0.1% to 20%, 0.5% to 10%, 1% to 5%, 10% to 50%, 20% to 40%, or 30% to 35%.

The total mass of the releasing agent in the depot can be within a range from 1 mg to 200 mg, 10 mg to 100 mg, 10 mg to 50 mg, 20 mg to 50 mg, 20 mg to 40 mg, or 25 mg to 35 mg. In some embodiments, the total mass of the releasing agent is less than or equal to 200 mg, 150 mg, 100 mg, 90 mg, 80 mg, 70 mg, 60 mg, 50 mg, 45 mg, 40 mg, 35 mg, 30 mg, 25 mg, 20 mg, 15 mg, 10 mg, 5 mg, or 1 mg.

In some embodiments, the ratio of the mass of the releasing agent to the mass of the polymer in therapeutic region is no more than 1:1, 1:1.5, 1:2, 1:3, 1:4, 1:5, 1:6, 1:7, 1:8, 1:9, 1:10, 1:11, 1:12, 1:13, 1:14, 1:15, or 1:16. In some embodiments, the ratio of the mass of the releasing agent to the mass of the polymer to the mass of the therapeutic agent in the therapeutic region is within a range from 0.1:10:20 to 2:10:20, from 0.1:10:20 to 1:10:20, from 0.1:10:20 to 0.5:10:20, from 0.5:10:20 to 0.1:10:20, from 0.5:10:20 to 1:10:20, from 1:10:20 to 10:10:20, from 1:10:20 to 5:10:20, from 2:10:20 to 5:10:20, or from 5:10:20 to 10:10:20. In other embodiments, the therapeutic region may not include any releasing agent.

In some embodiments, the ratio of the mass of the releasing agent to the mass of the polymer in an individual control region is at least 2:1, 1.5:1, 1:1, 1:1.5, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, or 1:25. In other embodiments, the control region may not include any releasing agent.

### 4. Additives

The depots of the present technology (e.g., the depots 100a-570 of FIGS. 1A-5H) can optionally include one or more additives. The additive can be any component that influences the characteristics and/or performance of the depot, such as the release profile, mechanical properties (e.g., flexibility, strength, surface roughness), shelf stability, degradation rate, etc. In some embodiments, the therapeutic region and the control regions each include an additive (or combination of additives), which can be the same or different additive (or combination of additives) in the same or different amount, concentration, and/or mass percentage. In some embodiments, the control regions include an additive and the therapeutic region does not include an additive. In some embodiments, the therapeutic region includes an additive and the control regions do not include an additive.

For example, in some embodiments, the additive is or includes a plasticizer or other suitable excipient that increases the flexibility of the depot, makes it easier to form the depot into a desired shape, and/or reduces friction on one or more surfaces of the depot. Advantages of flexible depots can include, for example, improved patient comfort (e.g., the patient is less likely to feel the depot after implantation), reducing tissue irritation at the implant site (e.g., for soft tissue applications or bony applications where soft tissue is present around the implant site), allowing the depot to mold and/or flex around hard structures at the implant site (e.g., bone, other implanted devices), allowing the depot to bend to fit into tight spaces (e.g., trocars for minimally invasive procedures, small spaces within the patient's body), and/or reducing the likelihood of damage to the depot during manufacturing and/or surgical procedures. Moreover, in embodiments where the implantable depot includes a therapeutic region only, the absence of any control regions may make the depot more susceptible to fracturing. In such embodiments, the addition of a plasticizer can improve the flexibility of the therapeutic region to reduce the likelihood of fracture during manufacturing, handling, etc. For example, the depots herein may be bent at an angle of at least 5°, 10°, 15°, 20°, 30°, or 45° without fracturing.

In some embodiments, the depots herein have a flexural modulus less than or equal to 600 MPa, 500 MPa, 400 MPa, 300 MPa, 200 MPa, 100 MPa, 75 MPa, 50 MPa, 40 MPa, 30 MPa, 20 MPa, or 10 MPa; and/or within a range from 1 MPa to 5 MPa, 1 MPa to 10 MPa, 1 MPa to 20 MPa, 1 MPa to 50 MPa, 1 MPa to 100 MPa, 1 MPa to 400 MPa, 5 MPa to 10 MPa, 5 MPa to 20 MPa, 5 MPa to 50 MPa, 5 MPa to 100 MPa, 5 MPa to 400 MPa, 10 MPa to 20 MPa, 10 MPa to 50 MPa, 10 MPa to 100 MPa, 10 MPa to 400 MPa, 20 MPa to 50 MPa, 20 MPa to 100 MPa, 20 MPa to 400 MPa, 50 MPa to 100 MPa, 50 MPa to 400 MPa, or 100 MPa to 400 MPa. The flexural modulus of a depot can be measured, for example, using a three-point bending test at room temperature (e.g., 20-25° C.) or physiological temperature (e.g., 37° C.). In some embodiments, the depot remains flexible for an extended period of time post-manufacturing. For instance, the flexural modulus of the depot can be maintained for at least 1 day, 2 days, 5 days, 7 days, 10 days, 14 days, 1 month, 2 months, 3 months, 4 months, 5 months, 6 months, or 1 year post-manufacturing (e.g., when stored at room temperature). In some embodiments, the flexural modulus is maintained for at least 1 day, 2 days, 5 days, 7 days, 10 days, 14 days, 20 days, 30 days, or 1 month post-manufacturing under accelerated aging conditions (e.g., 40° C.).

The plasticizer can be a non-volatile or low-volatility liquid, or a solid substance. The plasticizer can have any suitable molecular weight, such as a molecular weight less than or equal to 20 kDa, 10 kDa, 5 kDa, 2 kDa, 1 kDa, 900 Da, 800 Da, 700 Da, 600 Da, 500 Da, 400 Da, 300 Da, 200 Da, or 100 Da. The plasticizer can be or include a hydrophobic (water insoluble) substance, such as a triglyceride, (e.g., Miglyol, tricaprilin), a fatty acid ester (e.g., ethyl hexanoate, isopropyl palmitate, isopropyl myristate), a lactic acid ester (e.g., lactic acid doecyl ester), a citrate (e.g., acetyltriethyl citrate, tributyl citrate, acetyltributyl citrate (O-acetyl citrate)), diethyl phthalate (DEP), dibutyl sebacate, acetylated monoglyceride, or benzyl benzoate. The plasticizer can be or include a hydrophilic (water soluble) substance, such as triethyl citrate, a polyethylene glycol, a polysorbate, a propylene glycol, glycerol triacetate (triacetin), benzyl alcohol, glycerol or a glycerol derivative (e.g., glycerol formal), or ethyl lactate. In some embodiments, the plasticizer is selected from the group consisting of a triglyceride (e.g., Miglyol, tricaprilin), a fatty acid ester (e.g., ethyl hexanoate, ethyl lactate, isopropyl palmitate, isopropyl myristate), a lactic acid ester (e.g., lactic acid doecyl ester), a citrate (e.g., acetyltriethyl citrate, tributyl citrate, acetyltributyl citrate (O-acetyl citrate)), a phthalate (e.g., diethyl phthalate), a glycerol ester (e.g., glycerol triacetate (triacetin)), a sebacate (e.g., dibutyl sebacate), a monoglyceride ester (e.g., acetylated monoglyceride), a benzyl derivative (e.g., benzyl benzoate, benzyl alcohol), a polyethylene glycol (e.g., a polypropylene glycol), a polysorbate (e.g., polysorbate 20, polysorbate 80), a diol, and a triol (e.g., glycerol).

In some embodiments, the plasticizer is selected to be miscible and/or at least partially soluble with the polymer(s) of the depot. The miscibility and/or solubility between the plasticizer and the polymer may influence the extent of the plasticizing effect, e.g., the degree to which the flexibility of the depot is enhanced by addition of the plasticizer. If the plasticizer is miscible with and/or sufficiently soluble in the polymer, the plasticizer can occupy intermolecular spaces between polymer chains, reducing the intermolecular forces along the polymer chains, expanding the intermolecular spacing and free volume, and thus increasing the polymer chain mobility and/or decreasing the glass transition temperature (T_{g}) of the polymer. Miscibility and/or solubility can be estimated based on the Hansen solubility parameters (HSPs) of the plasticizer and polymer, for example. The HSPs can be used to calculate the relative energy difference (RED) between the plasticizer and polymer in accordance with techniques known to those of skill in the art (e.g., as described in Vebber et al., J. Appl. Polym. Sci. 2014, 131, 39696, which is incorporated by reference herein in its entirety). Specifically, the RED can be calculated from the equation

$$RED = \frac{{R}_{a}}{{R}_{0}} ,$$

where Rₐ is the HSP distance between the plasticizer and polymer (which can be calculated from the individual HSPs of the plasticizer and polymer), and R₀ is the interaction radius of the polymer. RED<1 indicates that the plasticizer and polymer will likely be soluble with each other, RED=1 indicates that plasticizer and polymer will likely be partially soluble with each other, and RED>1 indicates that the plasticizer and polymer will likely be insoluble with each other. In some embodiments, the plasticizer and polymer of the depots described herein have a RED less than or equal to 1.5, 1.4, 1.3, 1.2, 1.1, 1, 0.9, 0.8, 0.7, 0.6, 0.5, 0.4, 0.3, 0.2, or 0.1.

In some embodiments, the plasticizer is selected to avoid leaching from the depot during and/or after manufacturing. Leaching may be correlated to the vapor pressure of the plasticizer, e.g., plasticizers with a higher vapor pressure are more volatile and thus may have a greater tendency to migrate to the surface of the depot and evaporate to the surrounding environment. In some embodiments, the plasticizer used in the depots described herein have a vapor pressure less than or equal to 1 Pa, 0.9 Pa, 0.8 Pa, 0.7 Pa, 0.6 Pa, 0.5 Pa, 0.4 Pa, 0.3 Pa, 0.2 Pa, or 0.1 Pa at 25° C. Immiscibility and/or low solubility between the plasticizer and polymer may also promote plasticizer leaching.

The characteristics of the plasticizer can be selected to provide a desired release profile for the therapeutic agent while conferring flexibility to the depot. For instance, more hydrophilic plasticizers may promote uptake of water into the depot, which may increase the release rate of the therapeutic agent. Conversely, more hydrophobic plasticizers may reduce uptake of water into the depot, which may decrease the release rate of the therapeutic agent. The hydrophobicity/hydrophilicity of the plasticizer can also influence miscibility with the polymer, in that hydrophilic plasticizers may be miscible with hydrophilic polymers, while hydrophobic plasticizers may be miscible with hydrophobic polymers. The hydrophobicity of the plasticizer and polymer can be quantified based on their log P values, with higher log P corresponding to greater hydrophobicity and lower log P values corresponding to greater hydrophilicity. In some embodiments, the plasticizer used in the depots described herein has a log P value within a range from −1.5 to 6, 0 to 4, or 2 to 4.

In some embodiments, the depot includes a single type of plasticizer. For example, the single plasticizer can be triacetin, diethyl phthalate, or benzyl benzoate. Alternatively, the depot can include a plurality of different types of plasticizers, such as two, three, four, five, or more different types of plasticizers. For example, a depot may include a first plasticizer, such as triacetin, and a second plasticizer, such as glycerol. The first plasticizer can be a “primary” plasticizer that is miscible with and/or highly soluble in the polymer, and the second plasticizer can be a “secondary” plasticizer that is immiscible with and/or less soluble in the polymer. A secondary plasticizer can provide additional enhancements to depot properties such as improving or maintaining flexibility, modulating drug release kinetics, etc. Optionally, a depot may include additional plasticizers, such as a third plasticizer, fourth plasticizer, etc. For example, a depot may include a first plasticizer, such as triacetin, a second plasticizer, such as benzyl benzoate, and a third plasticizer, such as glycerol. One or more plasticizers may be present in any suitable portion of the depot, such as the therapeutic region only, the control region(s) only, or both the therapeutic region and the control region(s). The therapeutic region may have the same plasticizer(s) as the control region(s) or may have different plasticizer(s) than the control region(s).

In some embodiments, the mass percent of the plasticizer(s) in the depot, collectively or individually, is greater than or equal to 0.1%, 0.25%, 0.5%, 0.75%, 1%, 1.5%, 2%, 2.5%, 3%, 3.5%, 4%, 4.5%, 5%, 6%, 7%, 8%, 9%, 10%, 14%, 15%, or 20%; and/or is no more than 20%, 15%, 14%, 10%, 9%, 8%, 7%, 6%, 5%, 4.5%, 3.5%, 3%, 2.5%, 2%, 1.5%, 1%, 0.75%, 0.5%, 0.25%, or 0.1%; and/or is within a range from 0.1% to 20%, 0.5% to 10%, 1% to 20%, 1% to 15%, 1% to 10%, 1% to 5%, 5% to 20%, 5% to 15%, 5% to 10%, 10% to 20%, or 10% to 15%. In some embodiments, the ratio of plasticizer(s) to polymer by mass in the depot, collectively or individually, is greater than or equal to 0.5:10, 1:10, 1.5:10, 2:10, 2.5:10, 3:10, 3.5:10, 4:10, 4.5:10, 5:10, 6:10, 7:10, 8:10, 9:10, or 10:10; and/or is less than or equal to 10:10, 9:10, 8:10, 7:10, 6:10, 5:10:4.5:10, 4:10, 3.5:10, 3:10, 2.5:10, 2:10, 1.5:10, 1:10, or 0.5:10.

In some embodiments, the mass percent of the plasticizer(s) in the therapeutic region, collectively or individually, is greater than or equal to 0.1%, 0.25%, 0.5%, 0.75%, 1%, 1.5%, 2%, 2.5%, 3%, 3.5%, 4%, 4.5%, 5%, 6%, 7%, 8%, 9%, 10%, 14%, 15%, or 20%; and/or is no more than 20%, 15%, 14%, 10%, 9%, 8%, 7%, 6%, 5%, 4.5%, 3.5%, 3%, 2.5%, 2%, 1.5%, 1%, 0.75%, 0.5%, 0.25%, or 0.1%; and/or is within a range from 0.1% to 20%, 0.5% to 10%, 1% to 20%, 1% to 15%, 1% to 10%, 1% to 5%, 5% to 20%, 5% to 15%, 5% to 10%, 10% to 20%, or 10% to 15%. In some embodiments, the ratio of plasticizer(s) to polymer by mass in the therapeutic region, collectively or individually, is greater than or equal to 0.5:10, 1:10, 1.5:10, 2:10, 2.5:10, 3:10, 3.5:10, 4:10, 4.5:10, 5:10, 6:10, 7:10, 8:10, 9:10, or 10:10; and/or is less than or equal to 10:10, 9:10, 8:10, 7:10, 6:10, 5:10:4.5:10, 4:10, 3.5:10, 3:10, 2.5:10, 2:10, 1.5:10, 1:10, or 0.5:10. In other embodiments, however, the therapeutic region may not include any plasticizers.

In embodiments where the depot includes one or more control regions, the mass percent of the plasticizer(s) in an individual control region, collectively or individually, can be greater than or equal to 0.1%, 0.25%, 0.5%, 0.75%, 1%, 1.5%, 2%, 2.5%, 3%, 3.5%, 4%, 4.5%, 5%, 6%, 7%, 8%, 9%, 10%, 14%, 15%, or 20%; and/or can be no more than 20%, 15%, 14%, 10%, 9%, 8%, 7%, 6%, 5%, 4.5%, 3.5%, 3%, 2.5%, 2%, 1.5%, 1%, 0.75%, 0.5%, 0.25%, or 0.1%; and/or can be within a range from 0.1% to 20%, 0.5% to 10%, 1% to 20%, 1% to 15%, 1% to 10%, 1% to 5%, 5% to 20%, 5% to 15%, 5% to 10%, 10% to 20%, or 10% to 15%. In some embodiments, the ratio of plasticizer(s) to polymer by mass in an individual control region, collectively or individually, is greater than or equal to 0.5:10, 1:10, 1.5:10, 2:10, 2.5:10, 3:10, 3.5:10, 4:10, 4.5:10, 5:10, 6:10, 7:10, 8:10, 9:10, or 10:10; and/or is less than or equal to 10:10, 9:10, 8:10, 7:10, 6:10, 5:10:4.5:10, 4:10, 3.5:10, 3:10, 2.5:10, 2:10, 1.5:10, 1:10, or 0.5:10. In other embodiments, however, the control region(s) may not include any plasticizers.

In embodiments where the depot includes a first plasticizer (e.g., triacetin) and a second plasticizer (e.g., glycerol), the ratio of the first plasticizer to the second plasticizer by mass can be greater than or equal to 20:1, 15:1, 10:1, 9:1, 8:1, 7:1, 6:1, 5:1, 4:1, 3:1, 2:1, 1:1, 1:2, 3:1, 4:1, 5:1, 6:1, 7:1, 8:1, 9:1, 10:1, 15:1, or 20:1; and/or can be no more than 20:1, 15:1, 10:1, 9:1, 8:1, 7:1, 6:1, 5:1, 4:1, 3:1, 2:1, 1:1, 1:2, 3:1, 4:1, 5:1, 6:1, 7:1, 8:1, 9:1, 10:1, 15:1, or 20:1.

In embodiments where the depot includes a first plasticizer (e.g., triacetin), a second plasticizer (e.g., benzyl benzoate), and a third plasticizer (e.g., glycerol), the ratio of the first plasticizer to the second plasticizer by mass can be greater than or equal to 20:1, 15:1, 10:1, 9:1, 8:1, 7:1, 6:1, 5:1, 4:1, 3:1, 2:1, 1:1, 1:2, 3:1, 4:1, 5:1, 6:1, 7:1, 8:1, 9:1, 10:1, 15:1, or 20:1; and/or can be no more than 20:1, 15:1, 10:1, 9:1, 8:1, 7:1, 6:1, 5:1, 4:1, 3:1, 2:1, 1:1, 1:2, 3:1, 4:1, 5:1, 6:1, 7:1, 8:1, 9:1, 10:1, 15:1, or 20:1. The ratio of the second plasticizer to the third plasticizer by mass can be greater than or equal to 20:1, 15:1, 10:1, 9:1, 8:1, 7:1, 6:1, 5:1, 4:1, 3:1, 2:1, 1:1, 1:2, 3:1, 4:1, 5:1, 6:1, 7:1, 8:1, 9:1, 10:1, 15:1, or 20:1; and/or can be no more than 20:1, 15:1, 10:1, 9:1, 8:1, 7:1, 6:1, 5:1, 4:1, 3:1, 2:1, 1:1, 1:2, 3:1, 4:1, 5:1, 6:1, 7:1, 8:1, 9:1, 10:1, 15:1, or 20:1.

As described herein, the presence of the plasticizer(s) can enhance the flexibility of the depot, e.g., to allow the depot to be bent or otherwise deformed without fracturing. Alternatively or in combination, the geometry of the depot can be selected to provide improved flexibility, such as by reducing the thickness of the depot. For example, the thickness can be less than or equal to 5 mm, 4 mm, 3 mm, 2.5 mm, 2 mm, 1.5 mm, 1 mm, 0.9 mm, 0.8 mm, 0.7 mm, 0.6 mm, 0.5 mm, 0.4 mm, 0.3 mm, 0.2 mm, or 0.1 mm; and/or within a range from 0.1 mm to 5 mm, 0.1 mm to 1 mm, 0.5 mm to 2 mm, 1 mm to 2 mm, or 1.5 mm to 2.5 mm.

Other examples of additives that may be incorporated in the depots described herein include, but are not limited to: antioxidants and/or pH modifiers to increase storage stability, hydrophilic additives to increase therapeutic agent release rate and/or polymer degradation rate, and/or hydrophobic additives to decrease therapeutic agent release rate and/or polymer degradation rate.

In some embodiments, the mass percent of the additive in the depot is no more than 50%, 20%, 15%, 10%, 9%, 8%, 7%, 6%, 5%, 4.5%, 3.5%, 3%, 2.5%, 2%, 1.5%, 1%, 0.75%, 0.5%, 0.25%, or 0.1%. The mass percent of the additive in the depot can be within a range from 0.1% to 20%, 0.5% to 10%, or 1% to 5%. In some embodiments, the mass percent of the additive in the therapeutic region is no more than 50%, 20%, 15%, 10%, 9%, 8%, 7%, 6%, 5%, 4.5%, 3.5%, 3%, 2.5%, 2%, 1.5%, 1%, 0.75%, 0.5%, 0.25%, or 0.1%. The mass percent of the additive in the therapeutic region can be within a range from 0.1% to 20%, 0.5% to 10%, or 1% to 5%. In some embodiments, the mass percent of the additive in an individual control region is no more than 50%, 45%, 40%, 35%, 30%, 25%, 20%, 15%, 10%, 9%, 8%, 7%, 6%, 5%, 4.5%, 3.5%, 3%, 2.5%, 2%, 1.5%, 1%, 0.75%, 0.5%, 0.25%, or 0.1%. The mass percent of the additive in the control region can be within a range from 0.1% to 20%, 0.5% to 10%, 1% to 5%, 10% to 50%, 20% to 40%, or 30% to 35%.

### II. Release Profile and Pharmacokinetics

The depots of the present technology (e.g., the depots 100a-570 of FIGS. 1A-5H) can be configured to deliver a therapeutic agent according to a desired release profile. As described elsewhere herein, the release profile of a depot can be tuned by adjusting the geometry and/or composition of the depot. For example, depots having two control regions (e.g., the depot 100a of FIG. 1A) can exhibit slower release than depots having a single control region (e.g., the depot 100b of FIG. 1B) or no control regions (e.g., the depot 100c of FIG. 1C). The number and arrangement of control regions can affect the travel distance of the therapeutic agent, which in turn can correlate to the overall release rate of the therapeutic agent. In some embodiments, a depot having both upper and lower control regions (e.g., the depot 100a of FIG. 1A) releases the therapeutic agent primarily or entirely from the exposed lateral surfaces, such that the travel distance and/or overall release rate is determined primarily based on the lateral dimensions of the depot (e.g., width, length). In such embodiments, the presence of one or more holes can reduce the travel distance and/or increase the release rate from the depot, by providing more exposed surface area for release of the therapeutic agent. In some embodiments, a depot having no control regions (e.g., the depot 100c of FIG. 1C), releases the therapeutic agent primarily from the exposed upper and lower surfaces, such that the travel distance and/or release rate is determined primarily on the vertical dimensions of the depot (e.g., thickness).

Other factors that can influence the release rate include, but are not limited to, the characteristics of the therapeutic agent (e.g., hydrophobic therapeutic agents may release slower than hydrophilic therapeutic agents, larger particle sizes may reduce the release rate), the characteristics of the polymer (e.g., hydrophilic polymers may promote infiltration of physiological fluids into the depot, which can accelerate release), the presence of a releasing agent (e.g., release rate may be increased when a releasing agent is present), etc. Optionally, multiple depots having different geometries and/or compositions can be implanted to collectively produce a desired release profile.

The release profile can provide sustained, continuous release of the therapeutic agent over a desired treatment period or duration (e.g., the period after the depot is implanted in the body and/or immersed in fluid). The treatment period can be at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days. Alternatively or in combination, the treatment period can be less than or equal to 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days. The depots herein can release at least 70%, 75%, 80%, 85%, 90%, 95%, 96%, 97%, 98%, 99%, or 100% of the initial amount (e.g., mass) of the therapeutic agent in the depot over the treatment period.

The release profile of a depot can be measured using in vitro or in vivo techniques. Any description herein of a release profile of a depot can refer to in vitro release, in vivo release, or both, unless otherwise specified. The release profile of a depot can be measured in vitro by immersing the depot in a suitable elution medium (e.g., phosphate-buffered saline) at a controlled temperature (e.g., 37° C.) and pH (e.g., 7.4, 5.8), and measuring the amount of released therapeutic agent at various time points (e.g., using spectrophotometric techniques). When measuring in vitro release, the elution pH and/or other parameters can be configured to approximate in vivo physiologic conditions (e.g., release is measured at pH 7.4). Alternatively, the elution pH and/or other parameters can be selected based on other considerations. For example, as a product advances in development or manufacturing, an accelerated in vitro release process can be developed, e.g., to facilitate quality control testing. The accelerated in vitro release can be accomplished through an increase in temperature, the addition of a surfactant or organic co-solvent to the aqueous buffer, and/or by a change in pH. For example, accelerated in vitro release can be measured at pH 5.8.

The release profile of a depot can be measured in vivo by implanting the depot at a treatment site in a subject (e.g., an animal or human subject), collecting local and/or systemic samples from the subject at various time points (e.g., blood samples, plasma samples, synovial fluid samples), and measuring the amount of therapeutic agent in the sample (e.g., using liquid chromatography tandem mass spectrometry). Optionally, a cumulative in vivo release profile can be estimated from concentration data by assuming that the total area under the curve (e.g., AUC_{0-inf} or AUCₗₐₛₜ) of the concentration data corresponds to 100% release of the total therapeutic agent dose in the depot, then calculating the cumulative percentage release of the therapeutic agent at each study time point t₁ from the ratio of AUC₀₋ₜ₁ to the total AUC normalized to 100%. As yet another example, the in vivo release profile can be determined by explanting the depot from the treatment site at various time points, and measuring the amount of therapeutic agent remaining in the depot. For example, the depot can be immersed in an extraction medium (e.g., 5:3 v/v acetonitrile:methanol) to dissolve the depot and release any remaining therapeutic agent. The extraction medium can be fully evaporated, and the therapeutic agent can be reconstituted using a suitable solvent (e.g., methanol). The reconstituted sample can be analyzed via high-performance liquid chromatography (HPLC) to measure the amount of therapeutic agent in the sample.

In some embodiment, the depots herein are configured to release the therapeutic agent at different rates over the treatment period. For example, the depots herein can release the therapeutic agent at a first rate during a first time period of the treatment, and a second rate during a second, subsequent time period of the treatment. For example, the first period can be the first 1 day, 2 days, 3 days, 4 days, 5 days, 6 days, 7 days, 8 days, 9 days, 10 days, 11 days, 12 days, 13 days, or 14 days of the treatment period; and the second period can be the next 1 day, 2 days, 3 days, 4 days, 5 days, 6 days, 7 days, 8 days, 9 days, 10 days, 11 days, 12 days, 13 days, or 14 days after the first period. Stated differently, the first period can be the first 1 hour, 2 hours, 5 hours, 10 hours, 12 hours, 20 hours, 24 hours, 30 hours, 36 hours, 40 hours, 48 hours, 50 hours, 60 hours, 70 hours, 72 hours, 80 hours, 84 hours, 90 hours, 96 hours, 100 hours, 108 hours, 120 hours, 15o hours, 200 hours, 250 hours, 300 hours, 350 hours, 400 hours, 450 hours, or 500 hours of the treatment period; and the second period can be the next 1 hour, 2 hours, 5 hours, 10 hours, 12 hours, 20 hours, 24 hours, 30 hours, 36 hours, 40 hours, 48 hours, 50 hours, 60 hours, 70 hours, 72 hours, 80 hours, 84 hours, 90 hours, 96 hours, 100 hours, 108 hours, 120 hours, 150 hours, 200 hours, 250 hours, 300 hours, 350 hours, 400 hours, 450 hours, or 500 hours of the treatment period after the first treatment period. The first rate may be the same as or different than (e.g., less than or greater than) the second rate. In some embodiments, the first rate is at least 2-fold, 3-fold, 4-old, 5-fold, 6-fold, 7-fold, 8-fold, 9-fold, or 10-fold greater than the second rate, or vice-versa.

In some embodiments, the depot releases a first amount of the therapeutic agent over the first time period and a second amount of the therapeutic agent over the second time period. The first amount can be least 10%, 15%, 20%, 25%, 30%, 35%, 40%, 45%, 50%, 55%, or 60% of the initial amount (e.g., by mass) of the therapeutic agent in the depot; and/or the first amount can be no more than 80%, 75%, 70%, 65%, 60%, 55%, 50%, 45%, 40%, 35%, 30%, or 25% of the initial amount of the therapeutic agent in the depot. The second amount can be at least 5%, 10%, 15%, 20%, 25%, 30%, 35%, 40%, 45%, or 50% of the initial amount of the therapeutic agent in the depot; and/or the second amount can be no more than 60%, 55%, 50%, 45%, 40%, 35%, 30%, 25%, 20%, 15%, or 10% of the initial amount of the therapeutic agent in the depot. Optionally, the depot can release a third amount of the therapeutic agent over a third time period subsequent to the second time period. The third amount can be at least 1%, 2%, 5%, 10%, 15%, 20%, 25%, or 30% of the initial amount of the therapeutic agent in the depot; and/or the third amount can be no more than 40%, 35%, 30%, 25%, 20%, 15%, 10%, or 5% of the initial amount of the therapeutic agent in the depot.

For example, when measured in vitro at pH 5.8, the depot can exhibit the following release profile: the depot can release from 10% to 35% of the therapeutic agent over the first 5 hours to 10 hours of the treatment period; the depot can release from 5% to 65% of the therapeutic agent over the next 25 hours to 35 hours of the treatment period; and/or the depot can release from 1% to 60% of the therapeutic agent over the next 115 hours to 130 hours of the treatment period.

In some embodiments, when measured in vitro at pH 5.8, the depot exhibits the following release profile: the cumulative amount of therapeutic agent released over the first 6 hours to 8 hours of the treatment period is within a range from 5% to 40%, from 10% to 35%, or from 15% to 30% of the initial amount of the therapeutic agent in the depot; the cumulative amount of therapeutic agent released over the first 35 hours to 42 hours of the treatment period is within a range from 35% to 80%, from 37% to 77%, from 40% to 75%, or from 42% to 72% of the initial amount of the therapeutic agent in the depot; and/or the cumulative amount of therapeutic agent released over the first 159 hours to 161 hours of the treatment period is at least 60%, 70%, or 80% of the initial amount of the therapeutic agent in the depot.

In some embodiments, when measured in vitro at pH 5.8, the depot exhibits the following release profile: at least 10% of the therapeutic agent in the depot is released over the first 15 minutes, 30 minutes, 1 hour, 1.5 hours, 2 hours, 2.5 hours, 3 hours, 3.5 hours, 4 hours, 4.5 hours, or 5 hours of the treatment period; at least 20% of the therapeutic agent in the depot is released over the first 1 hour, 1.5 hours, 2 hours, 2.5 hours, 3 hours, 3.5 hours, 4 hours, 4.5 hours, 5 hours, 5.5 hours, or 6 hours of the treatment period; at least 30% of the therapeutic agent in the depot is released over the first 5 hours, 5.5 hours, 6 hours, 6.5 hours, 7 hours, 7.5 hours, 8 hours, 8.5 hours, 9 hours, 9.5 hours, or 10 hours of the treatment period; at least 40% of the therapeutic agent in the depot is released over the first 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, or 15 hours of the treatment period; at least 50% of the therapeutic agent in the depot is released over the first 10 hours, 11 hours, 12 hours, 13 hours, 14 hours, 14.5 hours, 15 hours, 15.5 hours, 16 hours, 16.5 hours, 17 hours, 17.5 hours, 18 hours, 18.5 hours, 19 hours, 19.5 hours, or 20 hours of the treatment period; at least 60% of the therapeutic agent in the depot is released over the first 15 hours, 20 hours, 21 hours, 22 hours, 22.5 hours, 23 hours, 23.5 hours, 24 hours, 24.5 hours, 25 hours, 25.5 hours, 26 hours, 26.5 hours, 27 hours, 27.5 hours, 28 hours, 29 hours, or 30 hours of the treatment period; at least 70% of the therapeutic agent is released over the first 30 hours, 31 hours, 32 hours, 33 hours, 34 hours, 35 hours, 36 hours, 37 hours, 38 hours, 39 hours or 40 hours of the treatment period; at least 80% of the therapeutic agent is released over the first 50 hours, 52 hours, 54 hours, 55 hours, 56 hours, 57 hours, 58 hours, 59 hours, 60 hours, 62 hours, 64 hours, or 65 hours of the treatment period; and/or at least 90% of the therapeutic agent in the depot is released over the first 100 hours, 105 hours, 110 hours, 115 hours, 120 hours, 125 hours, 130 hours, 135 hours, 140 hours, 145 hours, or 150 hours of the treatment period.

In some embodiments, when measured in vitro at pH 5.8, the depot exhibits the following release profile: the cumulative amount of therapeutic agent released over the first hour of the treatment period is within a range from 10% to 60%, 20% to 50%, or 25% to 45% of the initial amount of the therapeutic agent in the depot; the cumulative amount of therapeutic agent released over the first 2 hours of the treatment period is within a range from 30% to 90%, 35% to 75%, or 40% to 50% of the initial amount of the therapeutic agent in the depot; the cumulative amount of therapeutic agent released over the first 3 hours of the treatment period is within a range from 40% to 99%, 50% to 80%, or 50% to 65% of the initial amount of the therapeutic agent in the depot; the cumulative amount of the therapeutic agent released over the first 4 hours of the treatment period is within a range from 50% to 99%, 55% to 85%, or 60% to 80%; and/or the cumulative amount of the therapeutic agent released over the first 5 hours of the treatment period is at least 60%, 70%, 75%, 80%, 85%, 90%, 95%, or 99% of the initial amount of the therapeutic agent in the depot.

In some embodiments, when measured in vitro at pH 5.8, the depot exhibits the following release profile: up to 10% of the therapeutic agent in the depot is released over the first 5 minutes, 10 minutes, 15 minutes, 20 minutes, 30 minutes, 45 minutes, or 1 hour of the treatment period; up to 20% of the therapeutic agent in the depot is released over the first 10 minutes, 15 minutes, 20 minutes, 30 minutes, 45 minutes, 1 hour, 1.5 hours, or 2 hours of the treatment period; up to 30% of the therapeutic agent in the depot is released over the first 15 minutes, 20 minutes, 30 minutes, 45 minutes, 1 hour, 1.5 hours, 2 hours, 2.5 hours, or 3 hours of the treatment period; up to 40% of the therapeutic agent in the depot is released over the first 30 minutes, 45 minutes, 1 hour, 1.5 hours, 2 hours, 2.5 hours, 3 hours, 3.5 hours, or 4 hours of the treatment period; up to 50% of the therapeutic agent in the depot is released over the first 45 minutes, 1 hour, 1.5 hours, 2 hours, 2.5 hours, 3 hours, 3.5 hours, 4 hours, 4.5 hours, 5 hours, or 6 hours of the treatment period; up to 60% of the therapeutic agent is released over the first 1 hour, 1.5 hours, 2 hours, 2.5 hours, 3 hours, 3.5 hours, 4 hours, 4.5 hours, 5 hours, 6 hours, 7 hours, or 8 hours of the treatment period; up to 70% of the therapeutic agent is released over the first 1.5 hours, 2 hours, 2.5 hours, 3 hours, 3.5 hours, 4 hours, 4.5 hours, 5 hours, 6 hours, 7 hours, 8 hours, 9 hours, or 10 hours of the treatment period; up to 80% of the therapeutic agent is released over the first 2 hours, 2.5 hours, 3 hours, 3.5 hours, 4 hours, 4.5 hours, 5 hours, 6 hours, 7 hours, 8 hours, 9 hours, 10 hours, 11 hours, or 12 hours of the treatment period; and/or up to 90% of the therapeutic agent in the depot is released over the first 2.5 hours, 3 hours, 3.5 hours, 4 hours, 4.5 hours, 5 hours, 6 hours, 7 hours, 8 hours, 9 hours, 10 hours, 11 hours, 12 hours, 13 hours, 14 hours, or 15 hours of the treatment period.

In some embodiments, when measured in vitro at pH 7.4 and/or in vivo, the depot exhibits the following release profile: the cumulative amount of therapeutic agent released over the first 24 hours of the treatment period is within a range from 1% to 25%, 1% to 10%, or 1% to 5% of the initial amount of the therapeutic agent in the depot; the cumulative amount of therapeutic agent released over the first 48 hours of the treatment period is within a range from 1% to 30%, 5% to 20%, or 5% to 15% of the initial amount of the therapeutic agent in the depot; the cumulative amount of therapeutic agent released over the first 72 hours of the treatment period is within a range from 10% to 35%, 10% to 25%, or 15% to 25% of the initial amount of the therapeutic agent in the depot; the cumulative amount of therapeutic agent released over the first 96 hours of the treatment period is within a range from 15% to 50%, 10% to 40%, or 10% to 30% of the initial amount of the therapeutic agent in the depot; the cumulative amount of therapeutic agent released over the first 120 hours of the treatment period is within a range from 20% to 60%, 25% to 50%, or 30% to 40% of the initial amount of the therapeutic agent in the depot; the cumulative amount of therapeutic agent released over the first 144 hours of the treatment period is within a range from 25% to 70%, 30% to 50%, or 35% to 45% of the initial amount of the therapeutic agent in the depot; the cumulative amount of therapeutic agent released over the first 7 days to 8 days of the treatment period is within a range from 30% to 70%, or 35% to 55% of the initial amount of the therapeutic agent in the depot; the cumulative amount of therapeutic agent released over the first 14 days of the treatment period is within a range from 50% to 90%, or 60% to 80% of the initial amount of the therapeutic agent in the depot; and/or the cumulative amount of therapeutic agent released over the first 21 days of the treatment period is within a range from 70% to 99%, or 85% to 95% of the initial amount of the therapeutic agent in the depot.

In some embodiments, when measured in vitro at pH 7.4 and/or in vivo, the depot exhibits the following release profile: up to 10% of the therapeutic agent in the depot is released over the first 4 hours, 12 hours, 24 hours, or 48 hours of the treatment period; up to 20% of the therapeutic agent in the depot is released over the first 24 hours, 48 hours, 72 hours, or 84 hours of the treatment period; up to 30% of the therapeutic agent in the depot is released over the first 48 hours, 72 hours, 120 hours, or 144 hours of the treatment period; up to 40% of the therapeutic agent in the depot is released over the first 120 hours, 144 hours, 168 hours, or 192 hours of the treatment period; up to 50% of the therapeutic agent in the depot is released over the first 7 days, 8 days, 9 days, or 10 days of the treatment period; up to 60% of the therapeutic agent in the depot is released over the first 10 days, 11 days, 12 days, or 13 days of the treatment period; up to 70% of the therapeutic agent in the depot is released over the first 13 days, 14 days, 15 days, or 16 days of the treatment period; up to 80% of the therapeutic agent in the depot is released over the first 16 days, 17 days, 18 days, or 19 days of the treatment period; and/or up to 90% of the therapeutic agent in the depot is released over the first 19 days, 20 days, 21 days, or 22 days of the treatment period.

In some embodiments, when measured in vitro at pH 7.4 and/or in vivo, the depot exhibits the following release profile: the cumulative amount of therapeutic agent released over the first hour of the treatment period is within a range from 5% to 40%, 10% to 30%, or 15% to 25% of the initial amount of the therapeutic agent in the depot; the cumulative amount of therapeutic agent released over the first 4 hours of the treatment period is within a range from 40% to 80%, 50% to 75%, 60% to 80%, or 40% to 60% of the initial amount of the therapeutic agent in the depot; the cumulative amount of therapeutic agent released over the first 24 hours of the treatment period is at least 60%, 70%, 75%, 80%, 85%, 90%, 95%, or 99% of the initial amount of the therapeutic agent in the depot; and/or the cumulative amount of therapeutic agent released over the first 48 hours of the treatment period is at least 80%, 85%, 90%, 95%, or 99% of the initial amount of the therapeutic agent in the depot.

In some embodiments, when measured in vitro at pH 7.4 and/or in vivo, the depot exhibits the following release profile: up to 10% of the therapeutic agent in the depot is released over the first 5 minutes, 10 minutes, 15 minutes, 20 minutes, 30 minutes, 45 minutes, or 1 hour of the treatment period; up to 20% of the therapeutic agent in the depot is released over the first 10 minutes, 15 minutes, 20 minutes, 30 minutes, 45 minutes, 1 hour, 1.5 hours, or 2 hours of the treatment period; up to 30% of the therapeutic agent in the depot is released over the first 15 minutes, 20 minutes, 30 minutes, 45 minutes, 1 hour, 1.5 hours, 2 hours, 2.5 hours, or 3 hours of the treatment period; up to 40% of the therapeutic agent in the depot is released over the first 30 minutes, 45 minutes, 1 hour, 1.5 hours, 2 hours, 2.5 hours, 3 hours, 3.5 hours, or 4 hours of the treatment period; up to 50% of the therapeutic agent in the depot is released over the first 45 minutes, 1 hour, 1.5 hours, 2 hours, 2.5 hours, 3 hours, 3.5 hours, 4 hours, 4.5 hours, 5 hours, or 6 hours of the treatment period; up to 60% of the therapeutic agent is released over the first 1 hour, 1.5 hours, 2 hours, 2.5 hours, 3 hours, 3.5 hours, 4 hours, 4.5 hours, 5 hours, 6 hours, 7 hours, or 8 hours of the treatment period; up to 70% of the therapeutic agent is released over the first 2 hours, 3 hours, 4 hours, 5 hours, 6 hours, 7 hours, 8 hours, 9 hours, 10 hours, 11 hours, 12 hours, 15 hours, or 20 hours of the treatment period; up to 80% of the therapeutic agent is released over the first 3 hours, 4 hours, 5 hours, 6 hours, 7 hours, 8 hours, 9 hours, 10 hours, 11 hours, 12 hours, 15 hours, 20 hours, 24 hours, or 30 hours of the treatment period; and/or up to 90% of the therapeutic agent in the depot is released over the first 5 hours, 6 hours, 7 hours, 8 hours, 9 hours, 10 hours, 11 hours, 12 hours, 15 hours, 20 hours, 24 hours, 30 hours, 36 hours, 40 hours, or 48 hours of the treatment period.

In some embodiments, the release profile of the therapeutic agent is a first order release profile (which can be modeled by the equation Qₜ=Q₀e^{-kt} where Qₜ is the amount of therapeutic agent released at time t, Q₀ is the initial amount of therapeutic agent in the depot, and k is the rate constant). Alternatively, the release profile can be a zero order release profile, a second order release profile, or any other suitable release profile known to those of skill in the art.

The depots described herein can be configured to release a larger amount of the therapeutic agent per day for a first time period than for a longer second time period. In some embodiments, the depot is configured to release the therapeutic agent for at least 14 days post-implantation (or post-immersion in a fluid), where a controlled burst of about 20% to about 50% of the therapeutic agent payload is released in the first 3 days to 5 days, and at least 80% of the remaining therapeutic agent payload is released at a slower rate over the last 10 days to 11 days. In some embodiments, at least 90% of the therapeutic agent payload is released by the end of 14 days.

A two-stage release profile may be especially beneficial in the context of treating pain resulting from a total knee arthroplasty (“TKA”). TKA patients typically experience the greatest pain within the first 1 day to 3 days following surgery (clinically referred to as “acute pain”) with increasingly less pain over the next 7 days to 10 days (clinically referred to as “subacute pain”). The acute period often overlaps or coincides with the patient's inpatient care (usually 1 day to 3 days), and the subacute period generally begins when the patient is discharged and returns home. The two-stage release profile can also be beneficial for other surgical applications, such as other orthopedic applications (e.g., ligament repair/replacement and other damage to the knee, shoulder, ankle, etc.) or non-orthopedic surgical applications, as described in greater detail below. Excessive pain following any surgery may extend inpatient care, cause psychological distress, increase opioid consumption, and/or impair patient participation in physical therapy, any of which may prolong the patient's recovery and/or mitigate the extent of recovery. Pain relief during the subacute period may be particularly complicated to manage, as patient compliance with the prescribed pain management regimen drops off when patients transition from an inpatient to home environment.

To address the foregoing challenges in post-surgical pain management, the depots of the present technology may have a release profile tailored to meet the pain management needs specific to the acute and subacute periods. For example, to address the greater acute pain that occurs immediately following surgery, the depot can be configured to release the therapeutic agent at a faster rate for the first 3 days to 5 days after implantation compared to the subsequent 9 days to 11 days. In some embodiments, the depot delivers a local anesthetic at a rate from about 150 mg/day to about 400 mg/day during this first, acute period. To address the diminishing pain during the subacute period, the depot can be configured to release the therapeutic agent at a slower rate for the remaining 9 days to 11 days. In some embodiments, the depot delivers a local anesthetic at a rate from about 50 mg/day to about 250 mg/day during this second, subacute period. In some embodiments, the rate of release continuously decreases throughout the first period and/or the second period. The timing of the stages of the release profile corresponding to the acute period and the subacute period, as well as the release rate for each stage, can be adjusted depending on the expected magnitude and duration of pain following the surgical procedure. For instance, the duration of the acute period and subacute period can be shorter for procedures that are expected to be less painful than TKA, such as soft tissue procedures (e.g., abdominal hernia repair).

The release profile of the depot can be tuned to release a therapeutic agent for other durations and/or at other release rates by adjusting the structure, composition, and/or the process by which the depot is manufactured. For example, in some embodiments, the depot is configured to release the therapeutic agent at a constant rate throughout the entire duration of release. In some embodiments, the depot is to release the therapeutic agent at a constant rate for a first time period and at a non-constant rate for a second time period (which may occur before or after the first time period).

In some embodiments, the depot is configured to release no more than 20%, 25%, 30%, 35%, 40%, 45%, 50%, 55%, 60%, 65%, or 70% of the therapeutic agent in the first day, 2 days, 3 days, 4 days, 5 days, 6 days, 8 days, 9 days, 10 days, 11 days, 12 days, or 13 days of the duration of release, and at least 75%, 80%, 85%, 90%, 95%, or 100% of the remaining therapeutic agent is released in the remaining days of the duration of release. The intended duration of release may be at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. Alternatively or in combination, the intended duration of release can be no more than 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In some embodiments, the depot is configured to release from 50 mg/day to 600 mg/day, from 100 mg/day to 500 mg/day, from 100 mg/day to 400 mg/day, or from about 100 mg/day to 300 mg/day of the therapeutic agent to the treatment site. In general, the release rate can be selected to deliver the desired dosage to provide the extent of pain relief needed at a given time after the surgical procedure, control toxicity, and deliver the therapeutic agent for a sufficient period of time for pain relief. In some embodiments, the depot is configured to release 50 mg, 100 mg, 150 mg, 200 mg, 250 mg, 300 mg, 350 mg, 400 mg, 450 mg, 500 mg, 600 mg, 700 mg, 800 mg, 900 mg, or 1000 mg of therapeutic agent within any day of the duration of release.

In some embodiments, the depot is configured to release from 50 mg/day to 600 mg/day, from 100 mg/day to 500 mg/day, from 100 mg/day to 400 mg/day, or from 100 mg/day to 300 mg/day of the therapeutic agent to the treatment site within a first time period of release. The depot can further be configured to release from 500 mg/day to 600 mg/day, from 100 mg/day to 500 mg/day, from 100 mg/day to 400 mg/day, or from 100 mg/day to 300 mg/day of the therapeutic agent to the treatment site within a second time period of release. The release rate during the first time period can be the same as, different than, less than, or greater than the release rate during the second time period. Moreover, the first time period can be longer or shorter than the second time period. The first time period can occur before or after the second time period.

In some embodiments, the depot is configured to release no more than 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg of the therapeutic agent within any day of a first time period of release. Alternatively or in combination, the depot can be configured to release at least 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg of the therapeutic agent within any day of the first time period of release. This may be useful for providing different degrees of pain relief at different times after the surgical procedure, and it may also be useful to control toxicity. In such embodiments, the depot can be configured to release at least 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 600 mg, 700 mg, 800 mg, 900 mg, or 1000 mg of the therapeutic agent within any day of a second time period of release. The first time period and/or the second time period can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

One or more depots of the present technology can be implanted at a treatment site in a subject (e.g., a human patient or in an animal model) in order to produce a desired level of therapeutic agent in vivo, such as a level at or above a therapeutic threshold and/or below a toxicity threshold. For example, when implanted, one or more depots of the present technology can produce a mean plasma concentration of the therapeutic agent greater than or equal to a therapeutic threshold of 5 ng/ml, 10 ng ml, 15 ng/ml, 20 mg/ml, 25 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 300 ng/ml, 400 ng/ml, 500 ng/ml, 600 ng/ml, 700 ng/ml, 800 ng/ml, 900 ng/ml, or 1000 ng/ml. Alternatively or combination, the depot(s) can produce a mean plasma concentration of the therapeutic agent less than or equal to a toxicity threshold of 9000 ng/ml, 8000 ng/ml, 7000 ng/ml, 6000 ng/ml, 5000 ng/ml, 4000 ng/ml, 3000 ng/ml, 2500 ng/ml, 2400 ng/ml, 2300 ng/ml, 2200 ng/ml, 2100 ng/ml, 2000 ng/ml, 1900 ng/ml, 1800 ng/ml, 1700 ng/ml, 1600 ng/ml, 1500 ng/ml, 1400 ng/ml, 1300 ng/ml, 1200 ng/ml, 1100 ng/ml, or 1000 ng/ml.

In some embodiments, when one or more depots of the present technology are implanted, the mean plasma concentration of the therapeutic agent reaches or exceeds the therapeutic threshold within the first 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days of the treatment period. The mean plasma concentration of the therapeutic agent can be maintained above the therapeutic threshold and/or below the toxicity threshold for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days; and/or for no more than 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days.

In some embodiments, when implanted, the depot(s) produce a mean Cₘₐₓ of the therapeutic agent that is less than or equal to 5000 ng/ml, 4000 ng/ml, 3000 ng/ml, 2000 ng/ml, 1000 ng/ml, 900 ng/ml, 800 ng/ml, 700 ng/ml, 600 ng/ml, 500 ng/ml, 400 ng/ml, 300 ng/ml, 200 ng/ml, 100 ng/ml, or 50 ng/ml. Alternatively or in combination, the mean Cₘₐₓ of the therapeutic agent can be greater than or equal to 50 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1000 ng/ml, 2000 ng/ml, 3000 ng/ml, 4000 ng/ml, or 5000 ng/ml. The depot(s) can produce a mean t_{1/2} of the therapeutic agent that is greater than or equal to 12 hours, 14 hours, 16 hours, 18 hours, 20 hours, 22 hours, 30 hours, 1 day, 2 days, 3 days, 4 days, 5 days, 6 days, or 7 days; and/or less than or equal to 7 days, 6 days, 5 days, 4 days, 3 days, 2 days, 1 day, 30 hours, 22 hours, 20 hours, 18 hours, 16 hours, 14 hours, 12 hours, or 8 hours. The depot(s) can produce a mean tₘₐₓ of the therapeutic agent that is at least 1 hour, 2 hours, 4 hours, 12 hours, 24 hours, 48 hours, 36 hours, 72 hours, 96 hours, 120 hours, 144 hours, or 168 hours; and/or no more than 144 hours, 120 hours, 96 hours, 72 hours, 36 hours, 48 hours, 24 hours, 12 hours, 4 hours, or 2 hours. The depot(s) can produce a mean tₗₐₛₜ of the therapeutic agent that is at least 7 days, 8 days, 9 days, 10 days, 11 days, 12 days, 13 days, 14 days, 15 days, 16 days, 20 days, 25 days, 30 days, 35 days, 40 days, or 45 days; and/or no greater than 30 days, 25 days, 20 days, 16 days, 15 days, 14 days, 13 days, 12 days, 11 days, 10 days, 9 days, 8 days, 7 days, 6 days, 5 days, 4 days, 3 days, 2 days, or 1 day.

In some embodiments, when implanted, the depot(s) produce a mean AUCₜ₁₋ₜ₂ of the therapeutic agent that is at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, or 8000 day-ng/ml; where the time period t1-t2 can be any of the following: 0 days to 1 day, 0 days to 2 days, 0 days to 3 days, 0 days to 4 days, 0 days to 5 days, 0 days to 6 days, 0 days to 7 days, 0 days to 14 days, 0 days to 21 days, 0 days to 30 days, 1 day to 7 days, 1 day to 6 days, 1 day to 5 days, 1 day to 4 days, 1 day to 3 days, 1 day to 2 days, 2 days to 7 days, 2 days to 6 days, 2 days to 5 days, 2 days to 4 days, 2 days to 3 days, 3 days to 7 days, 3 days to 6 days, 3 days to 5 days, 3 days to 4 days, 4 days to 7 days, 4 days to 6 days, 4 days to 5 days, 5 days to 7 days, 5 days to 6 days, 7 days to 14 days, 7 days to 21 days, 7 days to 30 days, 14 days to 21 days, 14 days to 30 days, 21 days to 30 days, or 0 days to the time point of the last measurable concentration of the therapeutic agent. The depot(s) can produce a mean AUCₗₐₛₜ of the therapeutic agent that is at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, 8000 day-ng/ml, 9000 day-ng/ml, 10,000 day-ng/ml, 11,000 day-ng/ml, 12,000 day-ng/ml, 13,000 day-ng/ml, 14,000 day-ng/ml, or 15,000 day-ng/ml.

In some embodiments, when one or more depots of the present technology are implanted in an animal model (e.g., mouse, rat, rabbit, dog, minipig, sheep), no acute toxicity is observed even when the total dosage of the therapeutic agent in the depot(s) exceeds the dosage expected to produce acute toxicity when administered via other routes (e.g., oral, subcutaneous, intravenous, para-periosteal). For example, no acute toxicity may be observed when one or more depots are implanted in an animal model with a total dosage of the therapeutic agent of at least 50 mg/kg, 100 mg/kg, 500 mg/kg, 1000 mg/kg, 1500 mg/kg, or 2000 mg/kg; and/or no more than 2000 mg/kg, 1500 mg/kg, 1000 mg/kg, or 500 mg/kg.

### III. Systems and Methods of Use

The depots of the present technology (e.g., the depots 100a-570 of FIGS. 1A-5H) can be used to treat a variety of injuries, conditions, or diseases, depending upon the nature of the therapeutic agent delivered as described above. The therapeutic agent can be delivered to specific areas of the patient's body depending upon the medical condition being treated. The depots of the present technology can be positioned in vivo proximate to the target tissue (e.g., bone, soft tissue, etc.) in the patient's body to provide a controlled, sustained release of a therapeutic agent for the treatment of a particular condition. This implantation can be associated with a surgery or intervention for acutely treating the particular condition, whereby the depot provides chronic, sustained pharmacological treatment following completion of the surgery or intervention. The depot can be a standalone element, or can be coupled to or integrated as part of an implantable device or prosthesis associated with the intervention or surgery.

The amount or dose of the therapeutic agent that will be effective in a patient in need thereof can depend on the specific nature of the condition, and can be determined by standard clinical techniques known in the art. In addition, in vitro or in vivo assays can optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and/or the severity of the particular problem being treated.

Some aspects of the present technology include a system including one or more depots (each of which could be any of the depots described herein) provided for implantation by a clinical practitioner. For example, a system can include one, two, three, four, five, six, seven, eight, nine, ten, or more implanted depots. Each depot can be configured for controlled release of a therapeutic agent to tissue proximate to the implantation site of the depot. Accordingly, the depots can collectively provide a desired dose of the therapeutic agent, such as a dose greater than or equal to 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, or 1800 mg. The dose provided by an individual depot or a set of depots can be expressed in terms of the mass of the therapeutic agent used in the depot(s), or in terms of the mass of another form of the therapeutic agent (e.g., the form of the active moiety or the established salt form). For example, the dose of bupivacaine in a depot formulated with bupivacaine hydrochloride monohydrate may be expressed in terms of the equivalent mass of bupivacaine free base (e.g., 595 mg of bupivacaine hydrochloride monohydrate is equivalent to 500 mg of bupivacaine free base) or in terms of the equivalent mass of bupivacaine hydrochloride (e.g., 595 mg bupivacaine hydrochloride monohydrate is equivalent to 563 mg of bupivacaine hydrochloride).

In embodiments where the system includes multiple depots, some or all of the depots in the system can be identical and/or some or all of the depots can differ from each other (e.g., with respect to geometry, composition, and/or release profile). For example, the system can include at least one depot having a release profile that provides for an immediate release of a therapeutic agent, and at least one other depot having a release profile that provides for a delayed release of the therapeutic agent.

Many depots of the present technology are configured to be implanted at a surgical site to treat postoperative pain at or near the site. As used herein, the term “pain” includes nociception and the sensation of pain, both of which can be assessed objectively and subjectively, using pain scores and other methods well-known in the art, such as opioid usage, as described in further detail below. Pain can include allodynia (e.g., increased response to a normally non-noxious stimulus) or hyperalgesia (e.g., increased response to a normally noxious or unpleasant stimulus), which can in turn be thermal or mechanical (tactile) in nature. In some embodiments, pain is characterized by thermal sensitivity, mechanical sensitivity, and/or resting pain. The pain can be primary or secondary pain, as is well-known in the art. Exemplary types of pain reducible, preventable or treatable by the methods and compositions disclosed herein include, without limitation, postoperative pain, for example, from the back in the lumbar regions (lower back pain) or cervical region (neck pain), leg pain, radicular pain (experienced in the lower back and leg from lumbar surgery in the neck and arm from cervical surgery), or abdominal pain from abdominal surgery, and neuropathic pain of the arm, neck, back, lower back, leg, and related pain distributions resulting from disk or spine surgery. Neuropathic pain may include pain arising from surgery to the nerve root, dorsal root ganglion, or peripheral nerve.

In some embodiments, the pain includes “post-surgical pain,” “postoperative pain,” or “surgery-induced pain,” which are used herein interchangeably, and refer to pain arising in the recovery period of seconds, minutes, hours, days or weeks following a surgical procedure (e.g., hernia repair, orthopedic or spine surgery, etc.). Surgical procedures can include any procedure that penetrates beneath the skin and causes pain and/or inflammation to the patient. Surgical procedures can be performed at various sites in a patient's body. For example, surgery may be performed at a patient's knees, hips, upper extremities, lower extremities, neck, spine, shoulders, chest, nasal/sinus region, abdomen, and/or pelvic region.

Some embodiments of the present technology include one or more depots (e.g., having the same or different configuration and/or dosing) that are positioned at or near a surgical site of a knee joint to treat pain associated with a total knee replacement surgery, also known as TKA. In some instances, it may be beneficial to position one or more of the depots within the joint capsule. In some embodiments, one or more depots are positioned at or near the suprapatellar pouch, specifically under the periosteum and attached to the quadriceps tendon. Additional areas for placement of one or more depots may include generally the medial and lateral gutters (including optional fixation to tissue at the medial or lateral side of the respective gutter), on the femur, on the tibia (e.g., posterior attachment to the tibial plateau, at or near the anterior tibia to anesthetize infrapatellar branches of the saphenous nerve). In some embodiments, one or more depots are positioned adjacent to at least one of a posterior capsule of the knee, a superior region of the patella, and/or the arthrotomy incision into the knee capsule. In some embodiments, one or more depots are positioned at or near the saphenous nerve, the adductor canal, and/or the femoral nerve. In some embodiments, one or more depots are positioned at or near an infrapatellar branch of the saphenous nerve, one or more genicular nerves of the knee, a superior region of the patella. It may be desirable to position the depot(s) within the knee capsule but away from any articulating portions of the knee joint itself.

In some embodiments, one or more depots are positioned at or near one or more nerves innervating an anterior knee capsule. For example, the depot(s) may be configured to be positioned at or near a superolateral genicular branch from the vastus lateralis, a superomedial genicular branch from the vastus medialis, a medial (retinacular) genicular branch from the vastus intermedius, an inferolateral genicular branch from the common peroneal nerve, an inferomedial genicular branch from the saphenous nerve, and/or a lateral (retinacular) genicular branch from the common peroneal nerve. Instead of or in addition to the placement of depots within the intracapsular space, one or more depots may be placed at an extracapsular position. In some embodiments, the depot(s) are implanted adjacent to one or more extracapsular nerves. In some embodiments, one or more depots are positioned along or adjacent the subcutaneous skin incision.

So as not to interfere or overlap with a peripheral nerve block administered perioperatively to the patient, one or more of the depots may optionally include a delayed release capability for 6 hours to 24 hours following implantation. In some embodiments, one or more depots placed in the adductor canal and knee capsule are configured to have a delay in the release of therapeutic agent of at least 24 hours.

In some embodiments, the depots of the present technology utilize regional procedures for controlling pain following TKA. Such procedures can include local anesthetic infiltration between the popliteal artery and capsule of the knee (IPACK) block. An IPACK block procedure typically involves scanning the popliteal fossa using a probe proximal the popliteal crease, and injecting an analgesic (e.g., 20 ml of 0.25% ropivacaine) between the patient's popliteal artery and femur. Unlike other known procedures (e.g., adductor canal block (ACB) and femoral nerve catheter (FNC) block) for treating postoperative pain following TKA, IPACK block targets only the terminal branches of the sciatic nerve. In doing so, analgesia and/or other therapeutic agents can be provided to the posterior knee region without causing distal neurologic deficits. In some embodiments, the depots of the present technology are implanted using a combination of the IPACK block procedure and the ACB or FNC block procedures. For example, patients can preoperatively receive one or more depots utilizing an FNC block, and then receive one or more additional depots utilizing a postoperative IPACK block. Utilizing the IPACK block procedure with depots of the present technology can advantageously provide adequate analgesia following TKA, promote improved physical therapy performance, reduce the incident of foot drop, reduce opioid consumption, and/or better control posterior knee pain following TKA, e.g., relative to ACB, FNC block, or other known techniques for pain management following TKA, often allowing for earlier hospital discharge.

The depots disclosed herein can be used to treat postoperative pain associated with other knee surgeries. For example, one or more depots may be used to treat postoperative pain associated with an ACL repair surgery, a medial collateral ligament (“MCL”) surgery, and/or a posterior cruciate ligament (“PCL”) surgery. For ACL repair, one or more depots may be positioned to delivery analgesic the femoral and/or sciatic nerves, while for PCL repair surgery, one or more depots may be positioned parasacral to deliver analgesic to the sciatic nerve. The one or more depots may be used to treat postoperative pain associated with a partial knee replacement surgery, total knee replacement surgery, and/or a revision surgery of a knee replacement surgery. In such procedures, one or more depots can be placed contiguous to the joint or repair site to provide a local block, or else may suitably positioned to provide a regional block by delivering an analgesic to one or more of the femoral nerve or the sciatic nerve, for example via placement in the adductor canal.

In addition to the knee-related surgeries described above, embodiments of the depots disclosed herein can be used to treat postoperative pain associated with other orthopedic surgeries, such as procedures involving the ankle, hip, shoulder, wrist, hand, spine, legs, or arms. For at least some of these surgical procedures, an analgesic can be provided to deliver a local block or a regional block to treat postoperative pain. For a local block, one or more depots can be attached under direct vision in open surgery, for example during joint arthroplasty, open reduction and internal fixation (ORIF) surgery, ligament reconstruction, etc. In procedures involving a joint, one or more depots can be positioned at the joint capsule (e.g., at or near the intracapsular and/or extracapsular space of the joint) and/or adjacent soft tissues spaced apart from articulating surfaces to avoid the depot interfering with joint movement or being damaged by contact with articulating surfaces. In procedures involving fracture repair or ligament repair, one or more depots can be positioned at or adjacent to the repair site to provide a local block. For a regional block, one or more depots can be deposited at a treatment site adjacent to the target nerve via ultrasound guidance using a blunt trocar catheter or other suitable instrument. In some embodiments, it can be beneficial to combine delivery of an analgesic or other therapeutic agents via the depot(s) with delivery of NSAIDs, a long-acting narcotic delivered pre-operatively, and/or acetaminophen. The sustained, controlled, release of an analgesic via the one or more depots can work in concert with these other therapeutic agents to provide a reduction in postoperative pain associated with orthopedic and other surgical procedures.

For example, one or more depots can be used to treat postoperative pain associated with foot and/or ankle surgeries, such as ankle arthroplasty (including ankle revision, ankle replacement, and total ankles replacement), ankle fusion, hindfoot fusion, ligament reconstruction, corrective osteotomies (e.g., bunionectomy, pes planus surgery), or ORIF of ankle or foot fractures. In treating postoperative pain associated with such surgeries, one or more depots can be configured and positioned adjacent to the joint or repair site to provide a local block. Additionally or alternatively, one or more depots can be placed parasacral or at another suitable location to target one or more of the subgluteal sciatic nerve, popliteal sciatic nerve, deep peroneal nerve, or the superficial peroneal nerve. In some embodiments, depots positioned to treat postoperative pain associated with ankle or foot surgeries have a release profile configured to deliver therapeutically beneficial levels of analgesic for a period of 3 days to 7 days.

In another example, one or more depots can be used to treat postoperative pain associated with hip surgeries, such as hip arthroplasty (including hip revision, partial hip replacement, and total hip replacement) or ORIF of hip fractures. In treating postoperative pain associated with such surgeries, one or more depots can be configured and positioned adjacent to the joint or repair site to provide a local block. Additionally or alternatively, a regional block can be provided by placing depots in the psoas compartment, lumbar paravertebral space, fascia iliaca, or other suitable location to target one or more of the lumbar plexus, sacral plexus, femoral nerve, sciatic nerve, superior gluteal nerve, or obturator nerve. In some embodiments, it may be beneficial to secure the one or more depot(s) (e.g., using sutures, fasteners, or other fixation mechanisms) to maintain an anterior position of the depot, thereby preventing or reducing exposure of analgesic to motor nerves (e.g., sciatic or femoral nerves). In some embodiments, depots positioned to treat postoperative pain associated with hip surgeries have a release profile configured to deliver therapeutically beneficial levels of analgesic for a period of 5 days to 7 days, or 7 days to 10 days, depending on the particular surgical procedure.

Postoperative pain associated with shoulder and upper-arm surgeries can likewise be treated using one or more depots as disclosed herein. Examples of such surgeries include shoulder arthroplasty (including shoulder revision, partial shoulder replacement, and total shoulder replacement), upper-arm fracture repair (e.g., scapular, humerus), ligament/tendon repair (e.g., rotator cuff, labrum, biceps, etc.), or ORIF of fractures of the shoulder or upper arm. In treating postoperative pain associated with such surgeries, one or more depots can be configured and positioned adjacent to the joint or repair site to provide a local block. Additionally or alternatively, one or more depots can be configured and positioned to target the brachial plexus by placing one or more depots in the cervical paravertebral space, interscalene, or supraclavicular space. In some embodiments, interscalene placement of the depots can avoid exposure of analgesic to native cartilage, thereby reducing the risk of chondrotoxicity. In some embodiments, depots positioned to treat postoperative pain associated with shoulder or upper-arm related surgeries have a release profile configured to deliver therapeutically beneficial levels of analgesic for a period of 3 days to 7 days.

In another example, one or more depots as described herein can be used to treat postoperative pain associated with elbow surgeries, such as elbow arthroplasty (including elbow revision, partial elbow replacement, and total elbow replacement), ligament reconstruction, or ORIF of fractures of the elbow. In treating postoperative pain associated with such surgeries, one or more depots can be positioned adjacent to the joint or repair site to provide a local block. Additionally or alternatively, one or more depots can be configured and positioned to target the brachial plexus nerves, for example by being placed at or near the cervical paravertebral space, infraclavicular, or axillary position, or other suitable location. In some embodiments, depots positioned to treat postoperative pain associated with elbow surgeries have a release profile configured to deliver therapeutically beneficial levels of analgesic for a period of 3 days to 7 days.

Postoperative pain associated with wrist and hand surgeries can also be treated using one or more depots as described herein. Examples of wrist and hand surgeries include wrist arthroplasty (including wrist revision, partial wrist replacement, and total wrist replacement), wrist fusion, carpal tunnel surgery, and ORIF of fractures of the wrist. In treating postoperative pain associated with such surgeries, one or more depots can be configured and positioned adjacent to the wrist joint or repair site to provide a local block. Additionally or alternatively, one or more depots can be configured and positioned to target the target the ulnar, median, radial, and cutaneous forearm nerves, for example via placement at the antecubital fossa, cervical paravertebral space, infraclavicular, or axillary position. In some embodiments, depots positioned to treat postoperative pain associated with wrist and hand surgeries have a release profile configured to deliver therapeutically beneficial levels of analgesic for a period of 3 days to 7 days.

The depots disclosed herein can likewise be used to treat postoperative pain from other orthopedic surgeries, such as spine surgeries (e.g., laminectomy, spinal fusion), procedures to treat bone fractures (e.g., hip fracture, radius fracture, ulna fracture, tibial fracture, fibular fracture, ankle fracture). For example, postoperative pain associated with spinal fusion can be treated via placement of one or more depots subcutaneously or in the paravertebral space. In treatment of postoperative pain associated with fibular fracture repair, one or more depots can be configured and placed to target the sciatic nerve and/or the popliteal sciatic nerve, for example, being placed parasacral. Various other placements and configurations are possible to provide therapeutic relief from postoperative pain associated with orthopedic surgical procedures.

The depots disclosed herein may be used to treat postoperative pain associated with other types of surgeries besides orthopedic surgeries. For example, the depots may be used to treat postoperative pain for chest-related surgery; breast-related surgery; gynecological or obstetric surgery; general surgery; abdominal surgery; urological surgery; ear, nose, and throat (ENT) surgery; oral and maxillofacial surgery; oncological surgery; or cosmetic surgery. For particular surgeries or classes of surgeries, one or more depots can be positioned at a treatment site to treat postoperative pain. The treatment site can be at or near the surgical site, or can be spaced apart from the surgical site (e.g., proximate to a target nerve or nerve bundle that innervates the surgical site).

For example, one or more depots as described herein can be used to treat postoperative pain associated with chest-related surgeries, such as a thoracotomy, sternotomy, esophageal surgery, cardiac surgery, lung resection, thoracic surgery, or other such procedure. In treating postoperative pain associated with such surgeries, one or more depots can be configured and positioned to target the intercostal nerves, for example, by being placed at or near the thoracic paravertebral space or other suitable location. Analgesics delivered to the intercostal nerves can reduce pain in a patient's chest area, thereby relieving postoperative pain associated with the above-noted chest-related surgical procedures.

In another example, one or more depots disclosed herein can be used to treat postoperative pain associated with breast-related surgeries, such as a mastectomy, breast augmentation (mammoplasty), breast reduction, breast reconstruction procedure, or other such procedures. To treat postoperative pain from such procedures, one or more depots can be positioned and configured to deliver analgesics or other therapeutic agents to the intercostal nerves, for example via placement at or near the patient's infraclavicular space or other suitable location. Additionally or alternatively, one or more depots can be positioned and configured to deliver analgesics or other therapeutic agents to the lateral pectoral nerve and/or the medial pectoral nerve, for example, via placement between the serratus anterior muscle and the latissimus dorsi muscle or other suitable location. As noted above, analgesics delivered to the intercostal nerves can reduce pain in a patient's chest area, while analgesics delivered to the lateral and/or medial pectoral nerves can reduce pain in the pectoralis major and pectoralis minor, thereby reducing postoperative pain associated with the above-noted chest-related surgical procedures.

As another example, one or more depots can be used to treat postoperative pain associated with general, abdominal, pelvic, and/or urological procedures. Examples of such procedures include proctocolectomy, colectomy, pancreatectomy, appendectomy, hemorrhoidectomy, cholecystectomy, kidney transplant, nephrectomy, radical prostatectomy, nephrectomy, gastrectomy, gastric surgeries, small bowel resection, splenectomy, laparotomy, laparoscopy, hernia repair (e.g., inguinal, ventral, umbilical, incisional), sigmoidectomy, colorectal resection, liver resection, enterostomy, rectum resection, kidney stone removal, cystectomy procedures, and gender reassignment surgeries. For such operations, postoperative pain can be treated by placing one or more depots to target nerves at the transverse abdominis plane (TAP). Analgesics delivered to the TAP can anesthetize the nerves that supply the anterior abdominal wall, thereby reducing postoperative pain in this region. In some embodiments, one or more depots are disposed between the internal oblique and transverse abdominis muscles. In some embodiments, one or more depots can be disposed at or adjacent to the abdominal wall, for example, being secured in place via sutures, fasteners, or other fixation mechanisms.

In some embodiments, one or more depots are used to treat postoperative pain associated with gynecological and obstetric surgeries, such as myomectomy, Caesarian section, hysterectomy (e.g., transvaginal hysterectomy), oophorectomy, pelvic floor reconstruction, or other such surgical procedures. For such procedures, the depot(s) can be configured and positioned to deliver analgesics or other therapeutic agents to one or more of the nerves innervating the pelvic and/or genital area, for example, the pudendal nerve, intercostal nerve, or other suitable nerve.

In some embodiments, one or more depots can be used to treat postoperative pain associated with ENT surgical procedures, for example, tonsillectomy, submucosal resection, rhinoplasty, sinus surgery, inner ear surgery, parotidectomy, submandibular gland surgery, or other such procedures. Similarly, one or more depots can be used to treat postoperative pain associated with oral and maxillofacial surgeries, for example, dentoalveolar surgery, dental implant surgery, orthognathic surgery, temporomandibular joint (TMJ) surgery, dental reconstruction surgeries, or other such procedures. For ENT surgical procedures and/or oral and maxillofacial surgical procedures, the depot(s) can be configured and positioned to deliver analgesics or other therapeutic agents to one or more of the nerves innervating regions affected by the surgical procedure, for example, the mandibular nerve, the mylohyoid nerve, lingual nerve, inferior alveolar nerve, buccal nerve, auriculotemporal nerve, anterior ethmoidal nerve, or other suitable nerve.

One or more depots can also be used to treat postoperative pain for other surgical procedures, for example oncological surgeries (e.g., tumor resection), cosmetic surgeries (e.g., liposuction, abdominoplasty), amputations, or other surgical procedures resulting in postoperative pain. Optionally, one or more depots can be used to treat pain for indications that may not be associated with a surgical procedure, such as treatment of neuromas or phantom limb pain.

The number of depots and the characteristics of individual depots (e.g., geometry, composition, release profile) can be selected to deliver the desired therapeutic benefits for the particular condition to be treated. For example, while a patient recovering from hard tissue surgeries (e.g., knee replacement surgery) may benefit from delivery of analgesics for a relatively long time period (e.g., at least 7 days, 14 days, or 21 days post-surgery), a patient recovering from other types of surgeries may not require the same level or duration of analgesic drug delivery. In some embodiments, a patient recovering from a soft tissue surgery (e.g., tonsillectomy, hernia repair, abdominoplasty, mammoplasty) may benefit from delivery of analgesics for a shorter time period, such as up to 4 days, 5 days, 6 days, or 7 days post-surgery. As such, depots delivered to a patient for treatment of postoperative pain following soft tissue surgeries may require fewer depots, depots having a smaller payload of therapeutic agent, depot(s) having a faster release profile (e.g., depots with fewer or no control regions), etc. In another example, the systemic therapeutic threshold of the therapeutic agent that correlates to the desired amount of pain relief may vary depending on the condition to be treated, and the number and characteristics of the depot(s) selected for implantation can be selected to provide therapeutic agent delivery at or above the systemic therapeutic threshold for the appropriate time period after surgery. Additionally, the number and characteristics of the depot(s) selected for implantation can be tailored to accommodate the target anatomical region for placement in the patient's body.

### IV. Clinical Outcomes

The efficacy of the depots of the present technology (e.g., the depots 100a-570 of FIGS. 1A-5H) in providing a therapeutic benefit can be evaluated using various metrics. For example, the efficacy of a depot or depots in providing pain relief via delivery of analgesic can be evaluated based on pain score, quality of recovery, opioid consumption and related side effects, and/or functional assessments such as range of motion testing, the Western Ontario and McMaster Universities Osteoarthritis (WOMAC) Index, and the Knee Injury and Osteoarthritis Outcome Score (KOOS), among others.

The Numeric Rating Scale (NRS) is a pain scoring system in which the patient assesses their pain on a scale from 0 (no pain) to 10 (worst possible pain). Pain can be measured at rest (NRS-R) or with activity (NRS-A). Any reference herein to an NRS score can encompass an NRS-R score, an NRS-A score, or a combination thereof. The NRS scores described herein can be measured at a time of day before the patient has consumed any opioids or other pain management medications, and/or at a time of day when the patient has not consumed any opioids or other pain management medications. In some embodiments, the NRS score of a patient who has received one or more depots of the present technology (“treatment patient”) is significantly lower than the NRS score of a patient who has not received any depots (“control patient”) at one or more time points after surgery. The time point can be 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after surgery. The NRS score of the treatment patient can be reduced by at least 10%, 20%, 30%, 40%, 50%, 60%, 70%, 80%, or 90% compared to the NRS score of the control patient at the same time point.

In some embodiments, at least 10%, 20%, 30%, 40%, 50%, 60%, 70%, 80%, 90%, or 100% of treatment patients are pain-free based on the NRS score (e.g., a NRS score of 0 or 1) at one or more time points after surgery, such as 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after surgery. A treatment patient may achieve a pain-free state faster than a control patient, e.g., by at least 1 day, 2 days, 3 days, 4 days, 5 days, 6 days, 7 days, 14 days, 28 days, or 30 days.

In some embodiments, postoperative pain is evaluated by comparing the NRS-R score to the NRS-A score of a patient at one or more time points. In the context of TKA, activity can redistribute the anesthetic within the synovial space of the knee, which may reduce the NRS-A score. Accordingly, the difference between the NRS-A score and the NRS-R score at a particular time point may be smaller for treatment patients versus control patients (e.g., at least 10%, 20%, 30%, 40%, 50%, 60%, 70%, 80%, or 90% smaller).

Optionally, postoperative pain can be assessed by comparing the AUC of the NRS score (“NRS AUC”) of a treatment patient versus the NRS AUC of a control patient over one or more time periods after surgery. The time period can be from 0 hours to 12 hours, from 0 hours to 24 hours, from 0 hours to 72 hours, from 0 hours to 96 hours, from 0 hours to 7 days, from 0 hours to 14 days, 0 hours to 15 days, from 0 hours to 30 days, from 12 hours to 24 hours, from 12 hours to 36 hours, from 12 hours to 72 hours, from 12 hours to 96 hours, from 12 hours to 7 days, from 12 hours to 10 days, from 12 hours to 14 days, from 12 hours to 21 days, from 12 hours to 30 days, from 1 day to 2 days, from 1 day to 4 days, from 1 day to 7 days, from 1 day to 14 days, from 1 day to 15 days, from 1 day to 21 days, from 1 day to 30 days, from 2 days to 3 days, from 2 days to 4 days, from 2 days to 7 days, from 2 days to 14 days, from 2 days to 15 days, from 2 days to 21 days, from 2 days to 30 days, from 3 days to 4 days, from 3 days to 7 days, from 3 days to 14 days, from 3 days to 15 days, from 3 days to 21 days, from 3 days to 30 days, from 4 days to 5 days, from 4 days to 7 days, from 4 days to 14 days, from 4 days to 15 days, from 4 days to 21 days, from 4 days to 30 days, from 5 days to 6 days, from 5 days to 7 days, from 5 days to 14 days, from 5 days to 15 days, from 5 days to 21 days, from 5 days to 30 days, from 6 days to 7 days, from 6 days to 14 days, from 6 days to 15 days, from 6 days to 21 days, from 6 days to 30 days, from 7 days to 8 days, from 7 days to 14 days, from 7 days to 15 days, from 7 days to 21 days, from 7 days to 30 days, from 8 days to 9 days, from 9 days to 10 days, from 10 days to 11 days, from 11 days to 12 days, from 12 days to 13 days, from 13 days to 14 days, from 14 days to 15 days, from 14 days to 21 days, from 14 days to 30 days, from 15 days to 21 days, from 15 days to 30 days, from 16 days to 21 days, from 16 days to 30 days, or from 21 days to 30 days after surgery. The NRS AUC of the treatment patient can be reduced by at least 10%, 20%, 30%, 40%, 50%, 60%, 70%, 80%, or 90% compared to the NRS AUC of the control patient over the same time period.

The efficacy of the depots of the present technology in treating pain can also be assessed based on consumption of supplemental opioid medications prescribed to the patient for pain management. In some embodiments, at least 10%, 20%, 30%, 40%, 50%, 60%, 70%, 80%, 90%, or 100% of treatment patients remain opioid-free over one or more time periods after surgery. Alternatively or in combination, the total amount of opioids consumed by a treatment patient can be reduced by at least 10%, 20%, 30%, 40%, 50%, 60%, 70%, 80%, or 90% compared to the total amount of opioids consumed by a control patient over the same time period after surgery. The total amount of opioids consumed by the treatment patient can be no more than 600 morphine milligram equivalents (MME), 550 MME, 500 MME, 450 MME, 400 MME, 350 MME, 300 MME, 250 MME, 200 MME, 150 MME, 100 MME, or 50 MME over a specified time period. The time period for assessing postoperative opioid consumption can be from 0 hours to 12 hours, from 0 hours to 24 hours, from 0 hours to 72 hours, from 0 hours to 96 hours, from 0 hours to 7 days, from 0 hours to 14 days, 0 hours to 15 days, from 0 hours to 30 days, from 12 hours to 24 hours, from 12 hours to 36 hours, from 12 hours to 72 hours, from 12 hours to 96 hours, from 12 hours to 7 days, from 12 hours to 10 days, from 12 hours to 14 days, from 12 hours to 21 days, from 12 hours to 30 days, from 1 day to 2 days, from 1 day to 4 days, from 1 day to 7 days, from 1 day to 14 days, from 1 day to 15 days, from 1 day to 21 days, from 1 day to 30 days, from 2 days to 3 days, from 2 days to 4 days, from 2 days to 7 days, from 2 days to 14 days, from 2 days to 15 days, from 2 days to 21 days, from 2 days to 30 days, from 3 days to 4 days, from 3 days to 7 days, from 3 days to 14 days, from 3 days to 15 days, from 3 days to 21 days, from 3 days to 30 days, from 4 days to 5 days, from 4 days to 7 days, from 4 days to 14 days, from 4 days to 15 days, from 4 days to 21 days, from 4 days to 30 days, from 5 days to 6 days, from 5 days to 7 days, from 5 days to 14 days, from 5 days to 15 days, from 5 days to 21 days, from 5 days to 30 days, from 6 days to 7 days, from 6 days to 14 days, from 6 days to 15 days, from 6 days to 21 days, from 6 days to 30 days, from 7 days to 8 days, from 7 days to 14 days, from 7 days to 15 days, from 7 days to 21 days, from 7 days to 30 days, from 8 days to 9 days, from 9 days to 10 days, from 10 days to 11 days, from 11 days to 12 days, from 12 days to 13 days, from 13 days to 14 days, from 14 days to 15 days, from 14 days to 21 days, from 14 days to 30 days, from 15 days to 21 days, from 15 days to 30 days, from 16 days to 21 days, from 16 days to 30 days, or from 21 days to 30 days after surgery.

In some embodiments, the time to first opioid consumption after surgery (e.g., time to rescue opioid) for a treatment patient is delayed compared to a control patient, such as by at least 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 96 hours, 5 days, 6 days, or 7 days. A treatment patient may not consume any opioids until at least 1 hour, 2 hours, 3 hours, 4 hours, 5 hours, 6 hours, 7 hours, 8 hours, 9 hours, 10 hours, 11 hours, 12 hours, 15 hours, 20 hours, 24 hours, 30 hours, 36 hours, 40 hours, or 48 hours after surgery. Treatment patients may also experience fewer or no opioid-related adverse events (e.g., nausea, vomiting, constipation, ileus) compared to control patients. In some embodiments, the percentage of treatment patients experiencing opioid-related adverse events is reduced by at least 10%, 20%, 30%, 40%, 50%, 60%, 70%, 80%, or 90% compared to control patients.

The efficacy of the depots of the present technology in treating pain can also be assessed based on a movement parameter, such as range of motion and/or other activity. For example, for TKA, range of motion can be assessed based on degrees of flexion and/or extension of the knee after surgery. In some embodiments, the time for a treatment patient to achieve a target degree of flexion and/or extension after surgery is reduced compared to a control patient, e.g., by at least 1 day, 2 days, 3 days, 4 days, 5 days, 6 days, 7 days, 14 days, 28 days, or 30 days. The target degree of flexion and/or extension can vary based on the activity being assessed (e.g., walking, sitting, going up stairs, etc.), and can be determined in accordance with standards known to those of skill in the art. The treatment patient can achieve the target degree of flexion and/or extension within 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after surgery. As another example, treatment patients may resume normal physical activity faster than control patients after surgery, such as at least 1 day, 2 days, 3 days, 4 days, 5 days, 6 days, 7 days, 14 days, 28 days, or 30 days. Other suitable movement parameters include overall activity levels (e.g., number of steps, time spent walking, time spent running, six-minute walk distance, etc.), gait (e.g., time to recovery of normal gait), and/or other metrics. Movement parameter measurements can be assessed based on change or rate of change in the measured values over time and/or comparison of the measured value to a control patient value, a value of healthy individuals (e.g., of a similar age, having a similar activity profile to the patient pre-surgery, etc.), and/or a particular patient's pre-operative level. In some embodiments, a patient's physical activity is tracked and assessed using a wearable or sensor, such as a fitness monitor.

In some embodiments, the efficacy of the depots of the present technology in treating pain is assessed based on compliance with a prescribed physical therapy regimen. Patients experiencing considerable postoperative pain will often miss or skip physical therapy sessions (e.g., onsite or virtual) and/or give considerably less effort as quantified by time, repetitions, flexion/extension, and/or other parameters. Treatment patients may, therefore, demonstrate greater compliance with physical therapy as compared to control patients based on one or more of these metrics. Poor compliance with physical therapy can result in the formation of adhesions and/or scar tissue that causes stiffness in the surgical area (e.g., the knee joint), which may require return to the hospital for a surgical manipulation of the knee. As such, rate or incidence of surgical manipulation can be another clinical endpoint for demonstrating benefit, e.g., the rate of surgical manipulation in treatment patients can be reduced by at least 10%, 20%, 30%, 40%, 50%, 60%, 70%, 80%, or 90% compared to control patients.

The efficacy of the depots of the present technology in treating pain can alternatively or additionally be assessed based on other factors. For example, treatment patients may be discharged from the hospital sooner than control patients, e.g., by at least 12 hours, 24 hours, 36 hours, 48 hours, 72 hours, 60 hours, or 96 hours. The discharge time may be related to the amount of pain that the patient is experiencing, in that patients experiencing more postoperative pain may be discharged later than patients experiencing less postoperative pain. In a further example, the rehospitalization rate of treatment patients can be reduced by at least 10%, 20%, 30%, 40%, 50%, 60%, 70%, 80%, or 90% compared to control patients. Rehospitalization may occur if the patient is experiencing prolonged and/or severe pain, if surgical revisions are needed, and/or other factors. In yet another example, the percentage of treatment patients who contact their surgeon or physician after discharge to seek treatment for postoperative pain can be reduced by at least 10%, 20%, 30%, 40%, 50%, 60%, 70%, 80%, or 90% compared to control patients.

As another example, treatment patients may exhibit improved recovery after surgery compared to control patients, as measured by one or more quality of recovery (QoR) scores. QoR scores allow the patient to provide a self-reported rating on recovery-related measures such as pain, physical comfort, physical independence, psychological support, emotional state, and mental well-being. QoR scores can be assessed using a longer form 40-item score (QoR-40) or a shorter-form 15-item score (QoR-15) derived from the QoR-40. In some embodiments, treatment patients exhibit improved QoR scores compared to control patients at one or more time points after surgery, such as 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after surgery.

In some embodiments, the efficacy of the depots of the present technology is evaluated using the WOMAC index, which is a set of standardized questionnaires used by health professionals to evaluate the condition of patients with joint pain from a variety of sources. The WOMAC measures five items for pain (score range 0-20), two for stiffness (score range 0-8), and 17 for functional limitation (score range 0-68). Physical functioning questions cover everyday activities such as stair use, standing up from a sitting or lying position, standing, bending, walking, getting in and out of a car, shopping, putting on or taking off socks, lying in bed, getting in or out of a bath, sitting, and heavy and light household duties. The questions on the WOMAC are a subset of the questions of the Hip disability and Osteoarthritis Outcome score (HOOS). Thus, a HOOS survey may also be used to determine a WOMAC score.

Some embodiments of the present technology include a method for treating a patient suffering from postsurgical pain at an anatomical region of the patient's body. The pain may be associated with a surgery at or near the anatomical region. In some embodiments, the method includes improving a WOMAC index total score of the patient by implanting one or more of the depots disclosed herein at a surgical site at the anatomical region. The method can include improving a WOMAC index pain sub-score, stiffness sub-score, and/or physical function sub-score. The WOMAC index total score and/or one or more of the sub-scores may be evaluated at set time intervals (weekly, monthly, bi-monthly, etc.) and compared to the patient's previous scores, the patient's pre-operative score, and/or the score of a patient of similar age, fitness, and health that underwent the same surgery but was not treated with one of the depots of the present technology.

The KOOS was developed as an extension of the WOMAC Index with the purpose of evaluating short-term and long-term symptoms and function in subjects with knee injury and osteoarthritis. The KOOS holds five separately scored subscales: pain, other symptoms, function in daily living (ADL), function in sport and recreation (sport/rec), and knee-related quality of life (QOL). The KOOS has been validated for several orthopedic interventions such as anterior cruciate ligament reconstruction, meniscectomy and total knee replacement. The effect size is generally largest for the subscale QOL followed by the subscale pain. In some embodiments, the method includes improving a KOOS score of the patient by implanting one or more of the depots disclosed herein at a surgical site at the anatomical region. The method can include improving a KOOS sub-score, including at least one of pain, other symptoms, function in daily living (ADL), function in sport and recreation (sport/rec), and/or knee-related quality of life (QOL). The KOOS score and/or one or more of the sub-scores may be evaluated at set time intervals (weekly, monthly, bi-monthly, etc.) and compared to the patient's previous scores, the patient's pre-operative score, and/or the score of a control patient (e.g., a patient of similar age, fitness, and health that underwent the same surgery but was not treated with one of the depots of the present technology).

In some embodiments, a method of treating a subject suffering from pain after a surgical procedure (e.g., a TKA or any of the other surgical procedures described herein) includes placing one or more of the depots described herein (e.g., one or more of the depots 100a-570 of FIGS. 1A-5H) in the subject (e.g., at or near the surgical site or another treatment site). The depot(s) can be configured such that a population of patients treated with the depot(s) (“treatment population”) exhibit at least one improved clinical outcome compared to a population of patients that were not treated with the depot(s) (“control population”). The improved clinical outcome can include any of the metrics described herein. For example, compared to the control population, the treatment population can exhibit one or more of the following improved outcomes: reduced mean NRS score, reduced mean difference between NRS-A and NRS-R scores, reduced mean NRS AUC, greater percentage of pain-free patients, reduced mean opioid consumption, greater percentage of opioid-free patients, longer mean time to first opioid consumption, reduced incidence of opioid-related adverse events, improved mean degrees of flexion and/or extension, reduced mean time to achieve a target degree of flexion and/or extension, reduced mean time to hospital discharge, reduced hospitalization rate, greater mean QoR scores, greater mean WOMAC scores, and/or greater mean KOOS scores. The improved clinical outcome can be assessed at any of the time points and/or periods after the surgical procedure described herein.

### EXAMPLES

The following examples are included to further describe some aspects of the present technology, and should not be used to limit the scope of the technology.

### Example 1: Configurations of Implantable Depots for Treating Postoperative Pain

This example describes three configurations for depots designed for implantation in a surgical site to treat postoperative pain: (1) a rectangular depot with notches, similar to the depot 200 illustrated in FIGS. 2A and 2B (“R300”); (2) a triangular depot with a single hole, similar to the depot 300 illustrated in FIGS. 3A and 3B (“T600”); and (3) a triangular depot with four holes, similar to the depot 400 illustrated in FIGS. 4A and 4B (“T500”). Each depot included a single therapeutic region positioned between two control regions. The dimensions of each depot are provided in Table 1 below.

TABLE 1
Depot Dimensions

| Dimension | R300 | T600 | T500 |
|---|---|---|---|
| Shape | Rectangular | Equilateral | Equilateral |
| | with Rounded | Triangle | Triangle |
| | Corners | with Rounded | with Rounded |
| | | Corners | Corners |
| Length (mm) | 27.5 | 30.5 | 28.2 |
| Width (mm) | 17.0 | NA | NA |
| Height (mm) | NA | 27.4 | 25.3 |
| Therapeutic Region | 980 | 1680 | 1950 |
| Thickness (μm) | | | |
| Single Control | 10 | 10 | 25 |
| Region Thickness | | | |
| (μm) | | | |
| Combined Control | 20 | 20 | 50 |
| Region Thickness | | | |
| (μm) | | | |
| Total Thickness | 1.0 | 1.7 | 2.0 |
| (mm) | | | |
| Rounded Corner | 2.5 | 3.7 | 3.4 |
| Radius (mm) | | | |
| Number of Notches | 4 | NA | NA |
| Notch Radius (mm) | 1.5 | NA | NA |
| Notch Area (mm²) | 14 | NA | NA |
| Number of Holes | NA | 1 | 4 |
| Hole Radius (mm) | NA | 1.5 | 0.75 |
| Total Hole Area | NA | 7 | 7 |
| (mm²) | | | |
| Top Surface Area | 448 | 521 | 446 |
| (mm²) | | | |
| Bottom Surface Area | 448 | 521 | 446 |
| (mm²) | | | |
| Total Perimeter (mm) | 91.6 | 101.8 | 104.4 |
| Total Lateral Surface | 92 | 173 | 209 |
| Area (mm²) | | | |
| Total Surface Area | 988 | 1215 | 1100 |
| (mm²) | | | |
| Therapeutic Region | 439.0 | 874.9 | 869.1 |
| Volume (mm³) | | | |
| Combined Control | 9.0 | 10.4 | 22.3 |
| Region Volume | | | |
| (mm³) | | | |
| Total Volume (mm³) | 448 | 885 | 891 |

Tables 2 and 3 below provide the dry mass compositions for the therapeutic region and control regions, respectively, in the R300, T600, and T500 depots. “BUP-HCl” refers to bupivacaine hydrochloride monohydrate (therapeutic agent), “PLGA5050” refers to PLGA 50:50 (polymer), and “PS20” refers to Polysorbate 20 (releasing agent).

TABLE 2
Therapeutic Region Dry Mass Composition

| Component | R300, T600, T500 |
|---|---|
| BUP-HCl | 64.5% |
| PLGA50:50 | 32.3% |
| PS20 | 3.2% |

TABLE 3
Control Region Dry Mass Composition

| Component | R300, T600 | T500 |
|---|---|---|
| PLGA5050 | 66.7% | 100% |
| PS20 | 33.3% | 0% |

Table 4 provides the theoretical dry mass compositions of the components in the R300, T600, and T500 depots. The theoretical percent composition of each component was calculated on a mass basis derived from on the respective thicknesses of the therapeutic and control regions, and the percent composition in each formulation. The calculation assumed that all component densities are equivalent (i.e., 1.0 g/cm³).

TABLE 4
Theoretical Depot Dry Mass Compositions

| Parameter | R300 | T600 | T500 |
|---|---|---|---|
| % Mass of Control Region | 2.0% | 1.2% | 2.5% |
| % Mass of Therapeutic Region | 98.0% | 98.8% | 97.5% |
| Total % Mass of BUP-HCl | 63.2% | 63.7% | 62.9% |
| Total % Mass of PLGA5050 | 32.9% | 32.7% | 34.0% |
| Total % Mass of PS20 | 3.8% | 3.6% | 3.1% |
| Mass of BUP-HCl (mg) | 300 | 600 | 595 |
| Mass of PLGA5050 (mg) | 156 | 308 | 321 |
| Mass of PS20 (mg) | 18 | 34 | 30 |
| Total Dry Mass (mg) | 475 | 941 | 946 |

### Example 2: Preparation and Characterization of Implantable Depots

This example describes the preparation and characterization of the R300, T600, and T500 depots. The therapeutic region in all three depots was formulated with PS20, PLGA5050, BUP-HCl, and acetone in a 1:10:20:30 ratio by mass. The control regions in R300 and T600 were formulated with PS20, PLGA5050, and acetone in a 1:2:6 ratio by mass. The control regions in T500 were formulated with PLGA5050 and acetone in a 1:4 ratio by mass.

The therapeutic regions for R300, T600, and T500 were manufactured by combining PS20, PLGA5050, and acetone, and mixing until the PLGA5050 was completely dissolved. BUP-HCl was then mixed into the polymer solution to create a dough-like consistency. The dough was portioned out into smaller quantities and stirred. Each portion underwent a series of heat compression steps to form a disk of the desired thickness. The disks were then dried.

After the disks dried, the control regions were applied to both sides of the disk. For the R300 and T600 depots, the control regions were formed by dissolving PLGA5050 and PS20 in acetone, then casting the polymer solution into thin films of the desired thickness. The thin films were then bonded to each side of the disk using heat compression. For the T500 depots, the control regions were formed by dissolving PLGA5050 in acetone, then dipping the disks into the polymer solutions. After the control regions were applied, individual depots were cut from the disks.

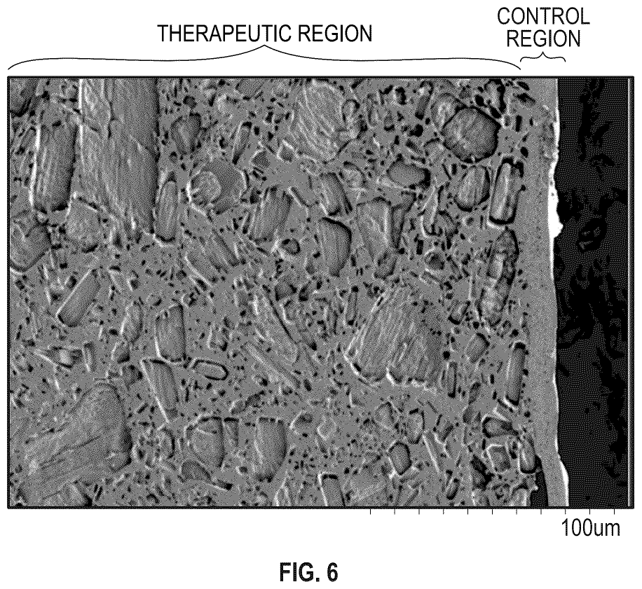

FIG. 6 is a scanning electron microscope (SEM) image of a portion of a R300 depot. The depot was cryomilled and sputter coated prior to imaging. As can be seen in FIG. 6, the therapeutic region is a two-phase structure with BUP-HCl crystals held together by PLGA5050. The control region is an approximately 10 μm thick layer over the therapeutic region.

### Example 3: In Vitro Release Profiles

This example describes in vitro release data for the T500 depot. In vitro elution studies were performed at pH 5.8 in phosphate-buffered saline (PBS). Depots were placed in baskets rotating at 10 RPM in 750 mL of elution media at 37° C. The elution media was periodically analyzed spectrophotometrically at 262 nm and the BUP-HCl concentration was quantified with a USP reference standard in a standalone sealed cuvette.

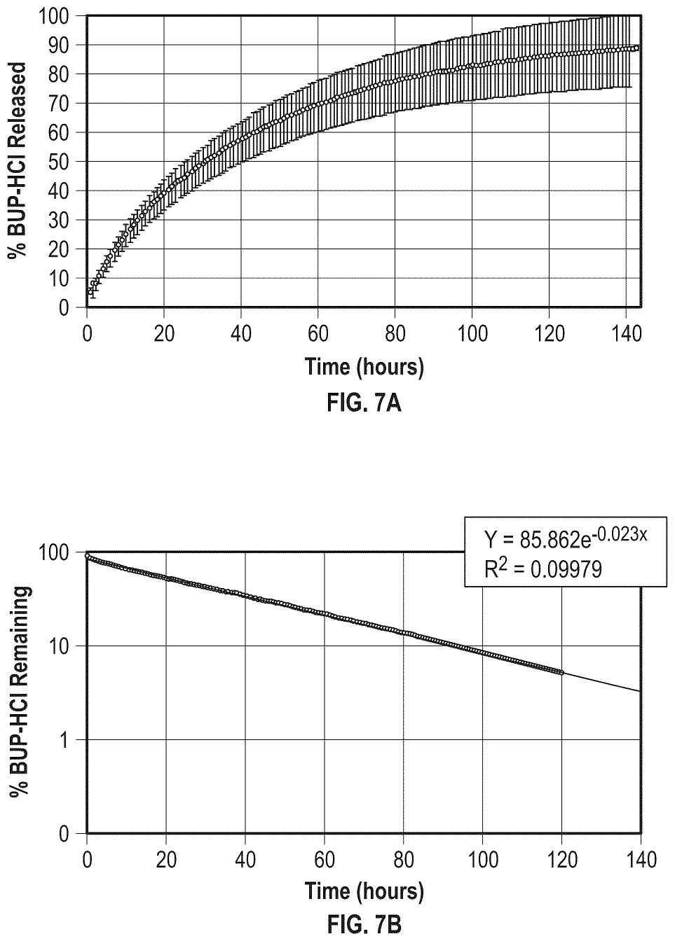

FIG. 7A is a graph showing the percentage of BUP-HCl released from the T500 depot over time. The release profile was highly consistent across the different depot samples tested (n=12), with a standard deviation of less than 10% for the first 80 hours of release.

FIG. 7B is a semi-log graph showing the percentage of BUP-HCl remaining in the T500 depot over time. As can be seen in the graphs, the in vitro elution of BUP-HCl from the T500 depot closely follows first order release kinetics (R²=0.9979), with an observed rate constant of 0.023 hr⁻¹ and a half-life of 30 hours.

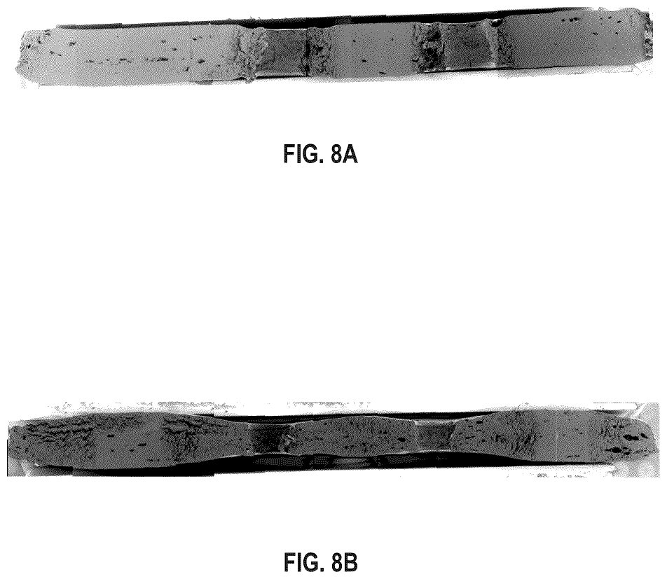

FIG. 8A is an SEM image of a T500 depot that is approximately 25% eluted at 50× magnification, and FIG. 8B is an SEM of a T500 depot that is approximately 75% eluted at 50× magnification. The depots were sectioned with a microtome before imaging. In the 75% eluted sample, the portions of the depot near the periphery and around the holes are significantly thinner compared to the 25% eluted sample, indicating that the BUP-HCl payload in those portions has been released. In contrast, the interior portions of the depot away from the periphery and holes have maintained their original thicknesses, indicating that the BUP-HCl payload is still present. These results show that the BUP-HCl release rate correlates to the travel distance, in that BUP-HCl molecules located closer to exposed surfaces of the depot elute faster than BUP-HCl molecules located further away from the exposed surfaces.

### Example 4: In Vivo Pharmacokinetics of Implantable Depots

This example describes in vivo pharmacokinetic data for R300 and T600 depots implanted in human subjects for treatment of postoperative pain after TKA. The safety and pharmacokinetics of R300 and T600 depots were investigated in an open-label 22 patient study. The patients were adult subjects between 18- and 80-years old undergoing primary unilateral TKA. One or more depots were placed in the knee capsule of each subject following the TKA procedure and before surgical closure of the knee capsule. Depending on dosage levels the depot(s) were placed in one or more of the following locations: the suprapatellar pouch, the medial gutter alongside the capsular tissue, and/or the lateral gutter alongside the capsular tissue. Table 5 below provides the depot configurations and bupivacaine dose for each cohort (in Examples 4-6 and the accompanying Figures, “BUP” or “bupivacaine” refers to bupivacaine free base).

TABLE 5
Cohorts for TKA Study

| | Number of | Depot | Total Bupivacaine | |
|---|---|---|---|---|
| Cohort | Subjects | Configuration | Dose | |
| 1 | 4 | 1 R300 | 252 | mg |
| 2 | 3 | 3 R300 | 756 | mg |
| 3A-3B | 9 | 6 R300 | 1512 | mg |
| 3C | 6 | 3 T600 | 1512 | mg |

Venous blood samples (4 mL) for plasma pharmacokinetic analysis were taken at various time intervals during surgery and within the first 24 hours post-surgery, then approximately every 4 hours from 24 to 96 hours post-surgery, then daily thereafter through Day 15, and at follow-up visits on Days 30, 45, and 60 (the Cohort 3C subjects had additional visits on Days 18, 21, 24, and 27). Bupivacaine was extracted from human plasma by protein precipitation with acetonitrile. Before the extraction, bupivacaine-d9 was added as an internal standard. A portion of the organic supernatant was transferred to a new 96-well plate and diluted with water. The samples were injected into a liquid chromatography tandem mass spectrometry (LC-MS/MS) system using an Agilent Zorbax SB-C18 column with a gradient mobile phase containing acetonitrile, water, and formic acid.

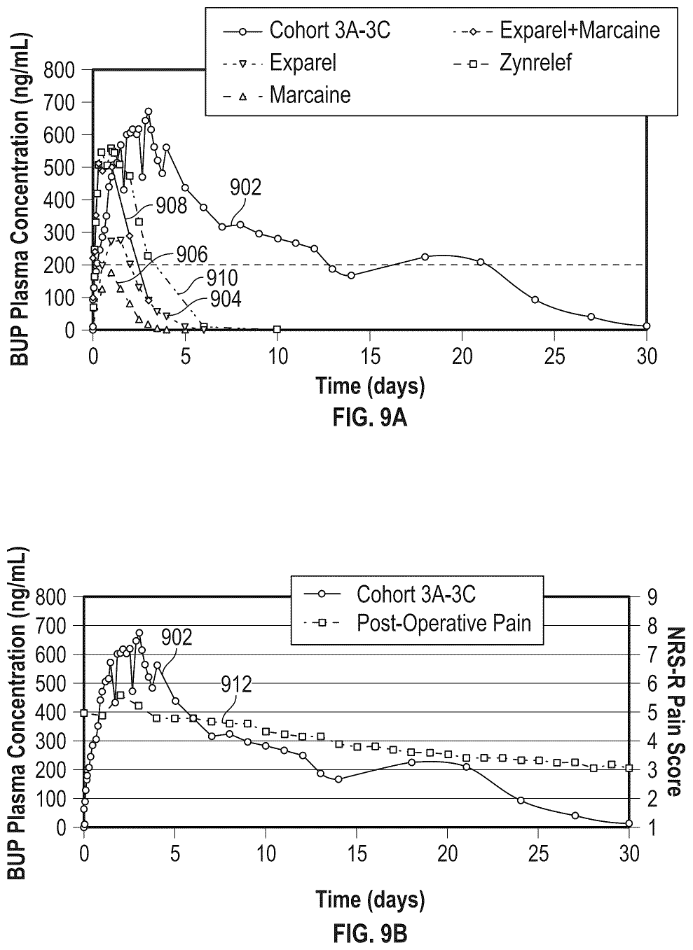

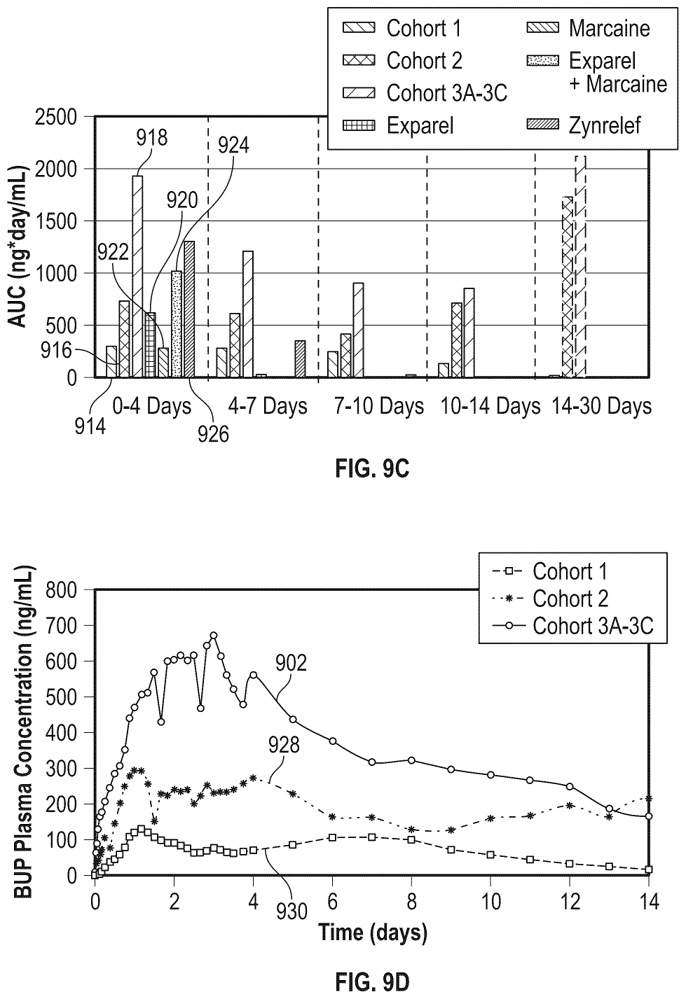

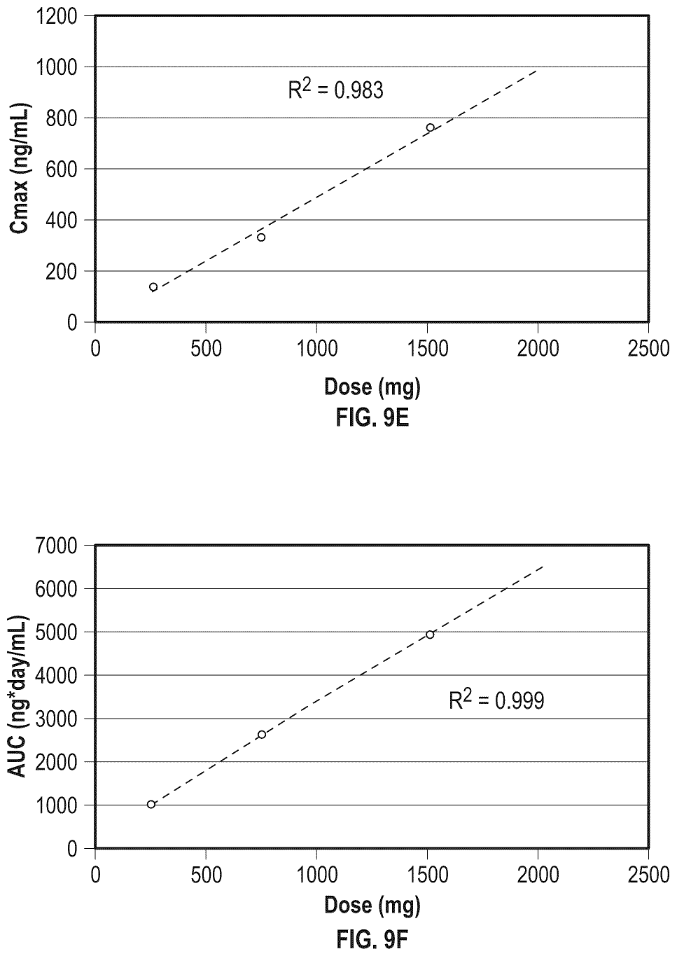

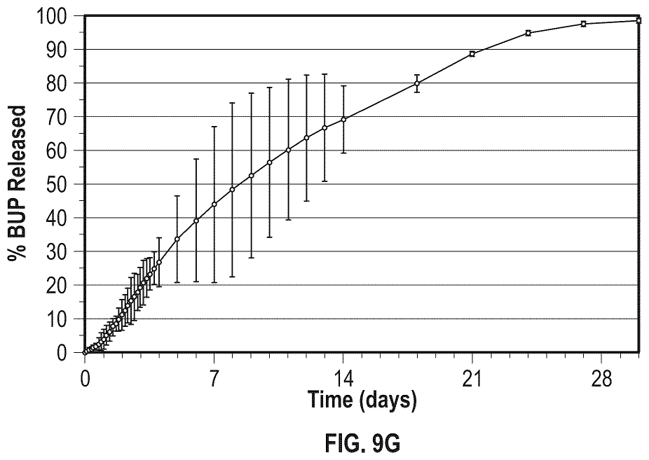

FIG. 9A is a graph showing the mean bupivacaine plasma concentration over time in subjects receiving R300 or T600 depots (line 902) compared to subjects treated with other bupivacaine formulations (lines 904-910), following TKA. Line 902 shows data from Cohort 3A-3C subjects (data for Days 1-14 and 30 are from all Cohort 3A-3C subjects; data for Days 18, 21, 24, and 27 are from Cohort 3C subjects only). Line 904 shows data from subjects treated with Exparel liposomal bupivacaine injection (n=24, 266 mg bupivacaine, Bramlett et al., The Knee 19 (2012), 530-536). Line 906 shows data from subjects treated with Marcaine bupivacaine injection (n=30, 133 mg bupivacaine, Bramlett et al.). Line 908 shows data from subjects treated with Exparel and Marcaine (n=11, 400 mg bupivacaine, Marino et al., The Journal of Arthroplasty 34 (2019) 495-500). Line 910 shows data from subjects treated with Zynrelef bupivacaine and meloxicam injection (n=58, 400 mg bupivacaine, Lachiewicz et al., The Journal of Arthroplasty 35 (2020) 2843-2851).

As shown in FIG. 9A, the mean bupivacaine plasma concentrations in subjects treated with R300 or T600 depots (line 902) remained near or above the 200 ng/ml therapeutic threshold through Day 21. In contrast, the mean bupivacaine plasma concentrations for subjects treated with other formulations fell below the therapeutic threshold within the first 3 to 5 days. This data demonstrates that implantable depots can provide continuous, sustained release of bupivacaine at therapeutic levels for significantly longer time periods than conventional formulations.

FIG. 9B is a graph showing the mean bupivacaine plasma concentration over time in subjects receiving R300 or T600 depots (line 902, left vertical axis) overlaid with NRS-R postoperative pain scores in primary TKA patients (line 912, right vertical axis) obtained from Force Therapeutics Database (n=103,818-296,286). As shown in FIG. 9B, pain scores are elevated immediately after TKA and gradually decrease over the next 30 days. In some instances, to allow for gentle recuperation and rehabilitation, local anesthetic should be present until pain scores drop below 4 (approximately 21 days after TKA). The release profile of the R300 and T600 depots matches the evolution in pain scores over time, by providing higher bupivacaine levels during the acute pain period (0-4 days) and lower bupivacaine levels that are sustained over throughout the recuperation period (4-30 days).

FIG. 9C is a graph showing the AUC of bupivacaine plasma concentration over various time periods in subjects receiving R300 or T600 depots (bars 914-918) compared to subjects treated with other bupivacaine formulations (bars 920-926). Bar 914 shows data from Cohort 1 subjects, bar 916 shows data from Cohort 2 subjects, and bar 918 shows data from Cohort 3A-3C subjects (the broken lines for bars 914-918 at 14-30 days indicates that these AUC values were calculated over a longer time interval (two weeks) compared to the other AUC values shown in FIG. 9C (three to four days)). Bar 920 shows data from subjects receiving Exparel, bar 922 shows data from subjects receiving Marcaine, bar 924 shows data from subjects receiving Exparel and Marcaine, and bar 926 shows data from subjects receiving Zynrelef. The data for the other formulations was obtained from the same sources as in FIG. 9A. As shown in FIG. 9B, the AUC values for subjects treated with R300 or T600 depots was comparable to the AUC values for other formulations during the acute period (0-4 days) and was superior to the other formulations through the recuperation period (4-30 days).

FIG. 9D is a graph showing the mean bupivacaine plasma concentrations in subjects receiving varying doses of bupivacaine from implantable depots. Specifically, line 902 shows data from subjects receiving 1512 mg bupivacaine (Cohorts 3A-3C), line 928 shows data from subjects receiving 756 mg bupivacaine (Cohort 2), and line 930 shows data from subjects receiving 252 mg bupivacaine (Cohort 1). FIG. 9E is a graph showing the relationship between Cₘₐₓ and bupivacaine dose, and FIG. 9F is a graph showing the relationship between AUC_{0-14d} and bupivacaine dose. The data in FIGS. 9D-9F show that the pharmacokinetic parameters of the R300 and T600 depots exhibit a linear dose response.

FIG. 9G is a graph showing the in vivo bupivacaine release profile in subjects receiving implantable depots. The release profile shown in FIG. 9G was estimated from the bupivacaine plasma concentration levels of subjects receiving 1512 mg bupivacaine (Cohorts 3A-3C). Briefly, the total area under the curve (AUC_{0-inf}) of the bupivacaine plasma concentration over time was assumed to correspond to 100% release of the total bupivacaine dose in the depot. The cumulative percentage of bupivacaine released over time was calculated at each study time point t₁ from the ratio of AUC₀₋ₜ₁ to AUC_{0-inf} normalized to 100%. As shown in FIG. 9G, after implantation, the depots exhibited sustained release of bupivacaine for more than 21 days. Approximately 50% of the total bupivacaine dose was released in the first 7 to 8 days, and approximately 90% of the total bupivacaine dose was released in the first 21 days. This data shows that the implantable depots are able to maintain sustained release of bupivacaine during the acute and recuperation periods following surgery.

### Example 5: Clinical Efficacy of Implantable Depots

This example describes postoperative pain and opioid consumption in patients treated with implantable depots following TKA (the Cohort 1-3C subjects of Example 4). While the primary endpoint of the study was bupivacaine concentration, exploratory analysis of clinical efficacy was also evaluated in in terms of NRS-R for pain intensity and opioid consumption.

Operative and postoperative medications for all Cohort 1 subjects, in addition to the implantable depots, included intrathecal morphine; an adductor canal block; a local infiltration cocktail consisting of ropivacaine, clonidine, ketorolac, and epinephrine; a long-acting opioid (Targin); and rescue opioids as needed (mostly oxycodone). These subjects also consumed acetaminophen and celecoxib for varying durations during the trial. In these subjects, no more than 255 mg of ropivacaine could be used in the local infiltration cocktail, adductor canal block, and/or in spinal anesthesia.

Operative and postoperative medications for all Cohort 2 subjects, in addition to the implantable depots, included intrathecal morphine; an adductor canal block; a local infiltration cocktail consisting of ropivacaine, clonidine, ketorolac, and epinephrine; a long-acting opioid (Targin); and rescue opioids as needed (mostly oxycodone). These subjects also consumed acetaminophen and celecoxib for varying durations during the trial. In these subjects, no more than 165 mg of ropivacaine could be used in the local infiltration cocktail, adductor canal block, and/or in spinal anesthesia.

Operative and post-operative medications for Cohort 3A-3C subjects (collectively, “Cohort 3 subjects”), in addition to the implantable depots, included intrathecal morphine in 6 of 15 subjects; a local infiltration cocktail consisting of clonidine, ketorolac, and epinephrine in 6 of 15 subjects and no local infiltration in the remaining 9 subjects; a long-acting opioid (Targin) for 3 of 15 subjects; and rescue opioids as needed (mostly oxycodone). These subjects also consumed acetaminophen and celecoxib for varying durations during the trial. In these subjects, ropivacaine was only permitted as the spinal anesthesia. No adductor canal blocks and no local infiltration with anesthetic was permitted.

NRS-R were conducted before surgery and after surgery at 30 minutes, 1, 2, 3, 4, 6, 9, 12, 15, 18, 21, 24, 28, 32, 36, 40, 44, 48 52, 56, 60, 64, 68, 72, 76, 80, 84, 90, and 96 hours as well as at Days 6, 7, 8, 9, 10, 11, 12, 13, 14, 15, 30, 45, and 60. In addition, due to a protocol amendment, NRS-R for pain intensity was also conducted on Days 18, 21, 24, and 27 for the Cohort 3C subjects. Subjects were asked to complete an NRS-R for pain intensity prior to consuming any opioid from the surgical procedure through Day 15.

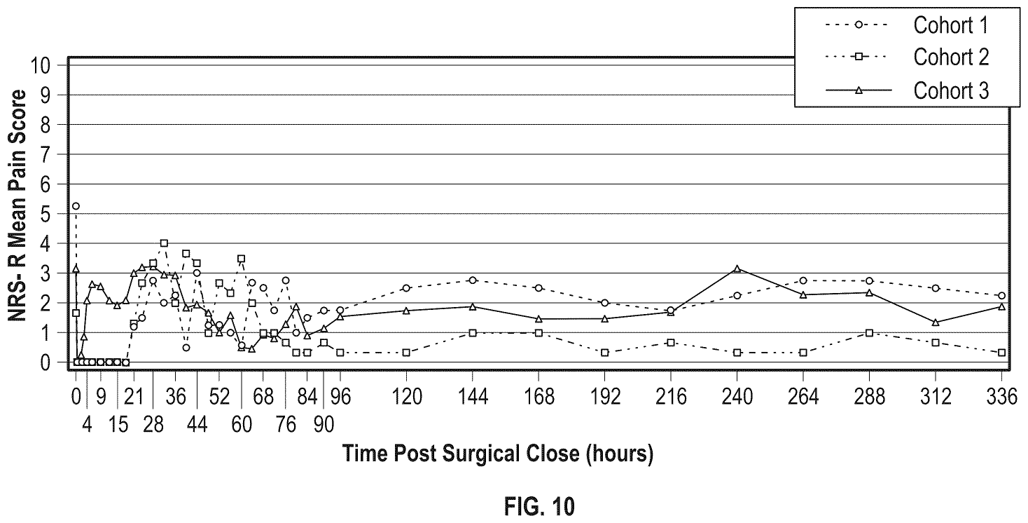

FIG. 10 is a graph showing the mean NRS-R pain scores for each of the cohorts (not adjusted for opioid consumption). There was no significant difference in pain scores across the cohorts. However, in comparing the total amount of opioids consumed (64% less in Cohort 3 versus Cohort 1, see Table 8 below) as well as the limited set of pain scores, it appears patients will consume opioids to try to maintain a tolerable pain intensity level during the first two weeks after a TKA surgery. Overall, patient pain was manageable with pain intensity scores generally at 3 or lower.

The AUC of the NRS-R for pain intensity adjusted for opioid use was calculated for each day and cumulatively through the end of each day using the trapezoidal method. Table 6 below shows the AUC for the first 72 hours. The AUC was comparable between Cohorts 1 and 3; however, all subjects in Cohort 1, Cohort 2, and 6 of 15 subjects in Cohort 3 received intrathecal morphine, which lowered the AUC in those subjects in the first approximately 24 hours.

TABLE 6
Pain Intensity Mean AUC Through Hour 72

| | | Mean Pain | Standard | | | |
|---|---|---|---|---|---|---|
| Cohort | | Intensity | Deviation | | | |
| (dose) | N | AUC_{0-72 h} | (SD) | Median | Min | Max |
| 1 (252 | 4 | 145.02 | 129.598 | 161.57 | 0 | 256.9 |
| mg) | | | | | | |
| 2 (756 | 3 | 157.77 | 167.046 | 140.56 | 0 | 332.8 |
| mg) | | | | | | |
| 3 (1512 | 15 | 184.10 | 110.927 | 198.40 | 8.1 | 406.6 |
| mg) | | | | | | |

Table 7 below shows the AUC for the first 15 days (two weeks post-surgery). The AUC was comparable between Cohorts 1 and 3; however, Cohort 3 subjects consumed 64% less opioids over two weeks (see Table 8 below) and received no operative adjunct anesthetics (adductor canal block, or local infiltration of anesthetic) compared to Cohort 1 subjects.

TABLE 7
Pain Intensity Mean AUC Through Day 15

| | | Mean Pain | | | | |
|---|---|---|---|---|---|---|
| Cohort | | Intensity | | | | |
| (dose) | N | AUC_{0-15 d} | SD | Median | Min | Max |
| 1 (252 | 4 | 912.68 | 1008.720 | 791.41 | 35.4 | 2032.6 |
| mg) | | | | | | |
| 2 (756 | 3 | 502.43 | 473.115 | 567.88 | 0 | 939.4 |
| mg) | | | | | | |
| 3 (1512 | 15 | 918.74 | 688.25 | 822.37 | 88.1 | 1992.9 |
| mg) | | | | | | |

Table 8 below shows patient opioid consumption through the first two weeks after surgery. Every instance of opioid consumption was tracked from the day of the TKA surgical procedure (Day 1) through Day 15. The preliminary interim analysis showed that 80% of subjects (12/15) in Cohort 3 at the 1,512 mg dose discontinued use of all opioids for TKA knee pain within the first two weeks after TKA surgery (Table 1) compared to the literature of 52.8% (Runner et al., The Journal of Arthroplasty 35 (2020), S158-S162). No subjects in Cohort 2 (756 mg) consumed opioids after Day 15 and half of the Cohort 1 (252 mg) subjects (50%) continued consuming opioids beyond Day 15.

TABLE 8
Postsurgical Opioid Consumption Through Day 15

| | | | | Number (%) | |
|---|---|---|---|---|---|
| | | | % Opioid | Of Subjects | |
| | | Total Average | Reduction | Discontinued | |
| Cohort | | MME Through | From | Opioids On Or | |
| (dose) | N | Day 15 (SD) | Cohort 1 | Before Day 15 | |
| 1 (252 mg) | 4 | 488.13 (222.695) | — | 2 | (50%) |
| 2 (756 mg) | 3 | 401.67 (136.961) | 18% | 3 | (100%) |
| 3 (1512 mg) | 15 | 176.77 (156.197) | 64% | 12 | (80%) |

The results showed that as the bupivacaine dose increased, the postsurgical opioid consumption in MME decreased in the first two weeks after surgery. Literature indicates the average MME consumed after a TKA is between 428 MME and nearly 700 MME (Runner et al.; Ruddell et al., The Journal of Bone and Joint Surgery 103 (2021), 106-114). The Cohort 3 subjects consumed less than half of the total opioids reported in the literature (176.77 MME vs 428 MME). 20% of the Cohort 3 subjects (3/15) did consume additional opioids, as needed for knee pain, after the first two weeks and these additional opioids (beyond two weeks) are not captured in the 176.77 MME reported in Table 8.

Of the 15 Cohort 3 subjects, one subject did not consume any opioids (6.7%). Of the remaining subjects that did consume opioids, the time to first consumption was 6.283 hours (95% CI; 3.117, 22.533) as calculated by Kaplan-Meyer.

### Example 6: Simulated Pharmacokinetics of Implantable Depots

This example describes simulated pharmacokinetic data of implantable depots for treating postoperative pain after shoulder surgery, bunionectomy, and inguinal hernia repair. The simulated data was generated by calculating Cₘₐₓ and Tₘₐₓ scaling factors from pharmacokinetic data for other bupivacaine formulations that used the same bupivacaine dosages in treating TKA and another indication of interest (“new indication”). Specifically, the Cₘₐₓ scaling factor was computed by taking the ratio of the Cₘₐₓ in the TKA pharmacokinetic data to the Cₘₐₓ in the pharmacokinetic data for the new indication. The Cₘₐₓ scaling factor was then multiplied with the Cohort 3 bupivacaine plasma concentration data across all time points to obtain scaled bupivacaine plasma concentration data for the new indication. Similarly, the Tₘₐₓ scaling factor was computed by taking the ratio of the Tₘₐₓ in the TKA pharmacokinetic data to the Tₘₐₓ in the pharmacokinetic data for the new indication. The Tₘₐₓ scaling factor was then multiplied with the Cohort 3 bupivacaine time data across all bupivacaine plasma concentration data to derive scaled time data for the new indication. The bupivacaine plasma concentration data could be scaled further based on the linear Cₘₐₓ-dose relationship obtained in Example 4 above to simulate the pharmacokinetics resulting from different bupivacaine dosages. The resulting data provides a rough simulation of the pharmacokinetics of the implantable depots when implanted at other anatomical locations for treating other indications.

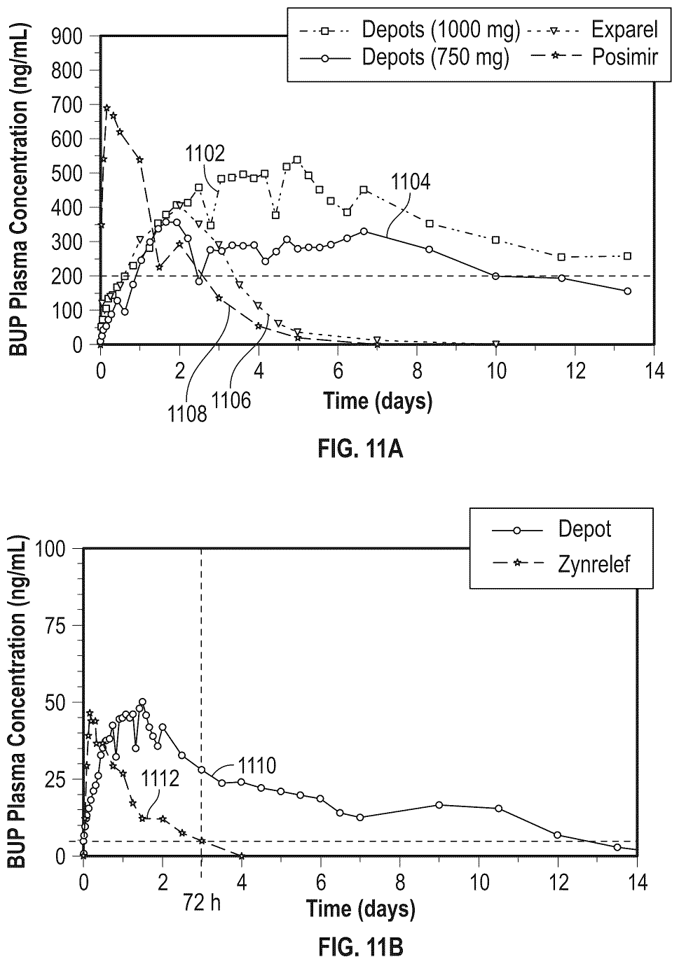

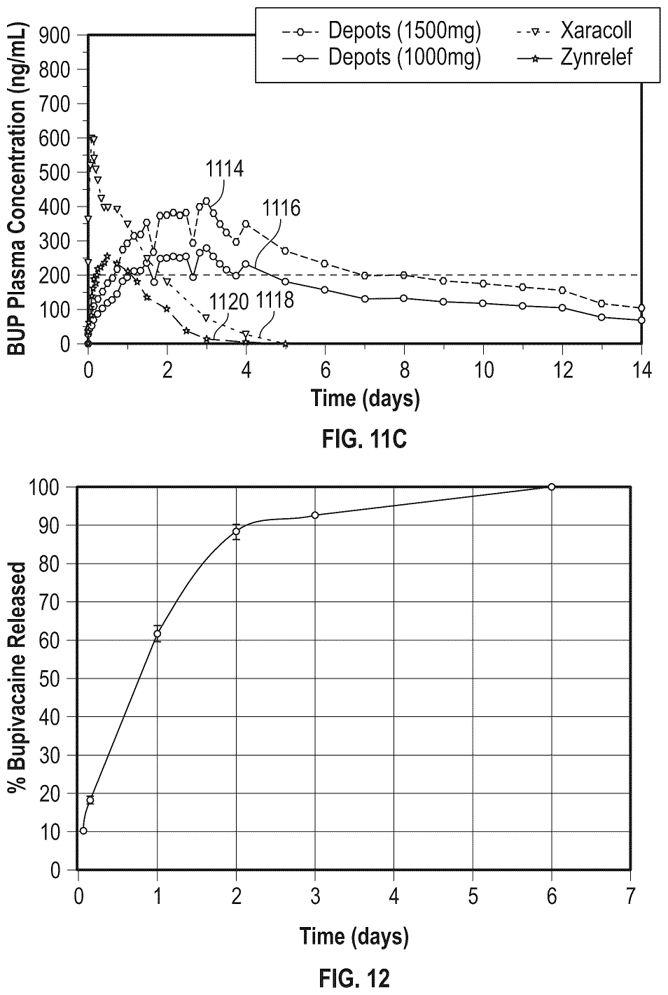

FIG. 11A is a graph showing simulated mean bupivacaine plasma concentrations over time for subjects treated with implantable depots (lines 1102 and 1104) versus actual mean bupivacaine plasma concentrations in subjects treated with other bupivacaine formulations (lines 1106 and 1108), following shoulder surgery. Specifically, line 1102 shows simulated bupivacaine levels for subjects treated with depots containing a 1000 mg dose of bupivacaine (e.g., two T500 depots), line 1104 shows simulated bupivacaine levels for subjects treated with depots containing a 750 mg dose of bupivacaine (e.g., three R300 depots), line 1106 shows bupivacaine levels in subjects treated with Exparel (266 mg bupivacaine, Patel et al., Pain Medicine 21 (2020), 387-400), and line 1108 shows bupivacaine levels in subjects treated with Posimir (bupivacaine extended release solution, 660 mg bupivacaine, FDA Briefing Document, Meeting of Anesthetic and Analgesic Drug Products Advisory Committee (2020)). As shown in FIG. 11A, the implantable depots are expected to produce bupivacaine plasma levels above the 200 ng/ml therapeutic threshold for over 10 days, thus covering the acute and subacute pain periods following shoulder surgery. In contrast, bupivacaine plasma levels in subjects treated with the Exparel and Posimir formulations drop below the therapeutic threshold in the first 3 to 4 days after surgery.

FIG. 11B is a graph showing simulated mean bupivacaine plasma concentrations over time for subjects treated with an implantable depot (line 1110) versus actual mean bupivacaine plasma concentrations in subjects treated with another bupivacaine formulation (line 1112), after bunionectomy. Specifically, line 1110 shows simulated bupivacaine levels for subjects treated with a depot containing a 250 mg dose of bupivacaine (e.g., one R300 depot), while line 1112 shows bupivacaine levels in subjects treated with Zynrelef (60 mg bupivacaine, Viscusi et al., ASRA poster (2017), Viscusi et al., ESRA poster (2017)). Zynrelef is approved for postoperative analgesia up to 72 hours following bunionectomy. As shown in FIG. 11B, the implantable depot is expected to maintain bupivacaine plasma levels at or above the levels produced by Zynrelef at the 72 hour time point for over 12 days, as well as at or above the estimated 5 ng/ml therapeutic threshold, thus covering the acute and subacute pain periods following bunionectomy.

FIG. 11C is a graph showing simulated mean bupivacaine plasma concentrations over time for subjects treated with implantable depots (lines 1114 and 1116) versus actual mean bupivacaine plasma concentrations in subjects treated with other bupivacaine formulations (lines 1118 and 1120), following open inguinal hernia repair. Specifically, line 1118 shows simulated bupivacaine levels for subjects treated with depots containing a 1500 mg dose of bupivacaine (e.g., three T500 depots), line 1116 shows simulated bupivacaine levels for subjects treated with depots containing a 1000 mg dose of bupivacaine (e.g., two T500 depots), line 1118 shows bupivacaine levels in subjects treated with Xaracoll (bupivacaine implant, 266 mg BUP, Leiman et al., Advances in Therapy 38 (2021), 691-706), and line 1120 shows bupivacaine levels in subjects treated with Zynrelef (300 mg bupivacaine, Viscusi et al., ESRA poster (2017)). As shown in FIG. 11C, the implantable depots are expected to produce bupivacaine plasma levels above the 200 ng/ml therapeutic threshold for over 7 days, thus covering the acute and subacute pain periods following open inguinal hernia repair. In contrast, bupivacaine plasma levels in subjects treated with the Exparel and Posimir formulations drop below the therapeutic threshold in the first 1 to 2 days after surgery.

### Example 7: In Vitro Release from Bupivacaine Free Base Depots without Control Regions

This example describes in vitro release from a depot containing bupivacaine free base (“BUPFB”). The depot included a therapeutic region only, without any control regions (similar to the depot 100c of FIG. 1C). The composition and geometry of the depot is listed in Table 9 below.

TABLE 9
Depot Composition and Geometry

| | | | Control | | |
|---|---|---|---|---|---|
| | | Number of | Region | Therapeutic | |
| Sample | BUPFB | Control | Compo- | Region | Depot |
| Name | Content | Regions | sition | Composition | Geometry |
| C100- | 100 mg | 0 | N/A | 3.2% PS20 | Circle |
| FB-TR | | | | 32.3% PLGA5050 | Outer |
| | | | | 64.5% BUPFB | diameter: |
| | | | | | 14 mm |

The therapeutic region was prepared by mixing PS20, PLGA5050, BUPFB, and acetone in a 1:10:20:30 ratio by mass. A thermal press was used to compress and dry the formulation into large circular discs. The larger discs were subsequently cut into smaller circular discs (similar to the depot 550 of FIG. 5F) with an outer diameter of 14 mm and targeted drug loading of 100 mg BUPFB.

FIG. 12 is a graph illustrating cumulative in vitro release of bupivacaine from the C100-FB-TR depot (n=3). The release data was obtained using an accelerated in vitro release test. Samples were immersed in phosphate buffer at pH 5.8. At predetermined time points, aliquots of the buffer were drawn and analyzed using UV-Vis spectroscopy to quantify the amount of bupivacaine released. As shown in FIG. 12, the C100-FB-TR depot exhibited controlled release of the payload over 6 days.

### Example 8: In Vitro Release from Bupivacaine Free Base Depots with Varying Control Regions

This example describes in vitro release from BUPFB depots including 2, 1, and no control regions. The compositions and geometry of the depots are listed in Table 10 below.

TABLE 10
Depot Compositions and Geometry

| | | Number of | Control | Therapeutic | |
|---|---|---|---|---|---|
| Sample | BUPFB | Control | Region | Region | Depot |
| Name | Content | Regions | Composition | Composition | Geometry |
| R300- | 300 mg | 0 | N/A | 3.2% PS20 | Rectangle |
| FB-TR | | | | 32.3% PLGA5050 | 27 mm × 17 |
| | | | | 64.5% BUPFB | mm × 1 mm |
| R300- | 300 mg | 1 | 33.3% PS20 | 3.2% PS20 | Rectangle |
| FB-1CR | | | 66.7% PLGA5050 | 32.3% PLGA5050 | 27 mm × 17 |
| | | | | 64.5% BUPFB | mm × 1 mm |
| R300- | 300 mg | 2 | 33.3% PS20 | 3.2% PS20 | Rectangle |
| FB-2CR | | | 66.7% PLGA5050 | 32.3% PLGA5050 | 27 mm × 17 |
| | | | | 64.5% BUPFB | mm × 1 mm |

The therapeutic regions of the R300-FB-TR, R300-FB-1CR, and R300-FB-2CR were prepared according to the process of Example 7, except that the therapeutic regions were cut into rectangles.

The R300-FB-TR depot did not include any control regions, the R300-FB-1CR depot included a single control region (similar to the depot 100b of FIG. 1B), and the R300-FB-2CR depot included two control regions (similar to the depot 100a of FIG. 1A). The control regions were prepared by mixing PS20, PLGA5050, and acetone in a 1:2:6 ratio by mass. The control regions were then formed using a solvent casting process in which the formulation was spread thinly across a polytetrafluoroethylene block (PTFE) block and the acetone flashed off. The control region was then applied to the therapeutic region via a heat compression using a thermal press.

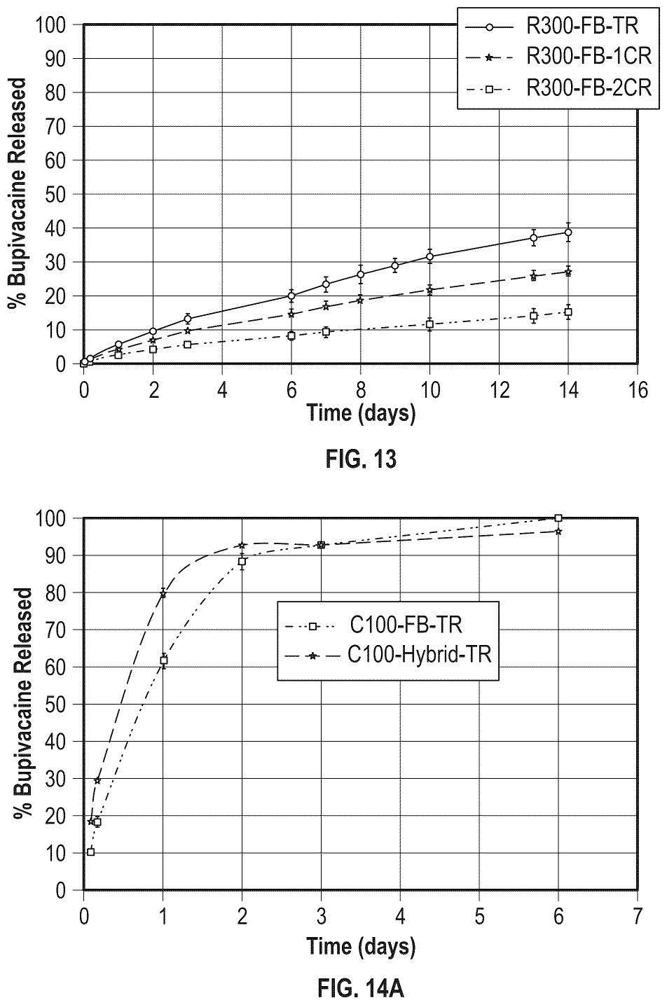

FIG. 13 is a graph illustrating cumulative in vitro release of bupivacaine from the depots (n=2 for each depot type). The depots were immersed in a phosphate buffer at pH 7.4. At predetermined time points, the depots were removed from the pH 7.4 buffer and placed into fresh pH 7.4 buffer. The buffer was analyzed using UV-Vis spectroscopy to quantify the amount of bupivacaine released at each time point. As shown in FIG. 13, all three depots exhibited controlled release over a period of 14 days. The release rate was slowed as the number of control regions increased.

### Example 9: In Vitro Release from Bupivacaine Free Base, Salt, and Hybrid Depots

This example describes in vitro release from depots formulated with BUPFB, bupivacaine hydrochloride monohydrate (“BUP-HCl”), or a mixture of BUPFB and BUP-HCl (“hybrid” depots). The compositions and geometry of the depots are listed in Table 11 below.

TABLE 11
Depot Compositions and Geometry

| | | | Control | | |
|---|---|---|---|---|---|
| | | Number of | Region | Therapeutic | |
| Sample | BUPFB | Control | Compo- | Region | Depot |
| Name | Content | Regions | sition | Composition | Geometry |
| C100- | 100 mg | 0 | N/A | 3.2% PS20 | Circle |
| FB-TR | | | | 32.3% PLGA5050 | Outer |
| | | | | 64.5% BUPFB | diameter: |
| | | | | | 14 mm |
| C100- | 100 mg | 0 | N/A | 3.2% PS20 | Circle |
| hybrid- | | | | 32.3% PLGA5050 | Outer |
| TR | | | | 32.25% BUP-HCl | diameter: |
| | | | | 32.25% BUPFB | 14.6 mm |
| T400- | 400 mg | 0 | N/A | 3.2% PS20 | Triangle |
| salt-TR | | | | 32.3% PLGA5050 | 25 mm × |
| | | | | 64.5% BUP-HCl | 25 mm × |
| | | | | | 1.5 mm |

The depots were prepared according to the methods described in Example 7 above, except for the following changes: (1) for the C100-hybrid-TR depot, the therapeutic region was formulated using a mixture of PS20, PLGA5050, BUP-HCl, BUPFB, and acetone in a 1:10:10:10:30 ratio by mass, with a targeted drug loading of 50 mg BUPFB and 60 mg BUP-HCl (equivalent to 100 mg BUPFB); and (2) for the T400-salt-TR depot, the therapeutic region was formulated using a mixture of PS20, PLGA5050, BUP-HCl, and acetone in a 1:10:20:30 ratio by mass, with a targeted drug loading of 480 mg BUP-HCl (equivalent to 400 mg BUPFB), and cut into a triangular shape.

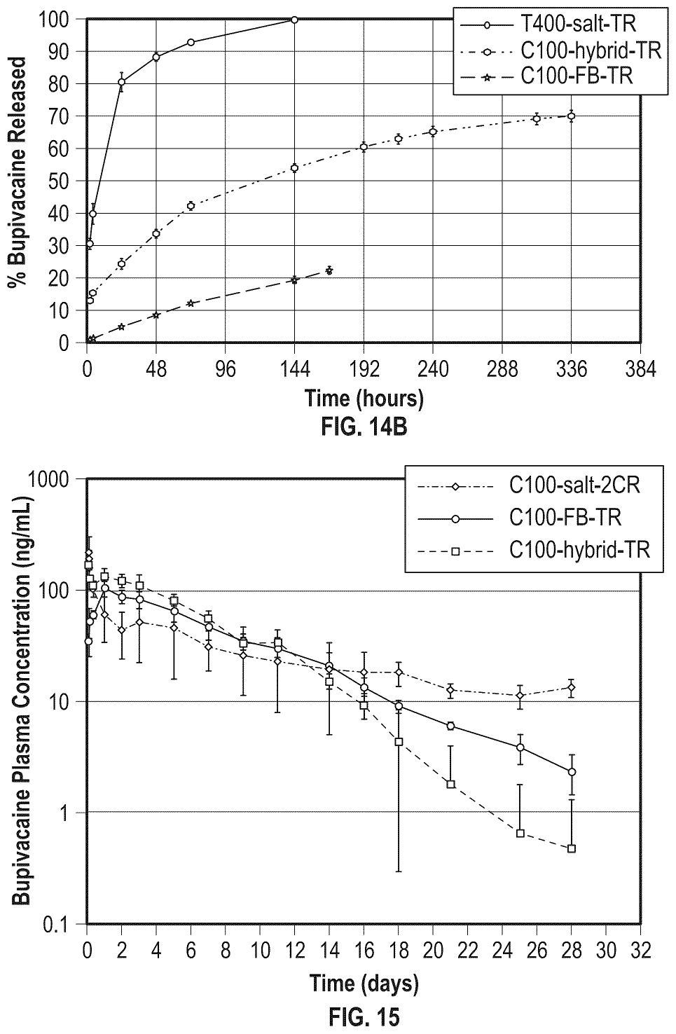

FIG. 14A is a graph illustrating cumulative in vitro release of bupivacaine from the C100-FB-TR and C100-hybrid-TR depots (n=3 for each depot type). The release data was obtained using the accelerated in vitro release test described in Example 7 above. As shown in FIG. 14A, the hybrid depot exhibited faster release than the depot formulated with BUPFB.

FIG. 14B is a graph illustrating cumulative in vitro release of bupivacaine from all three depots. The release data was obtained using the accelerated in vitro release test described in Example 7 above. As shown in FIG. 14B, the depot formulated with BUP-HCl only (T400-salt-TR, n=5) released the fastest, followed by the hybrid depot (C100-hybrid-TR, n=6), then the depot formulated with BUPFB only (C100-FB-TR, n=3).

### Example 10: In Vivo Release from Bupivacaine Free Base, Salt, and Hybrid Depots

This example describes in vivo release in a rabbit subcutaneous model from depots with different forms of bupivacaine. The compositions and geometry of the depots are listed in Table 12 below.

TABLE 12
Depot Compositions and Geometry

| | | Number of | Control | Therapeutic | |
|---|---|---|---|---|---|
| Sample | BUPFB | Control | Region | Region | Depot |
| Name | Content | Regions | Composition | Composition | Geometry |
| C100- | 100 mg | 0 | N/A | 3.2% PS20 | Circle |
| FB-TR | | | | 32.3% PLGA5050 | Outer |
| | | | | 64.5% BUPFB | diameter: |
| | | | | | 14 mm |
| C100- | 100 mg | 0 | N/A | 3.2% PS20 | Circle |
| hybrid- | | | | 32.3%P LGA5050 | Outer |
| TR | | | | 32.25% BUP-HCl | diameter: |
| | | | | 32.25% BUPFB | 14.6 mm |
| C100- | 100 mg | 2 | 100% PLGA5050 | 3.2% PS20 | Circle |
| salt-2CR | | | | 32.3% PLGA5050 | Outer |
| | | | | 64.5% BUP-HCl | diameter: |
| | | | | | 15.3 mm |

The C100-FB-TR and C100-hybrid-TR depots were prepared as described above in Example 9. The C100-salt-2CR depot contained a therapeutic region with bupivacaine hydrochloride monohydrate (BUP-HCl) and two control regions (similar to the depot 100a of FIG. 1A). The therapeutic region of the C100-salt-2CR depot was prepared as described in Example 7 above, except using BUP-HCl rather than BUPFB for the therapeutic region. The control region of the C100-salt-2CR depot was prepared via a dip coating process using a formulation comprising PLGA5050 and acetone in a mass ratio of 2:9. In this dip coating process, the large disc manufactured using the thermal press method described in Example 7 was dipped in its entirety into a vessel containing the 2:9 PLGA:acetone dip coating formulation. The targeted drug loading for the C100-salt-2CR depot was 120 mg BUP-HCl (equivalent to 100 mg BUPFB).

FIG. 15 is a semilog graph illustrating in vivo release of bupivacaine from the depots in a rabbit subcutaneous model. 4 rabbits were each implanted with two depots in the subcutaneous space along the dorsal region. Only one subcutaneous pocket was created for the two depots. Blood draws were performed at predetermined time points (baseline, 1, 3, 8, 24, 48, 72, 120, 168, 216, 264, 336, 384, 432, 504, 600, and 672 hours). A bupivacaine assay was performed on each aliquot to quantify the plasma concentration of bupivacaine free base at each time point. As shown in FIG. 15, the hybrid depot (C100-hybrid-TR, n=3) released faster than the BUPFB depot (C100-FB-TR, n=4). The presence of control regions extended the release duration, even when the hydrophilic BUP-HCl form (C100-salt-2CR, n=4) was used.

### Example 11: In Vitro Release from Bupivacaine Free Base Depots with Varying Therapeutic Loading

This example describes in vitro release from depots formulated with varying amounts of BUPFB. The compositions and geometry of the depots are listed in Table 13 below.

TABLE 13
Depot Compositions and Geometry

| | | Number | Control | | |
|---|---|---|---|---|---|
| | | of | Region | Therapeutic | |
| Sample | BUPFB | Control | Compo- | Region | Depot |
| Name | Content | Regions | sition | Composition | Geometry |
| R300- | 300 mg | 0 | N/A | 3.2% PS20 | Rectangle |
| FB-TR | | | | 32.3% PLGA5050 | 27 mm × 17 |
| | | | | 64.5% BUPFB | mm × 1 mm |
| R300- | 300 mg | 0 | N/A | 2% PS20 | Rectangle |
| FB2-TR | | | | 19.6% PLGA5050 | 27 mm × 17 |
| | | | | 78.4% BUPFB | mm × 1 mm |

The depots were prepared as described in Example 8 above, except that the therapeutic region of the R300-FB2-TR depot included a mixture of PS20, PLGA, BUPFB, and acetone in a 1:10:40:30 ratio by mass.

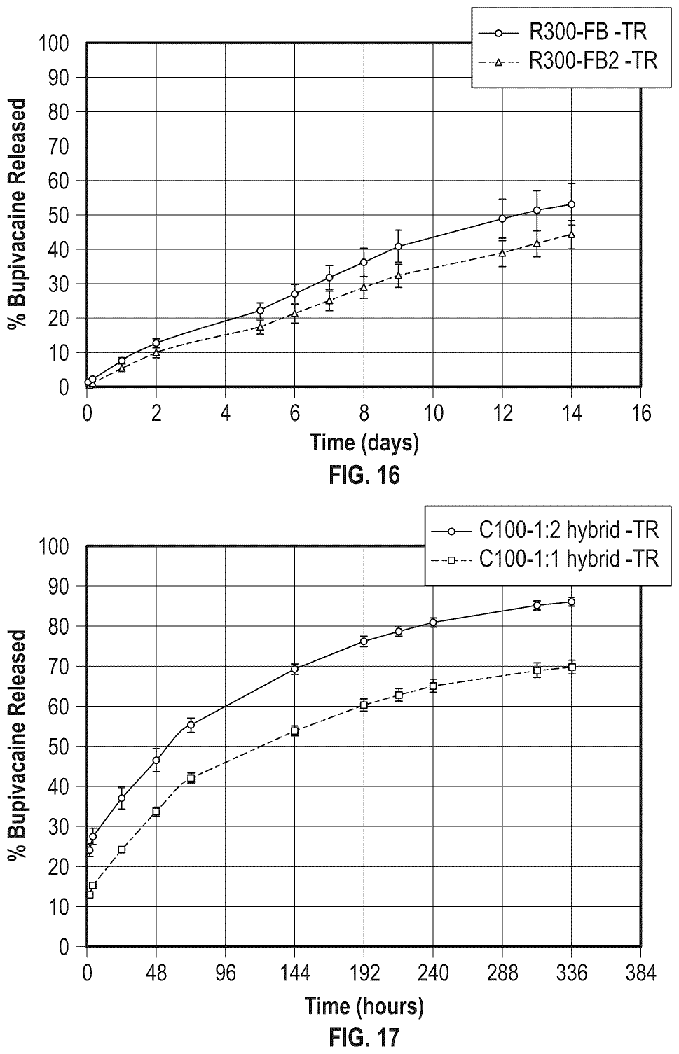

FIG. 16 is a graph illustrating cumulative in vitro release of bupivacaine from the depots. In vitro release testing was performed at pH 7.4 using the method of Example 8. As shown in FIG. 16, both depots exhibited controlled release over a period of 2 weeks. The depot with higher BUPFB loading (R300-FB2-TR, 78.4% BUPFB, n=2) exhibited slightly slower release than the depot with lower BUPFB loading (R300-FB-TR, 64.5% BUPFB, n=2).

### Example 12: In Vitro Release from Depots with Varying Bupivacaine Free Base:Salt Ratios

This example describes in vitro release from depots formulated with varying BUPFB:BUP-HCl ratios. The compositions and geometry of the depots are listed in Table 14 below.

TABLE 14
Depot Compositions and Geometry

| | | Number | Control | | |
|---|---|---|---|---|---|
| | | of | Region | Therapeutic | |
| Sample | BUPFB | Control | Compo- | Region | Depot |
| Name | Content | Regions | sition | Composition | Geometry |
| C100- | 100 mg | 0 | N/A | 3.2% PS20 | Circle |
| 1:2hybrid- | | | | 32.3% PLGA5050 | Outer |
| TR | | | | 41.9% BUP-HCl | diameter: |
| | | | | 22.6% BUPFB | 14 mm |
| C100- | 100 mg | 0 | N/A | 3.2% PS20 | Circle |
| 1:1hybrid- | | | | 32.3% PLGA5050 | Outer |
| TR | | | | 32.25% BUP-HCl | diameter: |
| | | | | 32.25% BUPFB | 14 mm |

The depots were prepared as described in Example 7 above, except that (1) the therapeutic region of the C100-1:1hybrid-TR depot was formulated using a mixture of PS20, PLGA5050, BUP-HCl, BUPFB, and acetone in a 1:10:10:10:30 ratio by mass, with a targeted drug loading of 50 mg BUPFB and 60 mg BUP-HCl (equivalent to 100 mg BUPFB); and (2) the therapeutic region of the C100:1:2hybrid-TR depot was formulated using a mixture of PS20, PLGA5050, BUP-HCl, BUPFB, and acetone in a 1:10:13.7:30 ratio by mass, with a targeted drug loading of 39 mg BUPFB and 72.5 mg BUP-HCl (equivalent to 100 mg BUPFB).

FIG. 17 is a graph illustrating cumulative in vitro release of bupivacaine from the depots. In vitro release testing was performed at pH 7.4 using the method of Example 8. As shown in FIG. 17, the depot with the higher BUPFB:BUP-HCl ratio (C100-1:1hybrid-TR, n=6) released more slowly than the depot with the lower BUPFB:BUP-HCl ratio (C100-1:2hybrid-TR, n=6).

### Example 13: Travel Distance Modeling

This example describes modeling techniques for determining the travel distance of a therapeutic agent for various depot geometries.

In some embodiments, the depots described herein release the therapeutic agent (e.g., bupivacaine) with first order kinetics under sink conditions (e.g., PBS at pH 5.8), such that the half-life (t_{1/2}) is related to the observed rate constant (k_{obs}) by the equation t_{1/2}=ln (2)/k_{obs}. The half-life can be determined experimentally using the in vitro elution techniques described herein. The half-life is expected to vary with the geometry of the depot, including the mean travel distance of the therapeutic agent to the nearest exposed surface of the depot. Two modeling approaches were developed to validate the relationship between release rates and travel distance: a Monte Carlo statistical approach and a geometric/calculus approach.

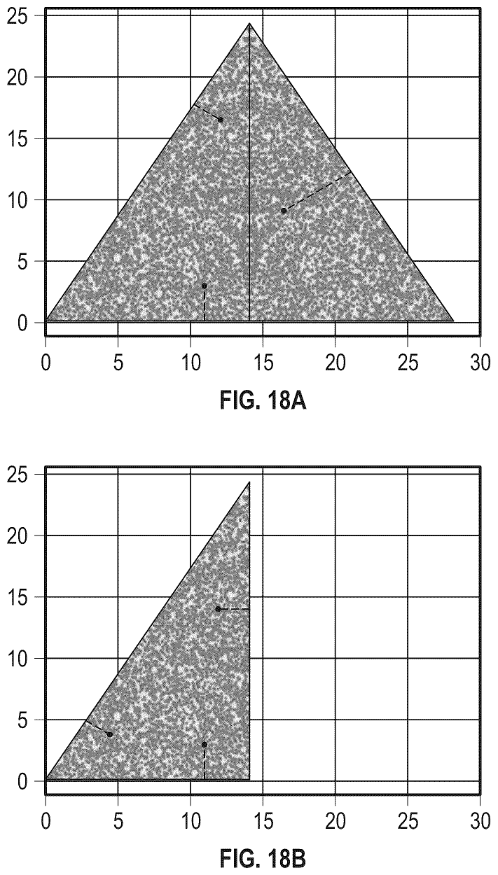

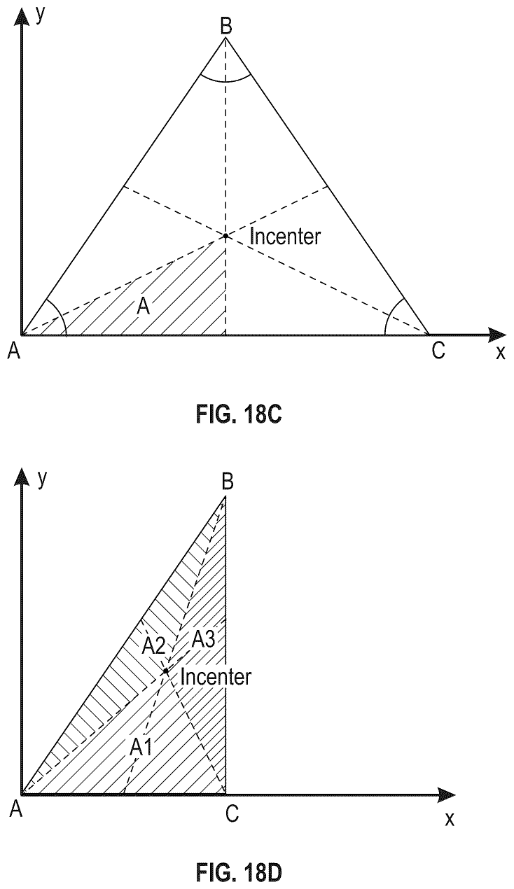

FIGS. 18A and 18B illustrate the Monte Carlo approach applied to two depot geometries with upper and lower control regions: an equilateral triangle (“T500”) (FIG. 18A) and a right triangle (“T250”) (FIG. 18B). A Monte Carlo model was developed using Python and operated as follows: for each depot geometry, a number of pseudo-random points were added to the interior of the depot. The travel distance from each point to the closest edge was then determined (3 examples are shown in FIGS. 18A and 18B), and the average of the travel distances for all points was then calculated. The ratio of the average travel distances for T500 and T250 was then compared to the ratio of the observed rate constants (or the ratio of the t_{1/2} values). Both ratios were approximately 1.4, which indicates that the average travel distance is directly proportional to the observed rate constant. The model can be used to predict relative release rates of the therapeutic agent from different depot geometries (e.g., different shapes, with or without holes, with or without control regions).

FIGS. 18C and 18D illustrate the geometric/calculus approach applied to the T500 (FIG. 18C) and T250 (FIG. 18D) depot geometries. The depots are geometrically divided into smaller areas using the furthest point from every edge (the incenter), and the integral over each smaller area is taken. The integrand is the known formula for shortest distance from a point to a line. The integral outputs the average minimum distance to an edge for an infinite number of points. The output of the integral is expected to match the output of the Monte Carlo simulation.

### Example 14: In Vitro Release from Implantable Depots with Different Geometries

This example describes in vitro release from depots with different geometries, as listed in Table 15 below.

TABLE 15
Depot Geometries

| | | Dimensions | Average | | Bupivacaine | |
|---|---|---|---|---|---|---|
| | | (length × height/ | Travel | | Content | |
| Name | Geometry | width × thickness) | Distance | | (BUPFB | |
| T500 | Equilateral | 28.2 mm × 25.3 mm × | 1.56 | mm | 500 | mg |
| | triangle | 2 mm | | | | |
| T250 | Right | 14. mm × 25.3 mm × | 1.33 | mm | 250 | mg |
| | triangle | 2 mm | | | | |
| R588 | Rectangle | 79.4 mm × 6.5 mm × | 1.52 | mm | 588 | mg |
| | | 2 mm | | | | |
| R267 | Rectangle | 52.9 mm × 4.3 mm × | 1.01 | mm | 267 | mg |
| | | 2 mm | | | | |
| S92 | Square | 9 mm × 9 mm × 2 mm | 1.5 | mm | 92 | mg |

Each depot included a therapeutic region and two control regions (similar to the depot 100a of FIG. 1A). The therapeutic region was formulated with PS20, PLGA5050, BUP-HCl monohydrate, and acetone in a 1:10:20:30 ratio by mass. After mixing, the therapeutic region formulation was compressed and dried. The control regions were formulated with PLGA5050 and acetone in a 1:4 ratio by mass, and were applied to the therapeutic region by dip coating. The constructs were then cut into the desired shapes.

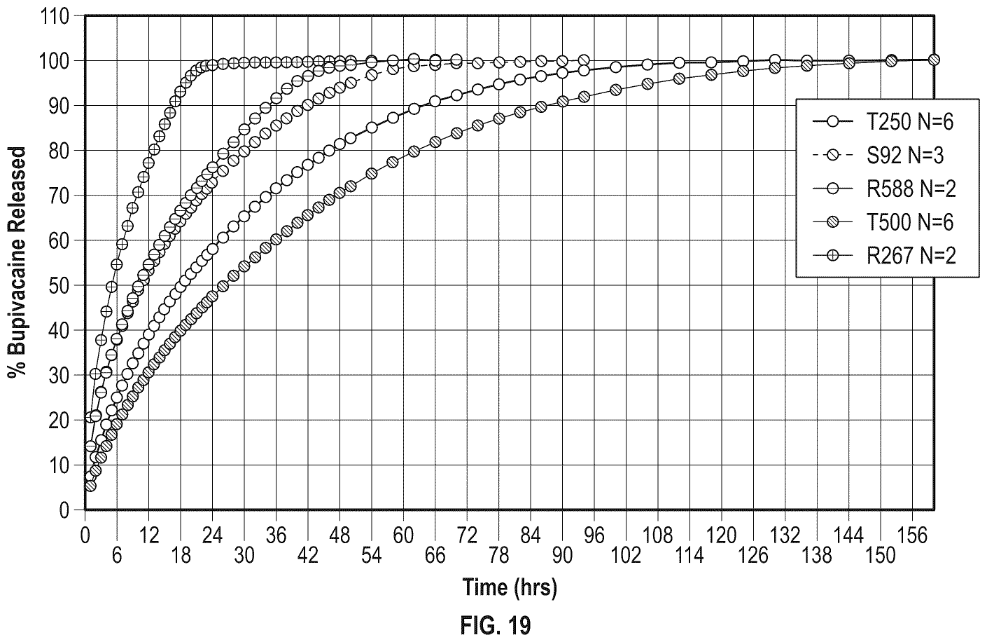

FIG. 19 is a graph illustrating cumulative in vitro release of bupivacaine from each depot geometry. The release data was obtained using an accelerated in vitro release test. Samples were immersed in phosphate buffer at pH 5.8 and 37° C., in baskets stirring at 10 RPM. At predetermined time points, aliquots of the buffer were drawn and analyzed using UV-Vis spectroscopy at 263 nm to quantify the amount of bupivacaine released. As shown in FIG. 19, the depot geometry had a significant effect on bupivacaine release rate. Specifically, the release rates were generally correlated with travel distance, in that the fastest release was observed in the R267 depots, which had the shortest average travel distance (1.01 mm), while the slowest release was observed in the T500 depots, which had the longest average travel distance (1.56 mm). The technique used to cut the depots into their final shape also affected the release rate, due to “rollover” of the edges of the control regions onto the sidewalls of the therapeutic region after cutting. The T250 and T500 depots were cut using a steel rule die that produced more rollover, whereas the other depots were hand cut using a blade that produced little or no rollover. Thus, the T250 depot exhibited slower release than the R588 depot, despite having a shorter travel distance (1.33 mm versus 1.52 mm).

### Example 15: In Vitro Release from Implantable Depots with Different Bupivacaine Particle Sizes

This example describes in vitro release from depots formulated with bupivacaine having different particle sizes.

Triangular depots (similar to the depot 400 of FIG. 4A) (“T500”) were formulated with a therapeutic region and two control regions (similar to the depot 100a of FIG. 1A), using the process described in Example 14. Three different BUP-HCl powders were used to formulate the therapeutic region: (1) unmilled BUP-HCl powder with an average particle diameter greater than 300 μm, (2) BUP-HCl powder having a D50 value of 29 μm, and (3) BUP-HCl powder having a D50 value of 2 μm. The bupivacaine dosage in each therapeutic region was 500 mg BUPFB equivalents.

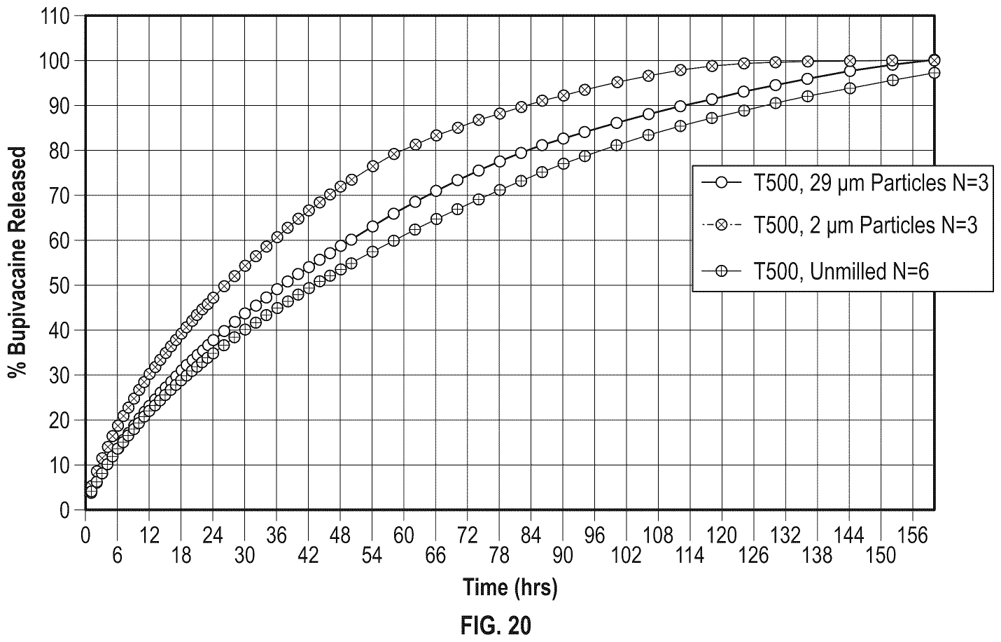

FIG. 20 is a graph illustrating cumulative in vitro release of bupivacaine from depots formulated with each particle size. The release data was obtained using the accelerated in vitro release test described in Example 14. As shown in FIG. 20, the bupivacaine release rate from the depots was affected by the particle size, with smaller particle sizes producing faster release.

### Example 16: In Vitro Release from Bupivacaine Hydrochloride Depots with and without Control Regions

This example describes in vitro release from depots formulated with BUP-HCl, with and without control regions.

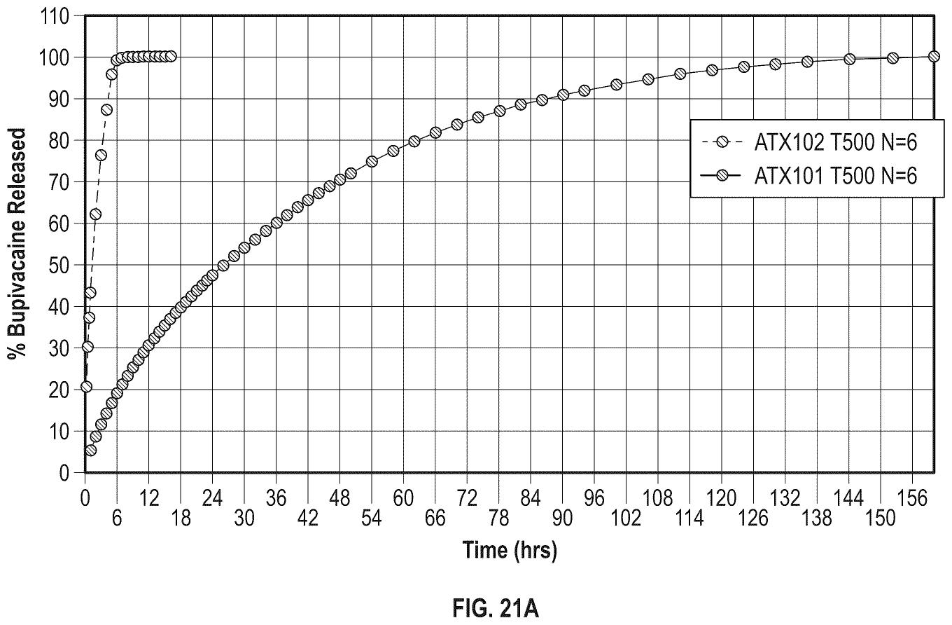

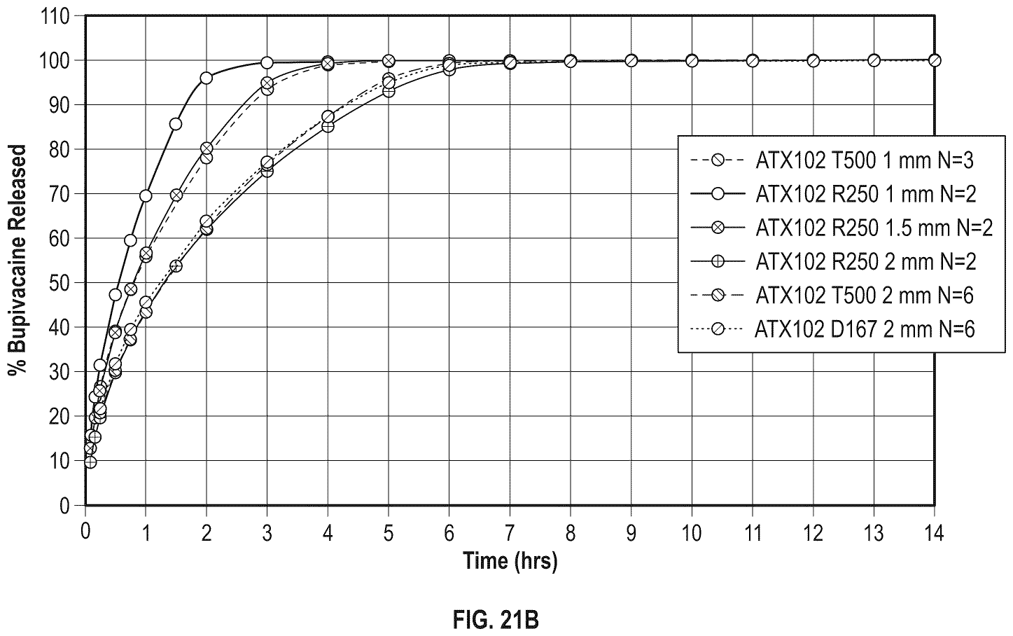

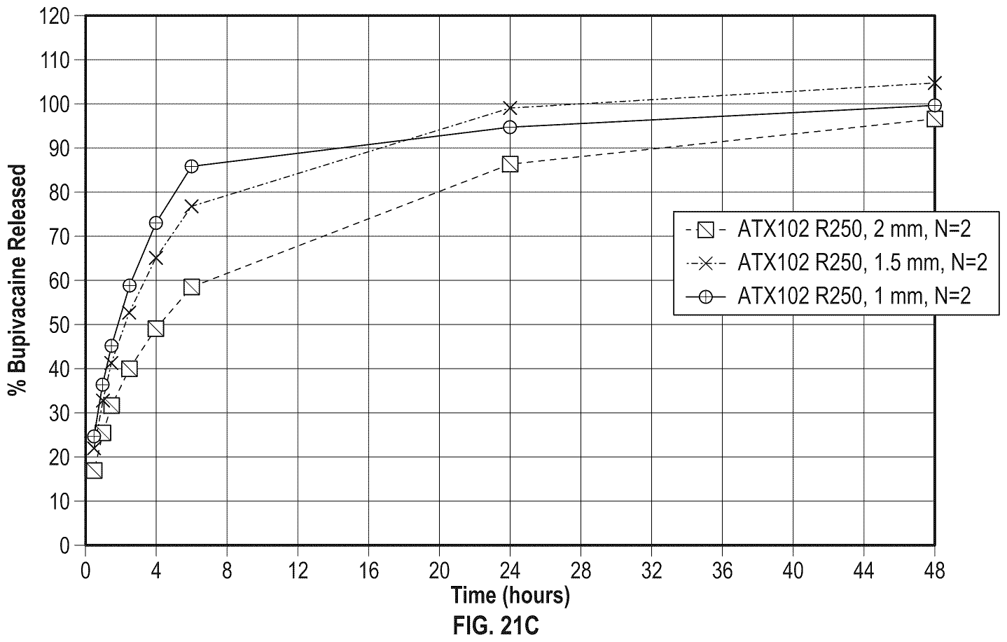

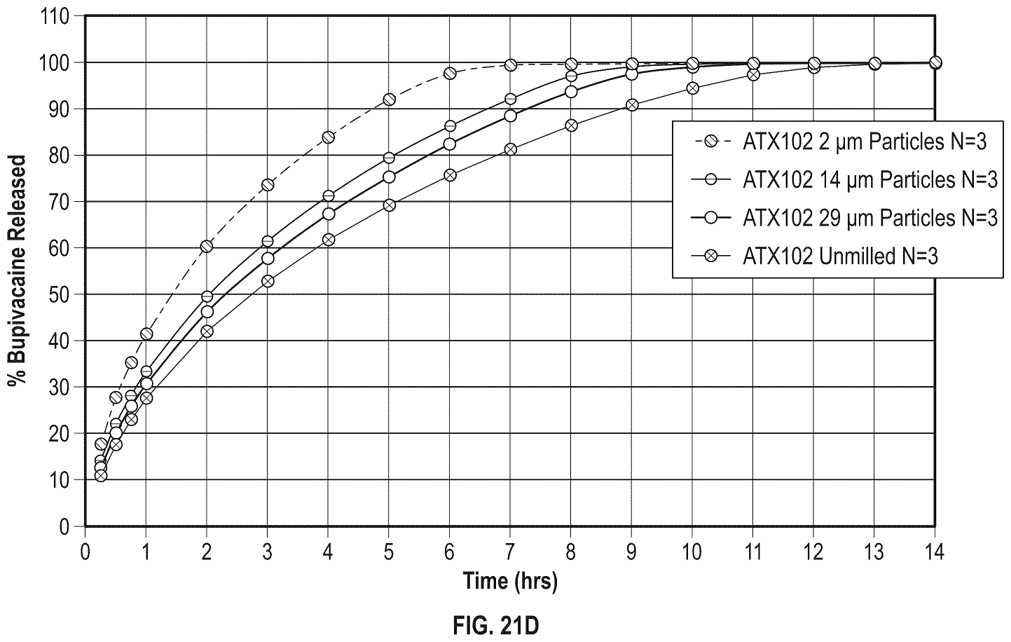

FIG. 21A is a graph illustrating cumulative in vitro release of bupivacaine from depots with control regions (“ATX101”) versus depots without control regions (“ATX102”) at pH 5.8. The ATX101 depots included a therapeutic region with two control regions (similar to the depot 100a of FIG. 1A), and were formulated using the process of Example 14. The ATX102 depots included a therapeutic region without any control regions (similar to the depot 100c of FIG. 1C), and were formulated using the process of Example 14, except that the dip coating procedure was omitted. The ATX101 and ATX102 depots in FIG. 21A had a triangular shape with a dosage of 500 mg BUPFB equivalents. Release data was obtained using the accelerated in vitro release test described in Example 14. As shown in FIG. 21A, the ATX102 depots exhibited significantly faster release rates than the ATX101 depots, with 100% release at approximately 6 hours for the ATX102 depots, versus 100% release at approximately 152 hours for the ATX101 depots.

FIG. 21B is a graph illustrating cumulative in vitro release of bupivacaine from ATX102 depots having different geometries and thicknesses at pH 5.8, as listed in Table 16 below. The ATX102 depots were manufactured using the process of Example 14, except that the dip coating procedure was omitted. Release data was obtained using the pH 5.8 accelerated in vitro release test described in Example 14.

TABLE 16
Depot Geometries

| | | | Bupivacaine Content |
|---|---|---|---|
| Name | Shape | Thickness | (BUPFB equivalents) |
| T500 | Triangle | 2 mm, 1 mm | 500 mg |
| R250 | Rectangle | 2 mm, 1.5 mm, 1 mm | 250 mg |
| D167 | Diamond | 2 mm | 167 mg |

As shown in FIG. 21B, the release rate was affected primarily by the thickness of the depot, with thinner depots exhibiting faster release. The shape of the depot and bupivacaine content had less effect on the release rate, as evidenced by the 2 mm thick T500, R250, and D167 depots having similar release profiles. The results are consistent with the bupivacaine being released primarily from the exposed upper and lower surfaces of the depots.

FIG. 21C is a graph illustrating cumulative in vitro release of bupivacaine from ATX102 depots having different thicknesses at pH 7.4. ATX102 depots were formulated with a rectangular shape and 250 mg BUPFB equivalents, with thicknesses of 2 mm, 1.5 mm, and 1 mm. Release data was obtained using samples immersed in phosphate buffer at pH 7.4 and 37° C. At predetermined time points, aliquots of the buffer were drawn and analyzed using UV-Vis spectroscopy at 263 nm to quantify the amount of bupivacaine released. As shown in FIG. 21C, the release rate was affected by the depot thickness, with thinner depots generally exhibiting faster release, consistent with the trends observed for the pH 5.8 accelerated release test.

FIG. 21D is a graph illustrating cumulative in vitro release from ATX102 depots formulated with different bupivacaine particle sizes at pH 5.8. Triangular ATX102 depots with a 2 mm thickness were manufactured with different BUP-HCl powders: (1) unmilled BUP-HCl powder with an average particle diameter greater than 300 μm, (2) BUP-HCl powder having a D50 value of 29 μm, (3) BUP-HCl powder having a D50 value of 14 μm, and (4) BUP-HCl powder having a D50 value of 2 μm. The bupivacaine dosage in each depot was 500 mg BUPFB equivalents. Release data was obtained using the pH 5.8 accelerated in vitro release test described in Example 14. As shown in FIG. 21D, the bupivacaine release rate from the depots was affected by the particle size, with smaller particle sizes producing faster release.

### Example 17: In Vivo Release from Depots with Bupivacaine Hydrochloride Depots with and without Control Regions

This example describes in vivo release from depots formulated with bupivacaine hydrochloride, with and without control regions. ATX101 depots with two control regions and ATX102 depots without control regions were tested in a rabbit subcutaneous implantation model, a minipig abdominal hernia repair model, and a dog subcutaneous implantation model.

For the rabbit study, the following depot configurations were tested: (1) a single ATX101 depot having an equilateral triangle shape with a target dosage of 500 mg BUPFB equivalents (“T500”) (n=3), and (2) a single ATX102 depot having a diamond shape with a target dosage of 167 mg BUPFB equivalents (“D167”) (n=3). The depots were implanted subcutaneously in the dorsal region for 28 days. Blood draws were performed at predetermined time points (pre-dose, 30 minutes, 1, 3, 8, 24, 48, 72, 120, 168, 216, 264, 336, 384, 456, 528, 600, and 672 hours post-dose). A bupivacaine assay was performed on each aliquot to quantify the plasma concentration of bupivacaine at each time point.

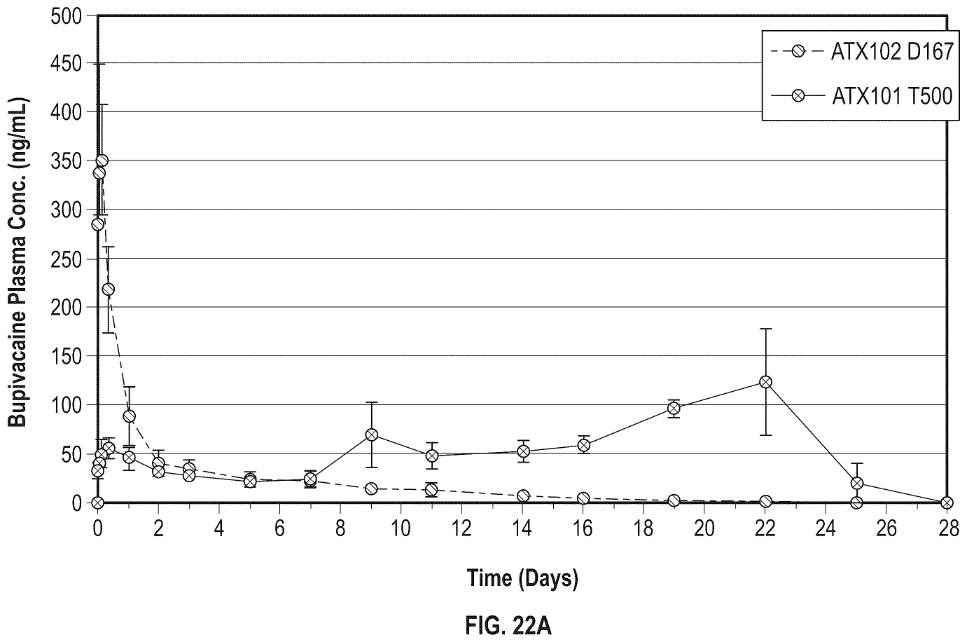

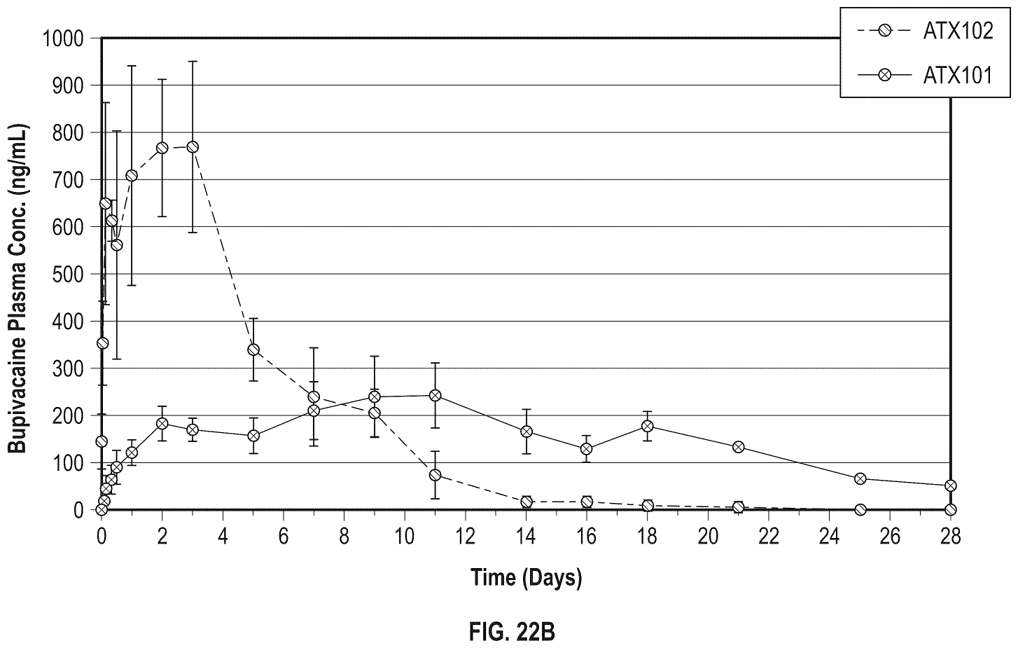

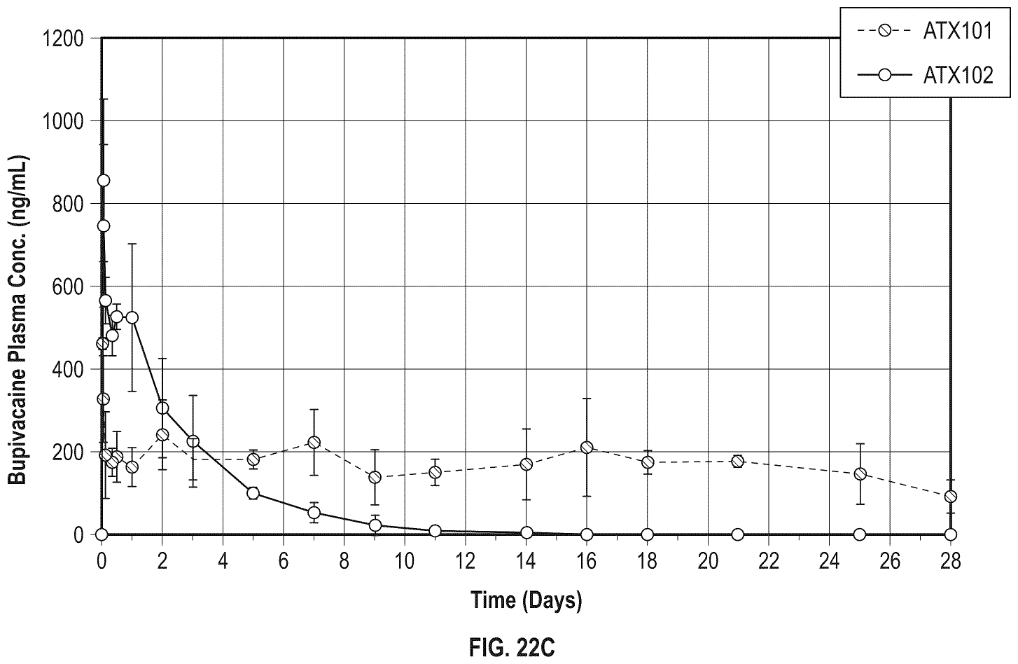

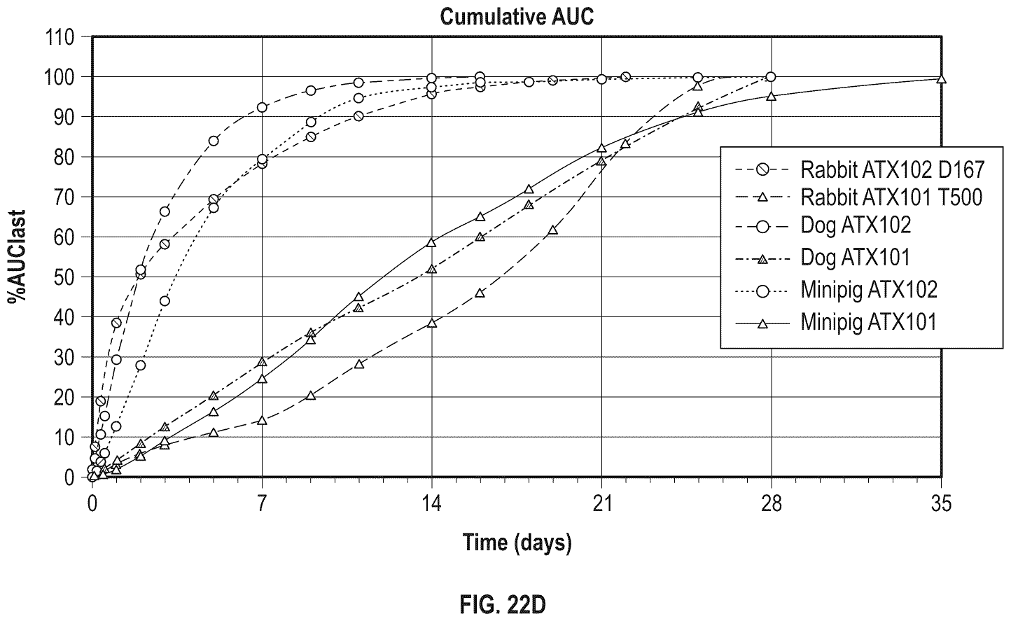

FIG. 22A is a graph showing the mean bupivacaine plasma concentration in rabbits over time, and Table 17 below illustrates selected pharmacokinetic parameters (Cₘₐₓ and AUCₗₐₛₜ are reported as mean±standard deviation; Tₘₐₓ and Tₗₐₛₜ are reported as median (min−max)). The ATX102 depots exhibited earlier and higher Cₘₐₓ values, and faster elimination, compared to the ATX101 depots.

TABLE 17
Pharmacokinetic Parameters for Rabbit Study

| | Dosage | Tₘₐₓ | Cₘₐₓ | AUCₗₐₛₜ | Tₗₐₛₜ |
|---|---|---|---|---|---|
| Group | (mg) | (hours) | (ng/ml) | (hr-ng/ml) | (Days) |
| ATX101 | 500 | 528 | 132 ± | 34,357 ± | 25 |
| T500 | | (216-528) | 45 | 10,840 | (22-28) |
| ATX102 | 167 | 1.5 | 368 ± | 12,450 ± | 19 |
| D167 | | (0.5-3) | 80 | 3,003 | (14-22) |

For the minipig studies, the following depot configurations were tested: (1) four ATX101 depots, each having an equilateral triangle shape and a dosage of 500 mg BUPFB equivalents, for a total target dosage of 2000 mg BUPFB equivalents (n=3); and (2) four ATX102 depots, each having an equilateral triangle shape and a dosage of 500 mg BUPFB, for a total target dosage of 2000 mg BUPFB equivalents (n=3). The depots were implanted in the subcutaneous and pre-peritoneal layers of the abdominal region adjacent to a prolene mesh for 28 days. Blood draws were performed at predetermined time points (30 minutes; 1, 3, 8, 12 hours; 1, 2, 3, 5, 7, 9, 11, 14, 16, 18, 21, 25, and 28 days post-dose). A bupivacaine assay was performed on each aliquot to quantify the plasma concentration of bupivacaine at each time point.

FIG. 22B is a graph showing the mean bupivacaine plasma concentration in minipigs over time, and Table 18 below illustrates selected pharmacokinetic parameters (Cₘₐₓ and AUCₗₐₛₜ are reported as mean±standard deviation; Tₘₐₓ and Tₗₐₛₜ are reported as median (min−max)). The ATX102 depots exhibited earlier and higher Cₘₐₓ values, and faster elimination, compared to the ATX101 depots.

TABLE 18
Pharmacokinetic Parameters for Minipig Studies

| | Dosage | Tₘₐₓ | Cmₐₓ | AUCₗₐₛₜ | Tₗₐₛₜ |
|---|---|---|---|---|---|
| Group | (mg) | (days) | (ng/ml) | (hr-ng/ml) | (Days) |
| ATX101 | 2000 | 11 | 262 ± | 104,000 ± | 35 |
| | | (9-11) | 76 | 15,300 | (35-42) |
| ATX102 | 2000 | 2 | 814 ± | 114,000 ± | 25 |
| | | (1-3) | 148 | 25,500 | (21-28) |

For the dog study, the following depot configurations were tested: (1) two ATX101 depots, each having an equilateral triangle shape and a dosage of 500 mg BUPFB equivalents, for a total target dosage of 1000 mg BUPFB equivalents (n=3); and (2) a single ATX102 depot having an equilateral triangle shape and a target dosage of 500 mg BUPFB (n=3). The depots were implanted subcutaneously in the dorsal region for 28 days. Blood draws were performed at predetermined time points (pre-dose, 30 minutes, 1, 3, 8, 24, 48, 72, 120, 168, 216, 264, 336, 384, 432, 504, 600, and 672 hours post-dose). A bupivacaine assay was performed on each aliquot to quantify the plasma concentration of bupivacaine at each time point.

FIG. 22C is a graph showing the mean bupivacaine plasma concentration in dogs over time, and Table 19 below illustrates selected pharmacokinetic parameters (Cₘₐₓ and AUCₗₐₛₜ are reported as mean±standard deviation; Tₘₐₓ and Tₗₐₛₜ are reported as median (min−max)). The ATX102 depots exhibited earlier and higher Cₘₐₓ values, and faster elimination, compared to the ATX101 depots.

TABLE 19
Pharmacokinetic Parameters for Dog Study

| | Dosage | Tₘₐₓ | Cₘₐₓ | AUCₗₐₛₜ | Tₗₐₛₜ |
|---|---|---|---|---|---|
| Group | (mg) | (hours) | (ng/ml) | (hr-ng/ml) | (Days) |
| ATX101 | 1000 | 0.5 | 462 ± | 116,000 ± | 28 |
| | | (0.5-0.5) | 244 | 26,900 | (28-28) |
| ATX102 | 500 | 1 | 858 ± | 44,300 ± | 16 |
| | | (1-1) | 196 | 10,200 | (16-16) |

FIG. 22D is a graph illustrating cumulative AUCₗₐₛₜ profiles for various animal models. The AUC data shown in FIG. 22D are from the following study groups: (1) rabbits implanted with an ATX101 T500 depot (FIG. 22A and Table 17), (2) rabbits implanted with an ATX102 D167 depot (FIG. 22A and Table 17), (3) minipigs implanted with four ATX101 depots (FIG. 22B and Table 18), (4) minipigs implanted with four ATX102 depots (FIG. 22B and Table 18), (5) dogs implanted with two ATX101 depots (FIG. 22C and Table 19), and (6) dogs implanted with an ATX102 depot (FIG. 22C and Table 19). As shown in FIG. 22D, the ATX102 depots exhibited faster release than the ATX101 depots across the various animal models.

### Example 18: In Vivo Release from Combinations of Different Depot Types

This example describes in vivo release studies performed in a minipig hernia repair model implanted with a combination of two different types of depots: depots with two control regions (“ATX101”) and depots without control regions (“ATX102”).

The following depot configurations were tested: (1) twelve ATX101 T500 depots, for a total target dosage of 6000 mg BUPFB equivalents (n=3); (2) eight ATX101 T500 depots and four ATX102 T500 depots, for a total target dosage of 6000 mg BUPFB equivalents (n=3); and (3) four ATX101 T500 depots, eight ATX101 T250 depots, and four ATX102 T500 depots, for a total target dosage of 6000 mg BUPFB equivalents (n=3). Implantation and blood draws were performed according to the procedure described in Example 17.

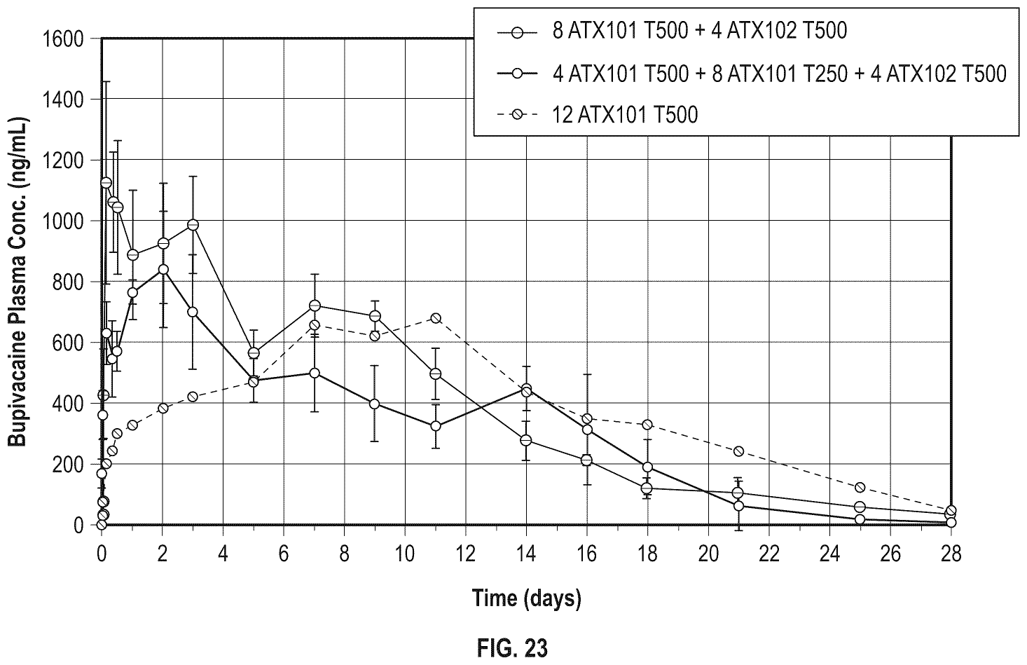

FIG. 23 is a graph showing the mean bupivacaine plasma concentration in minipigs over time, and Table 20 below illustrates selected pharmacokinetic parameters (Cₘₐₓ and AUCₗₐₛₜ are reported as mean±standard deviation; Tₘax and Tₗₐₛₜ are reported as median (min−max)). The Cₘₐₓ values were shifted earlier in animals implanted with a combination of ATX101 and ATX102 depots (Tₘₐₓ=6 hours to 2 days), compared to animals implanted with ATX101 depots alone (Tₘₐₓ=approximately 11 days). The AUC values were comparable for animals implanted with a combination of ATX101 and ATX102 depots, compared to animals implanted with ATX101 depots alone.

TABLE 20
Pharmacokinetic Parameters for Minipig Study

| | Dosage | Tₘₐₓ | Cₘₐₓ | AUCₗₐₛₜ | Tₗₐₛₜ |
|---|---|---|---|---|---|
| Group | (mg) | (days) | (ng/ml) | (hr-ng/ml) | (Days) |
| 12 ATX101 | 6000 | 11 | 751 ± | 241,000 ± | 28 |
| T500 | | (9-11) | 171 | 46,100 | (28-42) |
| 8 ATX101 | 6000 | 0.125 | 1250 ± | 264,000 ± | 28 |
| T500 + | | (0.125-0.5) | 226 | 20,200 | (28-28) |
| 4 ATX102 | | | | | |
| T500 | | | | | |
| 4 ATX101 | 6000 | 2 | 897 ± | 214,000 ± | 21 |
| T500 + | | (1-3) | 141 | 10,300 | (21-28) |
| 8 ATX101 | | | | | |
| T250 + | | | | | |
| 4 ATX102 | | | | | |
| T500 | | | | | |

### Example 19: Toxicology Studies with High Dosage Bupivacaine Free Base Depots

This example describes toxicology studies performed using depots containing BUPFB, with no control regions.

Three depot configurations were tested: (1) an equilateral triangle (similar to the depot 400 of FIG. 4A) containing 500 mg BUPFB (“T500”), (2) a right triangle (similar to the depot 470 of FIG. 4H) containing 250 mg BUPFB (“T250”), and (3) a diamond (similar to the depot 570 of FIG. 5H) containing 167 mg BUPFB (“D167”). The depots were prepared by mixing PS20, PLGA5050, BUPFB, and acetone in a 1:10:20:30 ratio by mass. The formulation was compressed and dried using a thermal press, then cut into the desired shapes. The resulting depots included the therapeutic region only, with no control regions (similar to the depot 100c of FIG. 1C).

The depots were implanted subcutaneously along the dorsal region of male Sprague Dawley rats, as listed in Table 21 below. Blood draws were performed at predetermined time points (pre-dose, 30 minutes, 1, 3, 8, 24, 48, 72, 120, 168, 216, 264, 336, 384, 432, 504, 600, and 672 hours post-dose). A bupivacaine assay was performed on each aliquot to quantify the plasma concentration of bupivacaine free base at each time point.

TABLE 21
Groups for Toxicology Study

| | | Total Dosage | Dosage | Number of |
|---|---|---|---|---|
| Group | Treatment | (mg) | (mg/kg) | Animals |
| 1 (low dose) | 1 T500 depot | 500 | 1328 ± 20.8 | n = 3 |
| 2 (mid dose) | 1 T500 depot, | 667 | 1769 ± 35.5 | n = 3 |
| | 1 D167 depot | | | |
| 3 (high dose) | 3 T250 depots | 750 | 2023 ± 54.6 | n = 3 |

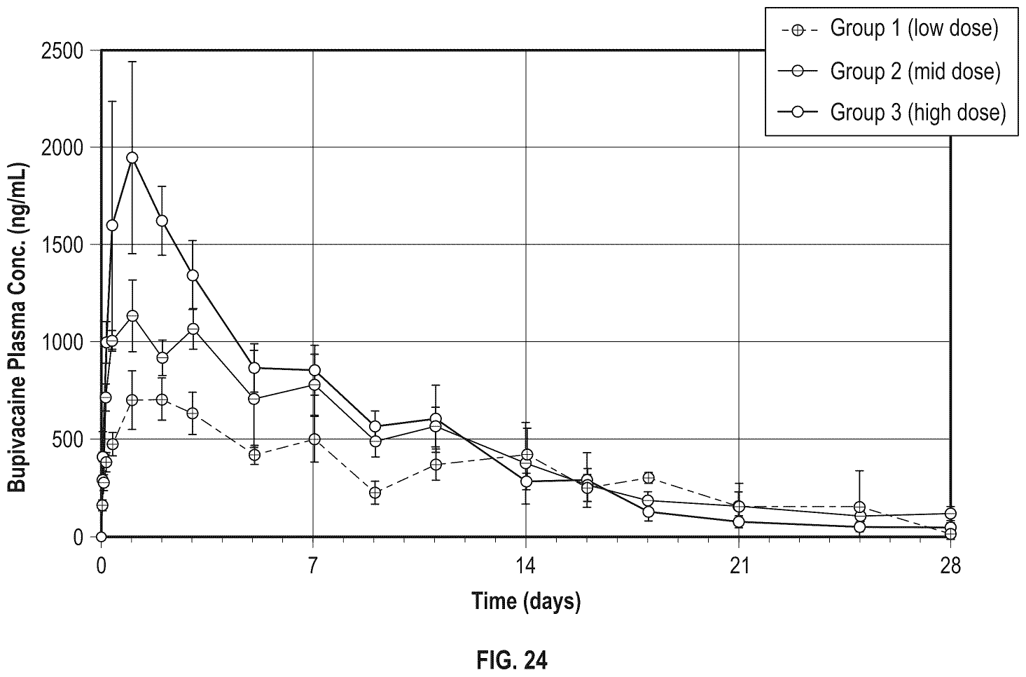

FIG. 24 is a graph showing the mean bupivacaine plasma concentration over time, and Table 22 below illustrates selected pharmacokinetic parameters (Cₘₐₓ and AUCₗₐₛₜ are reported as mean±standard deviation; Tₘₐₓ and Tₗₐₛₜ are reported as median (min−max)).

TABLE 22
Summary of Pharmacokinetic Parameters for Toxicology Study

| Group | Tₘₐₓ (h) | Cₘₐₓ (ng/ml) | AUCₗₐₛₜ (hr-ng/ml) | Tₗₐₛₜ (Days) |
|---|---|---|---|---|
| 1 (low dose) | 48 | 730 ± | 218.28 ± | 25 |
| | (24-48) | 139 | 26.328 | (25-28) |
| 2 (mid dose) | 24 | 1160 ± | 300.752 ± | 28 |
| | (8-72) | 161 | 51.900 | (28-28) |
| 3 (high dose) | 24 | 1987 ± | 359.406 ± | 28 |
| | (24-48) | 434 | 12.940 | (28-28) |

Sustained, controlled bupivacaine delivery was observed for all depot configurations, with no clinical signs of acute toxicity during the in-life period even for doses up to approximately 2000 mg/kg (the LD₅₀ values for bupivacaine in rats is 6 mg/kg (intravenous) or 43 mg/kg (subcutaneous)). Plasma concentrations of bupivacaine peaked at approximately 2000 ng/ml.

### Example 20: Depots with Plasticizers

This example describes preparation and characterization of depots formulated with various types of plasticizers.

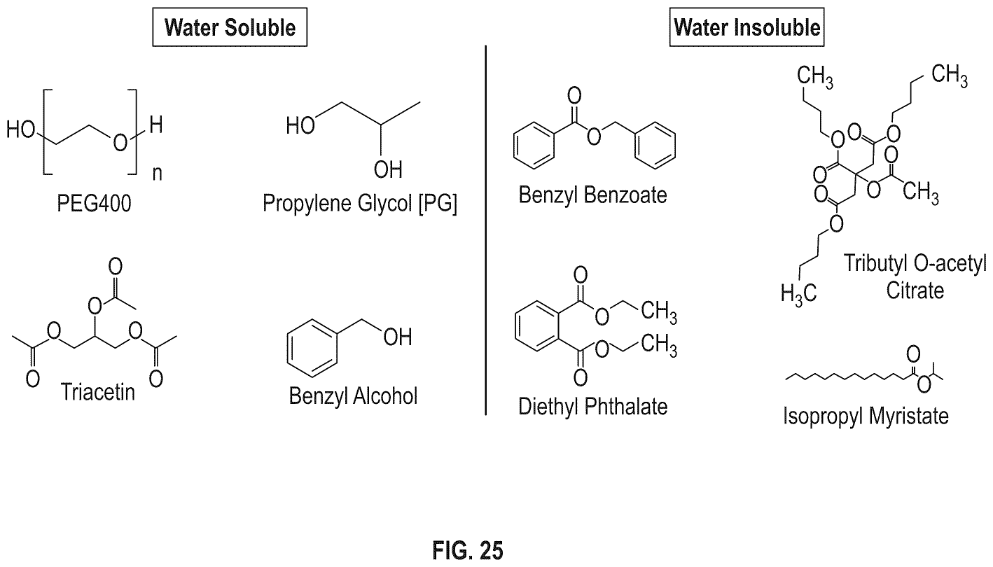

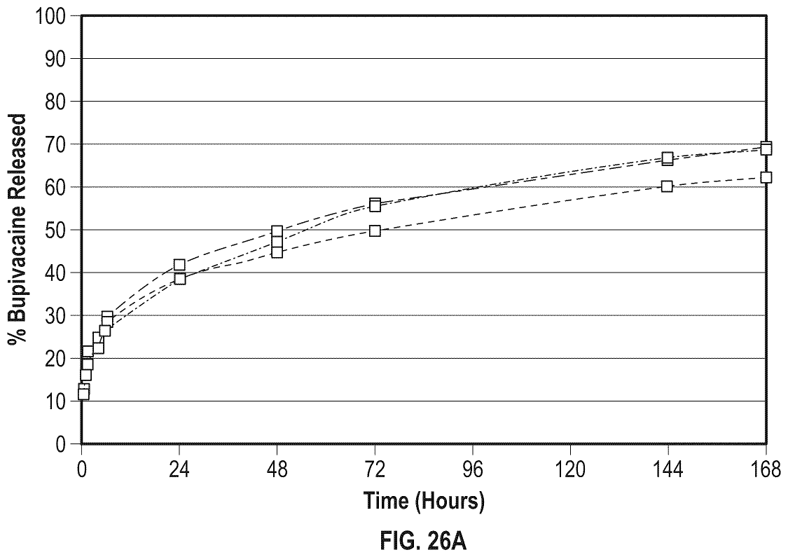

Depots composed of a therapeutic region only were formulated using at different plasticizer loadings, as listed in Tables 23A-23E below. Eight plasticizers were tested, four of which were water soluble (PEG400, propylene glycol (PG), triacetin, benzyl alcohol) and four of which were water insoluble (benzyl benzoate, diethyl phthalate, tributyl O-acetyl citrate, and isopropyl myristate). The chemical structures of the plasticizers are shown in FIG. 25. For each depot, the plasticizer was added to the PLGA5050 before mixing with acetone. The mixture was shaken overnight to obtain a homogenous solution. Micronized bupivacaine hydrochloride monohydrate (BUP-HCl) (D50 value from 6-12 μm) was then added to the solution to create a dough-like consistency. The dough underwent a series of heat compression steps to achieve the specified thickness and remove most of the acetone, and was then cut into the final depot shape (Table 24). The dimensions of the depots were varied to maintain a constant drug loading (300 mg BUP-HCl per depot).

TABLE 23A
Single Plasticizer Formulation - 14 wt % Loading

| Component | Plasticizer | PLGA5050 | BUP-HCl | Acetone |
|---|---|---|---|---|
| Mass Ratio | 5 | 10 | 20 | 30 |
| % Weight in Depot | 14.3 | 28.6 | 57.1 | N/A |

TABLE 23B
Single Plasticizer Formulation - 10 wt % Loading

| Component | Plasticizer | PLGA5050 | BUP-HCl | Acetone |
|---|---|---|---|---|
| Mass Ratio | 3.5 | 10 | 20 | 30 |
| % Weight in Depot | 10.5 | 29.9 | 59.7 | N/A |

TABLE 23C
Single Plasticizer Formulation - 7 wt % Loading

| Component | Plasticizer | PLGA5050 | BUP-HCl | Acetone |
|---|---|---|---|---|
| Mass Ratio | 2.5 | 10 | 20 | 30 |
| % Weight in Depot | 7.7 | 30.8 | 61.5 | N/A |

TABLE 23D
Single Plasticizer Formulation - 3.2 wt % Loading

| Component | Plasticizer | PLGA5050 | BUP-HCl | Acetone |
|---|---|---|---|---|
| Mass Ratio | 1 | 10 | 20 | 30 |
| % Weight | 3.2 | 32.3 | 64.5 | N/A |

TABLE 23E
No Plasticizer Formulation

| Component | PS20 | PLGA5050 | BUP-HCl | Acetone |
|---|---|---|---|---|
| Mass Ratio | 1 | 10 | 20 | 30 |
| % Weight | 3.2 | 32.3 | 64.5 | N/A |

TABLE 24
Depot Geometries

| Shape | Length | Width | Thickness | |
|---|---|---|---|---|
| Rectangular | 23.7 mm | 10.5 mm | 2 | mm |
| Rectangular | 33.5 mm | 14.9 mm | 1 | mm |
| Rectangular | 37.5 mm | 16.7 mm | 0.8 | mm |
| Rectangular | 43.3 mm | 19.2 mm | 0.6 | mm |

For in vitro release studies at pH 7.4, depots were immersed in 100 mL of pH 7.4 PBS buffer in a 37° C. water bath (static). Buffer samples were collected at 0.5 hour, 1 hour, 1.5 hours, 2.5 hours, 4 hours, 6 hours, and every 24 hours over a period of 168 hours, and replaced with fresh buffer. Buffer samples were analyzed using UV-Vis spectroscopy to quantify the amount of bupivacaine released.

For in vitro release studies at pH 5.8, depots were immersed in 750 mL of pH 5.8 buffer in a 37° C. water bath, with a spindle attached to the vessel spinning at 10 RPM. Buffer samples were collected at 5 minutes, 15 minutes, 30 minutes, 45 minutes, and every 1 hour over a period of 24 hours. Buffer samples were analyzed using UV-Vis spectroscopy to quantify the amount of bupivacaine released.

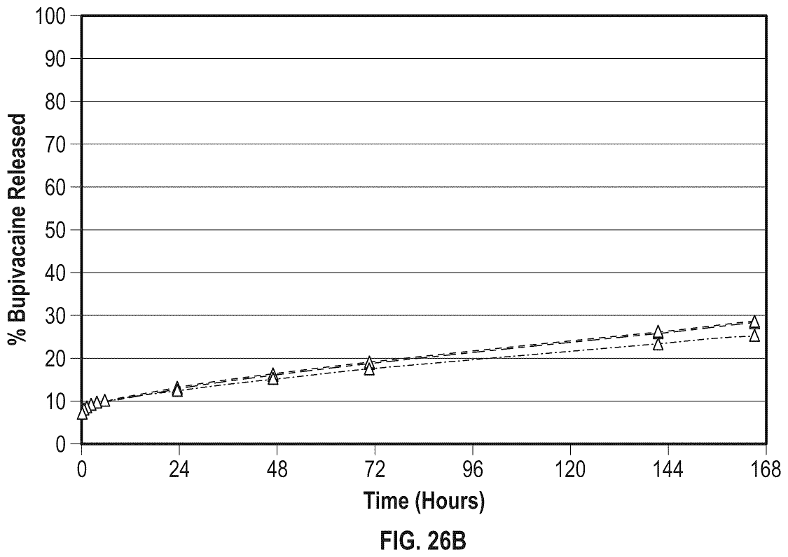

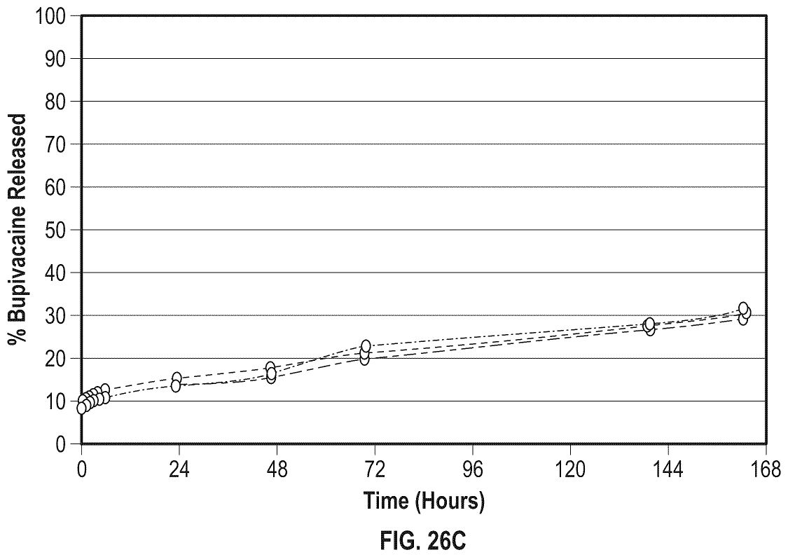

FIGS. 26A-26C are graphs illustrating in vitro release at pH 7.4 for depots formulated with triacetin (FIG. 26A), diethyl phthalate (FIG. 26B), and benzyl benzoate (FIG. 26C) at 14 wt % loading (n=3 for each plasticizer). All depots were rectangular with a 2 mm thickness. Release was normalized to 100% based on the total initial drug loading in the depot (300 mg BUP-HCl). At 14 wt % loading, only 3 out of the 8 tested plasticizers (triacetin, diethyl phthalate, and benzyl benzoate) produced depots that had improved flexibility (see Table 25B below) and exhibited controlled release of drug over time. The other 5 plasticizers (PEG400, propylene glycol, benzyl alcohol, tributyl O-acetyl citrate, isopropyl myristate) produced depots with poorer flexibility or the plasticizers precipitated out over time. Without wishing to be bound by theory, it is hypothesized that improved flexibility is generally correlated with miscibility of the plasticizer in PLGA5050 and with low volatility of the plasticizer. The estimated RED for triacetin and diethyl phthalate in PLGA5050 is less than 1 (0.68 for triacetin and 0.80 for diethyl phthalate); whereas the estimated RED for propylene glycol and isopropyl myristate is greater than 1 (1.49 for propylene glycol and 1.44 for isopropyl myristate). Triacetin, benzyl benzoate, and diethyl phthalate have relative low vapor pressures at 25° C. (0.33 Pa for triacetin, 0.03 Pa for benzyl benzoate, and 0.28 Pa for diethyl phthalate); whereas benzyl alcohol and PEG400 have relatively high vapor pressures (12.53 for benzyl alcohol, <10 for PEG400).

As shown in FIGS. 26A-26C, the observed release rates for depots formulated with triacetin, diethyl phthalate, and benzyl benzoate were slower than those of depots formulated without plasticizers. Without wishing to be bound by theory, it is hypothesized that the presence of the hydrophobic benzene rings in the diethyl phthalate and benzyl benzoate reduced water penetration into the depot, thus slowing the release rate (log P for benzyl benzoate is 3.97, log P for diethyl phthalate is 2.47).

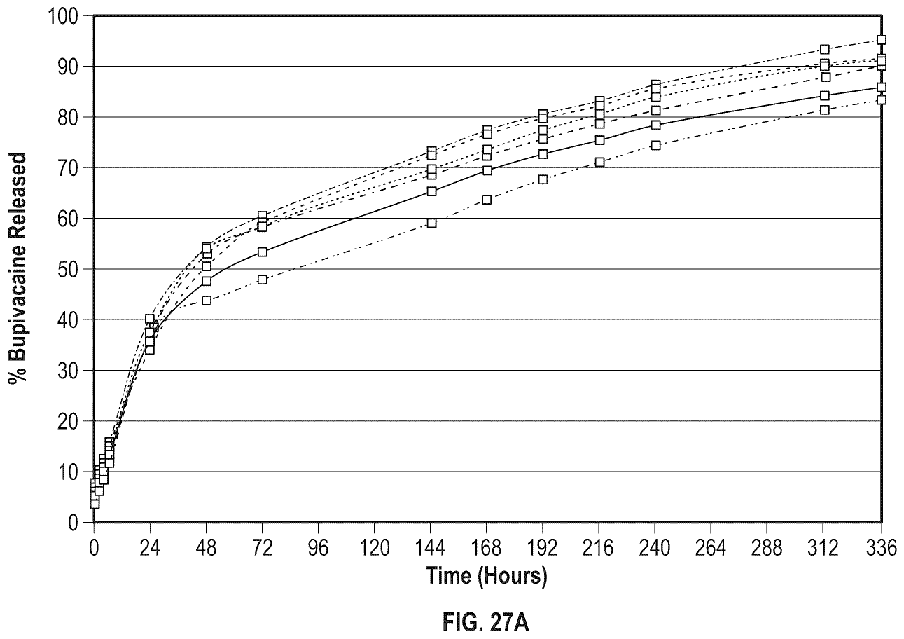

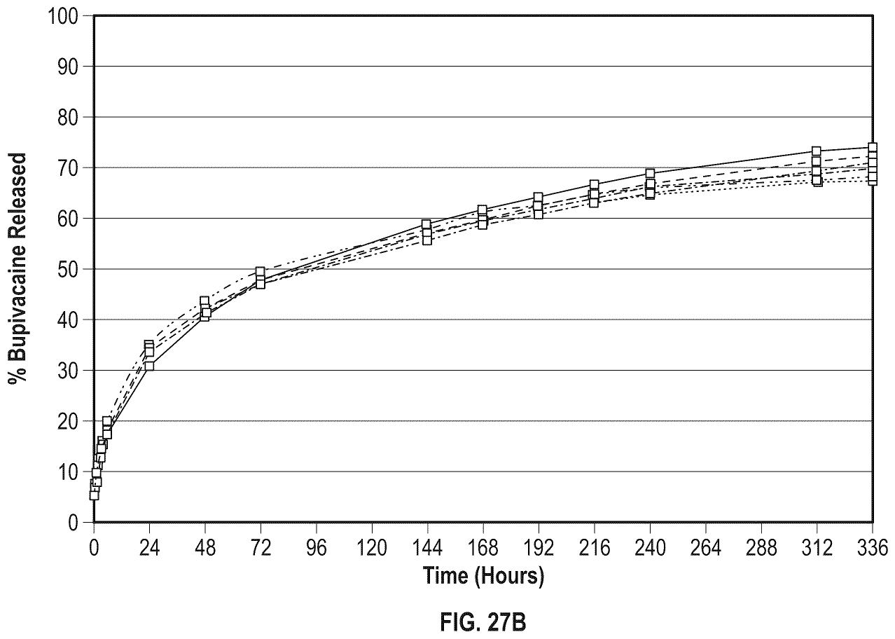

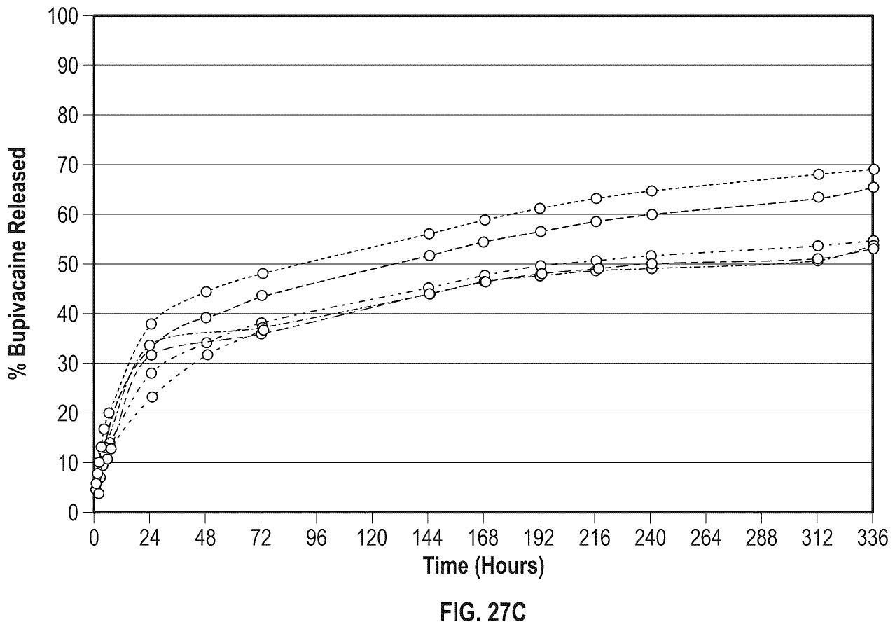

FIGS. 27A-27C are graphs illustrating in vitro release at pH 7.4 for depots formulated with triacetin (FIG. 27A), diethyl phthalate (FIG. 27B), and benzyl benzoate (FIG. 27C) at 3.2 wt % loading (n=6 for each plasticizer). All depots were rectangular with a 2 mm thickness. Release was normalized to 100% based on the total initial drug loading in the depot (300 mg BUP-HCl). At 3.2 wt % loading, an increased release rate was observed, but the resulting depots did not exhibit improved flexibility (see Table 25C below).

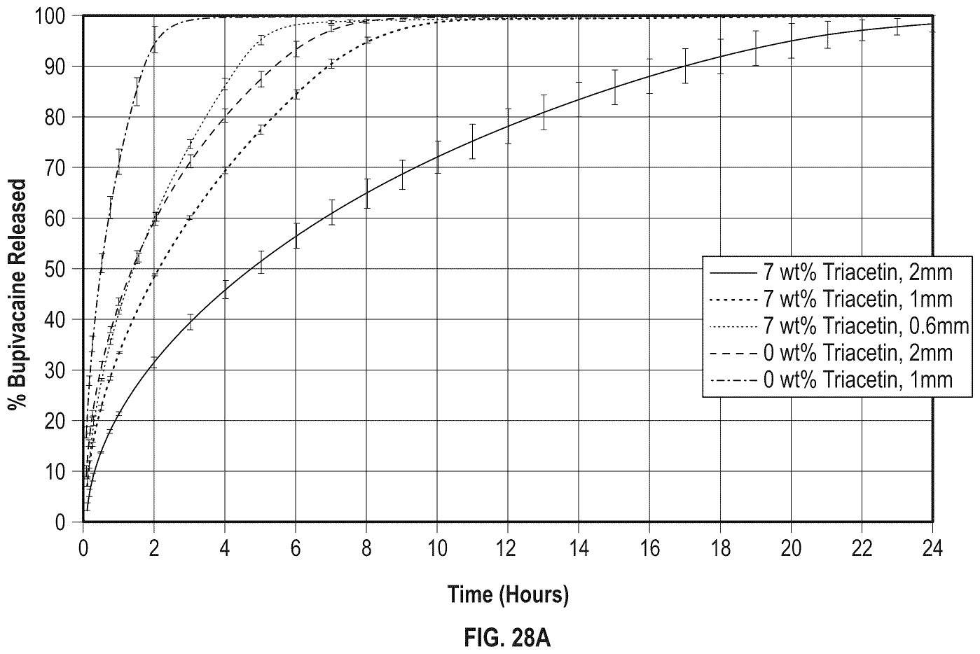

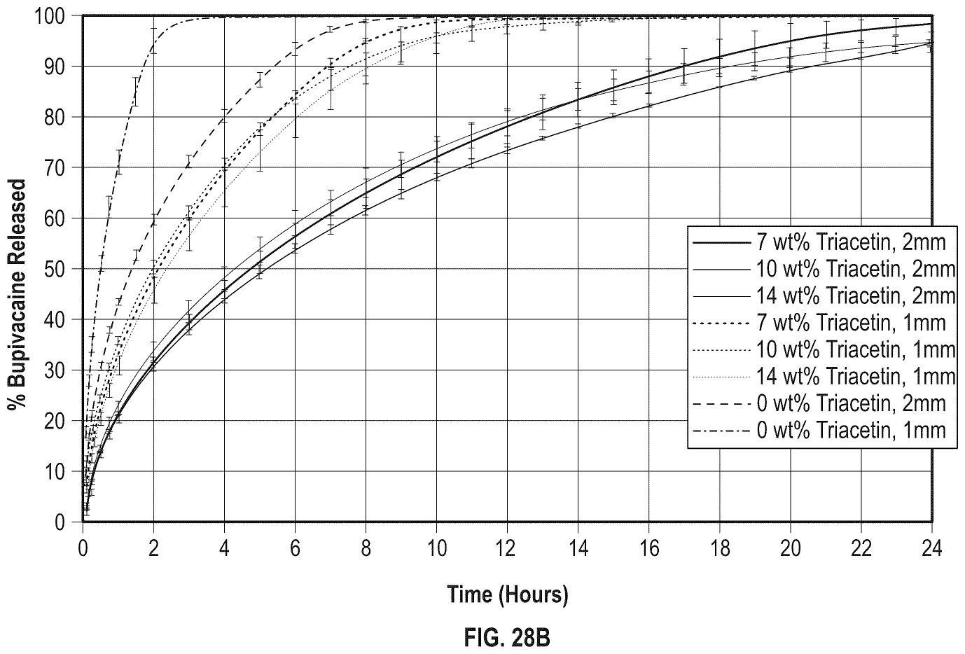

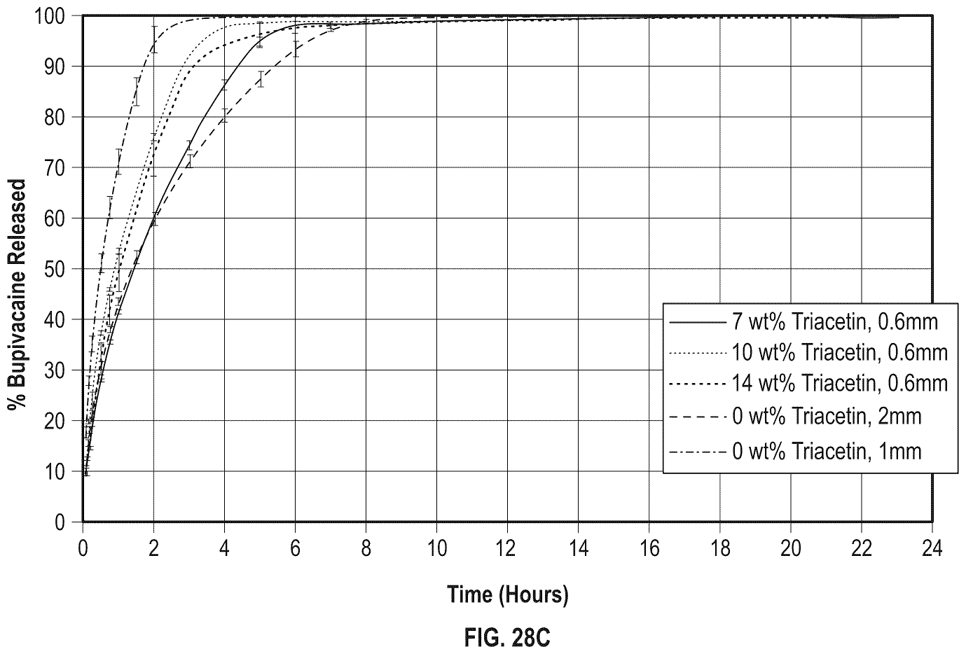

FIG. 28A-28C are graphs illustrating in vitro release at pH 5.8 for depots with various plasticizer loadings and varying thicknesses. Depots were rectangular with a thickness of 2 mm, 1 mm or 0.6 mm. Depots were formulated with 0 wt %, 7 wt %, 10 wt %, or 14 wt % triacetin (n=3 for 0 wt % depots, n=2 for all other depots). For 1 mm and 0.6 mm depots, release was normalized to 100% at t=24 hours (based on the assumption that 100% of the drug was eluted by 24 hours). For 2 mm depots, release was normalized to 100% based on the amount of drug remaining in the depot at the 24 hour time point as quantified by HPLC (“HPLC extracted dose”). The addition of triacetin resulted in a slower release rate for depots of equivalent thickness. Thinner depots had a faster release rate, independent of the amount of triacetin loading. The flexibility of the depots was dependent on the depot thickness, with thinner depots having greater flexibility than thicker depots. For the 2 mm thick depot, the flexibility decreased with lower loading of triacetin. These results demonstrate that the drug release profile could be controlled by varying the plasticizer loading and depot thickness.

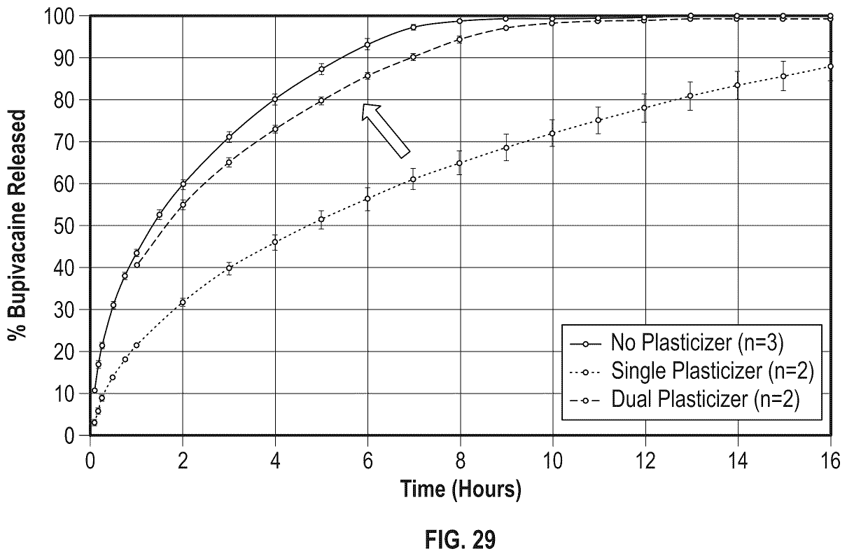

FIG. 29 is a graph illustrating in vitro release at pH 5.8 for depots with no plasticizer, a single plasticizer, or dual plasticizers. Depots were rectangular with a 2 mm thickness. Depots were formulated with no plasticizer, 7 wt % triacetin (“single plasticizer”), or 7 wt % triacetin/1 wt % glycerol (“dual plasticizer”). For the dual plasticizer depots, the plasticizer:PLGA5050:BUP-HCl:acetone mass ratio was 2.64:10:20:30, with a glycerol:triacetin mass ratio of 0.33:2.31. Release was normalized to 100% at t=16 hours for the no plasticizer depots, to 100% at t=160 hours for the dual plasticizer depots, and based on the HPLC extracted dose for the single plasticizer depots. As indicated by the arrow in FIG. 29, the addition of glycerol increased the release rate while maintaining the flexibility of the depot. Without wishing to be bound by theory, it is hypothesized that the lack of hydroxyl and ionizable groups in triacetin results in a low polarity, which may reduce water penetration into the depot and slow down the release rate. The addition of glycerol, which includes three hydroxyl groups which can form hydrogen bonds with water molecules, may promote water penetration into the depot to increase the release rate.

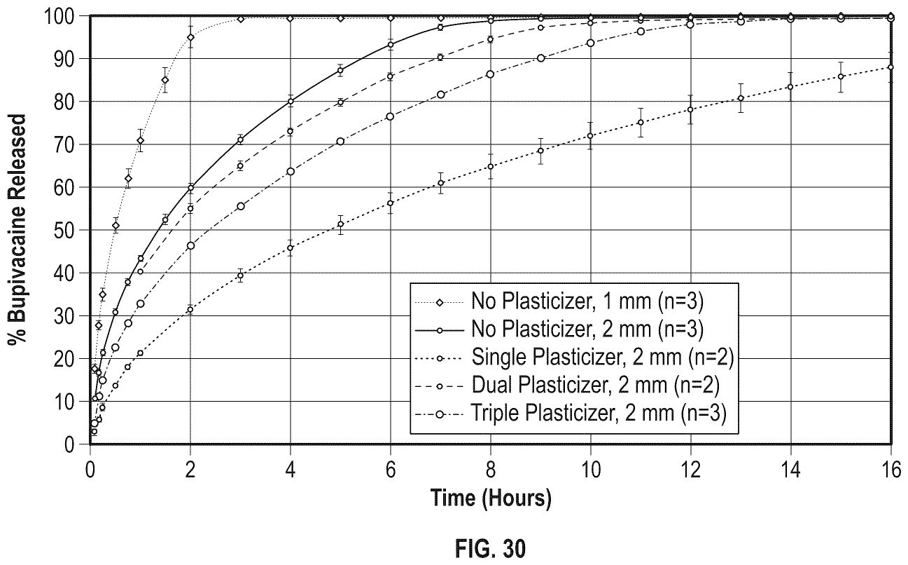

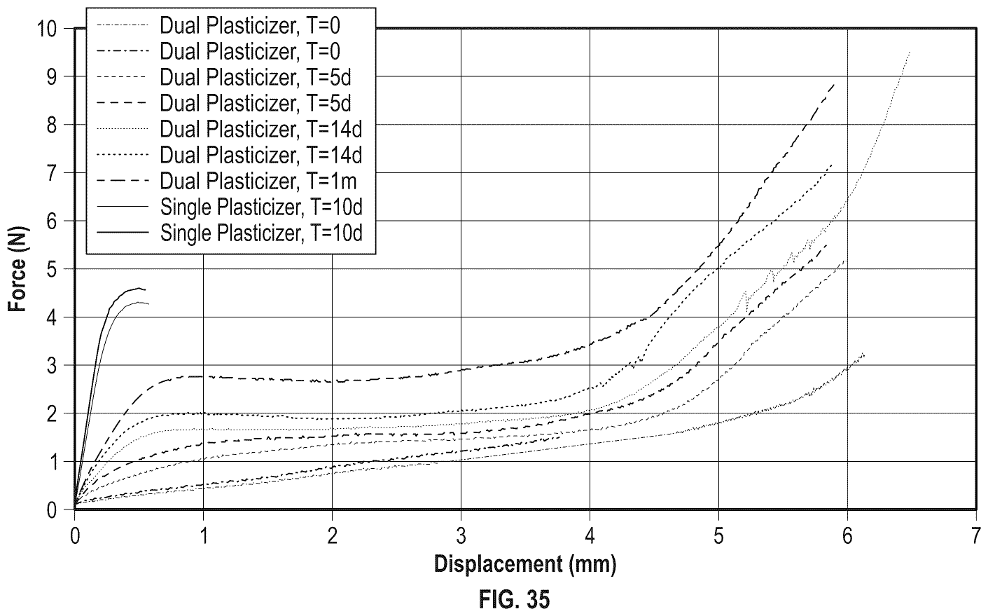

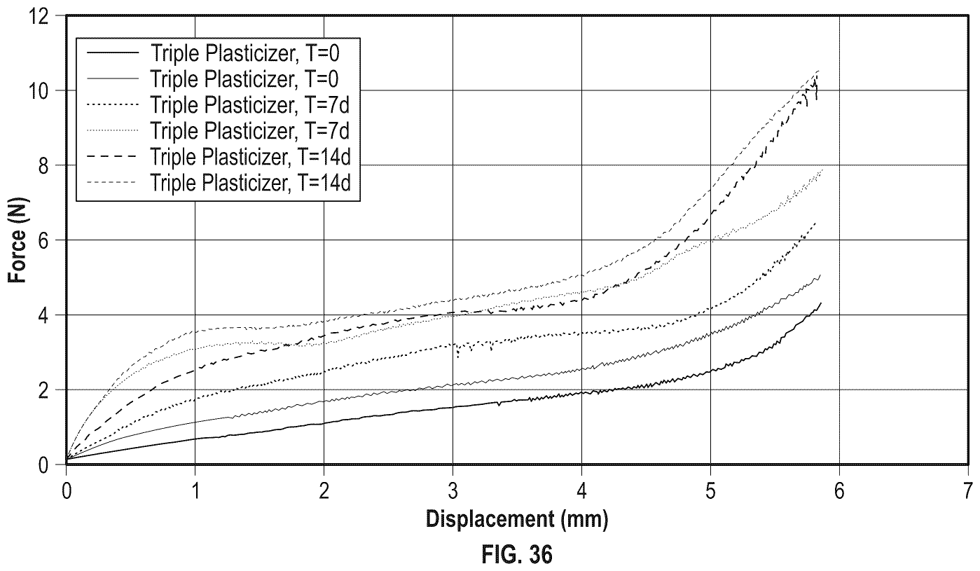

FIG. 30 is a graph illustrating in vitro release at pH 5.8 for depots with no plasticizer, a single plasticizer, dual plasticizers, or triple plasticizers. Depots were rectangular with a 2 mm or 1 mm thickness. Depots were formulated with no plasticizer, 7 wt % triacetin (“single plasticizer”), 7 wt % triacetin/1 wt % glycerol (“dual plasticizer”), or 1.4 wt % triacetin/1.4 wt % benzyl benzoate/0.4 wt % glycerol (“triple plasticizer”). For the triple plasticizer depots, the plasticizer:PLGA5050:BUP-HCl:acetone mass ratio was 1:10:20:30, with a benzyl benzoate:triacetin:glycerol mass ratio of 0.44:0.44:0.125. Release was normalized to 100% at t=16 hours for the no plasticizer depots, to 100% at t=24 hours for the triple plasticizer depots, to 100% at t=160 hours for the dual plasticizer depots, and based on the HPLC extracted dose for the single plasticizer depots. As shown in FIG. 30, the single plasticizer depots (triacetin only) exhibited the slowest release rate. The addition of glycerol in the dual plasticizer depots increased the release rate. The addition of benzyl benzoate in the triple plasticizer depots slowed the release rate. Depots with dual and triple plasticizers remained flexible for up to one month (see FIGS. 35 and 36 below), whereas depots with single plasticizers were no longer flexible after 10 days see FIG. 35 below).

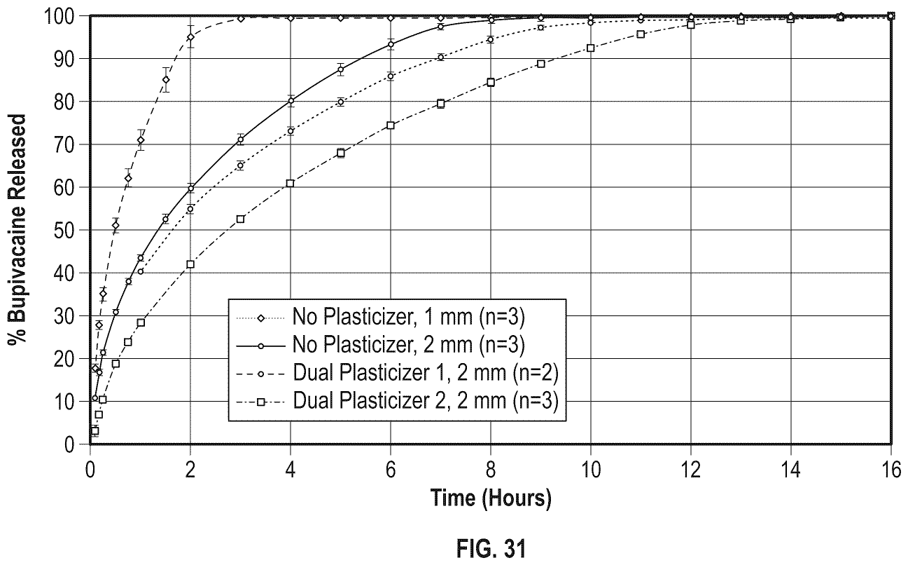

FIG. 31 is a graph illustrating in vitro release at pH 5.8 for depots with no plasticizer or dual plasticizers. Depots were rectangular with a 2 mm or 1 mm thickness. Depots were formulated with no plasticizer, 7 wt % triacetin/1 wt % glycerol (“dual plasticizer 1”), or 2.8 wt % triacetin/0.4 wt % glycerol (“dual plasticizer 2”). For the dual plasticizer 2 depots, the plasticizer:PLGA5050:BUP-HCl:acetone mass ratio was 1:10:20:30, with a triacetin:glycerol mass ratio of 0.88:0.125. Dual plasticizer 2 depots were formulated with unmilled BUP-HCl, whereas all other depots were formulated with micronized BUP-HCl. Release was normalized to 100% at t=16 hours for the no plasticizer depots, to 100% at t=24 hours for the dual plasticizer 2 depots, and to 100% at t=160 hours for the dual plasticizer 1 depots. As shown in FIG. 31, the dual plasticizer 2 depots using the unmilled BUP-HCl exhibited a slower release rate and were also slightly less flexible compared to the triple plasticizer depots up to one month.

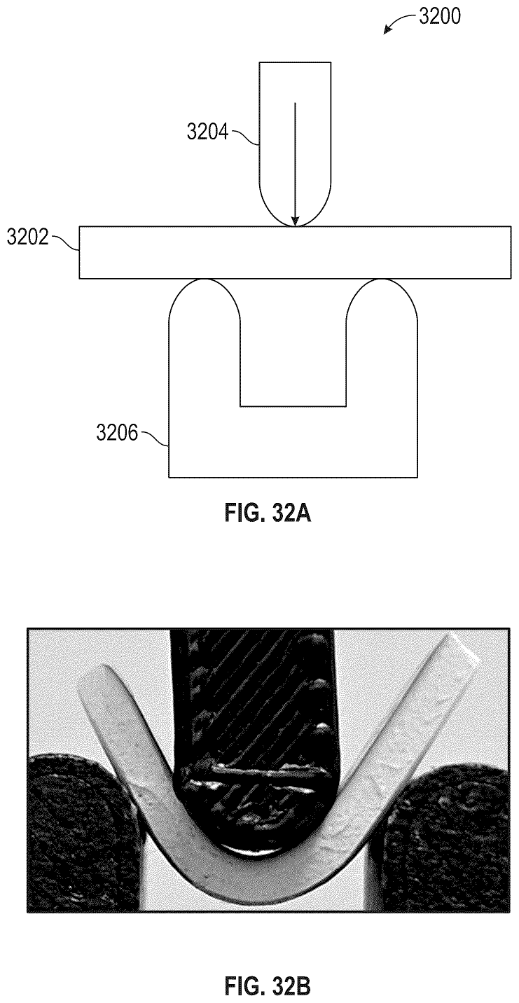

FIG. 32A illustrates a setup 3200 for mechanical testing of depots. Three-point bend testing was performed by placing a test sample 3202 lengthwise between an upper jig 3204 and a U-shaped lower jig 3206. The distance between the two supports of the lower jig 3206 was approximately 15.5 mm. During testing, a downward force was applied to bend the test sample 3202 by lowering the upper jig 3204 at a rate of 0.5 mm/min. The upper jig 3204 was attached to a tensile tester to register the force exerted as the displacement increased. Testing was performed at room temperature (20-25° C.). The flexural modulus of the depots was calculated from the initial linear portion of the force-displacement curve, using the equation

$$E = \frac{{L}^{3} F}{4 b {h}^{3} d} ,$$

where E is the flexural modulus, L is the length of the test sample (corresponding to the distance between the two supports of the lower jig 3206), F is the force applied to the sample, b is the width of the test sample, h is the thickness of the sample, and d is the displacement of the sample.

FIG. 32B is an image of a plasticizer-loaded depot during mechanical testing. The depot was a triple plasticizer depot formulated with 1.4 wt % triacetin/1.4 wt % benzyl benzoate/0.4 wt % glycerol. As shown in FIG. 32B, the depot illustrated highly flexible behavior and was capable of significant bending without fracturing.

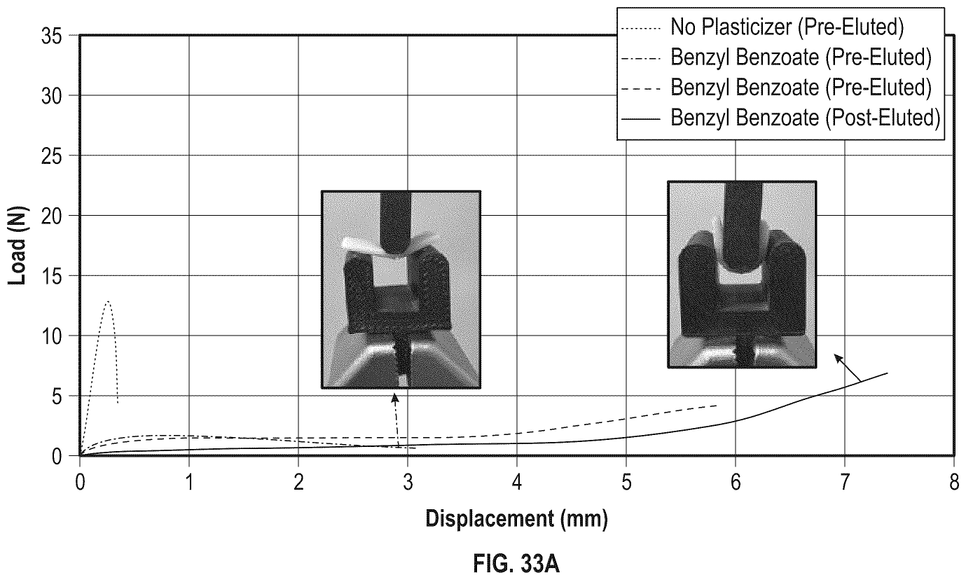

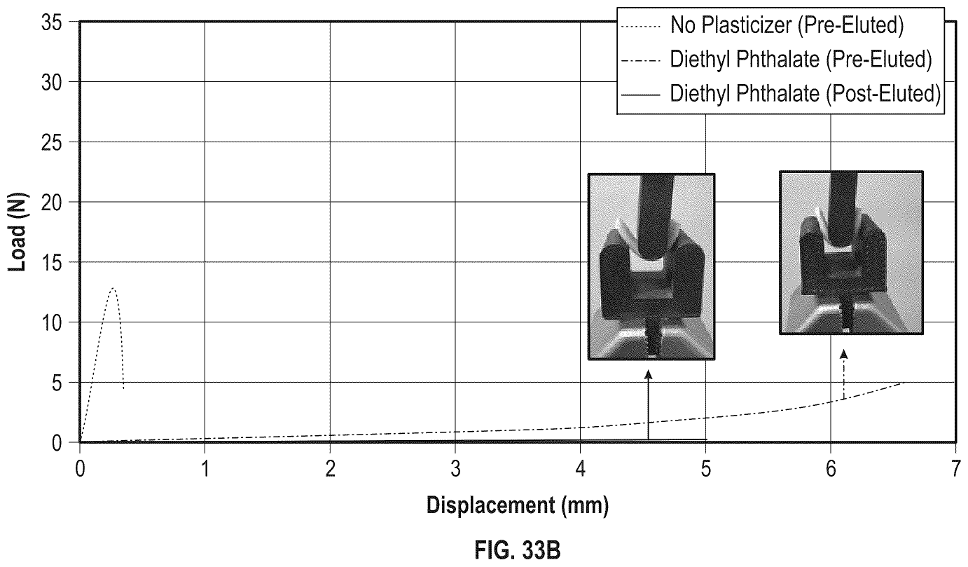

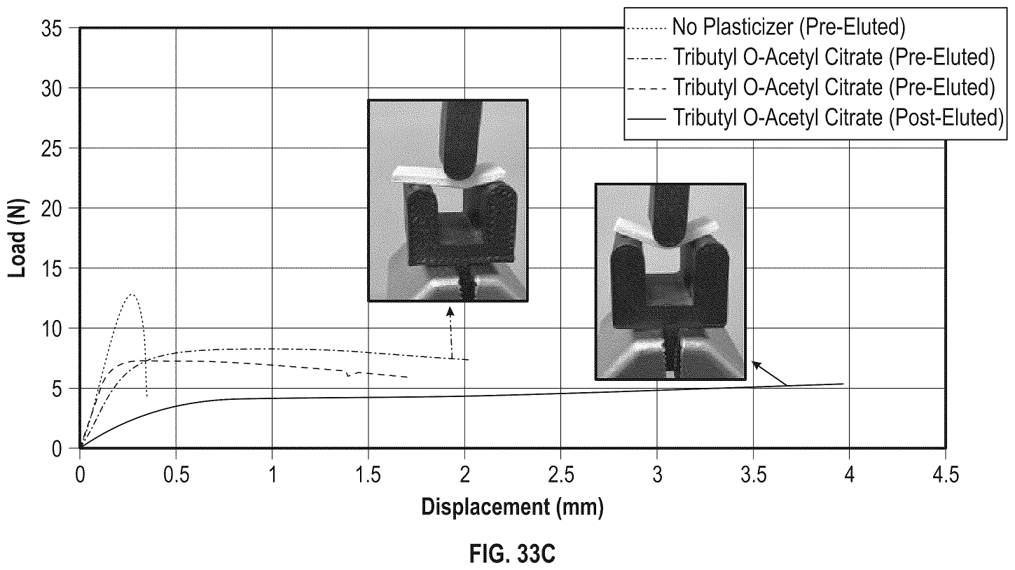

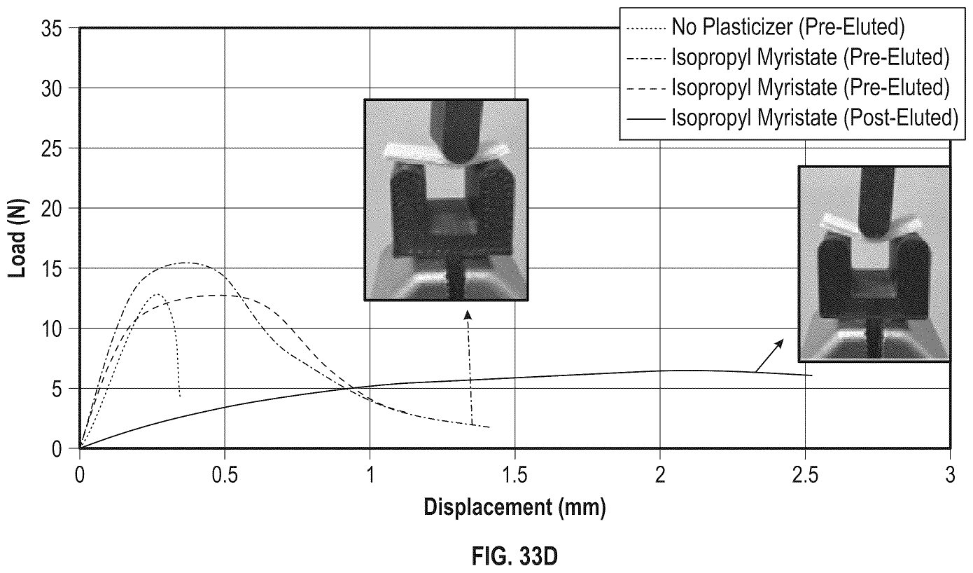

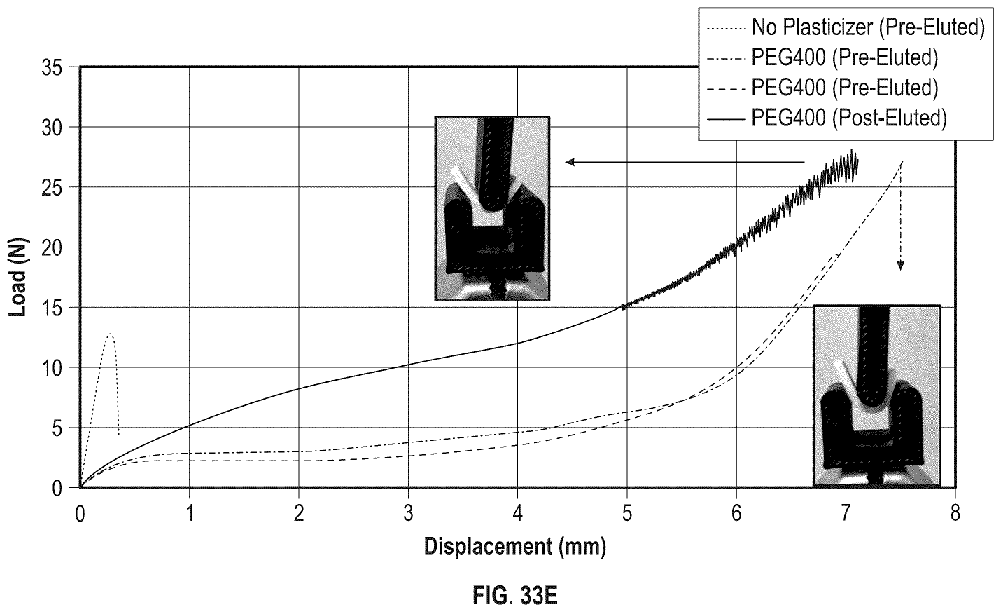

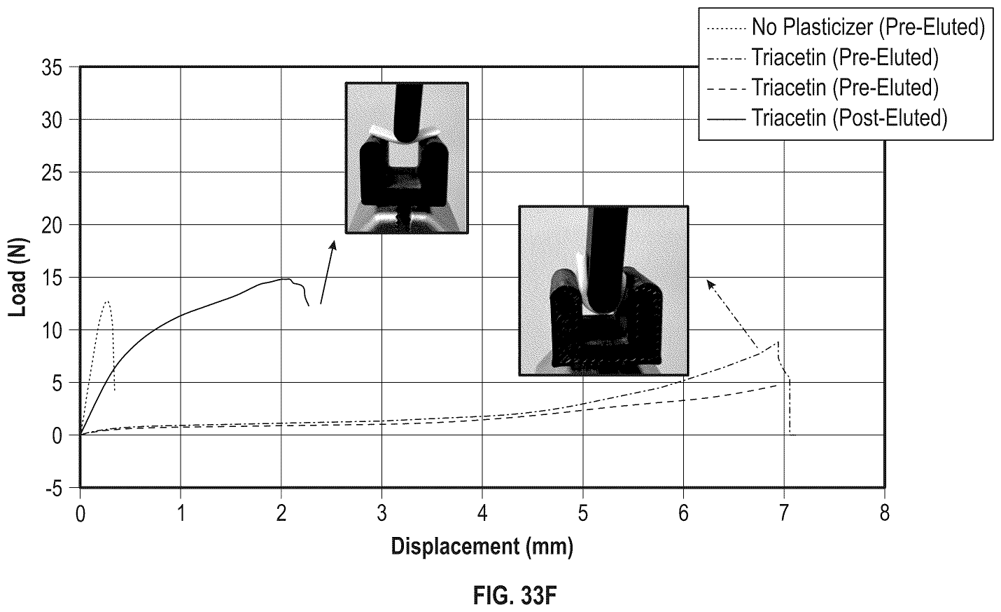

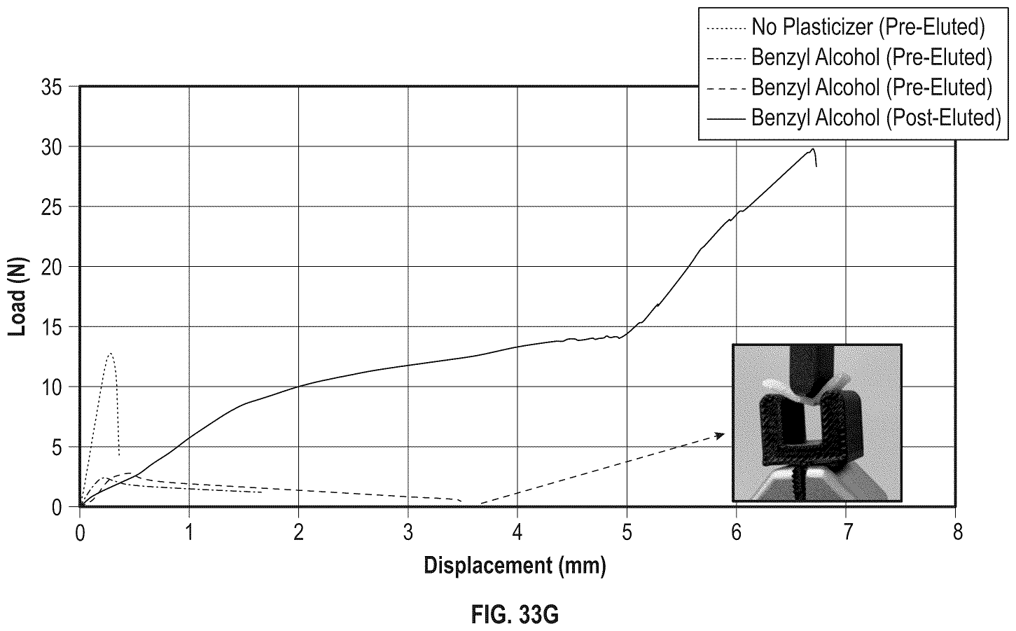

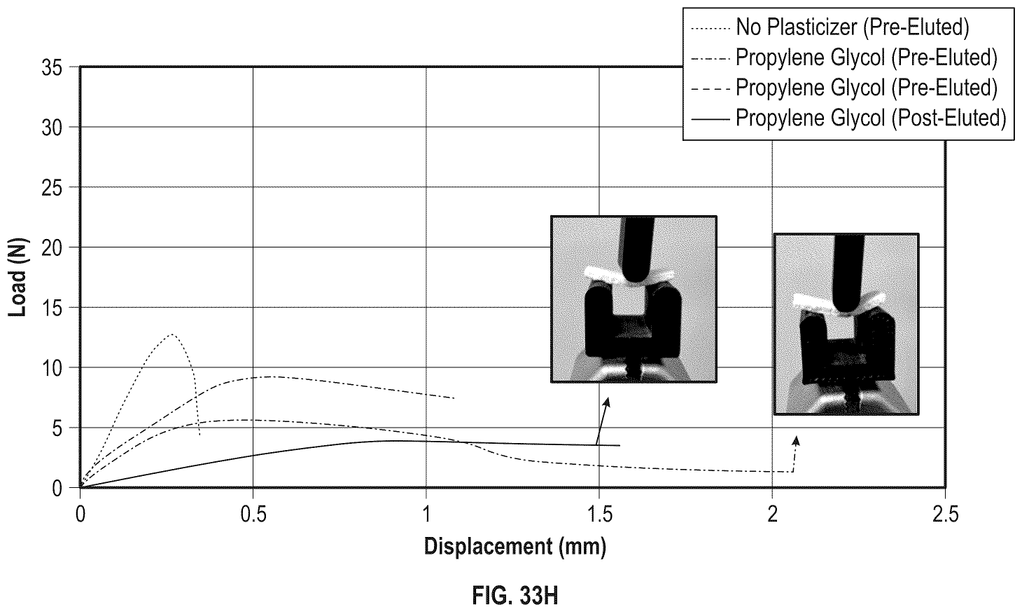

FIGS. 33A-33H are graphs illustrating force-displacement curves for depots formulated with benzyl benzoate (FIG. 33A), diethyl phthalate (FIG. 33B), tributyl O-acetyl citrate (FIG. 33C), isopropyl myristate (FIG. 33D), PEG400 (FIG. 33E), triacetin (FIG. 33F), benzyl alcohol (FIG. 33G), and propylene glycol (FIG. 33H) at 14 wt % loading. Depots without plasticizer were also tested as controls. Depots were rectangular with a 2 mm thickness. Testing was performed before the depots were subjected to in vitro release testing (“pre-eluted”) and after in vitro release testing at pH 5.8 for 16 to 24 hours (“post-eluted”). As shown in FIGS. 33A-33H, depots without plasticizer were not flexible and fractured with less than 1 mm displacement (see also Table 25A below). All plasticizers at 14 wt % loading provided increased flexibility compared with the depots without plasticizers, but only benzyl benzoate (FIG. 33A), diethyl phthalate (FIG. 33B), and triacetin (FIG. 33F) provided the greatest flexibility pre- and post-elution, with no leaching of the plasticizer or no drug precipitation post-manufacturing (see also Table 25B below). It was observed that the post-eluted triacetin depots were significantly more flexible upon immediate removal from the 37° C. water bath, and became less flexible upon drying and cooling to room temperature during the mechanical testing procedure. Thus, although the mechanical testing data for post-eluted triacetin depots in FIG. 33F showed decreased flexibility compared to pre-eluted depots, empirical observations of the post-eluted depots at 37° C. suggested that these depots maintained their flexibility under physiological conditions.

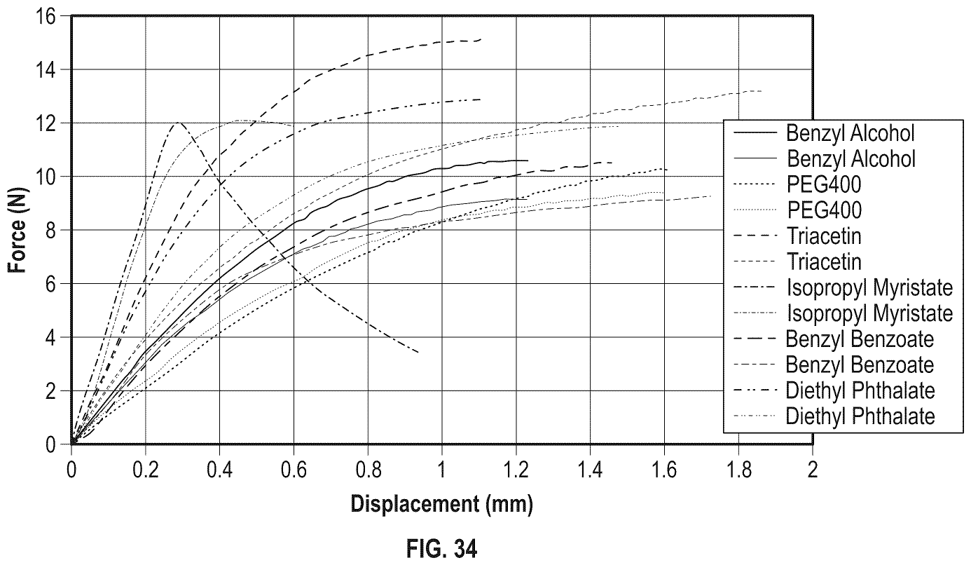

FIG. 34 is a graph illustrating force-displacement curves for depots formulated with 3.2 wt % loading of various plasticizers. Depots were rectangular with a 2 mm thickness and were pre-eluted. Depots with the lower 3.2 wt % plasticizer loading were not flexible compared to the depots with 14 wt % plasticizer loading (FIGS. 33A-33H) (see also Table 25C below).

FIG. 35 is a graph illustrating force-displacement curves for depots with single or dual plasticizers at various time points post-manufacturing. Depots were formulated with 14 wt % triacetin (“single plasticizer”) or 7 wt % triacetin/1 wt % glycerol (“dual plasticizer”). Depots were rectangular with a 2 mm thickness. Testing was performed immediately after manufacturing (“t=0”), 5 days post-manufacturing (“t=5 d”), 10 days post-manufacturing (“t=10 d”), 14 days post-manufacturing (“t=14 d”), or 1 month post-manufacturing (“t=1 m”). Depots were stored at room temperature (20-25° C.) for the specified time period post-manufacturing. As shown in FIG. 35, the single plasticizer depots were no longer flexible after 10 days, even with a higher 14 wt % loading. For the dual plasticizer depots, there was a slight decrease in flexibility at 1 month post-manufacturing, but the depots still remained flexible overall (see also Table 25D below). These results demonstrate that the secondary plasticizer (glycerol) was beneficial to provide flexibility for longer durations after manufacturing.

FIG. 36 is a graph illustrating force-displacement curves for depots with triple plasticizers at various time points post-manufacturing. Depots were formulated with 1.4 wt % triacetin/1.4 wt % benzyl benzoate/0.4% glycerol. Depots were rectangular with a 2 mm thickness. Testing was performed immediately after manufacturing (“t=0”), 7 days post-manufacturing (“t=7 d”), or 14 days post-manufacturing (“t=14 d”). All depots remained flexible through 14 days post-manufacturing (see also Table 25E below).

Tables 25A-25G below summarize the mechanical testing results for depots with various compositions and structures.

TABLE 25A
Flexural Moduli of Depots without Plasticizers

| Depot | | Thickness | Flexural Modulus | |
|---|---|---|---|---|
| Formulation | Plasticizer | (mm) | (MPa) | Flexible? |
| No plasticizer | N/A | 2 | 597 | No |
| | | | 442 | No |
| | | | 555 | No |
| | | 1 | 862 | No |
| | | | 687 | No |
| | | | 587 | No |

TABLE 25B
Flexural Moduli of Depots with 14 wt % Plasticizer

| | | | Flexural | |
|---|---|---|---|---|
| Depot | | Thickness | Modulus | |
| Formulation | Plasticizer | (mm) | (MPa) | Flexible? |
| Single | PEG400 | 2 | 52 | Slightly |
| plasticizer, 14 | | | 48 | Slightly |
| wt % loading | Triacetin | 2 | 5 | Yes |
| | | | 4 | Yes |
| | Benzyl alcohol | 2 | 137 | No |
| | | | 83 | No |
| | Propylene | 2 | 186 | No |
| | glycol | | 231 | No |
| | Diethyl | 2 | 3 | Yes |
| | phthalate | | | |
| | Tributyl O- | 2 | 250 | No |
| | acetyl citrate | | 311 | No |
| | Benzyl | | 33 | Yes |
| | benzoate | 2 | 22 | Yes |
| | Isopropyl | | | |
| | myristate | 2 | 746 | No |
| | | | 434 | No |

TABLE 25C
Flexural Moduli of Depots with 3.2 wt % Plasticizer

| | | | Flexural | |
|---|---|---|---|---|
| Depot | | Thickness | Modulus | |
| Formulation | Plasticizer | (mm) | (MPa) | Flexible? |
| Single | PEG400 | 2 | 115 | No |
| plasticizer, 3.2 | | | 129 | No |
| wt % loading | Triacetin | 2 | 340 | No |
| | | | 206 | No |
| | Benzyl alcohol | 2 | 171 | No |
| | | | 185 | No |
| | Diethyl | 2 | 304 | No |
| | phthalate | | 224 | No |
| | Benzyl | 2 | 180 | No |
| | benzoate | | 163 | No |
| | Isopropyl | 2 | 498 | No |
| | myristate | | 437 | No |

TABLE 25D
Flexural Moduli of Depots with Triacetin/Glycerol
at Different Time Points

| | | Thick- | Flexural | |
|---|---|---|---|---|
| Depot | | ness | Modulus | |
| Formulation | Time Point | (mm) | (MPa) | Remarks |
| 7 wt % | t = 0 days | 2 | 3 | Flexible up |
| triacetin | | | 4 | to 1 month |
| 1 wt % | t = 5 days | 2 | 17 | although |
| glycerol | | | 12 | more force |
| | t = 14 days | 2 | 36 | required |
| | | | 44 | to flex |
| | t = 30 days | 2 | 47 | |

TABLE 25E
Flexural Moduli of Depots with Benzyl Benzoate/Triacetin/Glycerol
at Different Time Points

| | | Thick- | Flexural | |
|---|---|---|---|---|
| Depot | | ness | Modulus | |
| Formulation | Time Point | (mm) | (MPa) | Remarks |
| 1.4 wt % benzyl | t = 0 days | 2 | 7 | Flexible up |
| benzoate | | | 5 | to 2 weeks |
| 1.4 wt % | t = 7 days | 2 | 17 | although |
| triacetin | | | 33 | more force |
| 0.4 wt % | t = 14 days | 2 | 33 | required |
| glycerol | | | 53 | to flex |

TABLE 25F
Flexural Moduli of Depots with Triacetin/Glycerol/Unmilled
BUP-HCl and Different Thicknesses at Different Time Points

| | | Thick- | Flexural | |
|---|---|---|---|---|
| Depot | | ness | Modulus | |
| Formulation | Time Point | (mm) | (MPa) | Remarks |
| 2.8 wt % | t = 0 days | 0.8 | 25 | Samples remained at |
| triacetin | | | 97 | similar flexibility after |
| 0.4 wt % | t = 1 month | 0.8 | 57 | 1 month in real time |
| glycerol | (real time) | | 149 | and accelerated aging |
| unmilled | t = 1 month | 0.8 | 36 | |
| BUP-HCl | (accelerated) | | 131 | |
| | t = 0 days | 1 | 28 | Flexibility decreased |
| | | | 25 | slightly after 1 month |
| | t = 1 month | 1 | 34 | accelerated aging |
| | (real time) | | 35 | |
| | t = 1 month | 1 | 51 | |
| | (accelerated) | | 132 | |
| | t = 0 days | 2 | 28 | |
| | | | 31 | Samples remained |
| | t = 1 month | 2 | 91 | flexible although more |
| | (real time) | | | force required to flex |
| | | | | after 1 month aging |

TABLE 25G
Flexural Moduli of Depots with Benzyl Benzoate/Triacetin/Glycerol
and Different Thicknesses at Different Time Points

| | | Thick- | Flexural | |
|---|---|---|---|---|
| Depot | | ness | Modulus | |
| Formulation | Time Point | (mm) | (MPa) | Remarks |
| 1.4 wt % | t = 0 days | 0.8 | 184 | Samples remained |
| benzyl | | | 113 | flexible after 1 month |
| benzoate | t = 1 month | 0.8 | 154 | of aging |
| 1.4 wt % | (real time) | | | |
| triacetin | t = 0 days | 1 | 147 | Samples remined |
| 0.4 wt % | | | 84 | flexible after 1 month |
| glycerol | t = 1 month | 1 | 111 | aging in real time, |
| | (real time) | | 89 | cracked upon bending |
| | t = 1 month | 1 | 457 | after 1 month |
| | (accelerated) | | 398 | accelerated aging |
| | t = 0 days | 2 | 7 | Samples remaining |
| | | | 5 | flexible after 1 month |
| | t = 1 month | 2 | 40 | aging but more force |
| | (real time) | | | required to flex |

The accelerated aging data in Tables 25F and 25G was performed by storing the depots in a stability chamber at a temperature of 40° C.+/−2° C. with a relative humidity of 75%+/−5%. The results shown in Tables 25F and 25G demonstrate that depots formulated with plasticizers remained flexible up to 1 month storage at room temperature; some formulations were also able to maintain flexibility under accelerated aging conditions.

Overall, these results demonstrate that certain plasticizers (triacetin, benzyl benzoate, diethyl phthalate) and depot configurations could provide improved flexibility compared to plasticizer-free depots while also allowing providing a desired drug release profile. Flexibility and controlled release could be maintained for extended periods of time with the use of dual- or triple-plasticizer formulations.

### ADDITIONAL EXAMPLES (I)

Several aspects of the present technology are set forth in the following examples.

Example I-1. An implantable depot for treating pain, the implantable depot comprising:
a therapeutic region comprising a polymer, an analgesic agent, and a plasticizer,
wherein, when implanted in vivo, the therapeutic region is configured to release the analgesic agent for a treatment period of at least 3 days, and
wherein the implantable depot has a flexural modulus within a range from 1 MPa to 400 MPa.

Example I-2. The implantable depot of Example I-1, wherein the plasticizer is hydrophilic.

Example I-3. The implantable depot of Example I-1, wherein the plasticizer is hydrophobic.

Example I-4. The implantable depot of any one of Examples I-1-3, wherein the plasticizer comprises one or more of a triglyceride, a fatty acid ester, a lactic acid ester, a citrate, a phthalate, a glycerol ester, a sebacate, a monoglyceride ester, a benzyl derivative, a polyethylene glycol, a polysorbate, a diol, or a triol.

Example I-5. The implantable depot of any one of Examples I-1-4, wherein the plasticizer comprises one or more of triacetin, diethyl phthalate, benzyl benzoate, or glycerol.

Example I-6. The implantable depot of any one of Examples I-1-5, wherein a relative energy difference (RED) between the plasticizer and the polymer is less than or equal to 1.

Example I-7. The implantable depot of any one of Examples I-1-6, wherein a vapor pressure of the plasticizer is less than or equal to 0.5 Pa at 25° C.

Example I-8. The implantable depot of any one of Examples I-1-7, wherein a log P value of the plasticizer is within a range from −1.5 to 6, 0 to 4, or 2 to 4.

Example I-9. The implantable depot of any one of Examples I-1-8, wherein the plasticizer constitutes from 0.1% to 20% of a total mass of the therapeutic region.

Example I-10. The implantable depot of any one of Examples I-1-9, wherein the plasticizer constitutes from 0.1% to 20% of a total mass of the implantable depot.

Example I-11. The implantable depot of any one of Examples I-1-10, wherein the therapeutic region includes only a single plasticizer.

Example I-12. The implantable depot of any one of Examples I-1-10, wherein the plasticizer is a first plasticizer, and the therapeutic region further comprises a second plasticizer.

Example I-13. The implantable depot of Example I-12, wherein the first plasticizer is triacetin and the second plasticizer is glycerol.

Example I-14. The implantable depot of Example I-12 or 13, wherein the therapeutic region further comprises a third plasticizer.

Example I-15. The implantable depot of Example I-14, wherein the first plasticizer is triacetin, the second plasticizer is glycerol, and the third plasticizer is benzyl benzoate.

Example I-16. The implantable depot of any one of Examples I-1-15, wherein therapeutic region has a first surface, a second surface opposite the first surface, and a lateral surface between the first and second surfaces.

Example I-17. The implantable depot of Example I-16, wherein the first surface, second, surface, and lateral surfaces of the therapeutic region are exposed.

Example I-18. The implantable depot of Example I-16, further comprising a first control region covering the first surface of the therapeutic region, the first control region comprising a polymer.

Example I-19. The implantable depot of Example I-18, wherein the first control region does not comprise any plasticizer.

Example I-20. The implantable depot of Example I-18, wherein the first control region comprises a plasticizer.

Example I-21. The implantable depot of any one of Examples I-18-20, further comprising a second control region covering the second surface of the therapeutic region, the second control region comprising a polymer.

Example I-22. The implantable depot of Example I-21, wherein the second control region does not comprise any plasticizer.

Example I-23. The implantable depot of Example I-21, wherein the second control region comprises a plasticizer.

Example I-24. The implantable depot of any one of Examples I-1-23, wherein the analgesic agent constitutes at least 50% of a total mass of the implantable depot.

Example I-25. The implantable depot of any one of Examples I-1-24, wherein the analgesic agent comprises bupivacaine.

Example I-26. The implantable depot of any one of Examples I-1-25, wherein the polymer comprises poly(lactide-co-glycolide).

Example I-27. The implantable depot of any one of Examples I-1-26, wherein the treatment period is no more than 7 days.

Example I-28. The implantable depot of any one of Examples I-1-26, wherein the treatment period is at least 7 days.

Example I-29. An implantable depot for treating pain, the implantable depot comprising:
a therapeutic region comprising a polymer and an analgesic agent, wherein the therapeutic region has a first surface, a second surface opposite the first surface, and a lateral surface between the first and second surfaces,
wherein, when implanted in vivo, the therapeutic region is configured to release the analgesic agent from the first, second, and lateral surfaces of the therapeutic region for a treatment period of no more than 7 days.

Example I-30. The implantable depot of Example I-29, wherein the first, second, and lateral surfaces of the therapeutic region are exposed.

Example I-31. The implantable depot of Examples I-29 or 30, wherein at least 50%, 60%, 70%, 75%, 80%, 85%, 90%, 95%, or 99% of the analgesic agent is released within the first day of the treatment period.

Example I-32. The implantable depot of any one of Examples I-29-31, wherein at least 50%, 60%, 70%, 75%, 80%, 85%, 90%, 95%, or 99% of the analgesic agent is released within the first 2 days of the treatment period.

Example I-33. The implantable depot of any one of Examples I-29-32, wherein at least 50%, 60%, 70%, 75%, 80%, 85%, 90%, 95%, or 99% of the analgesic agent is released within the first 3 days of the treatment period.

Example I-34. The implantable depot of any one of Examples I-29-33, wherein at least 50%, 60%, 70%, 75%, 80%, 85%, 90%, 95%, or 99% of the analgesic agent is released within the first 4 days of the treatment period.

Example I-35. The implantable depot of any one of Examples I-29-34, wherein, when implanted in vivo, the implantable depot produces a mean plasma concentration of the analgesic agent greater than or equal to a therapeutic threshold within the first 12 hours, 1 day, 2 days, 3 days, or 4 days of the treatment period.

Example I-36. The implantable depot of Example I-35, wherein the therapeutic threshold is 200 ng/ml.

Example I-37. The implantable depot of any one of Examples I-29-36, wherein, when implanted in vivo, the implantable depot produces a mean Tₘₐₓ of no more than 96 hours, 72 hours, 36 hours, 48 hours, 24 hours, or 12 hours.

Example I-38. The implantable depot of any one of Examples I-29-37, wherein when implanted in vivo, the implantable depot produces a mean Tₗₐₛₜ of no more than 14 days, 13 days, 12 days, 11 days, 10 days, 9 days, 8 days, 7 days, 6 days, 5 days, 4 days, 3 days, 2 days, or 1 day.

Example I-39. The implantable depot of any one of Examples I-29-38, wherein the analgesic agent constitutes at least 50% of a total mass of the implantable depot.

Example I-40. The implantable depot of any one of Examples I-29-39, wherein the analgesic agent comprises bupivacaine.

Example I-41. The implantable depot of any one of Examples I-29-40, wherein the polymer comprises poly(lactide-co-glycolide).

Example I-42. The implantable depot of any one of Examples I-29-41, wherein the analgesic agent and the polymer are discrete phases within the therapeutic agent.

Example I-43. The implantable depot of any one of Examples I-29-42, wherein the therapeutic region comprises a releasing agent.

Example I-44. The implantable depot of Example I-43, wherein the releasing agent constitutes no more than 5% of a total mass of the implantable depot.

Example I-45. The implantable depot of Example I-43 or Example I-44, wherein the releasing agent comprises polysorbate.

Example I-46. The implantable depot of any one of Examples I-29-45, wherein the therapeutic region further comprises a plasticizer.

Example I-47. The implantable depot of Example I-46, wherein the plasticizer is hydrophilic.

Example I-48. The implantable depot of Example I-46, wherein the plasticizer is hydrophobic.

Example I-49. The implantable depot of any one of Examples I-46-48, wherein the plasticizer comprises one or more of a triglyceride, a fatty acid ester, a lactic acid ester, a citrate, a phthalate, a glycerol ester, a sebacate, a monoglyceride ester, a benzyl derivative, a polyethylene glycol, a polysorbate, a diol, or a triol.

Example I-50. The implantable depot of any one of Examples I-46-49, wherein the plasticizer comprises one or more of triacetin, diethyl phthalate, benzyl benzoate, or glycerol.

Example I-51. The implantable depot of any one of Examples I-29-50, wherein the implantable depot has a flexural modulus within a range from 1 MPa to 400 MPa.

Example I-52. A method for treating pain in a subject after a surgical procedure, the method comprising placing one or more implantable depots of any one of Examples I-1-51 in the subject.

Example I-53. The method of Example I-52, wherein a mass of the analgesic agent in each depot is greater than or equal to 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, or 1800 mg.

Example I-54. The method of Example I-52 or Example I-53, wherein, when implanted in vivo, each depot continuously releases the analgesic agent over a time period of no more than 1 day, 2 days, 3 days, 4 days, 5 days, 6 days, 7 days, 8 days, 9 days, 10 days, 11 days, 12 days, 13 days, or 14 days.

Example I-55. The method of any one of Examples I-52-54, wherein, when implanted in vivo, the one or more depots produce a mean plasma concentration of the analgesic agent greater than or equal to 5 ng/ml, 10 ng ml, 15 ng/ml, 20 mg/ml, 25 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 300 ng/ml, 400 ng/ml, 500 ng/ml, 600 ng/ml, 700 ng/ml, 800 ng/ml, 900 ng/ml, or 1000 ng/ml.

Example I-56. The method of Example I-55, wherein the mean plasma concentration is maintained for a period of no more than 1 day, 2 days, 3 days, 4 days, 5 days, 6 days, 7 days, 8 days, 9 days, 10 days, 11 days, 12 days, 13 days, or 14 days.

Example I-57. The method of any one of Examples I-52-56, wherein, when implanted in vivo, the one or more depots produce a mean Cₘₐₓ of the analgesic agent less than or equal to 5000 ng/ml, 4000 ng/ml, 3000 ng/ml, 2000 ng/ml, 1000 ng/ml, 900 ng/ml, 800 ng/ml, 700 ng/ml, 600 ng/ml, 500 ng/ml, 400 ng/ml, 300 ng/ml, 200 ng/ml, 100 ng/ml, or 50 ng/ml.

Example I-58. The method of any one of Examples I-52-57, wherein, when implanted in vivo, the one or more depots produce a mean AUC_{0-14d} of the analgesic agent of at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, or 8000 day-ng/ml.

Example I-59. The method of any one of Examples I-52-58, wherein a mean NRS score of a treatment population treated with the one or more implantable depots is reduced by at least 10%, 20%, 30%, 40%, 50%, 60%, 70%, 80%, or 90% compared to a mean NRS score of a control population that has not been treated with the one or more implantable depots.

Example I-60. The method of Example I-59, wherein the mean NRS score of the treatment population and the mean NRS score of the control population are evaluated at 12 hours, 1 day, 2 days, 3 days, 4 days, 5 days, 6 days, 7 days, 8 days, 9 days, 10 days, 11 days, 12 days, 13 days, or 14 days after the surgical procedure.

Example I-61. The method of any one of Examples I-52-60, wherein at least 10%, 20%, 30%, 40%, 50%, 60%, 70%, 80%, 90%, or 100% of a treatment population treated with the one or more implantable depots is pain-free at a time point after the surgical procedure.

Example I-62. The method of Example I-61, wherein the time point is 12 hours, 1 day, 2 days, 3 days, 4 days, 5 days, 6 days, 7 days, 8 days, 9 days, 10 days, 11 days, 12 days, 13 days, or 14 days after the surgical procedure.

Example I-63. The method of any one of Examples I-52-62, wherein a mean NRS AUC of a treatment population treated with the one or more implantable depots is reduced by at least 10%, 20%, 30%, 40%, 50%, 60%, 70%, 80%, or 90% compared to a mean NRS AUC of a control population that has not been treated with the one or more implantable depots.

Example I-64. The method of Example I-63, wherein the mean NRS AUC of the treatment population and the mean NRS AUC of the control population are evaluated over a time period from 0 hours to 12 hours, from 0 hours to 24 hours, from 0 hours to 72 hours, from 0 hours to 96 hours, from 0 hours to 7 days, from 0 hours to 14 days, from 12 hours to 24 hours, from 12 hours to 36 hours, from 12 hours to 72 hours, from 12 hours to 96 hours, from 12 hours to 7 days, from 12 hours to 14 days, from 12 hours to 14 days, from 1 day to 2 days, from 1 day to 4 days, from 1 day to 7 days, from 1 day to 14 days, from 2 days to 3 days, from 2 days to 4 days, from 2 days to 7 days, from 2 days to 14 days, from 3 days to 4 days, from 3 days to 7 days, from 3 days to 14 days, from 4 days to 5 days, from 4 days to 7 days, from 4 days to 14 days, from 5 days to 6 days, from 5 days to 7 days, from 5 days to 14 days, from 6 days to 7 days, from 6 days to 14 days, from 7 days to 8 days, from 7 days to 14 days, from 8 days to 9 days, from 9 days to 10 days, from 10 days to 11 days, from 11 days to 12 days, from 12 days to 13 days, or from 13 days to 14 days after the surgical procedure.

Example I-65. The method of any one of Examples I-52-64, wherein at least 10%, 20%, 30%, 40%, 50%, 60%, 70%, 80%, 90%, or 100% of a treatment population treated with the one or more implantable depots is opioid-free over a time period after the surgical procedure.

Example I-66. The method of Example I-65, wherein the time period is from 0 hours to 12 hours, from 0 hours to 24 hours, from 0 hours to 72 hours, from 0 hours to 96 hours, from 0 hours to 7 days, from 0 hours to 14 days, from 12 hours to 24 hours, from 12 hours to 36 hours, from 12 hours to 72 hours, from 12 hours to 96 hours, from 12 hours to 7 days, from 12 hours to 14 days, from 1 day to 2 days, from 1 day to 4 days, from 1 day to 7 days, from 1 day to 14 days, from 2 days to 3 days, from 2 days to 4 days, from 2 days to 7 days, from 2 days to 14 days, from 3 days to 4 days, from 3 days to 7 days, from 3 days to 14 days, from 4 days to 5 days, from 4 days to 7 days, from 4 days to 14 days, from 5 days to 6 days, from 5 days to 7 days, from 5 days to 14 days, from 6 days to 7 days, from 6 days to 14 days, from 7 days to 8 days, from 7 days to 14 days, from 8 days to 9 days, from 9 days to 10 days, from 10 days to 11 days, from 11 days to 12 days, from 12 days to 13 days, or from 13 days to 14 days after the surgical procedure.

Example I-67. The method of any one of Examples I-52-66, wherein a mean total opioid consumption of a treatment population treated with the one or more implantable depots is reduced by at least 10%, 20%, 30%, 40%, 50%, 60%, 70%, 80%, or 90% compared to a mean total opioid consumption of a control population that has not been treated with the one or more depots.

Example I-68. The method of Example I-67, wherein the mean total opioid consumption of the treatment population and the mean total opioid consumption of the control population are evaluated over a time period from 0 hours to 12 hours, from 0 hours to 24 hours, from 0 hours to 72 hours, from 0 hours to 96 hours, from 0 hours to 7 days, from 0 hours to 14 days, from 12 hours to 24 hours, from 12 hours to 36 hours, from 12 hours to 72 hours, from 12 hours to 96 hours, from 12 hours to 7 days, from 12 hours to 14 days, from 1 day to 2 days, from 1 day to 4 days, from 1 day to 7 days, from 1 day to 14 days, from 2 days to 3 days, from 2 days to 4 days, from 2 days to 7 days, from 2 days to 14 days, from 3 days to 4 days, from 3 days to 7 days, from 3 days to 14 days, from 4 days to 5 days, from 4 days to 7 days, from 4 days to 14 days, from 5 days to 6 days, from 5 days to 7 days, from 5 days to 14 days, from 6 days to 7 days, from 6 days to 14 days, from 7 days to 8 days, from 7 days to 14 days, from 8 days to 9 days, from 9 days to 10 days, from 10 days to 11 days, from 11 days to 12 days, from 12 days to 13 days, or from 13 days to 14 days after the surgical procedure.

Example I-69. The method of any one of Examples I-52-68, wherein a mean total opioid consumption of a treatment population treated with the one or more implantable depots is no more than 600 morphine milligram equivalents (MME), 550 MME, 500 MME, 450 MME, 400 MME, 350 MME, 300 MME, 250 MME, 200 MME, 150 MME, 100 MME, or 50 MME.

Example I-70. The method of Example I-69, wherein the mean total opioid consumption of the treatment population is evaluated over a time period from 0 hours to 12 hours, from 0 hours to 24 hours, from 0 hours to 72 hours, from 0 hours to 96 hours, from 0 hours to 7 days, from 0 hours to 14 days, from 12 hours to 24 hours, from 12 hours to 36 hours, from 12 hours to 72 hours, from 12 hours to 96 hours, from 12 hours to 7 days, from 12 hours to 14 days, from 1 day to 2 days, from 1 day to 4 days, from 1 day to 7 days, from 1 day to 14 days, from 2 days to 3 days, from 2 days to 4 days, from 2 days to 7 days, from 2 days to 14 days, from 3 days to 4 days, from 3 days to 7 days, from 3 days to 14 days, from 4 days to 5 days, from 4 days to 7 days, from 4 days to 14 days, from 5 days to 6 days, from 5 days to 7 days, from 5 days to 14 days, from 6 days to 7 days, from 6 days to 14 days, from 7 days to 8 days, from 7 days to 14 days, from 8 days to 9 days, from 9 days to 10 days, from 10 days to 11 days, from 11 days to 12 days, from 12 days to 13 days, or from 13 days to 14 days after the surgical procedure.

Example I-71. The method of any one of Examples I-52-70, wherein a mean time to first opioid consumption of a treatment population treated with the one or more implantable depots is delayed by at least 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 96 hours, 5 days, 6 days, or 7 days compared to a mean time to first opioid consumption of a control population that has not been treated with the one or more implantable depots.

Example I-72. The method of any one of Examples I-52-71, wherein the surgical procedure is a soft tissue repair procedure.

Example I-73. The method of Example I-72, wherein the soft tissue repair procedure is an open inguinal hernia repair, an abdominoplasty, a laparotomy, a mammoplasty, or a ventral hernia repair.

Example I-74. The method of any one of Examples I-52-71, wherein the surgical procedure is an orthopedic procedure.

Example I-75. The method of Example I-74, wherein the orthopedic procedure is a total shoulder arthroplasty or a total hip arthroplasty.

Example I-76. The method of any one of Examples I-52-71, wherein the surgical procedure is a foot or ankle procedure.

Example I-77. The method of Example I-76, wherein the foot or ankle procedure is a bunionectomy, an ankle fracture repair, or a hindfoot fusion.

Example I-78. The method of any one of Examples I-52-71, wherein the surgical procedure is a gynecologic or pelvic procedure.

Example I-79. The method of Example I-78, wherein the gynecologic or pelvic procedure is a C-section, a hysterectomy, or an oophorectomy.

Example I-80. The method of any one of Examples I-52-71, wherein the surgical procedure is a thoracic procedure.

Example I-81. The method of Example I-80, wherein the thoracic procedure is a sternotomy or a thoracotomy.

Example I-82. The method of any one of Examples I-52-71, wherein the surgical procedure is a gastrointestinal procedure.

Example I-83. The method of Example I-82, wherein the gastrointestinal procedure is a colorectal resection.

### ADDITIONAL EXAMPLES (II)

Several aspects of the present technology are set forth in the following examples.

Example II-1. An implantable depot for treating pain in a subject after a surgical procedure, the implantable depot comprising:
a therapeutic region having a first surface, a second surface opposite the first surface, and a lateral surface between the first and second surfaces, wherein the therapeutic region comprises a first polymer and an analgesic agent;
a first control region covering the first surface of the therapeutic region to inhibit release of the analgesic agent from the first surface, wherein the first control region comprises a second polymer;
a second control region covering the second surface of the therapeutic region to inhibit release of the analgesic agent from the second surface, wherein the second control region comprises a third polymer; and
one or more holes extending through the first and second control regions and the therapeutic region to form one or more exposed portions of the therapeutic region spaced apart from the lateral surface,
wherein, when implanted in the subject, the implantable depot is configured to release the analgesic agent from the lateral surface and the one or more exposed portions of the therapeutic region.

Example II-2. The implantable depot of Example II-1, wherein the one or more holes are configured such that a maximum travel distance of the therapeutic agent is no more than 5 mm.

Example II-3. The implantable depot of Example II-1 or Example II-2, wherein the implantable depot has a triangular shape.

Example II-4. The implantable depot of any one of Examples II-1-3, wherein the implantable depot includes a plurality of holes.

Example II-5. The implantable depot of Example II-4, wherein the implantable depot includes four holes.

Example II-6. The implantable depot of Example II-5, wherein the four holes include:
a central hole located at or near a center of the implantable depot; and
three peripheral holes spaced apart from the central hole.

Example II-7. The implantable depot of any one of Examples II-1-6, wherein at least some of the one or more holes have different sizes.

Example II-8. The implantable depot of any one of Examples II-1-7, wherein at least some of the one or more holes have different shapes.

Example II-9. The implantable depot of any one of Examples II-1-6, wherein the one or more holes each have the same size and shape.

Example II-10. The implantable depot of any one of Examples II-1-3, wherein the implantable depot includes a single hole.

Example II-11. The implantable depot of any one of Examples II-1-10, wherein at least some of the one or more holes have a circular shape.

Example II-12. The implantable depot of any one of Examples II-1-11, wherein at least some of the holes have a width within a range from 1 mm to 5 mm.

Example II-13. The implantable depot of any one of Examples II-1-12, wherein the implantable depot has a total thickness within a range from 1.8 mm to 2.2 mm.

Example II-14. The implantable depot of any one of Examples II-1-13, wherein the therapeutic region has a first thickness, and the first and second control regions collectively have a second thickness less than the first thickness.

Example II-15. The implantable depot of Example II-14, wherein the first thickness is at least 95% of a total thickness of the implantable depot.

Example II-16. The implantable depot of Example II-14 or Example II-15, wherein the second thickness is no more than 5% of a total thickness of the implantable depot.

Example II-17. The implantable depot of any one of Examples II-14-16, wherein a ratio of the second thickness to the first thickness is no more than 1/35.

Example II-18. The implantable depot of any one of Examples II-14-17, wherein the first thickness is within a range from 1.75 mm to 2.25 mm, and the second thickness is within a range from 40 μm to 60 km.

Example II-19. The implantable depot of any one of Examples II-1-18, wherein the therapeutic region has a first volume, and the first and second control regions collectively have a second volume less than the first volume.

Example II-20. The implantable depot of Example II-19, wherein the first volume is at least 95% of a total volume of the implantable depot.

Example II-21. The implantable depot of Example II-19 or Example II-20, wherein the second volume is no more than 5% of a total volume of the implantable depot.

Example II-22. The implantable depot of any one of Examples II-19-21, wherein the first volume is at least 850 mm³ and the second volume is no more than 25 mm³.

Example II-23. The implantable depot of any one of Examples II-1-22, wherein the implantable depot includes at least three sides, and wherein a length of each side is within a range from 25 mm to 35 mm.

Example II-24. The implantable depot of any one of Examples II-1-23, wherein the analgesic agent constitutes at least 60% of a total mass of the depot.

Example II-25. The implantable depot of any one of Examples II-1-24, wherein the analgesic agent constitutes at least 60% of a total mass of the therapeutic region.

Example II-26. The implantable depot of any one of Examples II-1-25, wherein a total mass of the analgesic agent in the implantable depot is within a range from 540 mg to 660 mg.

Example II-27. The implantable depot of any one of Examples II-1-26, wherein the analgesic agent includes bupivacaine or ropivacaine.

Example II-28. The implantable depot of any one of Examples II-1-27, wherein the first, second, and third polymers collectively constitute no more than 35% of a total mass of the depot.

Example II-29. The implantable depot of any one of Examples II-1-28, wherein the first polymer constitutes no more than 35% of a total mass of the therapeutic agent.

Example II-30. The implantable depot of any one of Examples II-1-29, wherein the second polymer constitutes at least 95% of a total mass of the first control region, and the third polymer constitutes at least 95% of a total mass of the second control region.

Example II-31. The implantable depot of any one of Examples II-1-30, wherein a total mass of the first, second, and third polymers in the implantable depot is within a range from 300 mg to 350 mg.

Example II-32. The implantable depot of any one of Examples II-1-31, wherein the first, second, and third polymers are the same polymer.

Example II-33. The implantable depot of any one of Examples II-1-32, wherein the first, second, and third polymers are bioresorbable polymers.

Example II-34. The implantable depot of any one of Examples II-1-33, wherein one or more of the first, second, or third polymers are poly(lactide-co-glycolide).

Example II-35. The implantable depot of any one of Examples II-1-34, wherein the analgesic agent and the first polymer are discrete phases within the therapeutic region.

Example II-36. The implantable depot of any one of Examples II-1-35, wherein the therapeutic region includes a releasing agent.

Example II-37. The implantable depot of Example II-36, wherein the releasing agent constitutes no more than 5% of a total mass of the implantable depot.

Example II-38. The implantable depot of Example II-36 or Example II-37, wherein the releasing agent constitutes no more than 5% of a total mass of the therapeutic region.

Example II-39. The implantable depot of any one of Examples II-36-38, wherein a total mass of the releasing agent in the implantable depot is within a range from 20 mg to 40 mg.

Example II-40. The implantable depot of any one of Examples II-36-39, wherein the releasing agent is polysorbate.

Example II-41. The implantable depot of any one of Examples II-1-40, wherein, when implanted in vivo, the implantable depot is configured to continuously release the analgesic agent over a time period of at least 7 days, 14 days, 21 days, or 30 days.

Example II-42. The implantable depot of any one of Examples II-1-41, wherein the implantable depot is configured to release the analgesic agent at a first rate over a first time period and a second rate over a second time period, wherein the first rate is greater than the second rate.

Example II-43. The implantable depot of any one of Examples II-1-42, wherein the implantable depot is configured to release up to 20% of the analgesic agent over a first 72 hours after implantation.

Example II-44. The implantable depot of any one of Examples II-1-43, wherein the implantable depot is configured to release up to 50% of the analgesic agent over a first 7 days after implantation.

Example II-45. The implantable depot of any one of Examples II-1-44, wherein the implantable depot is configured to release at least 70% of the analgesic agent over a first 14 days after implantation.

Example II-46. A system for treating pain in a subject after a surgical procedure, the system comprising one or more of the implantable depots of any one of Examples II-1-45.

Example II-47. The system of Example II-46, wherein the system comprises a plurality of implantable depots.

Example II-48. The system of Example II-47, wherein the system comprises three implantable depots.

Example II-49. The system of Example II-46, wherein the system comprises a single implantable depot.

Example II-50. The system of any one of Examples II-46-49, wherein, when implanted in vivo, the one or more implantable depots produce a mean plasma concentration of the analgesic agent greater than or equal to 5 ng/ml, 10 ng ml, 15 ng/ml, 20 mg/ml, 25 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 300 ng/ml, 400 ng/ml, 500 ng/ml, 600 ng/ml, 700 ng/ml, 800 ng/ml, 900 ng/ml, or 1000 ng/ml,

Example II-51. The system of Example II-50, wherein the mean plasma concentration is maintained for a period of at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days.

Example II-52. The system of any one of Examples II-46-51, wherein, when implanted in vivo, the one or more implantable depots produce a mean Cₘₐₓ of the analgesic agent less than or equal to 5000 ng/ml, 4000 ng/ml, 3000 ng/ml, 2000 ng/ml, 1000 ng/ml, 900 ng/ml, 800 ng/ml, 700 ng/ml, 600 ng/ml, 500 ng/ml, 400 ng/ml, 300 ng/ml, 200 ng/ml, 100 ng/ml, or 50 ng/ml.

Example II-53. The system of any one of Examples II-46-52, wherein, when implanted in vivo, the one or more implantable depots produce a mean AUC_{0-14d} of the analgesic agent of at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, or 8000 day-ng/ml.

Example II-54. A method for treating pain in a subject after a surgical procedure, the method comprising placing the system of any one of Examples II-46-53 in the subject.

Example II-55. A method for treating pain in a subject after a surgical procedure, the method comprising:
placing one or more depots in the subject, wherein each depot comprises:
   a therapeutic region having a first surface, a second surface opposite the first surface, and a lateral surface between the first and second surfaces, wherein the therapeutic region comprises a first polymer and an analgesic agent;
   a first control region covering the first surface of the therapeutic region to inhibit release of the analgesic agent from the first surface, wherein the first control region comprises a second polymer;
   a second control region covering the second surface of the therapeutic region to inhibit release of the analgesic agent from the second surface, wherein the second control region comprises a third polymer; and
   one or more holes extending through the first and second control regions and the therapeutic region to form one or more exposed portions of the therapeutic region spaced apart from the lateral surface,
wherein each depot is configured to release the analgesic agent from the lateral surface and the one or more exposed portions of the therapeutic region.

Example II-56. The method of Example II-55, wherein the one or more depots comprise one, two, three, four, five, six, seven, eight, nine, or ten depots.

Example II-57. The method of Example II-55 or Example II-56, wherein a mass of the analgesic agent in each depot is within a range from 540 mg to 660 mg.

Example II-58. The method of any one of Examples II-55-57, wherein a mass of the analgesic agent in each depot is greater than or equal to 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, or 1800 mg.

Example II-59. The method of any one of Examples II-55-58, wherein, when implanted in vivo, each depot continuously releases the analgesic agent over a time period of at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days.

Example II-60. The method of any one of Examples II-55-59, wherein, when implanted in vivo, the one or more depots produce a mean plasma concentration of the analgesic agent greater than or equal to 5 ng/ml, 10 ng ml, 15 ng/ml, 20 mg/ml, 25 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 300 ng/ml, 400 ng/ml, 500 ng/ml, 600 ng/ml, 700 ng/ml, 800 ng/ml, 900 ng/ml, or 1000 ng/ml.

Example II-61. The method of Example II-60, wherein the mean plasma concentration is maintained for a period of at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days.

Example II-62. The method of any one of Examples II-55-61, wherein, when implanted in vivo, the one or more depots produce a mean Cₘₐₓ of the analgesic agent less than or equal to 1000 ng/ml, 900 ng/ml, 800 ng/ml, 700 ng/ml, 600 ng/ml, 500 ng/ml, 400 ng/ml, 300 ng/ml, 200 ng/ml, 100 ng/ml, or 50 ng/ml.

Example II-63. The method of any one of Examples II-55-62, wherein, when implanted in vivo, the one or more depots produce a mean AUC_{0-14d} of the analgesic agent of at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, or 8000 day-ng/ml.

Example II-64. The method of any one of Examples II-55-63, wherein a mean NRS score of a treatment population treated with the one or more depots is reduced by at least 10%, 20%, 30%, 40%, 50%, 60%, 70%, 80%, or 90% compared to a mean NRS score of a control population that has not been treated with the one or more depots.

Example II-65. The method of Example II-64, wherein the mean NRS score of the treatment population and the mean NRS score of the control population are evaluated at 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the surgical procedure.

Example II-66. The method of any one of Examples II-55-65, wherein at least 10%, 20%, 30%, 40%, 50%, 60%, 70%, 80%, 90%, or 100% of a treatment population treated with the one or more depots is pain-free at a time point after the surgical procedure.

Example II-67. The method of Example II-66, wherein the time point is 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the surgical procedure.

Example II-68. The method of any one of Examples II-55-67, wherein a mean NRS AUC of a treatment population treated with the one or more depots is reduced by at least 10%, 20%, 30%, 40%, 50%, 60%, 70%, 80%, or 90% compared to a mean NRS AUC of a control population that has not been treated with the one or more depots.

Example II-69. The method of Example II-68, wherein the mean NRS AUC of the treatment population and the mean NRS AUC of the control population are evaluated over a time period from 0 hours to 12 hours, from 0 hours to 24 hours, from 0 hours to 72 hours, from 0 hours to 96 hours, from 0 hours to 7 days, from 0 hours to 14 days, 0 hours to 15 days, from 0 hours to 30 days, from 12 hours to 24 hours, from 12 hours to 36 hours, from 12 hours to 72 hours, from 12 hours to 96 hours, from 12 hours to 7 days, from 12 hours to 14 days, from 12 hours to 14 days, from 12 hours to 30 days, from 1 day to 2 days, from 1 day to 4 days, from 1 day to 7 days, from 1 day to 14 days, from 1 day to 15 days, from 1 day to 30 days, from 2 days to 3 days, from 2 days to 4 days, from 2 days to 7 days, from 2 days to 14 days, from 2 days to 15 days, from 2 days to 30 days, from 3 days to 4 days, from 3 days to 7 days, from 3 days to 14 days, from 3 days to 15 days, from 3 days to 30 days, from 4 days to 5 days, from 4 days to 7 days, from 4 days to 14 days, from 4 days to 15 days, from 4 days to 30 days, from 5 days to 6 days, from 5 days to 7 days, from 5 days to 14 days, from 5 days to 15 days, from 5 days to 30 days, from 6 days to 7 days, from 6 days to 14 days, from 6 days to 15 days, from 6 days to 30 days, from 7 days to 8 days, from 7 days to 14 days, from 7 days to 15 days, from 7 days to 30 days, from 8 days to 9 days, from 9 days to 10 days, from 10 days to 11 days, from 11 days to 12 days, from 12 days to 13 days, from 13 days to 14 days, from 14 days to 15 days, from 14 days to 30 days, from 15 days to 30 days, or from 16 days to 30 days after the surgical procedure.

Example II-70. The method of any one of Examples II-55-69, wherein at least 10%, 20%, 30%, 40%, 50%, 60%, 70%, 80%, 90%, or 100% of a treatment population treated with the one or more depots is opioid-free over a time period after the surgical procedure.

Example II-71. The method of Example II-70, wherein the time period is from 0 hours to 12 hours, from 0 hours to 24 hours, from 0 hours to 72 hours, from 0 hours to 96 hours, from 0 hours to 7 days, from 0 hours to 14 days, 0 hours to 15 days, from 0 hours to 30 days, from 12 hours to 24 hours, from 12 hours to 36 hours, from 12 hours to 72 hours, from 12 hours to 96 hours, from 12 hours to 7 days, from 12 hours to 14 days, from 12 hours to 14 days, from 12 hours to 30 days, from 1 day to 2 days, from 1 day to 4 days, from 1 day to 7 days, from 1 day to 14 days, from 1 day to 15 days, from 1 day to 30 days, from 2 days to 3 days, from 2 days to 4 days, from 2 days to 7 days, from 2 days to 14 days, from 2 days to 15 days, from 2 days to 30 days, from 3 days to 4 days, from 3 days to 7 days, from 3 days to 14 days, from 3 days to 15 days, from 3 days to 30 days, from 4 days to 5 days, from 4 days to 7 days, from 4 days to 14 days, from 4 days to 15 days, from 4 days to 30 days, from 5 days to 6 days, from 5 days to 7 days, from 5 days to 14 days, from 5 days to 15 days, from 5 days to 30 days, from 6 days to 7 days, from 6 days to 14 days, from 6 days to 15 days, from 6 days to 30 days, from 7 days to 8 days, from 7 days to 14 days, from 7 days to 15 days, from 7 days to 30 days, from 8 days to 9 days, from 9 days to 10 days, from 10 days to 11 days, from 11 days to 12 days, from 12 days to 13 days, from 13 days to 14 days, from 14 days to 15 days, from 14 days to 30 days, from 15 days to 30 days, or from 16 days to 30 days after the surgical procedure.

Example II-72. The method of any one of Examples II-55-71, wherein a mean total opioid consumption of a treatment population treated with the one or more depots is reduced by at least 10%, 20%, 30%, 40%, 50%, 60%, 70%, 80%, or 90% compared to a mean total opioid consumption of a control population that has not been treated with the one or more depots.

Example II-73. The method of Example II-72, wherein the mean total opioid consumption of the treatment population and the mean total opioid consumption of the control population are evaluated over a time period from 0 hours to 12 hours, from 0 hours to 24 hours, from 0 hours to 72 hours, from 0 hours to 96 hours, from 0 hours to 7 days, from 0 hours to 14 days, 0 hours to 15 days, from 0 hours to 30 days, from 12 hours to 24 hours, from 12 hours to 36 hours, from 12 hours to 72 hours, from 12 hours to 96 hours, from 12 hours to 7 days, from 12 hours to 14 days, from 12 hours to 14 days, from 12 hours to 30 days, from 1 day to 2 days, from 1 day to 4 days, from 1 day to 7 days, from 1 day to 14 days, from 1 day to 15 days, from 1 day to 30 days, from 2 days to 3 days, from 2 days to 4 days, from 2 days to 7 days, from 2 days to 14 days, from 2 days to 15 days, from 2 days to 30 days, from 3 days to 4 days, from 3 days to 7 days, from 3 days to 14 days, from 3 days to 15 days, from 3 days to 30 days, from 4 days to 5 days, from 4 days to 7 days, from 4 days to 14 days, from 4 days to 15 days, from 4 days to 30 days, from 5 days to 6 days, from 5 days to 7 days, from 5 days to 14 days, from 5 days to 15 days, from 5 days to 30 days, from 6 days to 7 days, from 6 days to 14 days, from 6 days to 15 days, from 6 days to 30 days, from 7 days to 8 days, from 7 days to 14 days, from 7 days to 15 days, from 7 days to 30 days, from 8 days to 9 days, from 9 days to 10 days, from 10 days to 11 days, from 11 days to 12 days, from 12 days to 13 days, from 13 days to 14 days, from 14 days to 15 days, from 14 days to 30 days, from 15 days to 30 days, or from 16 days to 30 days after the surgical procedure.

Example II-74. The method of any one of Examples II-55-73, wherein a mean total opioid consumption of a treatment population treated with the one or more depots is no more than 600 morphine milligram equivalents (MME), 550 MME, 500 MME, 450 MME, 400 MME, 350 MME, 300 MME, 250 MME, 200 MME, 150 MME, 100 MME, or 50 MME.

Example II-75. The method of Example II-74, wherein the mean total opioid consumption of the treatment population is evaluated over a time period from 0 hours to 12 hours, from 0 hours to 24 hours, from 0 hours to 72 hours, from 0 hours to 96 hours, from 0 hours to 7 days, from 0 hours to 14 days, 0 hours to 15 days, from 0 hours to 30 days, from 12 hours to 24 hours, from 12 hours to 36 hours, from 12 hours to 72 hours, from 12 hours to 96 hours, from 12 hours to 7 days, from 12 hours to 14 days, from 12 hours to 14 days, from 12 hours to 30 days, from 1 day to 2 days, from 1 day to 4 days, from 1 day to 7 days, from 1 day to 14 days, from 1 day to 15 days, from 1 day to 30 days, from 2 days to 3 days, from 2 days to 4 days, from 2 days to 7 days, from 2 days to 14 days, from 2 days to 15 days, from 2 days to 30 days, from 3 days to 4 days, from 3 days to 7 days, from 3 days to 14 days, from 3 days to 15 days, from 3 days to 30 days, from 4 days to 5 days, from 4 days to 7 days, from 4 days to 14 days, from 4 days to 15 days, from 4 days to 30 days, from 5 days to 6 days, from 5 days to 7 days, from 5 days to 14 days, from 5 days to 15 days, from 5 days to 30 days, from 6 days to 7 days, from 6 days to 14 days, from 6 days to 15 days, from 6 days to 30 days, from 7 days to 8 days, from 7 days to 14 days, from 7 days to 15 days, from 7 days to 30 days, from 8 days to 9 days, from 9 days to 10 days, from 10 days to 11 days, from 11 days to 12 days, from 12 days to 13 days, from 13 days to 14 days, from 14 days to 15 days, from 14 days to 30 days, from 15 days to 30 days, or from 16 days to 30 days after the surgical procedure.

Example II-76. The method of any one of Examples II-55-75, wherein a mean time to first opioid consumption of a treatment population treated with the one or more depots is delayed by at least 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 96 hours, 5 days, 6 days, or 7 days compared to a mean time to first opioid consumption of a control population that has not been treated with the one or more depots.

Example II-77. The method of any one of Examples II-55-76, wherein the surgical procedure is a total knee arthroplasty (TKA), total shoulder arthroplasty, total hip arthroplasty, inguinal hernia repair, bunionectomy, mammoplasty, or abdominoplasty.

Example II-78. An implantable depot for treating pain in a subject, the implantable depot comprising:
a therapeutic region having a first surface, a second surface opposite the first surface, and a lateral surface between the first and second surfaces, wherein the therapeutic region includes a polymer and an analgesic agent, and wherein at least some of the analgesic agent is in a free base form,
wherein, when implanted in the subject, the implantable depot is configured to release the analgesic agent from at least the lateral surface of the therapeutic region over a release period of at least 3 days.

Example II-79. The implantable depot of Example II-78, wherein the analgesic agent comprises bupivacaine and the free base form comprises bupivacaine free base.

Example II-80. The implantable depot of Example II-78 or 79, wherein at least 10%, 20%, 30%, 40%, 50%, 60%, 70%, 80%, 90%, 95%, or 99% of the analgesic agent is in the free base form.

Example II-81. The implantable depot of any one of Examples II-78 to 80, wherein 100% of the analgesic agent is in the free base form.

Example II-82. The implantable depot of any one of Examples II-78 to 80, wherein at least some of the analgesic agent is in a salt form.

Example II-83. The implantable depot of Example II-82, wherein the analgesic agent comprises bupivacaine and the salt form comprises bupivacaine hydrochloride.

Example II-84. The implantable depot of Example II-82 or 83, wherein a ratio of the free base form to the salt form by mass is greater than or equal to 1:5, 1:4, 1:3, 1:2, 1:1, 2:1, 3:1, 4:1, or 5:1.

Example II-85. The implantable depot of any one of Examples II-78 to 84, wherein the therapeutic region includes a releasing agent.

Example II-86. The implantable depot of Example II-85, wherein the releasing agent is polysorbate.

Example II-87. The implantable depot of any one of Examples II-78-86, wherein the polymer is a first polymer and the depot further comprises a control region covering the first surface of the therapeutic region to inhibit release of the analgesic agent from the first surface, wherein the control region comprises a second polymer that is the same as or different than the first polymer.

Example II-88. The implantable depot of Example II-87, wherein the control region is a first control region and the implantable depot further comprises a second control region covering the second surface of the therapeutic region to inhibit release of the analgesic agent from the second surface, wherein the second control region comprises a third polymer that is the same or different than one or both of the first polymer and the second polymer.

Example II-89. The implantable depot of Example II-88, wherein the first, second, and third polymers are the same polymer.

Example II-90. The implantable depot of Example II-88 or 89, wherein one or more of the first, second, or third polymers are poly(lactide-co-glycolide).

Example II-91. The implantable depot of any one of Examples II-78 to 90, wherein the release period is at least 14 days, 21 days, 28 days, 30 days, 40 days, 50 days, 60 days, 70 days, 80 days, 90 days, 100 days, 110 days, or 120 days.

Example II-92. A system for treating pain in a subject, the system comprising one or more of the implantable depots of any one of any one of Examples II-78 to 91.

Example II-93. A method for treating pain, the method comprising:
implanting a depot in a subject, wherein the depot comprising a therapeutic region having a first surface, a second surface opposite the first surface, and a lateral surface between the first and second surfaces, the therapeutic region including a first polymer and an analgesic agent, and wherein at least some of the analgesic agent is in a free base form; and
releasing the analgesic agent from at least the lateral surface of the therapeutic agent over a release period of at least 3 days.

Example II-94. The method of Example II-93, wherein the pain comprises postoperative pain associated with a surgical procedure.

Example II-95. The method of Example II-94, wherein the surgical procedure comprises a knee surgery, a hip surgery, a shoulder surgery, a hernia repair surgery, a bunionectomy, a breast surgery, an abdominal surgery, a spine surgery, or a hemorrhoidectomy.

Example II-96. The method of any one of Examples II-93 to 95, wherein the analgesic agent comprises bupivacaine and the free base form comprises bupivacaine free base.

Example II-97. The method of any one of Examples II-93 to 96, wherein at least 10%, 20%, 30%, 40%, 50%, 60%, 70%, 80%, 90%, 95%, or 99% of the analgesic agent is in the free base form.

Example II-98. The method of any one of Examples II-93 to 98, wherein 100% of the analgesic agent is in the free base form.

Example II-99. The method of any one of Examples II-93 to 98, wherein at least some of the analgesic agent is in a salt form.

Example II-100. The method of Example II-99, wherein the analgesic agent comprises bupivacaine and the salt form comprises bupivacaine hydrochloride.

Example II-101. The method of Example II-99 or 100, wherein a ratio of the free base form to the salt form by mass is greater than or equal to 1:5, 1:4, 1:3, 1:2, 1:1, 2:1, 3:1, 4:1, or 5:1.

Example II-102. The method of any one of Examples II-93 to 101, wherein the therapeutic region includes a releasing agent.

Example II-103. The method of any one of Examples II-93 to 102, wherein the depot includes a control region covering the first surface of the therapeutic region to inhibit release of the analgesic agent from the first surface, wherein the control region comprises a second polymer.

Example II-104. The method of Example II-103, wherein the control region is a first control region, and wherein the depot further comprises a second control region covering the second surface of the therapeutic region to inhibit release of the analgesic agent from the second surface, wherein the second control region comprises a third polymer.

Example II-105. The method of Example II-104, wherein the first, second, and third polymers are the same polymer.

Example II-106. The method of any one of Examples II-93 to 105, wherein the release period is at least 14 days, 21 days, 28 days, 30 days, 40 days, 50 days, 60 days, 70 days, 80 days, 90 days, 100 days, 110 days, or 120 days.

Example II-107. An implantable depot for treating pain in a subject, the implantable depot comprising:
a therapeutic region having an outer surface, wherein the therapeutic region includes a first polymer and an analgesic agent, and wherein at least some of the analgesic agent is in a free base form,
a control region covering at least a portion of the surface of the therapeutic region to inhibit release of the analgesic agent from the surface, wherein the control region comprises a second polymer that is the same as or different than the first polymer.
wherein, when implanted in the subject, the implantable depot is configured to release the analgesic agent from at least the surface of the therapeutic region over a release period of at least 3 days.

Example II-108. The implantable depot of Example II-107, wherein the depot comprises an opening extending through at least a portion of a thickness of the depot such that a portion of the therapeutic region is exposed through the control region, and wherein, when implanted in the subject, the implantable depot is configured to release the analgesic agent through the openings.

### CONCLUSION

Although many of the embodiments are described above with respect to systems, devices, and methods for treating postoperative pain, the technology is applicable to other applications and/or other approaches. For example, the depots of the present technology may be used to treat postoperative pain associated with a veterinary procedure and/or surgery. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-36.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. As used herein, the phrase “and/or” as in “A and/or B” refers to A alone, B alone, and A and B.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1.-83. (canceled)

84. An implantable depot for treating pain, the implantable depot comprising:
a therapeutic region comprising a polymer, an analgesic agent, and a plasticizer,
wherein, when implanted in vivo, the therapeutic region is configured to release the analgesic agent for a treatment period of at least 3 days, and
wherein the implantable depot has a flexural modulus within a range from 1 MPa to 400 MPa.

85. The implantable depot of claim 84, wherein the plasticizer is hydrophilic.

86. The implantable depot of claim 84, wherein the plasticizer is hydrophobic.

87. The implantable depot of claim 84, wherein the plasticizer comprises one or more of a triglyceride, a fatty acid ester, a lactic acid ester, a citrate, a phthalate, a glycerol ester, a sebacate, a monoglyceride ester, a benzyl derivative, a polyethylene glycol, a polysorbate, a diol, or a triol.

88. The implantable depot of claim 84, wherein the plasticizer comprises one or more of triacetin, diethyl phthalate, benzyl benzoate, or glycerol.

89. The implantable depot of claim 84, wherein a relative energy difference (RED) between the plasticizer and the polymer is less than or equal to 1.

90. The implantable depot of claim 84, wherein a vapor pressure of the plasticizer is less than or equal to 0.5 Pa at 25° C.

91. The implantable depot of claim 84, wherein a log P value of the plasticizer is within a range from −1.5 to 6, 0 to 4, or 2 to 4.

92. The implantable depot of claim 84, wherein the plasticizer constitutes from 0.1% to 20% of a total mass of the therapeutic region.

93. The implantable depot of claim 84, wherein the plasticizer constitutes from 0.1% to 20% of a total mass of the implantable depot.

94. The implantable depot of claim 84, wherein the therapeutic region includes only a single plasticizer.

95. The implantable depot of claim 84, wherein the plasticizer is a first plasticizer, and the therapeutic region further comprises a second plasticizer.

96. The implantable depot of claim 95, wherein the first plasticizer is triacetin and the second plasticizer is glycerol.

97. The implantable depot of claim 95, wherein the therapeutic region further comprises a third plasticizer.

98. The implantable depot of claim 97, wherein the first plasticizer is triacetin, the second plasticizer is glycerol, and the third plasticizer is benzyl benzoate.

99. The implantable depot of claim 84, wherein therapeutic region has a first surface, a second surface opposite the first surface, and a lateral surface between the first and second surfaces.

100. The implantable depot of claim 99, wherein the first surface, second, surface, and lateral surfaces of the therapeutic region are exposed.

101. The implantable depot of claim 84, wherein the analgesic agent constitutes at least 50% of a total mass of the implantable depot.

102. The implantable depot of claim 84, wherein the analgesic agent comprises bupivacaine.

103. The implantable depot of claim 84, wherein the polymer comprises poly(lactide-co-glycolide).
